(19) United States
(12) Patent Application Publication
(10) Pub. No.: US 2026/0265252 A1
(43) Pub. Date: Sep. 10, 2026

(54) SOLID STATE FORMS OF IMIPRIDONE DERIVATIVE

(71) Applicant: CHIMERIX, INC., Durham, NC (US)

(72) Inventors: Biren Kishor Joshi, Cary, NC (US); Venkatraman Lakshmanan, Cary, NC (US); Aaron Leigh Downey, Chapel Hill, NC (US); Roy Wendell Ware, JR., Raleigh, NC (US)

(21) Appl. No.: 19/219,880

(22) Filed: May 27, 2025

Related U.S. Application Data
(60) Provisional application No. 63/652,638, filed on May 28, 2024.
(60) Provisional application No. 63/755,852, filed on Feb. 7, 2025.

(51) Int. Cl. C07D 471/14; A61K 31/519

(52) U.S. Cl. CPC C07D 471/14; A61K 31/519

(57) ABSTRACT

The present disclosure relates to polymorphic forms and materials, as well as hydrates and solvates thereof, of 7-benzyl-4-(2,4-difluorobenzyl)-2,4,6,7,8,9-hexahydroimidazo[1,2-a]pyrido[3,4-e]pyrimidin-5(1H)-one or salts thereof, also known as ONC206, that are useful in the treatment of one or more cancers, processes to produce such polymorphic forms and materials, and methods of using such forms and materials.

### CROSS-REFERENCE TO RELATED APPLICATIONS

This application is a U.S. non-provisional application under 35 USC § 111(a), which claims priority to U.S. Provisional Application Nos. 63/652,638, filed May 28, 2024, and 63/755,852, filed Feb. 7, 2025, the contents of each is hereby incorporated herein by reference.

### FIELD

The present disclosure relates to polymorphic forms of 7-benzyl-4-(2,4-difluorobenzyl)-2,4,6,7,8,9-hexahydroimidazo[1,2-a]pyrido[3,4-e]pyrimidin-5(1H)-one, also known as ONC206, that are useful in the treatment of one or more cancers, processes to produce such polymorphic forms, and methods of using.

### BACKGROUND

The majority of recurrent tumor types, such as CNS, neuroendocrine, and endometrial tumors, lack effective systemic therapy options following surgical resection and adjuvant radiotherapy. ONC201, which may be used interchangeably with ONC-201, and which may be referred to as NSC-350625 or TIC-10, is a founding member of the imipridone class of small molecules, and has induced durable tumor regressions in patients with diffuse midline glioma, H3 K27M-mutant (DMG H3K27M). One embodiment of ONC201 is the dihydrochloride salt.

ONC206, which may be used interchangeably with ONC-206, and which is 7-benzyl-4-(2,4-difluorobenzyl)-2,4,6,7,8,9-hexahydroimidazo[1,2-a]pyrido[3,4-e]pyrimidin-5(1H)-one, is the second compound from the imipridone class to enter clinical development. ONC206 is a DRD2 antagonist and ClpP agonist that exhibits differentiated receptor pharmacology and gene expression profiles in tumors relative to ONC201. One embodiment of ONC206 is the dihydrochloride salt.

The structures for each of compounds is as follows:

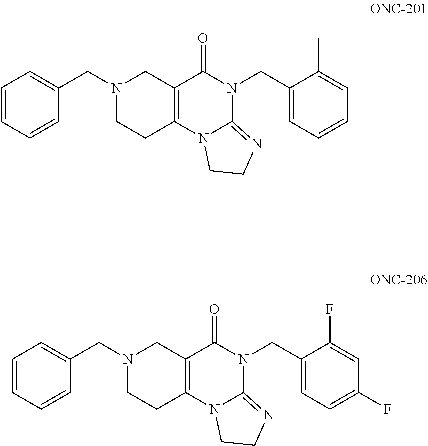 [SMILES: Cc1ccccc1CN1C(=O)C2=C(CCN(Cc3ccccc3)C2)N2CCN=C12 ; O=C1C2=C(CCN(Cc3ccccc3)C2)N2CCN=C2N1Cc1ccc(F)cc1F]

Physicochemical properties are important for a drug substance because they directly influence how a drug interacts with the body, affecting its absorption, distribution, metabolism, and excretion (ADME). The physicochemical properties of a drug substance determines how effectively a drug can reach its target site and exert its therapeutic effect. Factors like solubility, partition coefficient, particle size, and stability are key aspects that need to be carefully considered during drug development to ensure safe and effective administration. Unpredictable crystallization may result in poor solubility or poor stability.

There is a clear need in the art for a method of crystallization which produces high yields with easy to filter, pure crystals. The disclosed methods limit the formation of the impurities and to control the crystallization in order to have easy-to-filter crystal that are robust and scalable.

### BRIEF SUMMARY OF THE DISCLOSURE

One embodiment of the present disclosure includes a crystal form of 7-benzyl-4-(2,4-difluorobenzyl)-2,4,6,7,8,9-hexahydroimidazo[1,2-a]pyrido[3,4-e]pyrimidin-5(1H)-one or a salt form thereof.

In some embodiments throughout the present disclosure, the form may be partially crystalline wherein the crystalline order is disrupted based on hydration level.

In some embodiments, the crystalline form is a crystalline polymorph form of 7-benzyl-4-(2,4-difluorobenzyl)-2,4,6,7,8,9-hexahydroimidazo[1,2-a]pyrido[3,4-e]pyrimidin-5(1H)-one di-hydrochoride salt.

In some embodiments, the crystalline form is a hydrate or solvate.

One embodiment of the present disclosure includes a crystal form of 7-benzyl-4-(2,4-difluorobenzyl)-2,4,6,7,8,9-hexahydroimidazo[1,2-a]pyrido[3,4-e]pyrimidin-5(1H)-one or a salt form thereof, as a variable hydrate.

In some embodiments, the form contains up to 3 moles of water.

In some embodiments, the form contains from between about 2 and 3 moles of water.

One embodiment of the present disclosure includes a crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a powder X-ray diffraction pattern comprising a peak at diffraction angle (2Θ) of 21.8±0.2.

One embodiment of the present disclosure includes a crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a powder X-ray diffraction pattern comprising a peak at diffraction angle (2Θ) of 15.8±0.2.

In some embodiments, the crystalline polymorph form has a powder X-ray diffraction pattern comprising a peak at diffraction angle (2Θ) of 21.8±0.2 and 15.8±0.2.

In some embodiments, the crystalline polymorph form has a powder X-ray diffraction pattern comprising a peak at diffraction angles (2Θ) of 7.8±0.2, 11.4±0.2, and 15.6±0.2.

In some embodiments, the crystalline polymorph form has a powder X-ray diffraction pattern comprising a peak at diffraction angles (2Θ) 7.8±0.2, 11.4±0.2, 15.6±0.2, 15.8±0.2. and 21.8±0.2.

In some embodiments, the crystalline polymorph form has a powder X-ray diffraction pattern comprising a peak at diffraction angles (2Θ) of at one or more of 3.96±0.2 degrees two-theta, 5.8±0.2 degrees two-theta, 6.06±0.2 degrees two-theta, 7.82±0.2 degrees two-theta, 8.32±0.2 degrees two-theta, 11.54±0.2 degrees two-theta, ±0.2 degrees two-theta, 12.04±0.2 degrees two-theta, 12.84±0.2 degrees two-theta, 13.06±0.2 degrees two-theta, 13.96±0.2 degrees two-theta, 14.38±0.2 degrees two-theta, 14.6±0.2 degrees two-theta, 15.18±0.2 degrees two-theta, 15.58±0.2 degrees two-theta, 15.78±0.2 degrees two-theta, 16.66±0.2 degrees two-theta, 16.92±0.2 degrees two-theta, 17.08±0.2 degrees two-theta, 17.28±0.2 degrees two-theta, 18.04±0.2 degrees two-theta, 18.28±0.2 degrees two-theta, 18.92±0.2 degrees two-theta, 19.48±0.2 degrees two-theta, 19.76±0.2 degrees two-theta, 20.98±0.2 degrees two-theta, 21.5±0.2 degrees two-theta, 21.82±0.2 degrees two-theta, 22.88±0.2 degrees two-theta, 23.26±0.2 degrees two-theta, 24.12±0.2 degrees two-theta, 24.64±0.2 degrees two-theta, 25.08±0.2 degrees two-theta, 25.3±0.2 degrees two-theta, 25.8±0.2 degrees two-theta, 26.14±0.2 degrees two-theta, 26.4±0.2 degrees two-theta, 26.86±0.2 degrees two-theta, 27.4±0.2 degrees two-theta, 27.84±0.2 degrees two-theta, 28.16±0.2 degrees two-theta, 29.1±0.2 degrees two-theta, 29.56±0.2 degrees two-theta, 29.9±0.2 degrees two-theta, 30.32±0.2 degrees two-theta, 30.72±0.2 degrees two-theta, 31.4±0.2 degrees two-theta, 31.8±0.2 degrees two-theta, 33.7±0.2 degrees two-theta, 34.1±0.2 degrees two-theta, 35.18±0.2 degrees two-theta, and 36.48±0.2 degrees two-theta.

In some embodiments, the crystalline polymorph form has a powder X-ray diffraction pattern comprising one or more peaks essentially the same as shown in FIG. 8.

In some embodiments, the form is characterized with a DSC thermogram substantially the same as FIG. 10.

One embodiment of the present disclosure includes a crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a FT-IR spectrum comprising a signal at wavenumber 2584 cm-1.

In some embodiments, the crystalline polymorph form has an FT-IR spectrum having a signal at one or more of wavenumber 861 cm-1, 878 cm-1, 1265 cm-1, 1704 cm-1, 1305 cm-1, and 2584 cm-1.

In some embodiments, the crystalline polymorph form has an FT-IR spectrum substantially the same as FIG. 14.

One embodiment of the present disclosure includes a crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a FT-Raman spectrum comprising a signal at wavenumber 3055 cm-1.

In some embodiments, the crystalline polymorph form has an FT-Raman spectrum having a signal at one or more of wavenumber 239 cm-1, 790 cm-1, 1704 cm-1, and 3055 cm-1.

In some embodiments, the crystalline polymorph form has an FT-Raman spectrum substantially the same as FIG. 15.

One embodiment of the present disclosure includes a crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a LF-Raman spectrum comprising a signal at wavenumber 149 cm-1.

One embodiment of the present disclosure includes a crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a LF-Raman spectrum comprising a signal at wavenumber 240 cm-1.

In some embodiments, the crystalline polymorph form has an FT-Raman spectrum having a signal at one or more of wavenumber 92 cm-1, 149 cm-1, 240 cm-1, and 790 cm-1.

In some embodiments, the crystalline polymorph form has an LF-Raman spectrum substantially the same as FIG. 16.

One embodiment of the present disclosure includes a hydrated or solvated crystalline form of 7-benzyl-4-(2,4-difluorobenzyl)-2,4,6,7,8,9-hexahydroimidazo[1,2-a]pyrido[3,4-e]pyrimidin-5(1H)-one or a salt thereof.

In some embodiments, the form comprises a hydrate.

In some embodiments, the form comprises a di-hydrate.

In some embodiments, the form comprises a tri-hydrate.

In some embodiments, the form comprises a variable hydrate.

In some embodiments, the form is desiccated to an alternative dehydrate.

In some embodiments, the dehydrated form is partially crystalline.

In some embodiments, a dehydrated form is re-hydrated to an original crystalline form.

One embodiment of the present disclosure includes a form of any one of claims 1 to 32, further comprising a form selected from the group consisting of an initial form and one or more different forms converted from the initial form during manufacture, storage, or use, or a combination thereof.

In some embodiments, the form may be characterized as soluble according to ICH guidelines.

In some embodiments, the form may be characterized as having intermediate aqueous solubility.

In some embodiments, the form may be characterized as having low aqueous solubility.

In some embodiments, the form may be characterized by a single therapeutic dose is completely soluble in 250 mL or less of aqueous media in the pH range of 1.2 to 6.8 at 37° C.

One embodiment of the present disclosure includes a pharmaceutical composition comprising the crystalline form of any one of claims 1 to 37, and one or more pharmaceutically acceptable excipients.

In some embodiments, one or more of the pharmaceutically acceptable excipients has desiccating properties.

In some embodiments, the composition is packaged with a desiccant.

In some embodiments, the composition is substantially free of decomposition impurities.

In some embodiments, the composition is stable according to ICH guidelines.

In some embodiments, the composition is characterized as stable according to requirements in 21 CFR Part 211.166, or equivalent thereof.

In some embodiments, the composition is stable over one, three, or six months, based on accelerated testing performed at 25° C./60% RH.

In some embodiments, the composition is stable over one, three, or six months, based on accelerated testing performed at 40° C./75% RH.

### BRIEF DESCRIPTION OF THE FIGURES

FIG. 1 provides the X-ray powder diffraction (XRPD) pattern of Compound I Form 1.

FIG. 2 shows the differential scanning calorimetry (DSC) (top) and thermal gravimetric analysis (TGA) thermograph (bottom) for Compound I Form 1.

FIG. 3 shows the dynamic vapor sorption (DVS) for Compound I Form 1.

FIG. 4 provides an ¹⁹F ssNMR spectrum of Compound I Form 1.

FIG. 5 provides the Fourier Transform Infra Red (FT-IR) spectrum for Compound I Form 1.

FIG. 6 provides the Fourier Transform (FT) Raman spectrum for Compound I Form 1.

FIG. 7 shows the low-frequency (LF) Raman spectrum for Compound I Form 1.

FIG. 8A provides the XRPD pattern of Compound I, Form 2 (Sample 8673-23-02). FIG. 8B provides the XRPD pattern for Compound I, Form 1, (Sample 021100).

FIG. 9 shows the DSC for Compound I Form 2 from samples A (top), B (middle), and C (bottom).

FIG. 10 shows the TGA thermograph for Compound I Form 2 at a 2 g scale (top), 500 g scale (middle), and 40 kg scale (bottom).

FIG. 11 shows the DVS for Compound I Form 2.

FIG. 12 provides an ¹⁹F ssNMR spectrum of Compound I Form 2.

FIG. 13 provides the Fourier Transform Infra Red (FT-IR) spectrum for Compound I Form 2 (40 kg scale).

FIG. 14 provides the Fourier Transform (FT) Raman spectrum for Compound I Form 2 (40 kg scale).

FIG. 15 shows the low-frequency (LF) Raman spectrum for Compound I Form 2 (40 kg scale).

FIG. 16 provides the XRPD pattern of Compound I Form 3.

FIG. 17 shows the DSC (top) and TGA (bottom) thermograph for Compound I Form 3.

FIG. 18 provides the XRPD pattern of Compound I Form 4.

FIG. 19 shows the DSC (top) and TGA (bottom) thermograph for Compound I Form 4.

FIG. 20 provides the XRPD pattern of Compound I Form 5.

FIG. 21 shows the DSC (top) and TGA (bottom) thermograph for Compound I Form 5.

FIG. 22 provides the XRPD pattern of Compound I Material 6+Form 2 (A, top), Compound I Material 7+Form 2 (B), Compound I Material 8+Form 2 (C), Compound I Form 2 (D), and Compound I Form 1 (E, bottom).

FIG. 23 provides the XRPD pattern of Compound I Form 9.

FIG. 24 shows the DSC (top) and TGA (bottom) thermograph for Compound I Form 9.

FIG. 25 provides the XRPD pattern of Compound I Material 10+minor Form 2 (top), Compound I Form 2 (middle), and Compound I Form 1 (bottom).

FIG. 26 shows the DSC (top) and TGA (bottom) thermograph for Compound I Material 10+minor Form 2.

FIG. 27 provides the XRPD pattern of Compound I Form 11.

FIG. 28 shows the DSC (top) and TGA (bottom) thermograph for Compound I Form 11.

FIG. 29 provides the XRPD pattern of disordered crystalline Compound I Material 12+Form 2.

FIG. 30 provides the XRPD pattern of Compound I Material 13+Form 11.

FIG. 31 provides the XRPD pattern of Compound I Material 14.

FIG. 32 provides the XRPD patterns of Compound I Material 15 produced by anti-solvent addition (top) and grinding with Compound I Material 10 (bottom).

FIG. 33 provides the XRPD pattern of Compound I Material 16.

FIG. 34 provides the XRPD pattern of Compound I Material 17 from isopropyl acetate (top) and from methyl ethyl ketone (bottom).

FIG. 35 provides the XRPD pattern of Compound I Amorphous Form from a spray dried sample (top) and from a lyophilized sample (bottom).

FIG. 36 shows the DSC for Compound I Amorphous Form from a spray dried sample (top) and from a spray dried sample with reversed heat flow (bottom).

FIG. 37 shows the DSC thermograph for Compound I Amorphous Form from lyophilized sample (top) and from a lyophilized sample with reversed heat flow (bottom).

FIG. 38 shows the TGA thermograph for Compound I Amorphous Form from a spray dried sample (top) and from a lyophilized sample (bottom).

FIG. 39 provides the XRPD pattern of Compound I Adipic Acid Materia I.

FIG. 40 shows the DSC (top) and TGA (bottom) thermograph for Compound I Adipic Acid Material I.

FIG. 41 provides the XRPD pattern of Compound I Glutaric Acid Material 1+glutaric acid.

FIG. 42 provides the XRPD pattern of Compound I Glutaric Acid Material 2.

FIG. 43 shows the DSC (top) and TGA (bottom) thermograph for Compound I Glutaric Acid Material 2.

FIG. 44 provides the XRPD pattern of Compound I Succinic Acid Material 1+succinic acid+possible Compound I Form 1.

FIG. 45 provides the XRPD pattern of Compound I Vanillin Material 1+vanillin.

FIG. 46 provides the XRPD pattern of Compound I Mono-Maleate Material A.

FIG. 47 shows the DSC (top) and TGA (bottom) thermograph for Compound I Mono-Maleate Material A.

FIG. 48 provides the XRPD pattern of Compound I Form A.

FIG. 49 shows the DSC (top) and TGA (bottom) thermograph for Compound I Form A.

FIG. 50 shows the DVS for Compound I Form A.

FIG. 51 provides the XRPD pattern of Compound I Material C+L-Proline (top) and Compound I Material C (sucralose cocrystal attempt) (bottom).

FIG. 52 shows the DSC (top) and TGA (bottom) thermograph for Compound I Material C (sucralose cocrystal attempt).

FIG. 53 provides the XRPD pattern of Compound I Betaine HCl Material A.

FIG. 54 shows the DSC (top) and TGA (bottom) thermograph for Compound I Betaine HCl Material A.

FIG. 55 provides the XRPD pattern of Compound I Methyl Paraben Material A.

FIG. 56. shows the DSC (top) and TGA (bottom) thermograph for Compound I Methyl Paraben Material A.

FIG. 57 provides the XRPD pattern of Compound I Propyl Gallate Material A.

FIG. 58 shows the DSC (top) and TGA (bottom) thermograph for Compound I Propyl Gallate Material A.

FIG. 59 provides the XRPD pattern of Compound I Saccharin Material A+minor Compound I Form A.

FIG. 60 shows the DSC (top) and TGA (bottom) thermograph for Compound I Saccharin Material A+minor Compound I Form A.

FIG. 61 provides the XRPD pattern of Compound I Sorbitol Material A+sorbitol.

FIG. 62 provides the XRPD pattern of Compound I Zinc Chloride Material A+possible minor Compound I Form A.

FIG. 63 shows the DSC (top) and TGA (bottom) thermograph for Compound I Zinc Chloride Material A+possible minor Compound I Form A.

FIG. 64 provides the XRPD pattern of Compound I Besylate Form A.

FIG. 65 shows the DSC (top) and TGA (bottom) thermograph for Compound I Besylate Form A.

FIG. 66 shows the DVS for Compound I Besylate Form A.

FIG. 67 provides the XRPD pattern of Compound I Fumarate Form A.

FIG. 68 shows the DSC (top) and TGA (bottom) thermograph for Compound I Fumarate Form A.

FIG. 69 shows the DVS for Compound I Fumarate Form A.

FIG. 70 provides the XRPD pattern of Compound I Sulfate Form A.

FIG. 71 shows the DSC (top) and TGA (bottom) thermograph for Compound I Sulfate Form A.

FIG. 72 shows the DVS for Compound I Sulfate Form A.

FIG. 73 provides the XRPD pattern of Compound I Tosylate Form A.

FIG. 74 shows the DSC (top) and TGA (bottom) thermograph for Compound I Tosylate Form A.

FIG. 75 shows the DVS for Compound I Tosylate Form A.

FIG. 76 provides the XRPD pattern of Compound I Edisylate Form A.

FIG. 77 shows the DSC (top) and TGA (bottom) thermograph for Compound I Edisylate Form A.

FIG. 78 provides the XRPD pattern of Compound I Gentisate Form A.

FIG. 79 shows the DSC (top) and TGA (bottom) thermograph for Compound I Gentisate Form A.

FIG. 80 provides the XRPD pattern of Compound I Potential Benzoate Form A.

FIG. 81 provides the XRPD pattern of Compound I Potential Mesylate Form A

FIG. 82 provides the XRPD pattern of Compound I Potential Phosphate Form A

FIG. 83 provides the XRPD pattern of Compound I Potential IPA Solvate

FIG. 84 provides the XRPD pattern of Compound I Reaction with Oxalic Acid showing the unique material from reaction with oxalic acid.

### DETAILED DESCRIPTION OF THE DISCLOSURE

The present disclosure highlights the polymorphic forms of Compound I and the processes for preparing Compound 1. These polymorphic forms are useful in the treatment of one or more cancers.

### Definitions

The following definitions are meant to clarify, but not limit, the terms defined. If a particular term used herein is not specifically defined, such term should not be considered indefinite. Rather, terms are used within their accepted meanings.

“Compound I” or “ONC206” as used throughout this disclosure refers to 7-benzyl-4-(2,4-difluorobenzyl)-2,4,6,7,8,9-hexahydroimidazo[1,2-a]pyrido[3,4-e]pyrimidin-5(1H)-one or a salt thereof. Reference to the free base or salt form will be clear based on context. ONC-206 di-HCl and has the following structure:

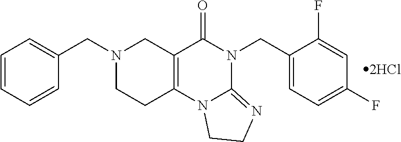 [SMILES: O=C1C2=C(CCN(Cc3ccccc3)C2)N2CCN=C2N1Cc1ccc(F)cc1F]

Compound I may be in the form of a pharmaceutically acceptable salt, solvate, and/or hydrate. Compound I and methods for making and using Compound I, tautomers of Compound I, stereoisomers of Compound I, deuterated derivatives of Compound I and its tautomers and stereoisomers, and pharmaceutically acceptable salts of any of the foregoing are disclosed in PCT/US2014/055373, hereby incorporated herein by reference.

As used herein, the term “active pharmaceutical ingredient” (“API”) or “therapeutic agent” refers to a biologically active compound.

The terms “patient” and “subject” are used interchangeably and refer to an animal including humans.

The terms “effective dose” and “effective amount” are used interchangeably herein and refer to that amount of a compound that produces the desired effect for which it is administered (e.g., improvement in pain or a symptom of pain, or lessening the severity of pain or a symptom of pain). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of one or more cancers (e.g., colorectal, brain, and glioblastoma), including but not limited to Gliomas, glioneuronal tumors, and neuronal tumors, Adult-type diffuse gliomas, Astrocytoma, IDH-mutant, Oligodendroglioma, IDH-mutant, and 1p/19q-codeleted, Glioblastoma, IDH-wildtype, Pediatric-type diffuse low-grade gliomas, Diffuse astrocytoma, MYB- or MYBL1-altered, Angiocentric glioma, Polymorphous low-grade neuroepithelial tumor of the young, Diffuse low-grade glioma, MARK pathway-altered, Pediatric-type diffuse high-grade gliomas, Diffuse midline glioma, H3 K27-altered, Diffuse hemispheric glioma, H3 G34-mutant, Diffuse pediatric-type high-grade glioma, H3-wildtype and IDH-wildtype, Infant-type hemispheric glioma, Circumscribed astrocytic glioma, Pilocytic astrocytoma, High-grade astrocytoma with piloid features, Pleomorphic xanthoastrocytoma, Subependymal giant cell astrocytoma, Chordoid glioma, Astroblastoma, MN 1-altered, Glioneuronal and neuronal tumors, Ganglioglioma, Desmoplastic infantile ganglioglioma/desmoplastic infantile astrocytoma, Dysembryoplastic neuroepithelial tumor, Diffuse glioneuronal tumor with oligodendroglioma-like features and nuclear clusters, Papillary glioneuronal tumor, Rosette-forming glioneuronal tumor, Myxoid glioneuronal tumor, Diffuse leptomeningeal glioneuronal tumor, Gangliocytoma, Multinodular and vacuolating neuronal tumor, Dysplastic cerebellar gangliocytoma (Lhermitte-Duclos disease), Central neurocytoma, Extraventricular neurocytoma, Cerebellar liponeurocytoma, Ependymal tumors, Supratentorial ependymoma, Supratentorial ependymoma, ZFTA fusion-positive, Supratentorial ependymoma, YAP1 fusion positive, Posterior fossa ependymoma, Posterior fossa ependymoma, group PFA, Posterior fossa ependymoma, group PFB, Spinal ependymoma, Spinal ependymoma, MYCN-amplified, Myxopapillary ependymoma, Subependymoma, Choroid plexus tumors, Choroid plexus papilloma, Atypical choroid plexus papilloma, Choroid plexus carcinoma, Embryonal tumors, Medulloblastoma, Medulloblastomas, molecularly defined, Medulloblastoma, WNT-activated, Medulloblastoma, SHH-activated and TP53-wildtype, Medulloblastoma, SHH-activated and TP53-mutant, Medulloblastoma, non-WNT/non-SHH, Medulloblastomas, histologically defined, Other CNS embryonal tumors, Atypical teratoid/rhabdoid tumor, Cribriform neuroepithelial tumor, Embryonal tumor with multilayered rosettes, CNS neuroblastoma, FOXR2-activated, CNS tumor with BCOR internal tandem duplication, CNS embryonal tumor, Pineal tumors, Pineocytoma, Pineal parenchymal tumor of intermediate differentiation, Pineoblastoma, Papillary tumor of the pineal region, Desmoplastic myxoid tumor of the pineal region, SMARCB1-mutant, Cranial and paraspinal nerve tumors, Schwannoma, Neurofibroma, Perineurioma, Hybrid nerve sheath tumor, Malignant melanotic nerve sheath tumor, Malignant peripheral nerve sheath tumor, Paraganglioma, Meningiomas, Meningioma, Mesenchymal, non-meningothelial tumors, Soft tissue tumors, Fibroblastic and myofibroblastic tumors, Solitary fibrous tumor, Vascular tumors, Hemangiomas and vascular malformations, Hemangioblastoma, Skeletal muscle tumors, Rhabdomyosarcoma, Uncertain differentiation, Intracranial mesenchymal tumor, FET-CREB fusion-positive, CIC-rearranged sarcoma, Primary intracranial sarcoma, DICER1-mutant, Ewing sarcoma, Chondro-osseous tumors, Chondrogenic tumors, Mesenchymal chondrosarcoma, Chondrosarcoma, Notochordal tumors, Chordoma (including poorly differentiated chordoma), Melanocytic tumors, Diffuse meningeal melanocytic neoplasms, Meningeal melanocytosis and meningeal melanomatosis, Circumscribed meningeal melanocytic neoplasms, Meningeal melanocytoma and meningeal melanoma, Hematolymphoid tumors, Lymphomas, CNS lymphomas, Primary diffuse large B-cell lymphoma of the CNS, Immunodeficiency-associated CNS lymphoma, Lymphomatoid granulomatosis, Intravascular large B-cell lymphoma, Miscellaneous rare lymphomas in the CNS, MALT lymphoma of the dura, Other low-grade B-cell lymphomas of the CNS, Anaplastic large cell lymphoma (ALK+/ALK−), T-cell and NK/T-cell lymphomas, Histiocytic tumors, Erdheim-Chester disease, Rosai-Dorfman disease, Juvenile xanthogranuloma, Langerhans cell histiocytosis, Histiocytic sarcoma, Germ ceil tumors, Mature teratoma, Immature teratoma, Teratoma with somatic-type malignancy, Germinoma, Embryonal carcinoma, Yolk sac tumor, Choriocarcinoma, Mixed germ cell tumor, Tumors of the sellar region, Adamantinomatous craniopharyngioma, Papillary craniopharyngioma, Pituicytoma, granular cell tumor of the sellar region, and spindle cell oncocytoma, Pituitary adenoma/PitNET, Pituitary blastoma, Metastases to the CNS, Metastases to the brain and spinal cord parenchyma, and Metastases to the meninges.

As used herein, the term “in combination with,” when referring to two or more compounds, agents, or additional active pharmaceutical ingredients, means the administration of two or more compounds, agents, or active pharmaceutical ingredients to the patient prior to, concurrently with, or subsequent to each other.

“Selected from” and “chosen from” are used interchangeably herein.

As used herein, the term “ambient conditions” means room temperature, open air condition and uncontrolled humidity condition. As used herein, the terms “room temperature” and “ambient temperature” mean 15° C. to 30° C.

As used herein, the term “solvent” refers to any liquid in which the product is at least partially soluble (solubility of product >2 g/L). As will be appreciated by those skilled in the art, low solubility is considered <2 g/L and limited solubility is from about 2-20 g/L.

The term “stable,” as used herein, refers to compounds or solid forms that are not substantially altered when subjected to conditions to allow for their production, detection, and preferably their recovery, purification, and use for one or more of the purposes disclosed herein.

The term “chemically stable,” as used herein, means that the solid form of Compound I does not decompose into one or more different chemical compounds when subjected to specified conditions, e.g., 40° C./75% relative humidity, for a specific period of time, e.g., 1 day, 2 days, 3 days, 1 week, 2 weeks, or longer. In some embodiments, less than 25% of the solid form of Compound I decomposes. In some embodiments, less than about 20%, less than about 15%, less than about 10%, less than about 5%, less than about 3%, less than about 1%, less than about 0.5%, less than about 0.1%, less than about 0/05%, of the form of Compound I decomposes under the conditions specified. In some embodiments, no detectable amount of the solid form of Compound I decomposes.

The term “physically stable,” as used herein, means that the solid form of Compound I does not change into one or more different physical forms of Compound I (e.g., different solid forms as measured by XRPD, DSC, etc.) when subjected to specific conditions, e.g., 40° C./75% relative humidity, for a specific period of time, e.g, 1 day, 2 days, 3 days, 1 week, 2 weeks, or longer. In some embodiments, less than 25% of the solid form of Compound I changes into one or more different physical forms when subjected to specified conditions. In some embodiments, less than about 20%, less than about 15%, less than about 10%, less than about 5%, less than about 3%, less than about 1%, less than about 0.5% of the solid form of Compound I changes into one or more different physical forms of Compound I when subjected to specified conditions. In some embodiments, no detectable amount of the solid form of Compound I changes into one or more physically different solid forms of Compound I.

As used herein, the term “pharmaceutically acceptable solid form” refers to a solid form of Compound I of this disclosure wherein the solid form (e. g., crystalline free form, crystalline salt, crystalline salt solvate, crystalline salt hydrate, and amorphous form) of Compound I is nontoxic and suitable for use in pharmaceutical compositions.

As used herein, the term “amorphous” refers to a solid material having no long-range order in the position of its molecules. Amorphous solids are generally rather isotropic, i.e., exhibit similar properties in all directions. Amorphous solids do not have definite melting points. Amorphous solids are generally glasses or supercooled liquids in which the molecules are arranged in a random manner so that there is no well-defined arrangement, e.g., molecular packing, and no long-range order. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. A comparison of XRPDs may be made of an amorphous material and crystalline material. In some embodiments, a solid material may comprise an amorphous compound, and the material may, for example, be characterized by a lack of sharp characteristic crystalline peak(s) in its XRPD spectrum (i.e., the material is not crystalline, but is amorphous, as determined by XRPD). Instead, one or several broad peaks (e.g., halos) may appear in the XRPD pattern of the material. A solid material, comprising an amorphous compound, may be characterized by, for example, a wider temperature range for the melting of the solid material, as compared to the range for the melting of a pure crystalline solid. Other techniques, such as, for example, solid state NMR may also be used to characterize crystalline or amorphous forms.

As used herein, the terms “crystal form,” “crystalline form,” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD) and single crystal X-ray diffraction. Accordingly, as used herein, the terms “crystalline Form [X] of Compound (I)” and “crystalline Form [C] potassium salt of Compound (I)” refer to unique crystalline forms that can be identified and distinguished from each other by one or more characterization techniques including, for example, XRPD. In some embodiments, the novel crystalline forms are characterized by an X-ray powder diffractogram having one or more signals at one or more specified degree two-theta values (°2Θ).

As used herein, the term “free form” refers to a non-ionized version of the compound in the solid state. Examples of free forms include free bases and free acids.

As used herein, the term “solvate” refers to a crystal form comprising one or more molecules of a compound of the present disclosure and, incorporated into the crystal lattice, one or more molecules of a solvent or solvents in stoichiometric or nonstoichiometric amounts. When the solvent is water, the solvate is referred to as a “hydrate.”

As used herein, the term “hydrate” refers to any crystalline Compound I that contains water in its crystal lattice. The stoichiometry of a Compound I hydrate can vary, i.e., Compound I can be a variable hydrate. For example, a hydrate of Compound I can be a quarter hydrate, hemihydrate, monohydrate, dihydrate, or a partially dehydrated form.

While the numerical ranges and parameters setting forth the broad scope of the disclosed subject matter are approximations, the numerical values set forth in the working examples are reported as precisely as possible. Any numerical value, however, inherently contains certain errors necessarily resulting from the standard deviation found in their respective testing measurements. In some embodiments, the term “about” means within a standard deviation using measurements generally acceptable in the art. In some embodiments, “about” means a range extending to +/−10% of the specified value.

It is to be understood that measurement of a given sample may vary (standard deviation) depending on the instrument used, the time and temperature of the sample when measured, and standard experimental errors. Therefore, the values, heights and relative intensity of the measurements will have an acceptable level of deviation. For example, the values may have an acceptable deviation of e.g., about 20%, 15%, 10%, 5%, 3%, 2% or 1%.

For XRPD measurements, the 2-theta (2Θ) values, d-spacing values, heights, and relative intensity of the peaks may each have an acceptable level of deviation. In some embodiments, the 2-theta values (°) or the d-spacing values (Å) of the XRPD pattern of the crystalline forms of the current disclosure may have an acceptable deviation of ±0.2 degrees and/or +0.2 Å. Further, the XRPD pattern of the crystalline forms of the disclosure may be identified by the characteristic peaks as recognized by one skilled in the art. For example, the crystalline forms of the disclosure may be identified by, e.g., two characteristic peaks, in some instances, three characteristic peaks, in another instance, five characteristic peaks. Therefore, the term “substantially” as set forth in a particular table or depicted or shown in a particular figure refers to any crystal which has an XRPD having the major or characteristic peaks as set forth in the tables/figures, as recognized by one skilled in the art to identify one form from another. Under most circumstances for XRPDs, peaks within the range of up to about 30° 2Θ are selected. Rounding algorithms are used to round each peak to the nearest 0.1° or 0.01° 2Θ, depending upon the instrument used to collect the data and/or the inherent peak resolution. Peak position variabilities are given to within +0.2° 2Θ.

Per USP guidelines, variable hydrates and solvates may display peak variances greater than +0.2° 20. By their very nature, variable hydrates and solvates are recognized to have varying unit cell dimensions, and as such, shifting occurs in peak positions of the measured XRPD patterns for these materials. In these unique materials, variance in 2Θ positions of greater than 0.2° is not unexpected. As such, peak position variances such as 0.2° are not applicable to these materials. See The United States Pharmacopeial Convention, Physicals Tests/X-Ray Powder Diffraction 941: page 825. In some embodiments, the 2-theta values (°) or the d-spacing values (A) of the XRPD pattern of the crystalline forms of the current disclosure may have an acceptable deviation of ±0.5 degrees and/or +0.5 Å; ±1.0 degrees and/or +1.0 Å; ±2.0 degrees and/or +2.0 Å.

It is also to be understood that the differential scanning calorimetry or thermogravimetric analysis thermograms of a given sample may vary (standard deviation) depending on the instrument used, the time and temperature of the sample when measured, and standard experimental errors. The temperature value itself may deviate by ±10° C., preferably ±5° C., preferably ±3° C. of the reference temperature.

It is also to be understood that the ¹⁹F ssNMR spectrums of a given sample may vary (standard deviation) depending on the instrument used, the time and temperature of the sample when measured, and standard experimental errors. The ppm value itself may deviate by ±0.2 ppm, preferably ±0.1 ppm, preferably ±0.05 ppm, preferably ±0.01 ppm of the reference ppm.

It is also to be understood that the Fourier Transform Infra Red (FT-IR), Fourier Transform (FT) Raman, and low-frequency (LF) Raman spectrums of a given sample may vary (standard deviation) depending on the instrument used, the time and temperature of the sample when measured, and standard experimental errors. The cm⁻¹ value itself may deviate by ±0.5 cm⁻¹, preferably ±0.2 cm⁻¹, preferably ±0.1 cm⁻¹, preferably ±0.05 cm⁻¹ of the reference cm⁻¹.

It is also to be understood that the dynamic vapor sorption (DVS) of a given sample may vary (standard deviation) depending on the instrument used, the time and temperature of the sample when measured, and standard experimental errors. The value itself may deviate by ±10%, preferably ±5%, preferably ±1%, preferably ±0.5% of the reference.

In some embodiments, a solid material may comprise a mixture of crystalline solids and amorphous solids. A solid material comprising an amorphous compound may also, for example, contain up to 30% of a crystalline solid. In some embodiments, a solid material prepared to comprise an amorphous compound may also, for example, contain up to 25%, 20%, 15%, 10%, 5%, or 2% of a crystalline solid. In embodiments wherein the solid material contains a mixture of crystalline solids and amorphous solids, the characterizing data, such as XRPD, may contain indicators of both crystalline and amorphous solids. In some embodiments, a crystalline form of this disclosure may contain up to 30% amorphous compound. In some embodiments, a crystalline preparation of Compound I may contain up to 25%, 20%, 15%, 10%, 5%, or 2% of an amorphous solid.

As used herein, the term “substantially amorphous” refers to a solid material having little or no long-range order in the position of its molecules. For example, substantially amorphous materials have less than 15% crystallinity (e.g., less than 10% crystallinity, less than 5% crystallinity, or less than 2% crystallinity). It is also noted that the term “substantially amorphous” includes the descriptor, “amorphous,” which refers to materials having no (0%) crystallinity.

As used herein, the term “substantially crystalline” refers to a solid material having little or no amorphous molecules. For example, substantially crystalline materials have less than 15% amorphous molecules (e.g., less than 10% amorphous molecules, less than 5% amorphous molecules, or less than 2% amorphous molecules). It is also noted that the term “substantially crystalline” includes the descriptor “crystalline,” which refers to materials that are 100% crystalline form.

As used herein, a crystalline form is “substantially pure” when it accounts for an amount by weight equal to or greater than 90% of the sum of all solid form(s) in a sample as determined by a method in accordance with the art, such as one or more of XRPD identification, computational analysis of diffractograms, quantitative XRPD, or other indexing or identification solutions. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the terms “X-ray powder diffractogram,” “X-ray powder diffraction pattern,” “XRPD pattern,” “XRPD spectrum” interchangeably refer to an experimentally obtained pattern plotting signal positions (on the abscissa) versus signal intensities (on the ordinate).

A “signal” or “peak” as used herein refers to a point in the XRPD pattern where the intensity as measured in counts is at a local maximum. An XRPD peak is identified by its angular value as measured in degrees 2Θ (°2Θ), depicted on the abscissa of an X-ray powder diffractogram, which may be expressed, for example, as “a signal at . . . degrees two-theta,” “a signal at [a] two-theta value(s) of . . . ” and/or “a signal at at least . . . two-theta value(s) selected from . . . .”

The repeatability of the measured angular values is in the range of ±0.2° 2Θ, i.e., the angular value can be at the recited angular value +0.2 degrees two-theta, the angular value −0.2 degrees two-theta, or any value between those two end points (angular value +0.2 degrees two-theta and angular value −0.2 degrees two-theta).

One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal maximum values in XRPD diffractograms (in degrees two-theta) generally mean that value is identified as ±0.2 degrees two-theta of the reported value, an art-recognized variance.

As used herein, the term “melting temperature”, “melting point”, or “Tm” refers to the temperature at which a crystalline material is in equilibrium with liquid phase.

As used herein, the term “dispersion” refers to a disperse system in which one substance, the dispersed phase, is distributed, in discrete units, throughout a second substance (the continuous phase or vehicle). The size of the dispersed phase can vary considerably (e.g., colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase or, a solid dispersion includes the drug constituting the dispersed phase, and the polymer constituting the continuous phase.

### DETAILED DESCRIPTION OF EMBODIMENTS

One embodiment of the disclosure provides solid forms of Compound I (e.g., crystalline forms, amorphous forms, solvates, hydrates, cocrystals), which can be used in the methods of treatment and pharmaceutical compositions described herein. In some embodiments, the disclosure provides amorphous forms of Compound I. In some embodiments, the disclosure provides crystalline forms of Compound I. In some embodiments, the disclosure provides solvate crystalline forms of Compound I. In some embodiments, the disclosure provides hydrate crystalline forms of Compound I.

### A. Compound I Form 1

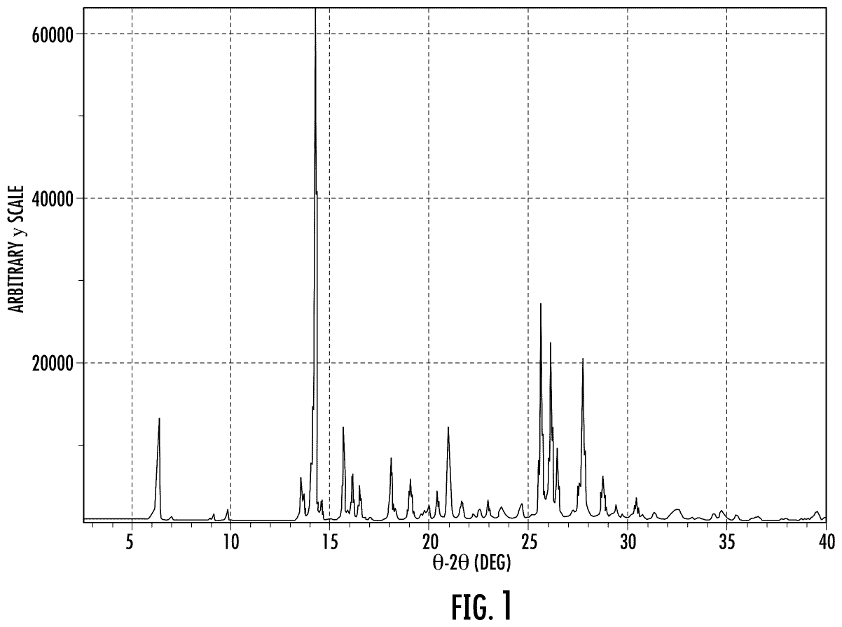

In some embodiments, the disclosure provides crystalline forms of Compound I. In some embodiments, the disclosure provides crystalline Compound I Form 1. In some embodiments, Compound I Form 1 is a variable hydrate. In some embodiments, Compound I Form 11 is a variable hydrate with up to 1.0 mole of water. FIG. 1 (top) provides an X-ray powder diffractogram of crystalline Compound I Form 1 with corresponding tabulated data shown in Table 1.

TABLE 1
XRPD Signals for Crystalline Compound I Form 1

| | | Intenisty | Relative |
|---|---|---|---|
| °2Θ (±0.2°) | d-spacing (Å) | (Counts) | Intensity |
| 6.37 | 13.86 | 12730 | 20 |
| 7 | 12.62 | 1079 | 2 |
| 9.13 | 9.68 | 1327 | 2 |
| 9.84 | 8.98 | 1899 | 3 |
| 13.55 | 6.53 | 5683 | 9 |
| 13.69 | 6.46 | 3771 | 6 |
| 14.27 | 6.2 | 63094 | 100 |
| 14.57 | 6.07 | 2902 | 5 |
| 15.71 | 5.64 | 11751 | 19 |
| 16.14 | 5.49 | 6129 | 10 |
| 16.53 | 5.36 | 4772 | 8 |
| 18.1 | 4.9 | 8109 | 13 |
| 18.32 | 4.84 | 2072 | 3 |
| 19.07 | 4.65 | 5568 | 9 |
| 19.99 | 4.44 | 2409 | 4 |
| 20.46 | 4.34 | 4059 | 6 |
| 21.01 | 4.22 | 11785 | 19 |
| 21.66 | 4.1 | 2940 | 5 |
| 22.24 | 3.99 | 1371 | 2 |
| 22.6 | 3.93 | 1919 | 3 |
| 23.01 | 3.86 | 2937 | 5 |
| 23.65 | 3.76 | 2279 | 4 |
| 24.68 | 3.6 | 2599 | 4 |
| 25.69 | 3.46 | 26967 | 43 |
| 26.17 | 3.4 | 22232 | 35 |
| 26.51 | 3.36 | 9260 | 15 |
| 27.81 | 3.21 | 20155 | 32 |
| 28.81 | 3.1 | 5775 | 9 |
| 29.45 | 3.03 | 2379 | 4 |
| 30.48 | 2.93 | 3253 | 5 |
| 31.37 | 2.85 | 1553 | 2 |
| 32.4 | 2.76 | 1883 | 3 |
| 34.41 | 2.6 | 1490 | 2 |
| 34.81 | 2.58 | 1799 | 3 |
| 35.53 | 2.52 | 1357 | 2 |
| 39.62 | 2.27 | 1737 | 3 |

In some embodiments, crystalline Compound I Form 1 is substantially pure. In some embodiments, crystalline Compound I Form 1 is substantially crystalline. In some embodiments, crystalline Compound I Form 1 is characterized by an X-ray powder diffractogram generated by an X-ray powder diffraction analysis with an incident beam of Cu Kα radiation.

In some embodiments, crystalline Compound I Form 1 is characterized by an X-ray powder diffractogram having a signal at one or more of 6.37±2 degrees two-theta, 7±2 degrees two-theta, 9.13±2 degrees two-theta, 9.84±2 degrees two-theta, 13.55±2 degrees two-theta, 13.69±2 degrees two-theta, ±2 degrees two-theta, 14.27±2 degrees two-theta, 14.57±2 degrees two-theta, 15.71±2 degrees two-theta, 16.14±2 degrees two-theta, 16.53±2 degrees two-theta, 18.1±2 degrees two-theta, 18.32±2 degrees two-theta, 19.07±2 degrees two-theta, 19.99±2 degrees two-theta, 20.46±2 degrees two-theta, 21.01±2 degrees two-theta, 21.66±2 degrees two-theta, 22.24±2 degrees two-theta, 22.6±2 degrees two-theta, 23.01±2 degrees two-theta, 23.65±2 degrees two-theta, 24.68±2 degrees two-theta, 25.69±2 degrees two-theta, 26.17±2 degrees two-theta, 26.51±2 degrees two-theta, 27.81±2 degrees two-theta, 28.81±2 degrees two-theta, 29.45±2 degrees two-theta, 30.48±2 degrees two-theta, 31.37±2 degrees two-theta, 32.4±2 degrees two-theta, 34.41±2 degrees two-theta, 34.81±2 degrees two-theta, 35.53±2 degrees two-theta, and 39.62±2 degrees two-theta.

In some embodiments, crystalline Compound I Form 1 is characterized by an X-ray powder diffractogram having a signal at one or more of 6.37±2 degrees two-theta, 9.13±2 degrees two-theta, 9.84±2 degrees two-theta, 13.55±2 degrees two-theta, and 14.27±2 degrees two-theta.

In some embodiments, crystalline Compound I Form 1 is characterized by an X-ray powder diffractogram having a signal at one or more of 6.37±2 degrees two-theta, 9.84±2 degrees two-theta, 13.55±2 degrees two-theta, and 14.27±2 degrees two-theta.

In some embodiments, crystalline Compound I Form 1 is characterized by an X-ray powder diffractogram having a signal at one or more of 6.37±2 degrees two-theta, 13.55±2 degrees two-theta, and 14.27±2 degrees two-theta.

In some embodiments, crystalline Compound I Form 1 is characterized by an X-ray powder diffractogram having a signal at one or more of 6.37±2 degrees two-theta, and 14.27±2 degrees two-theta.

In some embodiments, crystalline Compound I Form 1 is characterized by an X-ray powder diffractogram having a signal at 6.37±2 degrees two-theta. In some embodiments, crystalline Compound I Form 1 is characterized by an X-ray powder diffractogram having a signal at 9.13±2 degrees two-theta. In some embodiments, crystalline Compound I Form 1 is characterized by an X-ray powder diffractogram having a signal at 9.84±2 degrees two-theta. In some embodiments, crystalline Compound I Form 1 is characterized by an X-ray powder diffractogram having a signal at 13.55±2 degrees two-theta. In some embodiments, crystalline Compound I Form 1 is characterized by an X-ray powder diffractogram having a signal at 14.27±2 degrees two-theta.

In some embodiments, crystalline Compound I Form 1 is characterized by an X-ray powder diffractogram substantially similar to FIG. 1.

In some embodiments, crystalline Compound I Form 1 is characterized by a primitive monoclinic crystal system, P2₁/n1 space group and the following unit cell dimensions resulting from successful indexing of Form 1 XRPD:

| | | | |
|---|---|---|---|
| a | 18.0 ± 0.1 Å | α | 90 = 0.1° |
| b | 6.9 ± 0.1 Å | β | 95.5 = 0.1° |
| c | 19.5 ± 0.1 Å | γ | 90 + 0.1°. |

As noted hereinabove, standards of deviation provide an acceptable tolerance appreciated by those of ordinary skill in the art.

In some embodiments, Compound I Form 1 is characterized by a DSC having an onset of melting temperature of 210° C. The endotherm may be multiple, overlapping endotherms. In some embodiments, Compound I Form 1 is characterized by a DSC having a broad endotherm onset of melting temperature of 147° C.

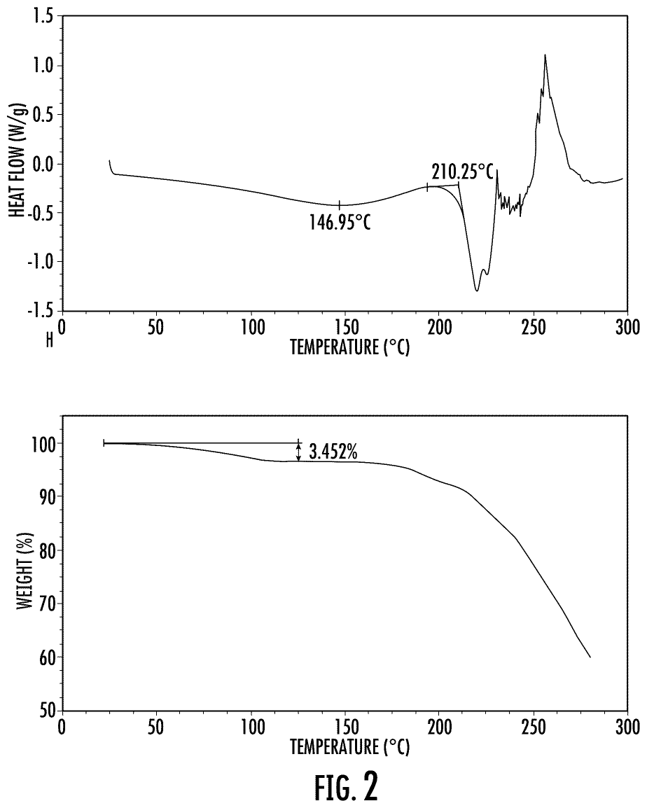

In some embodiments, Compound I Form 1 is characterized by a DSC substantially similar to FIG. 2 (top).

In some embodiments, Compound I Form 1 is also characterized by a weight loss of approximately 3.5% at temperatures up to 125° C., as measured by thermogravimetric analysis.

In some embodiments, Compound I Form 1 is characterized by a TGA substantially similar to FIG. 2 (bottom).

In some embodiments, Compound I Form 1 is also characterized as hygroscopic, evidenced by the water uptake of 0.5% at relative humidity of up to 65%, as measured by DVS. In some embodiments, Compound I Form 1 is also characterized as hygroscopic, evidenced by the water uptake of 6.7% at relative humidity of 65% to 85%, as measured by DVS. In some embodiments, Compound I Form 1 is also characterized as hygroscopic, evidenced by the water uptake of 0.3% at relative humidity of 85% to 95%, as measured by DVS. In some embodiments, Compound I Form 1 is also characterized as hygroscopic, evidenced by total water uptake of 7.5% (~2.2 moles), as measured by DVS.

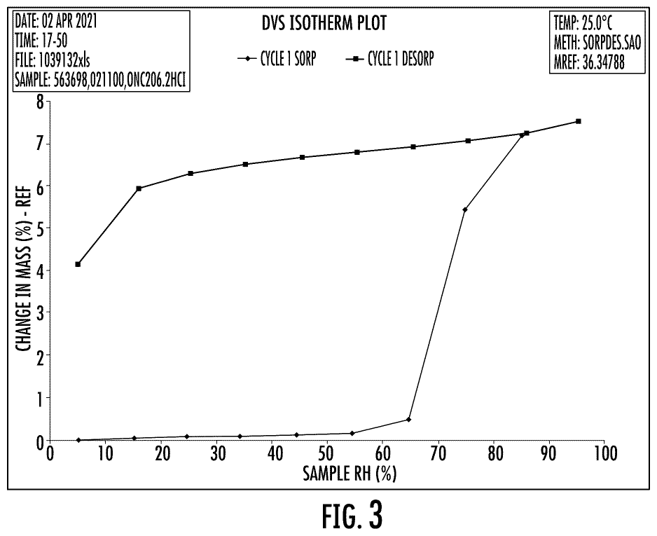

In some embodiments, Compound I Form 1 is characterized by a DVS substantially similar to FIG. 3.

In some embodiments, crystalline Compound I Form 1 is characterized by an ¹⁹F ssNMR spectrum having one or two peaks selected from −106.64 ppm and −112.38 ppm. In some embodiments a tolerance of ±0.2 ppm is provided. In some embodiments, crystalline Compound I Form 1 is characterized by an ¹⁹F ssNMR spectrum having a peak at −106.64 ppm. In some embodiments, crystalline Compound I Form 1 is characterized by an ¹⁹F ssNMR spectrum having a peak at −112.38 ppm.

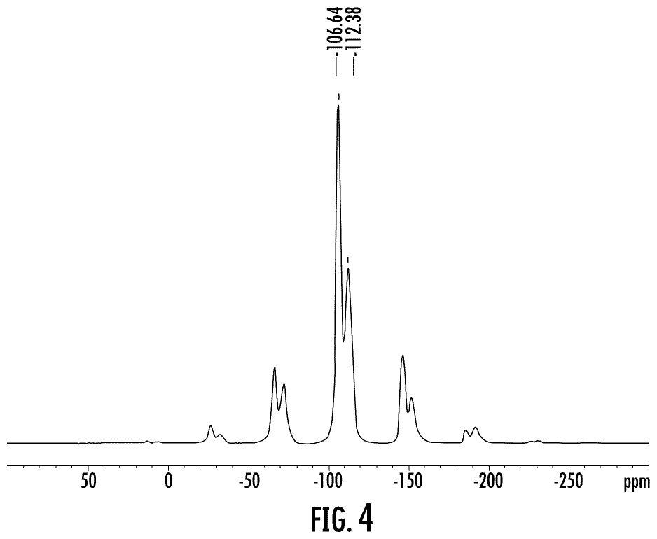

In some embodiments, crystalline Compound I Form 1 is characterized by a ¹⁹F ssNMR spectrum substantially similar to FIG. 4.

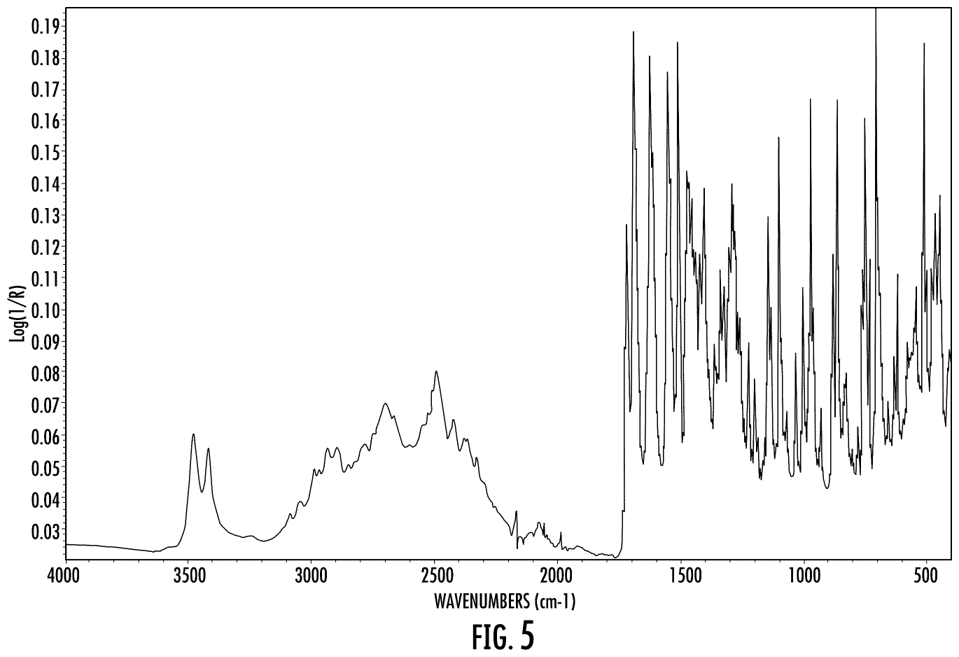

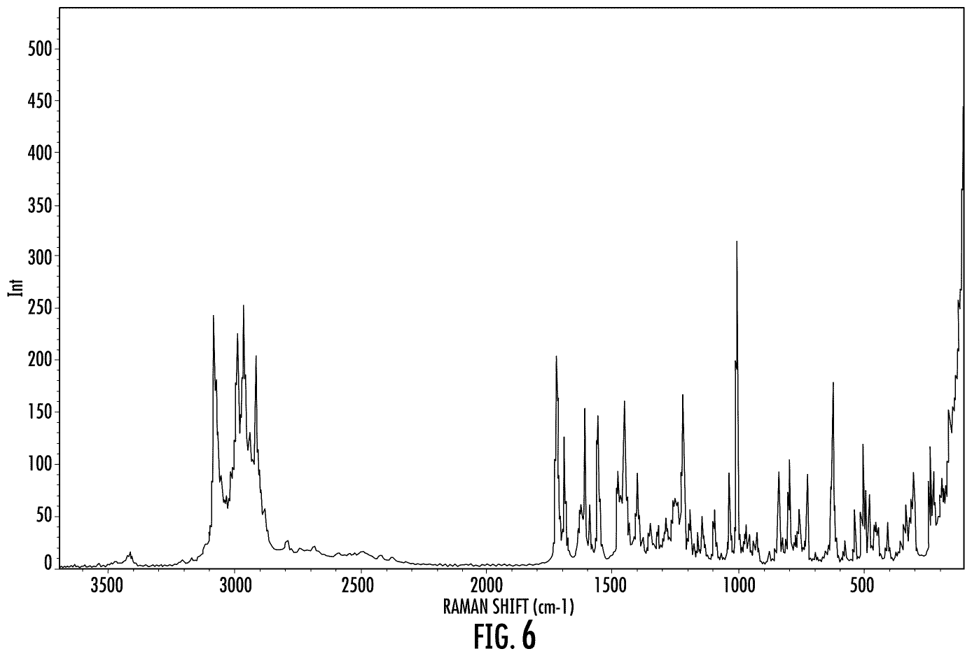

In some embodiments, Compound I Form 1 provides FT-IR spectrum of FIG. 5 showing crystalline Compound I Form 1 with corresponding tabulated data shown in Table 2.

TABLE 2
FT-IR Peaks for Crystalline Compound I Form 1

| Wavenumber (cm⁻¹) | Relative Intensity |
|---|---|
| 439 | 69 |
| 458 | 66 |
| 502 | 94 |
| 506 | 85 |
| 611 | 57 |
| 699 | 100 |
| 724 | 59 |
| 744 | 82 |
| 756 | 56 |
| 856 | 85 |
| 872 | 60 |
| 954 | 50 |
| 966 | 85 |
| 995 | 54 |
| 1025 | 44 |
| 1092 | 79 |
| 1127 | 51 |
| 1139 | 66 |
| 1190 | 39 |
| 1217 | 45 |
| 1254 | 49 |
| 1283 | 71 |
| 1296 | 61 |
| 1316 | 54 |
| 1330 | 57 |
| 1398 | 71 |
| 1415 | 60 |
| 1430 | 60 |
| 1447 | 69 |
| 1467 | 73 |
| 1505 | 94 |
| 1545 | 89 |
| 1605 | 75 |
| 1619 | 91 |
| 1683 | 96 |
| 1714 | 65 |
| 2483 | 41 |
| 2693 | 35 |
| 3411 | 28 |
| 3471 | 31 |

In some embodiments, crystalline Compound I Form 1 is characterized by an FT-IR spectrum having a signal at one or more of 439 cm⁻¹, 458 cm⁻¹, 502 cm⁻¹, 506 cm⁻¹, 611 cm⁻¹, 699 cm⁻¹, 724 cm⁻¹, 744 cm⁻¹, 756 cm⁻¹, 856 cm⁻¹, 872 cm⁻¹, 954 cm⁻¹, 966 cm⁻¹, 995 cm⁻¹, 1025 cm⁻¹, 1092 cm⁻¹, 1127 cm⁻¹, 1139 cm⁻¹, 1190 cm⁻¹, 1217 cm⁻¹, 1254 cm⁻¹, 1283 cm⁻¹, 1296 cm⁻¹, 1316 cm⁻¹, 1330 cm⁻¹, 1398 cm⁻¹, 1415 cm⁻¹, 1430 cm⁻¹, 1447 cm⁻¹, 1467 cm⁻¹, 1505 cm⁻¹, 1545 cm⁻¹, 1605 cm⁻¹, 1619 cm⁻¹, 1683 cm⁻¹, 1714 cm⁻¹, 2483 cm⁻¹, 2693 cm⁻¹, 3411 cm⁻¹, and 3471 cm⁻¹.

As noted hereinabove, standards of deviation provide an acceptable tolerance appreciated by those of ordinary skill in the art. Values provided should be considered to have acceptable standards of deviation.

In some embodiments, crystalline Compound I Form 1 is characterized by an FT-IR spectrum having a signal at one or more of 856 cm⁻¹, 872 cm⁻¹, 1283 cm⁻¹, 1714 cm⁻¹, and 2483 cm⁻¹.

In some embodiments, crystalline Compound I Form 1 is characterized by an FT-IR spectrum having a signal at one or more of 856 cm⁻¹, 872 cm⁻¹, 1283 cm⁻¹, and 1714 cm⁻¹.

In some embodiments, crystalline Compound I Form 1 is characterized by an FT-IR spectrum having a signal at 856 cm⁻¹. In some embodiments, crystalline Compound I Form 1 is characterized by an FT-IR spectrum having a signal at 872 cm⁻¹. In some embodiments, crystalline Compound I Form 1 is characterized by an FT-IR spectrum having a signal at 1283 cm⁻¹. In some embodiments, crystalline Compound I Form 1 is characterized by an FT-IR spectrum having a signal at 1714 cm⁻¹. In some embodiments, crystalline Compound I Form 1 is characterized by an FT-IR spectrum having a signal at 2483 cm⁻¹.

Compound I Form 1 is characterized by an FT-IR spectrum substantially similar to FIG. 5.

In some embodiments, Compound I Form 1 provides the FT-Raman spectrum of FIG. 6 showing crystalline Compound I Form 1 with corresponding tabulated data shown in Table 3.

TABLE 3
FT-Raman Peaks for Crystalline Compound I Form 1

| Wavenumber (cm⁻¹) | Relative Intensity |
|---|---|
| 156 | 48 |
| 187 | 27 |
| 218 | 29 |
| 235 | 36 |
| 299 | 29 |
| 332 | 19 |
| 472 | 22 |
| 491 | 24 |
| 498 | 37 |
| 534 | 18 |
| 621 | 57 |
| 723 | 28 |
| 755 | 18 |
| 794 | 33 |
| 833 | 29 |
| 1002 | 100 |
| 1030 | 28 |
| 1215 | 53 |
| 1395 | 29 |
| 1446 | 51 |
| 1551 | 46 |
| 1603 | 49 |
| 1685 | 39 |
| 1715 | 65 |
| 2909 | 65 |
| 2960 | 81 |
| 2980 | 71 |
| 3076 | 77 |

In some embodiments, crystalline Compound I Form 1 is characterized by an FT-Raman spectrum having a signal at one or more of 156 cm⁻¹, 187 cm⁻¹, 218 cm⁻¹, 235 cm⁻¹, 299 cm⁻¹, 332 cm⁻¹, cm⁻¹, 472 cm⁻¹, 491 cm⁻¹, 498 cm⁻¹, 534 cm⁻¹, 621 cm⁻¹, 723 cm⁻¹, 755 cm⁻¹, 794 cm⁻¹, 833 cm⁻¹, 1002 cm⁻¹, 1030 cm⁻¹, 1215 cm⁻¹, 1395 cm⁻¹, 1446 cm⁻¹, 1551 cm⁻¹, 1603 cm⁻¹, 1685 cm⁻¹, 1715 cm⁻¹, 2909 cm⁻¹, 2960 cm⁻¹, 2980 cm⁻¹, and 3076 cm⁻¹.

In some embodiments, crystalline Compound I Form 1 is characterized by an FT-Raman spectrum having a signal at one or more of 235 cm⁻¹, 794 cm⁻¹, 1715 cm⁻¹, and 2909 cm⁻¹.

In some embodiments, crystalline Compound I Form 1 is characterized by an FT-Raman spectrum having a signal at one or more of 235 cm⁻¹, 794 cm⁻¹, and 1715 cm⁻¹.

In some embodiments, crystalline Compound I Form 1 is characterized by an FT-Raman spectrum having a signal at 235 cm⁻¹. In some embodiments, crystalline Compound I Form 1 is characterized by an FT-Raman spectrum having a signal at 794 cm⁻¹. In some embodiments, crystalline Compound I Form 1 is characterized by an FT-Raman spectrum having a signal at 1715 cm⁻¹. In some embodiments, crystalline Compound I Form 1 is characterized by an FT-Raman spectrum having a signal at 2909 cm⁻¹.

Compound I Form 1 is characterized by an FT-Raman spectrum substantially similar to FIG. 6.

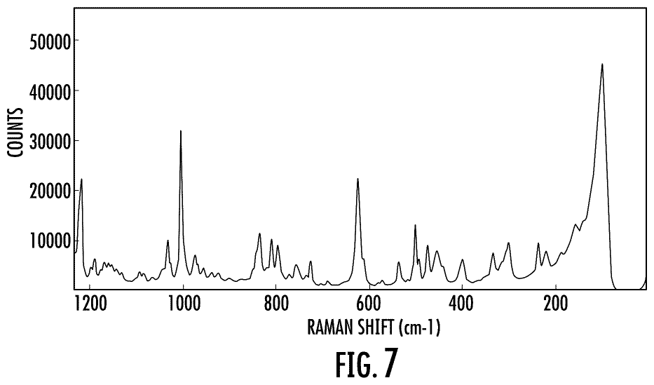

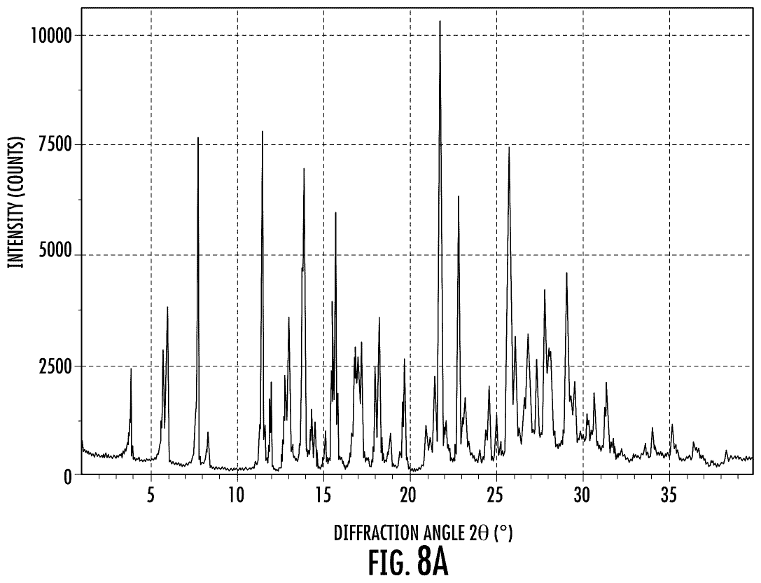

In some embodiments, Compound I Form 1 provides the LF Raman spectrum of FIG. 7 showing crystalline Compound I Form 1 with corresponding tabulated data shown in Table 4.

TABLE 4
LF Raman Peaks for Crystalline Compound I Form 1

| Wavenumber (cm⁻¹) | Relative Intensity |
|---|---|
| 98 | 54 |
| 156 | 3 |
| 219 | 11 |
| 235 | 19 |
| 298 | 23 |
| 332 | 14 |
| 399 | 17 |
| 453 | 17 |
| 473 | 21 |
| 499 | 40 |
| 535 | 16 |
| 571 | 3 |
| 622 | 60 |
| 687 | 3 |
| 724 | 15 |
| 755 | 10 |
| 772 | 2 |
| 795 | 14 |
| 808 | 20 |
| 833 | 29 |
| 972 | 12 |
| 954 | 5 |

In some embodiments, crystalline Compound I Form 1 is characterized by an LF Raman spectrum having a signal at one or more of 98 cm⁻¹, 622 cm⁻¹, 156 cm⁻¹, 219 cm⁻¹, 235 cm⁻¹, 298 cm⁻¹, 332 cm⁻¹, 399 cm⁻¹, 453 cm⁻¹, 473 cm⁻¹, 499 cm⁻¹, 535 cm⁻¹, 571 cm⁻¹, 622 cm⁻¹, 687 cm⁻¹, 724 cm⁻¹, 755 cm⁻¹, 772 cm⁻¹, 795 cm⁻¹, 808 cm⁻¹, 833 cm⁻¹, 972 cm⁻¹, and 954 cm⁻¹.

In some embodiments, crystalline Compound I Form 1 is characterized by an LF Raman spectrum having a signal at one or more of 98 cm⁻¹, 156 cm⁻¹, 235 cm⁻¹, 332 cm⁻¹, and 795 cm⁻¹.

In some embodiments, crystalline Compound I Form 1 is characterized by an LF Raman spectrum having a signal at one or more of 98 cm⁻¹ and 795 cm⁻¹.

In some embodiments, crystalline Compound I Form 1 is characterized by an LF Raman spectrum having a signal at one or more of 156 cm⁻¹, 235 cm⁻¹, and 332 cm⁻¹.

In some embodiments, crystalline Compound I Form 1 is characterized by an LF Raman spectrum having a signal at 98 cm⁻¹. In some embodiments, crystalline Compound I Form 1 is characterized by an LF Raman spectrum having a signal at 156 cm⁻¹. In some embodiments, crystalline Compound I Form 1 is characterized by an LF Raman spectrum having a signal at 235 cm⁻¹. In some embodiments, crystalline Compound I Form 1 is characterized by an LF Raman spectrum having a signal at 332 cm⁻¹. In some embodiments, crystalline Compound I Form 1 is characterized by an LF Raman spectrum having a signal at 795 cm⁻¹.

Compound I Form 1 is characterized by an LF-Raman spectrum substantially similar FIG. 7.

Another embodiment of the disclosure provides a method of making crystalline Compound I Form 1. In some embodiments, the method of making crystalline Compound I Form 1 comprises: (i) combining methyl 1-benzyl-4-oxopiperidine-3-carboxylate·HCl, NaHCO₃, and n-butanol (ii) stir for 30 min, (iii) drying for 2 hr over MgSO₄, (iv) adding N-(2,4-difluorobenzyl)-4,5-dihydro-1H-imidazol-2-amine and PPTS before stirring overnight, (v) refluxing and cooling to room temperature (rt) after 4 hr, (vi) adding 2 M HCl in dioxane-methyl t-butyl ether (MTBE), (vii) refluxing at 60-65° C. for 2 hr, (viii) cooling to rt, (ix) collecting the solids, (x) drying the solids in a vacuum oven at 55° C. for 4 hr, then room temperature for 60 h, (xi) adding ethanol to dried solids, (xii) heating until solids dissolve and cooling to rt, (xiii) stirring overnight, (xiv) stirring at 15° C. for 1 hr (xv) collecting solids, (xvi) drying the solids in a vacuum oven at 75° C. to yield crystalline Compound I Form 1.

In some embodiments, the method of making crystalline Compound I Form 1 comprises recrystallizing crude Compound I·2HCl in ethanol.

Another embodiment of the disclosure provides a method of making crystalline Compound I Form 1. In some embodiments, the method of making crystalline Compound I Form 1 comprises: (i) combining methyl 1-benzyl-4-oxopiperidine-3-carboxylate·HCl, K2CO3, and toluene (ii) stir for 30 min, (iii) adding N-(2,4-difluorobenzyl)-4,5-dihydro-1H-imidazol-2-amine and PPTS, (iv) refluxing at 110° C. for 3 hr (v) cooling to rt, (vi) distilling the organic layer and heating to 75° C., (vii) adding MTBE and cooling to rt, (viii) collecting the solids, (ix) (x) drying the solids in a vacuum oven at 40° C., (xi) adding toluene to dried solids and heating to 65° C., (xii) adding 2.5 M HCl in ethanol and cooling to rt overnight, (xiii) collecting the solids, (xiv) drying the solids in a vacuum oven at 75° C., (xv) refluxing the dried solids in IPA at 82° C. for 2 hr, (xvi) cooling to 20° C., (xvii) collecting the solids, (xviii) drying the solids in a vacuum at 75° C. for several days to yield crystalline Compound I Form 1.

In some embodiments, the method of making crystalline Compound I Form 1 comprises (i) slurrying crude Compound I·2HCl in refluxing IPA at 82° C. for 2 hrs, (ii) cooling to 20° C., (iii) collecting the solids, (iv) drying the solids in a vacuum at 75° C. for several days to yield crystalline Compound I Form 1.

In some embodiments, the method of making crystalline Compound I Form 1 comprises recrystallizing ONC201·2HCl in ethanol and drying under vacuum at 75° C. for 6 days to yield crystalline Compound I Form 1.

In some embodiments, the method of making crystalline Compound I Form 1 comprises (i) adding isopropyl acetate to ONC201·2HCl producing a white slurry, (ii) stirring at rt for 7 days, and (iii) centrifuging and removing the supernatant yield crystalline Compound I Form 1.

### B. Compound I Form 2

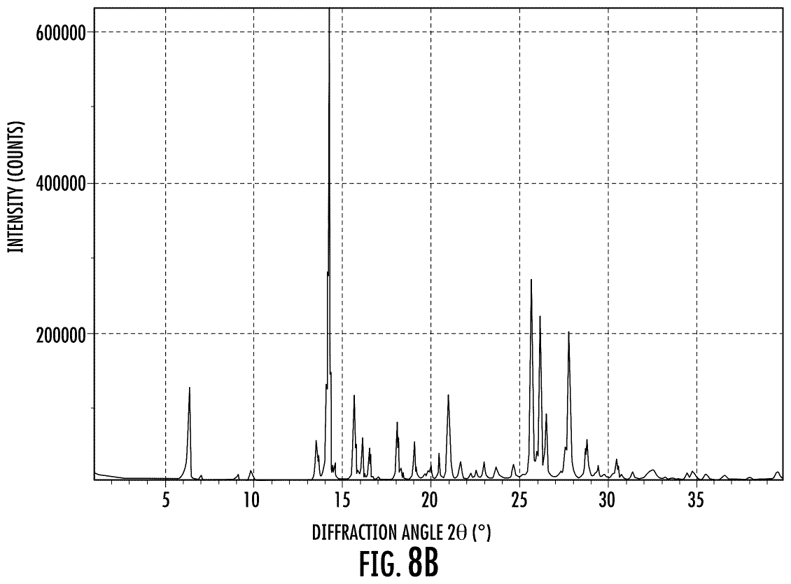

In some embodiments, the disclosure provides crystalline Compound I Form 2. In some embodiments, Compound I Form 2 is a variable hydrate. In some embodiments, Compound I Form 2 is a variable hydrate with up to 3 moles of water. In some embodiments, Compound I Form 2 is a dihydrate. FIG. 8 provides an X-ray powder diffractogram of crystalline Compound I Form 2 with corresponding tabulated data for FIG. 8 shown in Table 5.

TABLE 5
XRPD Signals for Crystalline Compound I Form 2

| | | Intenisty | Relative |
|---|---|---|---|
| °2Θ (±0.2°) | d-spacing (Å) | (Counts) | Intensity |
| 3.86 | 22.87 | 2748 | 26 |
| 5.72 | 15.44 | 3160 | 30 |
| 5.97 | 14.79 | 4146 | 39 |
| 7.74 | 11.41 | 7960 | 75 |
| 8.27 | 10.68 | 1281 | 12 |
| 11.45 | 7.72 | 8107 | 76 |
| 11.62 | 7.61 | 1438 | 14 |
| 11.95 | 7.4 | 2432 | 23 |
| 12.77 | 6.93 | 2604 | 25 |
| 12.99 | 6.81 | 3915 | 37 |
| 13.89 | 6.37 | 7255 | 68 |
| 14.31 | 6.18 | 1787 | 17 |
| 14.52 | 6.1 | 1496 | 14 |
| 15.11 | 5.86 | 1303 | 12 |
| 15.51 | 5.71 | 4276 | 40 |
| 15.71 | 5.64 | 6266 | 59 |
| 16.83 | 5.26 | 3133 | 29 |
| 17.01 | 5.21 | 3000 | 28 |
| 17.2 | 5.15 | 3335 | 31 |
| 17.97 | 4.93 | 2764 | 26 |
| 18.22 | 4.87 | 3897 | 37 |
| 18.85 | 4.7 | 1250 | 12 |
| 19.69 | 4.51 | 2929 | 28 |
| 20.92 | 4.24 | 1396 | 13 |
| 21.17 | 4.19 | 1129 | 11 |
| 21.43 | 4.14 | 2551 | 24 |
| 21.74 | 4.08 | 10626 | 100 |
| 22.04 | 4.03 | 1523 | 14 |
| 22.8 | 3.9 | 6635 | 62 |
| 23.18 | 3.83 | 2047 | 19 |
| 24.38 | 3.65 | 1326 | 12 |
| 24.57 | 3.62 | 2337 | 22 |
| 25 | 3.56 | 1684 | 16 |
| 25.22 | 3.53 | 1075 | 10 |
| 25.74 | 3.46 | 7751 | 73 |
| 26.07 | 3.42 | 3463 | 33 |
| 26.82 | 3.32 | 3518 | 33 |
| 27.34 | 3.26 | 2944 | 28 |
| 27.79 | 3.21 | 4514 | 42 |
| 29.06 | 3.07 | 4898 | 46 |
| 29.48 | 3.03 | 2447 | 23 |
| 30.25 | 2.95 | 1687 | 16 |
| 30.65 | 2.91 | 2150 | 20 |
| 31.35 | 2.85 | 2439 | 23 |
| 31.74 | 2.82 | 1118 | 11 |
| 34.03 | 2.63 | 1358 | 13 |
| 35.15 | 2.55 | 1422 | 13 |
| 36.42 | 2.46 | 1067 | 10 |

In some embodiments, crystalline Compound I Form 2 is substantially pure. In some embodiments, crystalline Compound I Form 2 is substantially crystalline. In some embodiments, crystalline Compound I Form 2 is characterized by an X-ray powder diffractogram generated by an X-ray powder diffraction analysis with an incident beam of Cu Kα radiation.

In some embodiments, crystalline Compound I Form 2 is characterized by an X-ray powder diffractogram having a signal at one or more of 3.86±2 degrees two-theta, 5.72±2 degrees two-theta, 5.97±2 degrees two-theta, 7.74±2 degrees two-theta, 8.27±2 degrees two-theta, 11.45±2 degrees two-theta, ±2 degrees two-theta, 11.62±2 degrees two-theta, 11.95±2 degrees two-theta, 12.77±2 degrees two-theta, 12.99±2 degrees two-theta, 13.89±2 degrees two-theta, 14.31±2 degrees two-theta, 14.52±2 degrees two-theta, 15.11±2 degrees two-theta, 15.51±2 degrees two-theta, 15.71±2 degrees two-theta, 16.83±2 degrees two-theta, 17.01±2 degrees two-theta, 17.2±2 degrees two-theta, 17.97±2 degrees two-theta, 18.22±2 degrees two-theta, 18.85±2 degrees two-theta, 19.69±2 degrees two-theta, 20.92±2 degrees two-theta, 21.17±2 degrees two-theta, 21.43±2 degrees two-theta, 21.74±2 degrees two-theta, 22.04±2 degrees two-theta, 22.8±2 degrees two-theta, 23.18±2 degrees two-theta, 24.38±2 degrees two-theta, 24.57±2 degrees two-theta, 25±2 degrees two-theta, 25.22±2 degrees two-theta, 25.74±2 degrees two-theta, 26.07±2 degrees two-theta, 26.82±2 degrees two-theta, 27.34±2 degrees two-theta, 27.79±2 degrees two-theta, 29.06±2 degrees two-theta, 29.48±2 degrees two-theta, 30.25±2 degrees two-theta, 30.65±2 degrees two-theta, 31.35±2 degrees two-theta, 31.74±2 degrees two-theta, 34.03±2 degrees two-theta, 35.15±2 degrees two-theta, and 36.42±2 degrees two-theta.

In some embodiments, crystalline Compound I Form 2 is characterized by an X-ray powder diffractogram having a signal at one or more of 5.72±2 degrees two-theta, 5.97±2 degrees two-theta, 11.45±2 degrees two-theta, 11.95±2 degrees two-theta, and 13.89±2 degrees two-theta.

In some embodiments, crystalline Compound I Form 2 is characterized by an X-ray powder diffractogram having a signal at one or more of 5.72±2 degrees two-theta, 5.97±2 degrees two-theta, 11.45±2 degrees two-theta, and 13.89±2 degrees two-theta.

In some embodiments, crystalline Compound I Form 2 is characterized by an X-ray powder diffractogram having a signal at one or more of 5.97±2 degrees two-theta, 11.45±2 degrees two-theta, and 13.89±2 degrees two-theta.

In some embodiments, crystalline Compound I Form 2 is characterized by an X-ray powder diffractogram having a signal at one or more of 11.45±2 degrees two-theta, and 13.89±2 degrees two-theta.

In some embodiments, crystalline Compound I Form 2 is characterized by an X-ray powder diffractogram having a signal at 5.72±2 degrees two-theta. In some embodiments, crystalline Compound I Form 2 is characterized by an X-ray powder diffractogram having a signal at 5.97±2 degrees two-theta. In some embodiments, crystalline Compound I Form 2 is characterized by an X-ray powder diffractogram having a signal at 11.45±2 degrees two-theta. In some embodiments, crystalline Compound I Form 2 is characterized by an X-ray powder diffractogram having a signal at 11.95±2 degrees two-theta. In some embodiments, crystalline Compound I Form 2 is characterized by an X-ray powder diffractogram having a signal at 13.89±2 degrees two-theta.

In some embodiments, crystalline Compound I Form 2 is characterized by an X-ray powder diffractogram substantially similar to FIG. 8.

In some embodiments, crystalline Compound I Form 2 is characterized by a C-centered monoclinic crystal system, Cc space group and the following unit cell dimensions resulting from successful indexing of Form 2 XRPD:

| | | | |
|---|---|---|---|
| a | 47.5 ± 0.1 Å | α | 90 ± 0.1° |
| b | 7.0 ± 0.1 Å | β | 105.9 ± 0.1° |
| c | 32.1 ± 0.1 Å | γ | 90 ± 0.1°. |

In some embodiments, Compound I Form 2 is characterized by a DSC having a broad endotherm peak temperature of 110° C. In some embodiments, Compound I Form 2 is characterized by a DSC having a small endotherm peak temperature of 180° C. In some embodiments, Compound I Form 2 is characterized by a DSC having a large endotherm peak temperature of 225° C. In some embodiments, Compound I Form 2 is characterized by a DSC having a broad endotherm peak temperature of 110° C., small endotherm peak temperature of 180° C. and/or a large endotherm peak temperature of 225° C.

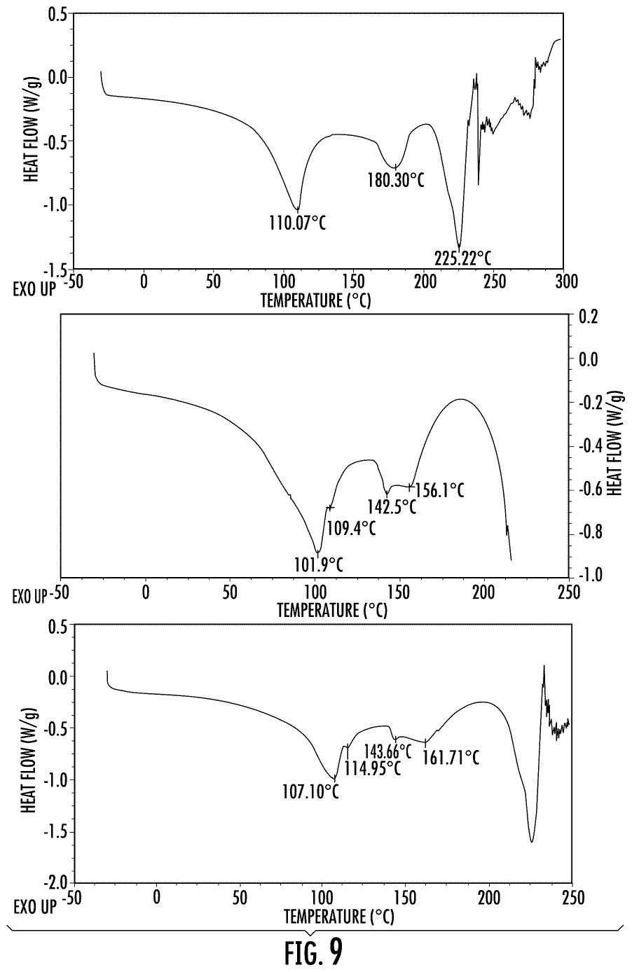

In some embodiments, Compound I Form 2 is characterized by a DSC substantially similar to FIG. 9 (top).

In some embodiments, Compound I Form 2 is characterized by a DSC having an endotherm peak temperature of 102° C. In some embodiments, Compound I Form 2 is characterized by a DSC having an endotherm peak temperature of 109° C. In some embodiments, Compound I Form 2 is characterized by a DSC having an endotherm peak temperature of 143° C. In some embodiments, Compound I Form 2 is characterized by a DSC having an endotherm peak temperature of 156° C. In some embodiments, Compound I Form 2 is characterized by a DSC having a broad overlapping endotherms with peak temperature of 102° C., 109° C., 143° C., and/or 156° C.

In some embodiments, Compound I Form 2 is characterized by a DSC substantially similar to FIG. 9 (middle).

In some embodiments, Compound I Form 2 is characterized by a DSC having an endotherm peak temperature of 107° C. In some embodiments, Compound I Form 2 is characterized by a DSC having an endotherm peak temperature of 115° C. In some embodiments, Compound I Form 2 is characterized by a DSC having an endotherm peak temperature of 144° C. In some embodiments, Compound I Form 2 is characterized by a DSC having an endotherm peak temperature of 162° C. In some embodiments, Compound I Form 2 is characterized by a DSC having a broad overlapping endotherms with peak temperature of 107° C., 115° C., 144° C., and/or 162° C.

In some embodiments, Compound I Form 2 is characterized by a DSC substantially similar to FIG. 9 (bottom).

In some embodiments, Compound I Form 2 is also characterized by a weight loss of 6.6% (1.9 moles of H₂O) up to 100° C., as measured by thermogravimetric analysis. In some embodiments, Compound I Form 2 is also characterized by a weight loss of approximately 3.5% (1.0 moles of H₂O) at temperatures 100° C. to 175° C., as measured by thermogravimetric analysis.

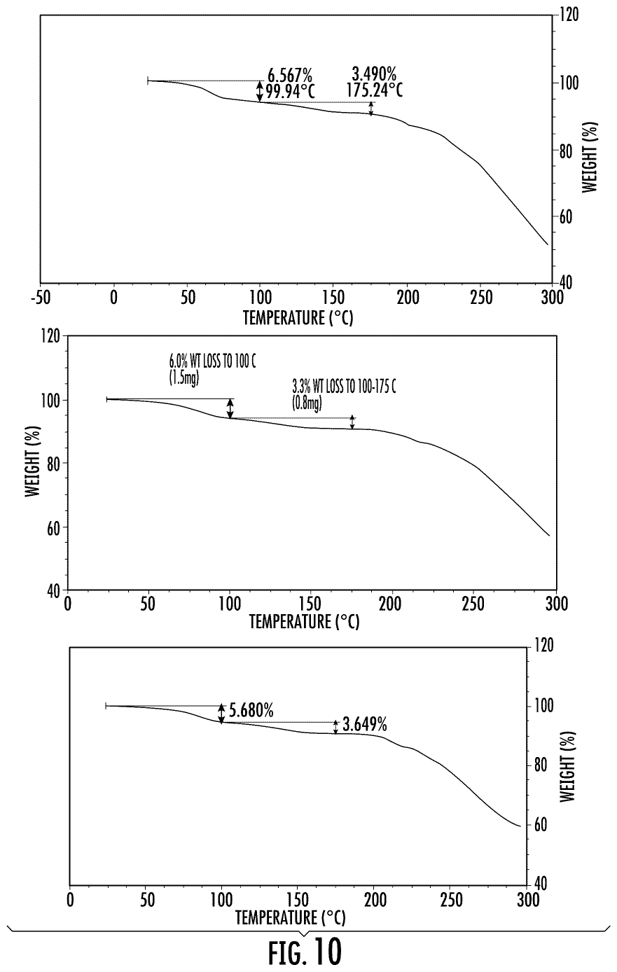

In some embodiments, Compound I Form 2 is characterized by a TGA substantially similar to FIG. 10 (top).

In some embodiments, Compound I Form 2 is also characterized by a weight loss of 6.0% (1.7 moles of H₂O) up to 100° C., as measured by thermogravimetric analysis. In some embodiments, Compound I Form 2 is also characterized by a weight loss of approximately 3.3% (0.9 moles of H₂O) at temperatures 100° C. to 175° C., as measured by thermogravimetric analysis.

In some embodiments, Compound I Form 2 is characterized by a TGA substantially similar to FIG. 10 (middle).

In some embodiments, Compound I Form 2 is also characterized by a weight loss of 5.7% (1.6 moles of H₂O) up to 100° C., as measured by thermogravimetric analysis. In some embodiments, Compound I Form 2 is also characterized by a weight loss of approximately 3.6% (1.0 moles of H₂O) at temperatures 100° C. to 175° C., as measured by thermogravimetric analysis.

In some embodiments, Compound I Form 2 is characterized by a TGA substantially similar to FIG. 10 (bottom).

In some embodiments, Compound I Form 2 is also characterized as hygroscopic, evidenced by the water uptake of 1.8% at relative humidity of 5% to 25%, as measured by DVS. In some embodiments, Compound I Form 2 is also characterized as hygroscopic, evidenced by the water uptake of 0.6% at relative humidity of 25% to 95%, as measured by DVS. In some embodiments, Compound I Form 2 is also characterized as hygroscopic, evidenced by total water uptake of 2.5% (~0.7 moles), as measured by DVS.

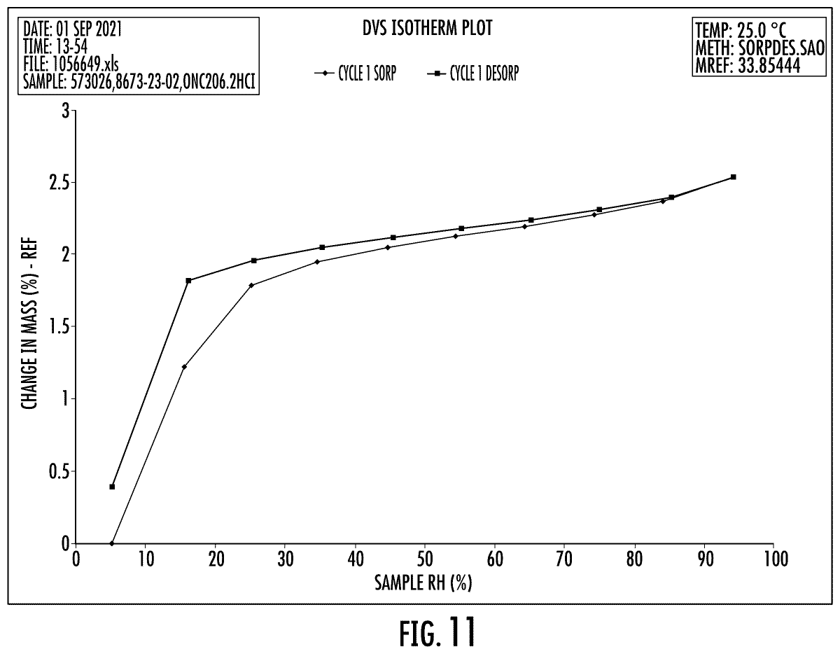

In some embodiments, Compound I Form 2 is characterized by a DVS substantially similar to FIG. 11.

In some embodiments, crystalline Compound I Form 2 is characterized by an ¹⁹F ssNMR spectrum having one or two peaks selected from −110.83±0.2 ppm and −115.29±0.2 ppm. In some embodiments, crystalline Compound I Form 2 is characterized by an ¹⁹F ssNMR spectrum having a peak at −110.83±0.2 ppm. In some embodiments, crystalline Compound I Form 2 is characterized by an ¹⁹F ssNMR spectrum having a peak at −115.29±0.2 ppm.

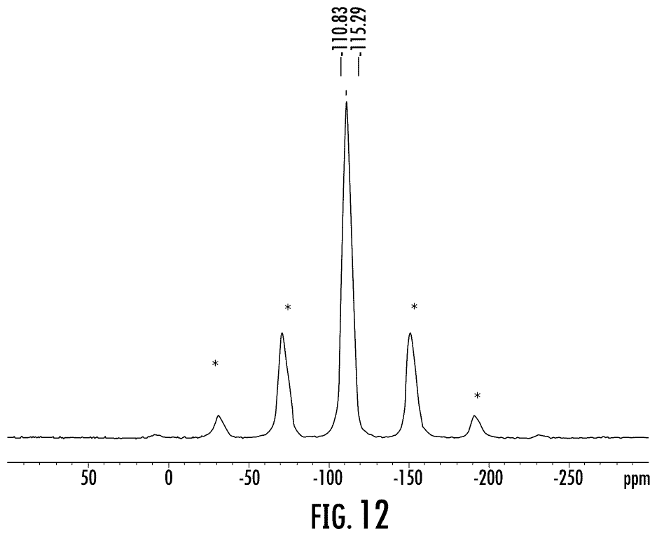

In some embodiments, crystalline Compound I Form 2 is characterized by a ¹⁹F ssNMR spectrum substantially similar to FIG. 12.

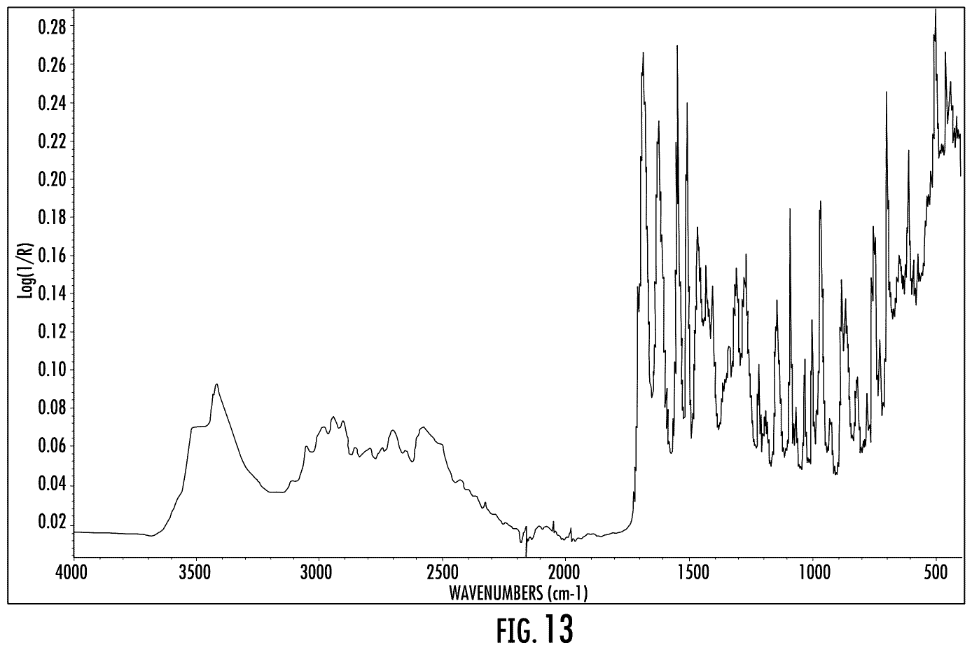

In some embodiments, Compound I Form 2 provides FT-IR spectrum of crystalline Compound I Form 2 of FIG. 13 with corresponding tabulated data shown in Table 6.

TABLE 6
FT-IR Peaks for Crystalline Compound I Form 2

| Wavenumber | Relative |
|---|---|
| (cm⁻¹) | Intensity |
| 436 | 87 |
| 457 | 92 |
| 499 | 100 |
| 608 | 75 |
| 697 | 85 |
| 743 | 58 |
| 756 | 59 |
| 861 | 47 |
| 878 | 51 |
| 964 | 65 |
| 996 | 42 |
| 1030 | 36 |
| 1089 | 64 |
| 1141 | 47 |
| 1215 | 36 |
| 1265 | 55 |
| 1305 | 53 |
| 1401 | 49 |
| 1430 | 53 |
| 1463 | 60 |
| 1507 | 83 |
| 1544 | 93 |
| 1622 | 79 |
| 1681 | 92 |
| 1704 | 49 |
| 2584 | 24 |
| 2942 | 26 |
| 3414 | 32 |

In some embodiments, crystalline Compound I Form 2 is characterized by an FT-IR spectrum having a signal at one or more of 436 cm⁻¹, 457 cm⁻¹, 499 cm⁻¹, 608 cm⁻¹, 697 cm⁻¹, 743 cm⁻¹, 756 cm⁻¹, 861 cm⁻¹, 878 cm⁻¹, 964 cm⁻¹, 996 cm⁻¹, 1030 cm⁻¹, 1089 cm⁻¹, 1141 cm⁻¹, 1215 cm⁻¹, 1265 cm⁻¹, 1305 cm⁻¹, 1401 cm⁻¹, 1430 cm⁻¹, 1463 cm⁻¹, 1507 cm⁻¹, 1544 cm⁻¹, 1622 cm⁻¹, 1681 cm⁻¹, 1704 cm⁻¹, 2584 cm⁻¹, 2942 cm⁻¹, and 3414 cm⁻¹.

In some embodiments, crystalline Compound I Form 2 is characterized by an FT-IR spectrum having a signal at one or more of 861 cm⁻¹, 878 cm⁻¹, 1265 cm⁻¹, 1704 cm⁻¹,” 1305 cm⁻¹, and 2584 cm⁻¹.

In some embodiments, crystalline Compound I Form 2 is characterized by an FT-IR spectrum having a signal at one or more of 861 cm⁻¹, 878 cm⁻¹, 1305 cm⁻¹,” 1265 cm⁻¹, and 1704 cm⁻¹.

In some embodiments, crystalline Compound I Form 2 is characterized by an FT-IR spectrum having a signal at 861 cm⁻¹. In some embodiments, crystalline Compound I Form 2 is characterized by an FT-IR spectrum having a signal at 878 cm⁻¹. In some embodiments, crystalline Compound I Form 2 is characterized by an FT-IR spectrum having a signal at 1265 cm⁻¹. In some embodiments, crystalline Compound I Form 2 is characterized by an FT-IR spectrum having a signal at 1305 cm⁻¹. In some embodiments, crystalline Compound I Form 2 is characterized by an FT-IR spectrum having a signal at 1704 cm⁻¹. In some embodiments, crystalline Compound I Form 2 is characterized by an FT-IR spectrum having a signal at 2584 cm⁻¹.

Compound I Form 2 is characterized by an FT-IR spectrum substantially similar to FIG. 13.

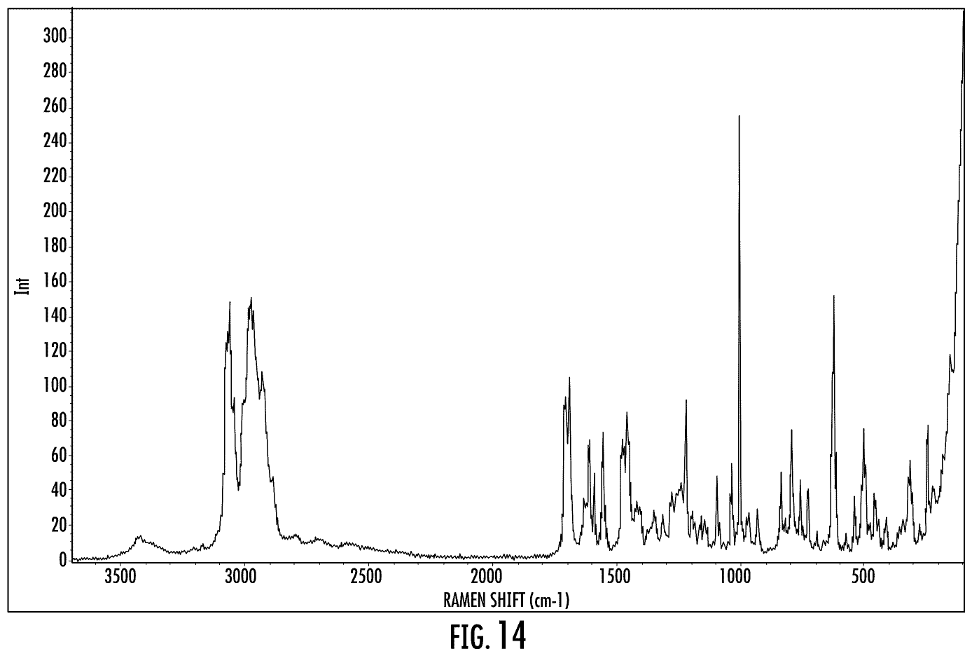

In some embodiments, Compound I Form 2 provides FT-Raman spectrum of FIG. 14 crystalline Compound I Form 2 with corresponding tabulated data shown in Table 7.

TABLE 7
FT-Raman Peaks for Crystalline Compound I Form 2

| Wavenumber | Relative |
|---|---|
| (cm⁻¹) | Intensity |
| 147 | 46 |
| 239 | 30 |
| 311 | 22 |
| 456 | 15 |
| 491 | 21 |
| 498 | 29 |
| 535 | 14 |
| 618 | 59 |
| 722 | 16 |
| 754 | 18 |
| 790 | 29 |
| 830 | 19 |
| 926 | 11 |
| 1002 | 100 |
| 1031 | 22 |
| 1090 | 19 |
| 1215 | 36 |
| 1452 | 33 |
| 1470 | 27 |
| 1549 | 28 |
| 1584 | 19 |
| 1602 | 27 |
| 1629 | 14 |
| 1685 | 41 |
| 1704 | 36 |
| 2972 | 59 |
| 3055 | 58 |

In some embodiments, crystalline Compound I Form 2 is characterized by an FT-Raman spectrum having a signal at one or more of 147 cm-1, 239 cm-1, 311 cm-1, 456 cm-1, 491 cm-1, 498 cm-1, 535 cm-1, 618 cm-1, 722 cm-1, 754 cm-1, 790 cm-1, 830 cm-1, 926 cm-1, 1002 cm-1, 1031 cm-1, 1090 cm-1, 1215 cm-1, 1452 cm-1, 1470 cm-1, 1549 cm-1, 1584 cm-1, 1602 cm-1, 1629 cm-1, 1685 cm-1, 1704 cm-1, 2972 cm-1, and 3055 cm-1.

In some embodiments, crystalline Compound I Form 2 is characterized by an FT-Raman spectrum having a signal at one or more of 239 cm-1, 790 cm-1, 1704 cm-1, and 3055 cm-1.

In some embodiments, crystalline Compound I Form 2 is characterized by an FT-Raman spectrum having a signal at one or more of 239 cm-1, 790 cm-1, and 1704 cm-1.

In some embodiments, crystalline Compound I Form 2 is characterized by an FT-Raman spectrum having a signal at 239 cm-1. In some embodiments, crystalline Compound I Form 2 is characterized by an FT-Raman spectrum having a signal at 790 cm-1. In some embodiments, crystalline Compound I Form 2 is characterized by an FT-Raman spectrum having a signal at 1704 cm-1. In some embodiments, crystalline Compound I Form 2 is characterized by an FT-Raman spectrum having a signal at 3055 cm-1.

In some embodiments, Compound I Form 2 is characterized by an FT-Raman spectrum substantially similar to FIG. 14.

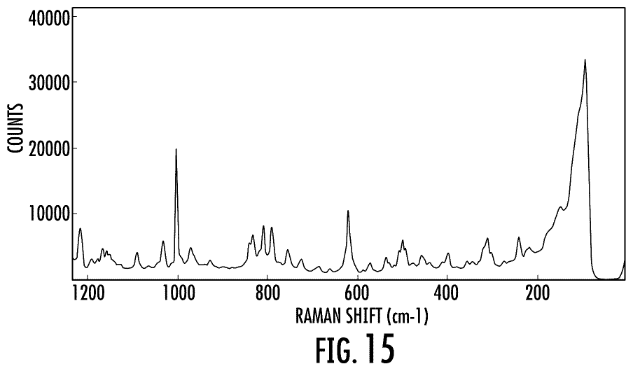

In some embodiments, Compound I Form 2 provides LF Raman spectrum of FIG. 15 crystalline Compound I Form 2 with corresponding tabulated data shown in Table 8.

TABLE 8
LF Raman Peaks for Crystalline Compound I Form 2

| Wavenumber | Relative |
|---|---|
| (cm⁻¹) | Intensity |
| 92 | 100 |
| 149 | 33 |
| 217 | 15 |
| 240 | 20 |
| 310 | 19 |
| 397 | 13 |
| 458 | 11 |
| 499 | 18 |
| 535 | 11 |
| 571 | 8 |
| 620 | 31 |
| 684 | 6 |
| 724 | 9 |
| 755 | 7 |
| 790 | 11 |
| 808 | 11 |
| 832 | 10 |
| 928 | 3 |
| 970 | 8 |

In some embodiments, crystalline Compound I Form 2 is characterized by an LF Raman spectrum having a signal at one or more of 92 cm⁻¹, 149 cm⁻¹, 217 cm⁻¹, 240 cm⁻¹, 310 cm⁻¹, 397 cm⁻¹, cm⁻¹, 458 cm⁻¹, 499 cm⁻¹, 535 cm⁻¹, 571 cm⁻¹, 620 cm⁻¹, 684 cm⁻¹, 724 cm⁻¹, 755 cm⁻¹, 790 cm⁻¹, 808 cm⁻¹, 832 cm⁻¹, 928 cm⁻¹, and 970 cm⁻¹.

In some embodiments, crystalline Compound I Form 2 is characterized by an LF Raman spectrum having a signal at one or more of 92 cm⁻¹, 149 cm⁻¹, 240 cm⁻¹, and 790 cm⁻¹.

In some embodiments, crystalline Compound I Form 2 is characterized by an LF Raman spectrum having a signal at one or more of 92 cm⁻¹ and 790 cm⁻¹.

In some embodiments, crystalline Compound I Form 2 is characterized by an LF Raman spectrum having a signal at one or more of 149 cm⁻¹ and 240 cm⁻¹.

In some embodiments, crystalline Compound I Form 2 is characterized by an LF Raman spectrum having a signal at 92 cm⁻¹. In some embodiments, crystalline Compound I Form 2 is characterized by an LF Raman spectrum having a signal at 149 cm⁻¹. In some embodiments, crystalline Compound I Form 2 is characterized by an LF Raman spectrum having a signal at 240 cm⁻¹. In some embodiments, crystalline Compound I Form 2 is characterized by an LF Raman spectrum having a signal at 790 cm⁻¹.

In some embodiments, Compound I Form 2 is characterized by an LF Raman spectrum substantially similar to FIG. 15.

Another embodiment of the disclosure provides a method of making crystalline Compound I Form 2. In some embodiments, the method of making crystalline Compound I Form 2 comprises: (i) combining methyl 1-benzyl-4-oxopiperidine-3-carboxylate·HCl, K₂CO₃, and toluene, (ii) stir for 30 min, (iii) adding N-(2,4-difluorobenzyl)-4,5-dihydro-1H-imidazol-2-amine and pyridinium p-toluenesulfonate (PPTS), (iv) refluxing at 110° C. for 8 hr (v) cooling to rt, (vi) diluting with water and stirring for 15 min, (vii) distilling the organic layer and heating to 80° C., (viii) cooling to 65° C., (ix) adding MTBE and cooling to rt, (x) collecting the solids, (xi) drying the solids in a vacuum oven at 40° C. for overnight, (xii) adding ethanol to dried solids and heating to 70° C., (xiii) adding conc. HCl and cooling to 50° C. for 1 hr, (xiv) cooling to 0° C. for 12 hr (xv) collecting solids, (xvi) drying the solids in a vacuum oven to yield crystalline Compound I Form 2.

In some embodiments, the method of making crystalline Compound I Form 2 comprises (i) adding ethanol and water to Compound I free base, (ii) heating to 70° C., (iii) adding conc. HCl and cooling to 50° C. for 1 hr, (iv) cooling to 0° C. for 12 hr (v) collecting solids, (vi) drying the solids in a vacuum oven to yield crystalline Compound I Form 2.

In some embodiments, the method of making crystalline Compound I Form 2 comprises (i) adding ethanol and water to Compound I Form 1, (ii) stirring at room temperature, (iii) collecting solids, (iv) drying the solids in a vacuum oven to yield crystalline Compound I Form 2.

In some embodiments, the method of making crystalline Compound I Form 2 comprises (i) adding Compound I Form 1 to ethanol and water at 70° C., (ii) cooling to 0° C., (iii) collecting solids, (iv) drying the solids in a vacuum oven to yield crystalline Compound I Form 2.

In some embodiments, the method of making crystalline Compound I Form 2 comprises (i) slurrying Compound I Form 1 in 91% ethanol:water, (ii) stirring at rt for 7 days, and (iii) centrifuging and removing the supernatant yield crystalline Compound I Form 2.

### C. Compound I Form 3

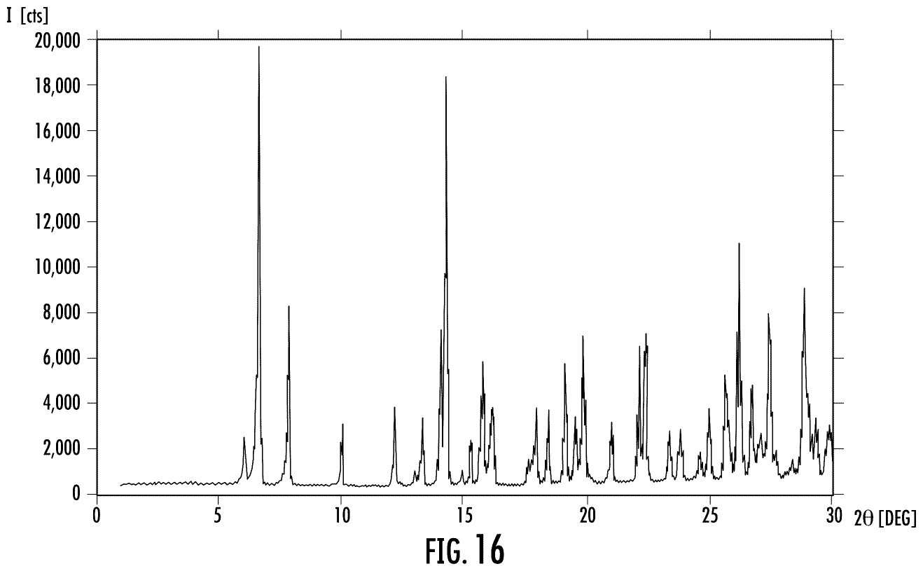

In some embodiments, the disclosure provides crystalline Compound I Form 3. In some embodiments, Compound I Form 3 is an acetonitrile solvate. In some embodiments, Compound I Form 3 is an acetonitrile solvate with up to 1.4 moles of acetonitrile. FIG. 16 provides an X-ray powder diffractogram of crystalline Compound I Form 3 with corresponding tabulated data for FIG. 16 shown in Table 9.

TABLE 9
XRPD Signals for Crystalline Compound I Form 3

| °2Θ | d-spacing | Intensity | Relative |
|---|---|---|---|
| (±0.2°) | (Å) | (Counts) | Intensity |
| 6.09 | 14.5 | 2540 | 13 |
| 6.67 | 13.24 | 19636 | 100 |
| 7.88 | 11.21 | 8253 | 42 |
| 10.07 | 8.78 | 3070 | 16 |
| 12.2 | 7.25 | 3871 | 20 |
| 13.02 | 6.79 | 1083 | 6 |
| 13.29 | 6.66 | 3371 | 17 |
| 14.06 | 6.29 | 7110 | 36 |
| 14.31 | 6.18 | 18378 | 94 |
| 14.91 | 5.94 | 1025 | 5 |
| 15.28 | 5.79 | 2369 | 12 |
| 15.78 | 5.61 | 5810 | 30 |
| 16.18 | 5.47 | 3749 | 19 |
| 17.95 | 4.94 | 3793 | 19 |
| 18.45 | 4.81 | 3694 | 19 |
| 19.11 | 4.64 | 5678 | 29 |
| 19.56 | 4.53 | 3383 | 17 |
| 19.88 | 4.46 | 6987 | 36 |
| 21.03 | 4.22 | 3162 | 16 |
| 22.16 | 4.01 | 6448 | 33 |
| 22.42 | 3.96 | 7035 | 36 |
| 23.37 | 3.8 | 2752 | 14 |
| 23.82 | 3.73 | 2791 | 14 |
| 24.64 | 3.61 | 1850 | 9 |
| 24.99 | 3.56 | 3780 | 19 |
| 25.66 | 3.47 | 5245 | 27 |
| 26.21 | 3.4 | 11028 | 56 |
| 26.74 | 3.33 | 4733 | 24 |
| 27.06 | 3.29 | 2613 | 13 |
| 27.45 | 3.25 | 7895 | 40 |
| 28.88 | 3.09 | 8999 | 46 |
| 29.35 | 3.04 | 3380 | 17 |
| 29.89 | 2.99 | 3027 | 15 |
| 32.63 | 2.74 | 2337 | 12 |
| 33.19 | 2.7 | 3006 | 15 |
| 35.12 | 2.55 | 1586 | 8 |
| 37.47 | 2.4 | 1104 | 6 |
| 37.91 | 2.37 | 896 | 5 |

In some embodiments, crystalline Compound I Form 3 is substantially pure. In some embodiments, crystalline Compound I Form 3 is substantially crystalline. In some embodiments, crystalline Compound I Form 3 is characterized by an X-ray powder diffractogram generated by an X-ray powder diffraction analysis with an incident beam of Cu Kα radiation.

In some embodiments, crystalline Compound I Form 3 is characterized by an X-ray powder diffractogram having a signal at one or more of 6.09±2 degrees two-theta, 6.67±2 degrees two-theta, 7.88±2 degrees two-theta, 10.07±2 degrees two-theta, 12.2±2 degrees two-theta, 13.02±2 degrees two-theta, ±2 degrees two-theta, 13.29±2 degrees two-theta, 14.06±2 degrees two-theta, 14.31±2 degrees two-theta, 14.91±2 degrees two-theta, 15.28±2 degrees two-theta, 15.78±2 degrees two-theta, 16.18±2 degrees two-theta, 17.95±2 degrees two-theta, 18.45±2 degrees two-theta, 19.11±2 degrees two-theta, 19.56±2 degrees two-theta, 19.88±2 degrees two-theta, 21.03±2 degrees two-theta, 22.16±2 degrees two-theta, 22.42±2 degrees two-theta, 23.37±2 degrees two-theta, 23.82±2 degrees two-theta, 24.64±2 degrees two-theta, 24.99±2 degrees two-theta, 25.66±2 degrees two-theta, 26.21±2 degrees two-theta, 26.74±2 degrees two-theta, 27.06±2 degrees two-theta, 27.45±2 degrees two-theta, 28.88±2 degrees two-theta, 29.35±2 degrees two-theta, 29.89±2 degrees two-theta, 32.63±2 degrees two-theta, 33.19±2 degrees two-theta, 35.12±2 degrees two-theta, 37.47 and 37.91±2 degrees two-theta.

In some embodiments, crystalline Compound I Form 3 is characterized by an X-ray powder diffractogram having a signal at one or more of 6.09±2 degrees two-theta, 6.67±2 degrees two-theta, 10.07±2 degrees two-theta, 12.2±2 degrees two-theta, and 14.31±2 degrees two-theta.

In some embodiments, crystalline Compound I Form 3 is characterized by an X-ray powder diffractogram having a signal at one or more of 6.09±2 degrees two-theta, 6.67±2 degrees two-theta, 10.07±2 degrees two-theta, and 12.2±2 degrees two-theta.

In some embodiments, crystalline Compound I Form 3 is characterized by an X-ray powder diffractogram having a signal at one or more of 6.09±2 degrees two-theta, 6.67±2 degrees two-theta, and 10.07±2 degrees two-theta.

In some embodiments, crystalline Compound I Form 3 is characterized by an X-ray powder diffractogram having a signal at one or more of 6.67±2 degrees two-theta, and 10.07±2 degrees two-theta.

In some embodiments, crystalline Compound I Form 3 is characterized by an X-ray powder diffractogram having a signal at 6.09±2 degrees two-theta. In some embodiments, crystalline Compound I Form 3 is characterized by an X-ray powder diffractogram having a signal at 6.67±2 degrees two-theta. In some embodiments, crystalline Compound I Form 3 is characterized by an X-ray powder diffractogram having a signal at 10.07±2 degrees two-theta. In some embodiments, crystalline Compound I Form 3 is characterized by an X-ray powder diffractogram having a signal at 12.2±2 degrees two-theta. In some embodiments, crystalline Compound I Form 3 is characterized by an X-ray powder diffractogram having a signal at 14.31±2 degrees two-theta.

In some embodiments, crystalline Compound I Form 3 is characterized by an X-ray powder diffractogram substantially similar to FIG. 16.

In some embodiments, crystalline Compound I Form 3 is characterized by a primitive monoclinic crystal system, P2₁/n space group and the following unit cell dimensions resulting from successful indexing of Form 3 XRPD:

| | | | |
|---|---|---|---|
| a | 13.6 ± 0.1 Å | α | 90 ± 0.1° |
| b | 7.0 ± 0.1 Å | β | 102.5 ± 0.1° |
| c | 29.7 ± 0.1 Å | γ | 90 ± 0.1°. |

In some embodiments, Compound I Form 3 is characterized by a DSC having a broad endotherm peak temperature of 88° C.

In some embodiments, Compound I Form 3 is characterized by a DSC having a broad endotherm peak temperature of 149° C.

In some embodiments, Compound I Form 3 is characterized by a DSC having an onset of melting temperature of 217° C. In some embodiments, Compound I Form 3 is characterized by a DSC having a broad endotherm peak temperature of 227° C. In some embodiments, Compound I Form 3 is characterized by a DSC having an onset of melting temperature of 217° C. and/or a peak temperature of 227° C.

In some embodiments, Compound I Form 3 is characterized by a DSC having two endotherms at peak temperatures of 88° C. and 149° C. In some embodiments, Compound I Form 3 is characterized by a DSC having three endotherms at peak temperatures of 83° C., 149° C., and 227° C. In some embodiments, Compound I Form 3 is characterized by a DSC having two broad endotherms at peak temperatures of 83° C. and 149° C. and having a large endotherm with an onset of melting temperature of 217° C. and/or a peak temperature of 227° C.

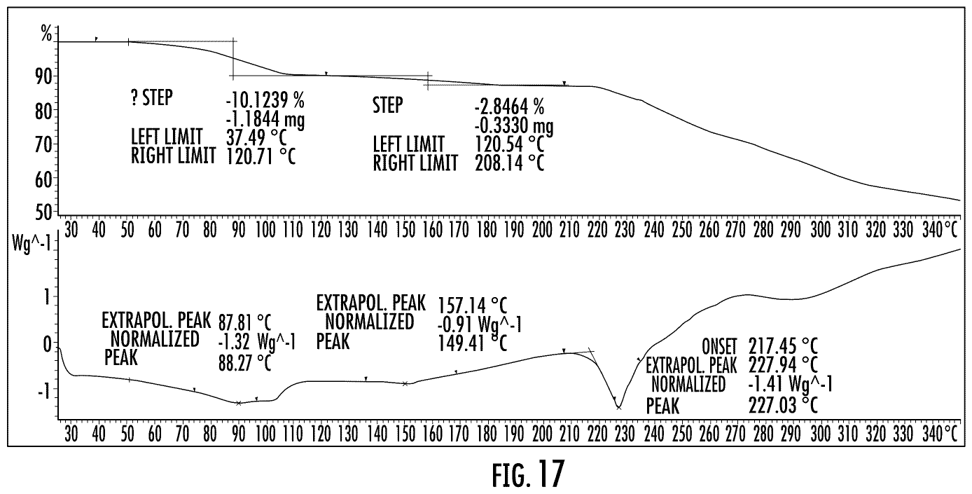

In some embodiments, Compound I Form 3 is characterized by a DSC substantially similar to FIG. 17.

In some embodiments, Compound I Form 3 is also characterized by a weight loss of 10.1% from 37° C. to 121° C., as measured by thermogravimetric analysis. In some embodiments, Compound I Form 3 is also characterized by a weight loss of approximately 2.9% at temperatures 121° C. to 208° C., as measured by thermogravimetric analysis.

In some embodiments, Compound I Form 3 is characterized by a TGA substantially similar to FIG. 17.

Another embodiment of the disclosure provides a method of making crystalline Compound I Form 3. In some embodiments, the method of making crystalline Compound I Form 3 comprises: slurrying Compound I Form 1 in acetonitrile at rt for 3 to yield crystalline Compound I Form 3.

### D. Compound I Form 4

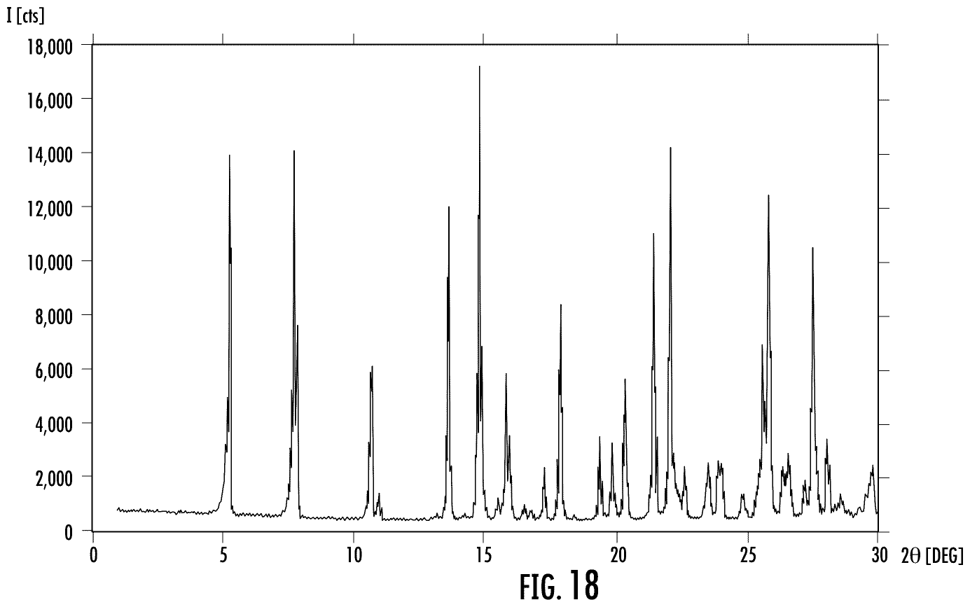

In some embodiments, the disclosure provides crystalline Compound I Form 4. In some embodiments, Compound I Form 4 is an isopropanol solvate. In some embodiments, Compound I Form 4 is an isopropanol solvate with up to 1 mole of isopropanol. In some embodiments, the solvate may be a variable solvate, for example with from about 1 to about 2 moles of solvate. FIG. 18 provides an X-ray powder diffractogram of crystalline Compound I Form 4 with the tabulated data for FIG. 18 shown in Table 10.

TABLE 10
XRPD Signals for Crystalline Compound I Form 4

| °2Θ | d-spacing | Intensity | Relative |
|---|---|---|---|
| (±0.2°) | (Å) | (Counts) | Intensity |
| 5.29 | 16.69 | 13925 | 81 |
| 7.73 | 11.43 | 14081 | 82 |
| 7.86 | 11.24 | 7648 | 44 |
| 10.67 | 8.28 | 6099 | 35 |
| 10.97 | 8.06 | 1441 | 8 |
| 13.63 | 6.49 | 11972 | 70 |
| 14.81 | 5.98 | 17197 | 100 |
| 15.48 | 5.72 | 1241 | 7 |
| 15.76 | 5.62 | 5804 | 34 |
| 15.93 | 5.56 | 3548 | 21 |
| 16.48 | 5.37 | 1014 | 6 |
| 16.75 | 5.29 | 796 | 5 |
| 17.25 | 5.14 | 2301 | 13 |
| 17.87 | 4.96 | 8426 | 49 |
| 19.36 | 4.58 | 3486 | 20 |
| 19.83 | 4.47 | 3250 | 19 |
| 20.33 | 4.36 | 5610 | 33 |
| 21.41 | 4.15 | 10995 | 64 |
| 22.03 | 4.03 | 14174 | 82 |
| 22.58 | 3.93 | 2400 | 14 |
| 23.49 | 3.78 | 2555 | 15 |
| 23.99 | 3.71 | 2523 | 15 |
| 24.81 | 3.59 | 1337 | 8 |
| 25.54 | 3.48 | 6872 | 40 |
| 25.79 | 3.45 | 12436 | 72 |
| 26.33 | 3.38 | 2390 | 14 |
| 26.54 | 3.36 | 2907 | 17 |
| 27.16 | 3.28 | 1853 | 11 |
| 27.46 | 3.25 | 10480 | 61 |
| 28.01 | 3.18 | 3359 | 20 |
| 28.3 | 3.15 | 1004 | 6 |
| 28.57 | 3.12 | 1354 | 8 |
| 29.74 | 3 | 2422 | 14 |
| 31.07 | 2.88 | 2129 | 12 |
| 31.41 | 2.85 | 2085 | 12 |
| 31.82 | 2.81 | 1626 | 9 |
| 32.17 | 2.78 | 1833 | 11 |
| 32.54 | 2.75 | 1079 | 6 |
| 32.79 | 2.73 | 1239 | 7 |
| 33.19 | 2.7 | 1354 | 8 |
| 36.77 | 2.44 | 1426 | 8 |
| 38.19 | 2.35 | 965 | 6 |
| 38.63 | 2.33 | 912 | 5 |

In some embodiments, crystalline Compound I Form 4 is substantially pure. In some embodiments, crystalline Compound I Form 4 is substantially crystalline. In some embodiments, crystalline Compound I Form 4 is characterized by an X-ray powder diffractogram generated by an X-ray powder diffraction analysis with an incident beam of Cu Kα radiation.

In some embodiments, crystalline Compound I Form 4 is characterized by an X-ray powder diffractogram having a signal at one or more of 5.29±2 degrees two-theta, 7.73±2 degrees two-theta, 7.86±2 degrees two-theta, 10.67±2 degrees two-theta, 10.97±2 degrees two-theta, 13.63±2 degrees two-theta, ±2 degrees two-theta, 14.81±2 degrees two-theta, 15.48±2 degrees two-theta, 15.76±2 degrees two-theta, 15.93±2 degrees two-theta, 16.48±2 degrees two-theta, 16.75±2 degrees two-theta, 17.25±2 degrees two-theta, 17.87±2 degrees two-theta, 19.36±2 degrees two-theta, 19.83±2 degrees two-theta, 20.33±2 degrees two-theta, 21.41±2 degrees two-theta, 22.03±2 degrees two-theta, 22.58±2 degrees two-theta, 23.49±2 degrees two-theta, 23.99±2 degrees two-theta, 24.81±2 degrees two-theta, 25.54±2 degrees two-theta, 25.79±2 degrees two-theta, 26.33±2 degrees two-theta, 26.54±2 degrees two-theta, 27.16±2 degrees two-theta, 27.46±2 degrees two-theta, 28.01±2 degrees two-theta, 28.3±2 degrees two-theta, 28.57±2 degrees two-theta, 29.74±2 degrees two-theta, 31.07±2 degrees two-theta, 31.41±2 degrees two-theta, 31.82±2 degrees two-theta, 32.17±2 degrees two-theta, 32.54±2 degrees two-theta, 32.79±2 degrees two-theta, 33.19±2 degrees two-theta, 36.77±2 degrees two-theta, 38.19±2 degrees two-theta, and 38.63±2 degrees two-theta.

In some embodiments, crystalline Compound I Form 4 is characterized by an X-ray powder diffractogram having a signal at one or more of 5.29±2 degrees two-theta, 7.73±2 degrees two-theta, 10.67±2 degrees two-theta, 13.63±2 degrees two-theta, and 14.81±2 degrees two-theta.

In some embodiments, crystalline Compound I Form 4 is characterized by an X-ray powder diffractogram having a signal at one or more of 7.73±2 degrees two-theta, 10.67±2 degrees two-theta, 13.63±2 degrees two-theta, and 14.81±2 degrees two-theta.

In some embodiments, crystalline Compound I Form 4 is characterized by an X-ray powder diffractogram having a signal at one or more of 7.73±2 degrees two-theta, 13.63±2 degrees two-theta, and 14.81±2 degrees two-theta.

In some embodiments, crystalline Compound I Form 4 is characterized by an X-ray powder diffractogram having a signal at one or more of 7.73±2 degrees two-theta, and 14.81±2 degrees two-theta.

In some embodiments, crystalline Compound I Form 4 is characterized by an X-ray powder diffractogram having a signal at 5.29±2 degrees two-theta. In some embodiments, crystalline Compound I Form 4 is characterized by an X-ray powder diffractogram having a signal at 7.73±2 degrees two-theta. In some embodiments, crystalline Compound I Form 4 is characterized by an X-ray powder diffractogram having a signal at 10.67±2 degrees two-theta. In some embodiments, crystalline Compound I Form 4 is characterized by an X-ray powder diffractogram having a signal at 13.63±2 degrees two-theta. In some embodiments, crystalline Compound I Form 4 is characterized by an X-ray powder diffractogram having a signal at 14.81±2 degrees two-theta.

In some embodiments, crystalline Compound I Form 4 is characterized by an X-ray powder diffractogram substantially similar to FIG. 18.

In some embodiments, crystalline Compound I Form 4 is characterized by a primitive monoclinic crystal system, P2₁/C space group and the following unit cell dimensions resulting from successful indexing of Form 4 XRPD:

| | | | |
|---|---|---|---|
| a | 17.9 ± 0.1 Å | α | 90 ± 0.1° |
| b | 7.0 ± 0.1 Å | β | 111.3 ± 0.1° |
| c | 24.1 ± 0.1 Å | γ | 90 ± 0.1°. |

In some embodiments, Compound I Form 4 is characterized by a DSC having a small broad endotherm peak temperature of 129° C.

In some embodiments, Compound I Form 4 is characterized by a DSC having a broad endotherm peak temperature of 150° C.

In some embodiments, Compound I Form 4 is characterized by a DSC having an onset of melting temperature of 224° C. In some embodiments, Compound I Form 4 is characterized by a DSC having a broad endotherm peak temperature of 231° C. In some embodiments, Compound I Form 4 is characterized by a DSC having an onset of melting temperature of 224° C. and/or a peak temperature of 231° C.

In some embodiments, Compound I Form 4 is characterized by a DSC having two endotherms at peak temperatures of 129° C. and 150° C. In some embodiments, Compound I Form 4 is characterized by a DSC having three endotherms at peak temperatures of 129° C., 150° C., and 231° C. In some embodiments, Compound I Form 4 is characterized by a DSC having two broad endotherms at peak temperatures of 129° C. and 150° C. and having a large endotherm with an onset of melting temperature of 224° C. and/or a peak temperature of 231° C.

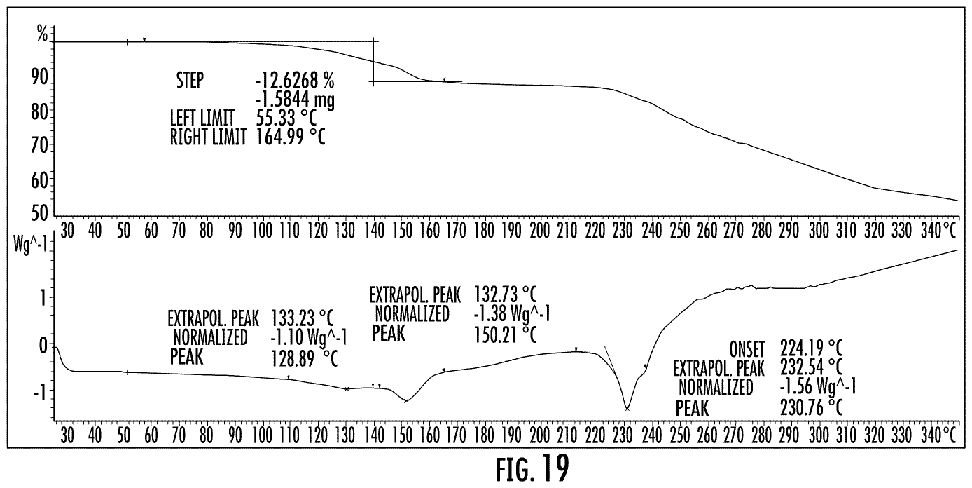

In some embodiments, Compound I Form 4 is characterized by a DSC substantially similar to FIG. 19.

In some embodiments, Compound I Form 4 is also characterized by a weight loss of 12.6% from 55° C. to 165° C., as measured by thermogravimetric analysis. In some embodiments, Compound I Form 4 is also characterized by a weight loss of approximately 12.6% at temperatures 121° C. to 208° C., as measured by thermogravimetric analysis.

In some embodiments, Compound I Form 4 is characterized by a TGA substantially similar to FIG. 19.

Another embodiment of the disclosure provides a method of making crystalline Compound I Form 4. In some embodiments, the method of making crystalline Compound I Form 4 comprises: slurrying Compound I Form 1 in isopropanol (IPA) at rt for 3 days to yield crystalline Compound I Form 4.

### E. Compound I Form 5

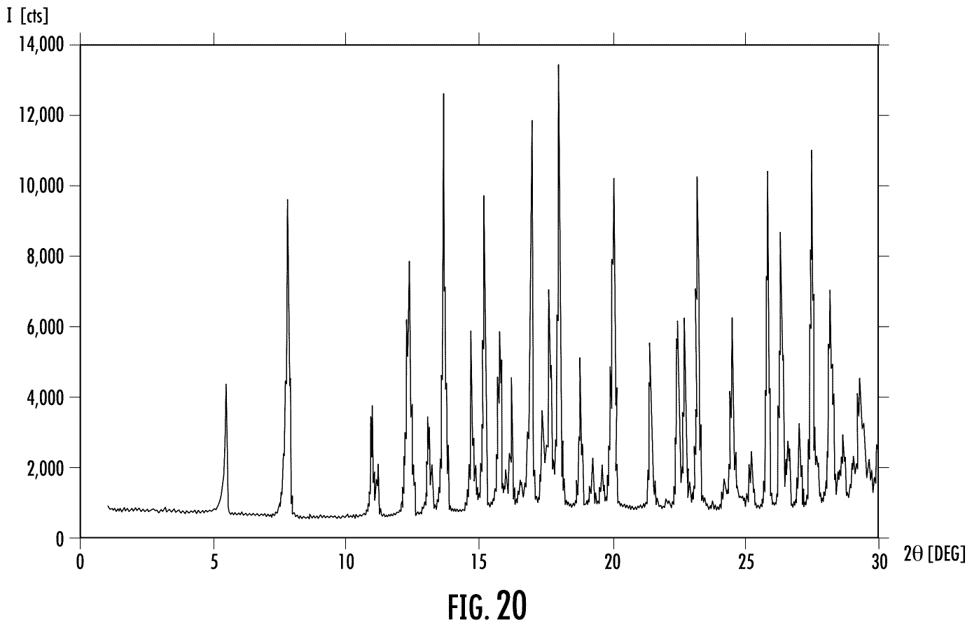

In some embodiments, the disclosure provides crystalline Compound I Form 5. In some embodiments, Compound I Form 5 is an N-methylpyrrolidone solvate. In some embodiments, Compound I Form 5 is an N-methylpyrrolidone solvate with up to 3 moles of N-methylpyrrolidone. FIG. 20 provides an X-ray powder diffractogram of crystalline Compound I Form 5 with corresponding tabulated data for FIG. 20 shown in Table 11.

TABLE 11
XRPD Signals for Crystalline Compound I Form 5

| °2Θ | d-spacing | Intensity | Relative |
|---|---|---|---|
| (±0.2°) | (Å) | (Counts) | Intensity |
| 5.49 | 16.08 | 4322 | 32 |
| 7.79 | 11.34 | 9540 | 71 |
| 11 | 8.04 | 3693 | 28 |
| 11.2 | 7.89 | 2053 | 15 |
| 12.29 | 7.2 | 6149 | 46 |
| 12.41 | 7.13 | 7809 | 58 |
| 12.54 | 7.05 | 2013 | 15 |
| 13.09 | 6.76 | 3341 | 25 |
| 13.24 | 6.68 | 1939 | 14 |
| 13.68 | 6.47 | 12590 | 94 |
| 14.73 | 6.01 | 5799 | 43 |
| 14.88 | 5.95 | 1922 | 14 |
| 15.2 | 5.82 | 9661 | 72 |
| 15.8 | 5.6 | 5806 | 43 |
| 16 | 5.53 | 1846 | 14 |
| 16.23 | 5.46 | 4516 | 34 |
| 16.58 | 5.34 | 1594 | 12 |
| 17 | 5.21 | 11823 | 88 |
| 17.4 | 5.09 | 3569 | 27 |
| 17.65 | 5.02 | 7001 | 52 |
| 18 | 4.92 | 13427 | 100 |
| 18.82 | 4.71 | 5061 | 38 |
| 19.31 | 4.59 | 2187 | 16 |
| 19.66 | 4.51 | 2019 | 15 |
| 20.09 | 4.42 | 10196 | 76 |
| 21.46 | 4.14 | 5480 | 41 |
| 22.5 | 3.95 | 6121 | 46 |
| 22.75 | 3.91 | 6226 | 46 |
| 23.23 | 3.83 | 10227 | 76 |
| 24.25 | 3.67 | 1586 | 12 |
| 24.55 | 3.62 | 6239 | 46 |
| 25.27 | 3.52 | 2366 | 18 |
| 25.87 | 3.44 | 10409 | 78 |
| 26.38 | 3.38 | 8645 | 64 |
| 26.63 | 3.34 | 2679 | 20 |
| 27.06 | 3.29 | 3170 | 24 |
| 27.53 | 3.24 | 10989 | 82 |
| 28.23 | 3.16 | 7010 | 52 |
| 28.7 | 3.11 | 2873 | 21 |
| 29.12 | 3.06 | 2265 | 17 |
| 29.3 | 3.05 | 4502 | 34 |
| 29.7 | 3.01 | 2171 | 16 |
| 30.02 | 2.97 | 2813 | 21 |
| 30.55 | 2.92 | 4041 | 30 |
| 31.69 | 2.82 | 1571 | 12 |
| 32.41 | 2.76 | 2284 | 17 |
| 32.74 | 2.73 | 2150 | 16 |
| 33.09 | 2.71 | 1303 | 10 |
| 34.31 | 2.61 | 1984 | 15 |
| 34.66 | 2.59 | 1840 | 14 |
| 35.02 | 2.56 | 1275 | 9 |
| 35.8 | 2.51 | 1245 | 9 |
| 36.95 | 2.43 | 1171 | 9 |
| 38.21 | 2.35 | 1298 | 10 |
| 39.18 | 2.3 | 1212 | 9 |

In some embodiments, crystalline Compound I Form 5 is substantially pure. In some embodiments, crystalline Compound I Form 5 is substantially crystalline. In some embodiments, crystalline Compound I Form 5 is characterized by an X-ray powder diffractogram generated by an X-ray powder diffraction analysis with an incident beam of Cu Kα radiation.

In some embodiments, crystalline Compound I Form 5 is characterized by an X-ray powder diffractogram having a signal at one or more of 5.49±2 degrees two-theta, 7.79±2 degrees two-theta, 11±2 degrees two-theta, 11.2±2 degrees two-theta, 12.29±2 degrees two-theta, 12.41±2 degrees two-theta, ±2 degrees two-theta, 12.54±2 degrees two-theta, 13.09±2 degrees two-theta, 13.24±2 degrees two-theta, 13.68±2 degrees two-theta, 14.73±2 degrees two-theta, 14.88±2 degrees two-theta, 15.2±2 degrees two-theta, 15.8±2 degrees two-theta, 16±2 degrees two-theta, 16.23±2 degrees two-theta, 16.58±2 degrees two-theta, 17±2 degrees two-theta, 17.4±2 degrees two-theta, 17.65±2 degrees two-theta, 18±2 degrees two-theta, 18.82±2 degrees two-theta, 19.31±2 degrees two-theta, 19.66±2 degrees two-theta, 20.09±2 degrees two-theta, 21.46±2 degrees two-theta, 22.5±2 degrees two-theta, 22.75±2 degrees two-theta, 23.23±2 degrees two-theta, 24.25±2 degrees two-theta, 24.55±2 degrees two-theta, 25.27±2 degrees two-theta, 25.87±2 degrees two-theta, 26.38±2 degrees two-theta, 26.63±2 degrees two-theta, 27.06±2 degrees two-theta, 27.53±2 degrees two-theta, 28.23±2 degrees two-theta, 28.7±2 degrees two-theta, 29.12±2 degrees two-theta, 29.3±2 degrees two-theta, 29.7±2 degrees two-theta, 30.02±2 degrees two-theta, 30.55±2 degrees two-theta, 31.69±2 degrees two-theta, 32.41±2 degrees two-theta, 32.74±2 degrees two-theta, 33.09±2 degrees two-theta, 34.31±2 degrees two-theta, 34.66±2 degrees two-theta, 35.02±2 degrees two-theta, 35.8±2 degrees two-theta, 36.95±2 degrees two-theta, 38.21±2 degrees two-theta, and 39.18±2 degrees two-theta.

In some embodiments, crystalline Compound I Form 5 is characterized by an X-ray powder diffractogram having a signal at one or more of 5.49±2 degrees two-theta, 7.79±2 degrees two-theta, 11±2 degrees two-theta, 12.41±2 degrees two-theta, and 15.2±2 degrees two-theta.

In some embodiments, crystalline Compound I Form 5 is characterized by an X-ray powder diffractogram having a signal at one or more of 7.79±2 degrees two-theta, 11±2 degrees two-theta, 12.41±2 degrees two-theta, and 15.2±2 degrees two-theta.

In some embodiments, crystalline Compound I Form 5 is characterized by an X-ray powder diffractogram having a signal at one or more of 7.79±2 degrees two-theta, 11±2 degrees two-theta, and 12.41±2 degrees two-theta.

In some embodiments, crystalline Compound I Form 5 is characterized by an X-ray powder diffractogram having a signal at one or more of 7.79±2 degrees two-theta, and 12.41±2 degrees two-theta.

In some embodiments, crystalline Compound I Form 5 is characterized by an X-ray powder diffractogram having a signal at 5.49±2 degrees two-theta. In some embodiments, crystalline Compound I Form 5 is characterized by an X-ray powder diffractogram having a signal at 7.79±2 degrees two-theta. In some embodiments, crystalline Compound I Form 5 is characterized by an X-ray powder diffractogram having a signal at 11±2 degrees two-theta. In some embodiments, crystalline Compound I Form 5 is characterized by an X-ray powder diffractogram having a signal at 12.41±2 degrees two-theta. In some embodiments, crystalline Compound I Form 5 is characterized by an X-ray powder diffractogram having a signal at 15.2±2 degrees two-theta.

In some embodiments, crystalline Compound I Form 5 is characterized by an X-ray powder diffractogram substantially similar to FIG. 20.

In some embodiments, crystalline Compound I Form 5 is characterized by a triclinic crystal system, P1₁ space group and the following unit cell dimensions resulting from successful indexing of Form 5 XRPD:

| | | | |
|---|---|---|---|
| a | 6.9 ± 0.1 Å | α | 88.2 ± 0.1° |
| b | 16.1 ± 0.1 Å | β | 83.8 ± 0.1° |
| c | 16.2 ± 0.1 Å | γ | 79.0 ± 0.1°. |

In some embodiments, Compound I Form 5 is characterized by a DSC having a small broad endotherm peak temperature of 104° C.

In some embodiments, Compound I Form 5 is characterized by a DSC having an endotherm peak temperature of 119° C.

In some embodiments, Compound I Form 5 is characterized by a DSC having a broad endotherm peak temperature of 215° C.

In some embodiments, Compound I Form 5 is characterized by a DSC having three endotherms at peak temperatures of 108° C., 119° C. and 215° C.

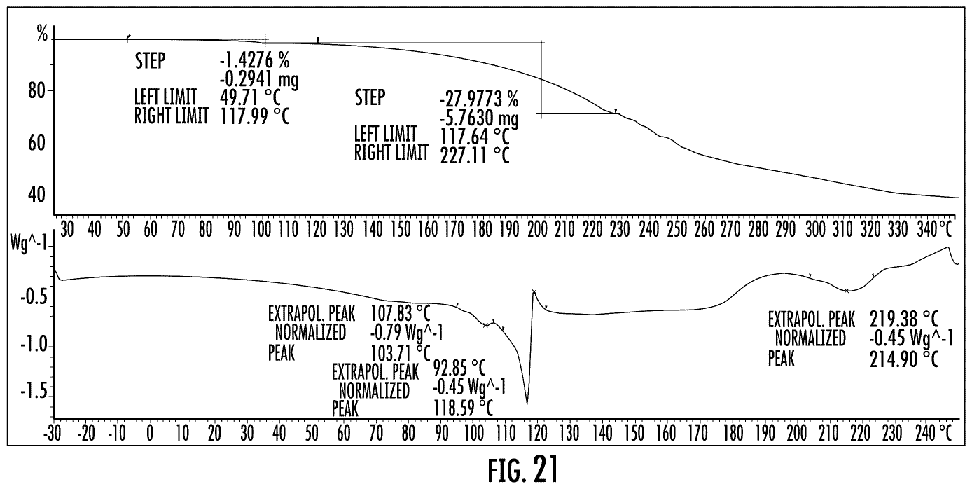

In some embodiments, Compound I Form 5 is characterized by a DSC substantially similar to FIG. 21.

In some embodiments, Compound I Form 5 is also characterized by a weight loss of 1.4% from 50° C. to 118° C., as measured by thermogravimetric analysis. In some embodiments, Compound I Form 5 is also characterized by a weight loss of approximately 28.0% at temperatures 118° C. to 227° C., as measured by thermogravimetric analysis.

In some embodiments, Compound I Form 5 is characterized by a TGA substantially similar to FIG. 21.

Another embodiment of the disclosure provides a method of making crystalline Compound I Form 5. In some embodiments, the method of making crystalline Compound I Form 5 comprises: slurrying Compound I Form 1 in N-methylpyrrolidone at rt for 3 days to yield crystalline Compound I Form 5.

### F. Compound I Material 6

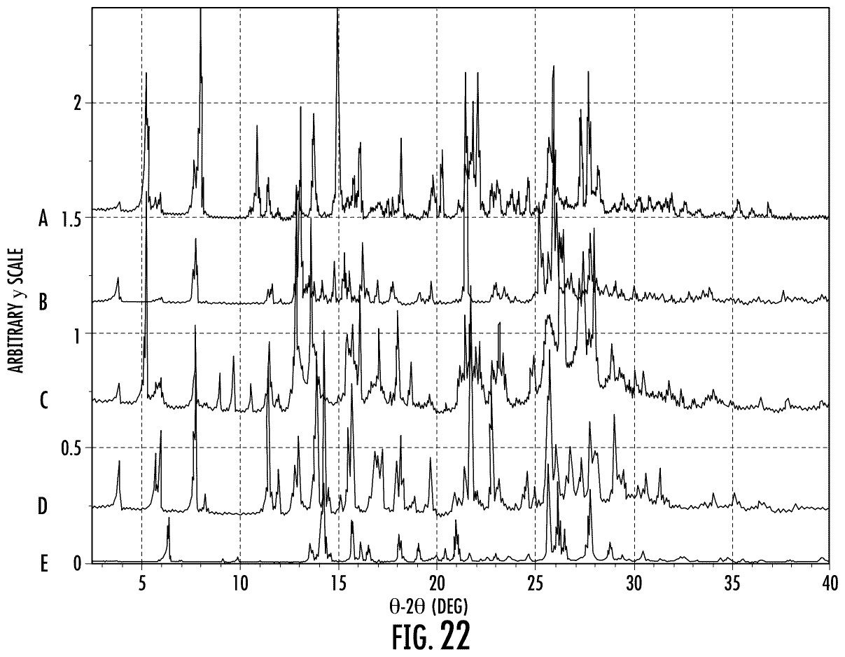

In some embodiments, the disclosure provides mixtures of Compound I Material 6+Form 2. In some embodiments, Compound I Material 6 is an ethanol solvate. FIG. 22 (A, top). provides an X-ray powder diffractogram of the Compound I Material 6+Form 2 mixture with corresponding tabulated data for FIG. 22 (A, top) shown in Table 12.

TABLE 12
XRPD Signals for Crystalline Compound I
Material 6 + Form 2

| °2Θ | d-spacing | Intensity | Relative |
|---|---|---|---|
| (±0.2°) | (Å) | (Counts) | Intensity |
| 3.85 | 22.93 | 1263 | 12 |
| 5.25 | 16.82 | 6927 | 66 |
| 5.35 | 16.51 | 4884 | 46 |
| 5.7 | 15.49 | 1405 | 13 |
| 5.95 | 14.84 | 1666 | 16 |
| 7.71 | 11.46 | 3069 | 29 |
| 7.99 | 11.06 | 10518 | 100 |
| 10.51 | 8.41 | 1040 | 10 |
| 10.88 | 8.13 | 4571 | 43 |
| 11.45 | 7.72 | 2320 | 22 |
| 11.95 | 7.4 | 991 | 9 |
| 12.99 | 6.81 | 1819 | 17 |
| 13.76 | 6.43 | 5140 | 49 |
| 14.99 | 5.91 | 10466 | 100 |
| 15.51 | 5.71 | 1454 | 14 |
| 15.79 | 5.61 | 2370 | 23 |
| 16.13 | 5.49 | 3875 | 37 |
| 17.05 | 5.2 | 1250 | 12 |
| 17.52 | 5.06 | 1361 | 13 |
| 17.78 | 4.98 | 1439 | 14 |
| 18.22 | 4.87 | 4006 | 38 |
| 19.39 | 4.57 | 871 | 8 |
| 19.84 | 4.47 | 2468 | 23 |
| 20.27 | 4.38 | 3474 | 33 |
| 21.11 | 4.21 | 1333 | 13 |
| 21.48 | 4.13 | 3399 | 32 |
| 21.86 | 4.06 | 5659 | 54 |
| 22.11 | 4.02 | 6863 | 65 |
| 22.38 | 3.97 | 1213 | 12 |
| 22.81 | 3.9 | 2005 | 19 |
| 23.06 | 3.85 | 2198 | 21 |
| 23.8 | 3.74 | 1849 | 18 |
| 24.15 | 3.68 | 1778 | 17 |
| 24.67 | 3.61 | 2359 | 22 |
| 25.07 | 3.55 | 995 | 9 |
| 25.7 | 3.46 | 4038 | 38 |
| 25.95 | 3.43 | 7619 | 72 |
| 27.34 | 3.26 | 5273 | 50 |
| 27.71 | 3.22 | 6915 | 66 |
| 28.21 | 3.16 | 2858 | 27 |
| 29.03 | 3.07 | 1145 | 11 |
| 29.45 | 3.03 | 1586 | 15 |
| 29.71 | 3 | 1111 | 11 |
| 30.28 | 2.95 | 1447 | 14 |
| 30.83 | 2.9 | 1489 | 14 |
| 31.27 | 2.86 | 1265 | 12 |
| 31.67 | 2.82 | 1364 | 13 |
| 31.94 | 2.8 | 1683 | 16 |
| 32.64 | 2.74 | 1269 | 12 |
| 33.39 | 2.68 | 916 | 9 |
| 35.35 | 2.54 | 1355 | 13 |
| 36 | 2.49 | 982 | 9 |
| 36.87 | 2.44 | 1192 | 11 |
| 37.99 | 2.37 | 767 | 7 |
| 32.64 | 2.74 | 1269 | 12 |
| 33.39 | 2.68 | 916 | 9 |
| 35.35 | 2.54 | 1355 | 13 |
| 36 | 2.49 | 982 | 9 |
| 36.87 | 2.44 | 1192 | 11 |
| 37.99 | 2.37 | 767 | 7 |

In some embodiments, crystalline Compound I Material 6+Form 2 is substantially crystalline. In some embodiments, crystalline Compound I Material 6+Form 2 is characterized by an X-ray powder diffractogram generated by an X-ray powder diffraction analysis with an incident beam of Cu Kα radiation.

In some embodiments, crystalline Compound I Material 6+Form 2 is characterized by an X-ray powder diffractogram having a signal at one or more of 3.85±2 degrees two-theta, 5.25±2 degrees two-theta, 5.35±2 degrees two-theta, 5.7±2 degrees two-theta, 5.95±2 degrees two-theta, 7.71±2 degrees two-theta, ±2 degrees two-theta, 7.99±2 degrees two-theta, 10.51±2 degrees two-theta, 10.88±2 degrees two-theta, 11.45±2 degrees two-theta, 11.95±2 degrees two-theta, 12.99±2 degrees two-theta, 13.76±2 degrees two-theta, 14.99±2 degrees two-theta, 15.51±2 degrees two-theta, 15.79±2 degrees two-theta, 16.13±2 degrees two-theta, 17.05±2 degrees two-theta, 17.52±2 degrees two-theta, 17.78±2 degrees two-theta, 18.22±2 degrees two-theta, 19.39±2 degrees two-theta, 19.84±2 degrees two-theta, 20.27±2 degrees two-theta, 21.11±2 degrees two-theta, 21.48±2 degrees two-theta, 21.86±2 degrees two-theta, 22.11±2 degrees two-theta, 22.38±2 degrees two-theta, 22.81±2 degrees two-theta, 23.06±2 degrees two-theta, 23.8±2 degrees two-theta, 24.15±2 degrees two-theta, 24.67±2 degrees two-theta, 25.07±2 degrees two-theta, 25.7±2 degrees two-theta, 25.95±2 degrees two-theta, 27.34±2 degrees two-theta, 27.71±2 degrees two-theta, 28.21±2 degrees two-theta, 29.03±2 degrees two-theta, 29.45±2 degrees two-theta, 29.71±2 degrees two-theta, 30.28±2 degrees two-theta, 30.83±2 degrees two-theta, 31.27±2 degrees two-theta, 31.67±2 degrees two-theta, 31.94±2 degrees two-theta, 32.64±2 degrees two-theta, 33.39±2 degrees two-theta, 35.35±2 degrees two-theta, 36±2 degrees two-theta, 36.87±2 degrees two-theta, and 37.99±2 degrees two-theta.

In some embodiments, crystalline Compound I Material 6+Form 2 is characterized by an X-ray powder diffractogram having a signal at one or more of 5.25±2 degrees two-theta, 5.35±2 degrees two-theta, 7.99±2 degrees two-theta, 10.88±2 degrees two-theta, and 14.99±2 degrees two-theta.

In some embodiments, crystalline Compound I Material 6+Form 2 is characterized by an X-ray powder diffractogram having a signal at one or more of 5.25±2 degrees two-theta, 5.35±2 degrees two-theta, 7.99±2 degrees two-theta, and 10.88±2 degrees two-theta.

In some embodiments, crystalline Compound I Material 6+Form 2 is characterized by an X-ray powder diffractogram having a signal at one or more of 5.25±2 degrees two-theta, 5.35±2 degrees two-theta, and 7.99±2 degrees two-theta.

In some embodiments, crystalline Compound I Material 6+Form 2 is characterized by an X-ray powder diffractogram having a signal at one or more of °2Θ (±0.2°)±2 degrees two-theta, and 5.25±2 degrees two-theta.

In some embodiments, crystalline Compound I Material 6+Form 2 is characterized by an X-ray powder diffractogram having a signal at 5.25±2 degrees two-theta. In some embodiments, crystalline Compound I Material 6+Form 2 is characterized by an X-ray powder diffractogram having a signal at 5.35±2 degrees two-theta. In some embodiments, crystalline Compound I Material 6+Form 2 is characterized by an X-ray powder diffractogram having a signal at 7.99±2 degrees two-theta. In some embodiments, crystalline Compound I Material 6+Form 2 is characterized by an X-ray powder diffractogram having a signal at 10.88±2 degrees two-theta. In some embodiments, crystalline Compound I Material 6+Form 2 is characterized by an X-ray powder diffractogram having a signal at 14.99±2 degrees two-theta.

In some embodiments, the Compound I Material 6+Form 2 mixture is characterized by an X-ray powder diffractogram substantially similar to FIG. 22 (A, top).

Another embodiment of the disclosure provides a method of making the Compound I Material 6+Form 2 mixture. In some embodiments, the method of making the Compound I Material 6+Form 2 mixture comprises: slurrying Compound I Form 1 in ethanol at rt for 10 days to yield a mixture of Compound I Material 6 and Compound I Form 2.

### G. Compound I Material 7

In some embodiments, the disclosure provides mixtures of Compound I Material 7+Form 2. FIG. 22 (B) provides an X-ray powder diffractogram of the Compound I Material 7+Form 2 mixture with corresponding tabulated data shown in Table 13.

TABLE 13
XRPD Signals for Crystalline Compound I
Material 7 + Form 2

| °2Θ | d-spacing | Intensity | Relative |
|---|---|---|---|
| (±0.2°) | (Å) | (Counts) | Intensity |
| 3.8 | 23.23 | 2092 | 10 |
| 6.01 | 14.69 | 478 | 2 |
| 7.63 | 11.58 | 801 | 4 |
| 7.76 | 11.38 | 3658 | 17 |
| 11.44 | 7.73 | 900 | 4 |
| 11.62 | 7.61 | 1048 | 5 |
| 12.87 | 6.87 | 4468 | 21 |
| 13.09 | 6.76 | 17381 | 81 |
| 13.32 | 6.64 | 1048 | 5 |
| 13.53 | 6.54 | 2078 | 10 |
| 13.78 | 6.42 | 2127 | 10 |
| 14.19 | 6.24 | 1915 | 9 |
| 14.36 | 6.16 | 435 | 2 |
| 14.78 | 5.99 | 4001 | 19 |
| 15.3 | 5.79 | 4548 | 21 |
| 15.55 | 5.69 | 2782 | 13 |
| 15.81 | 5.6 | 651 | 3 |
| 16.22 | 5.46 | 5503 | 26 |
| 16.47 | 5.38 | 1081 | 5 |
| 17.02 | 5.21 | 1664 | 8 |
| 17.77 | 4.99 | 1709 | 8 |
| 19.11 | 4.64 | 843 | 4 |
| 19.74 | 4.49 | 1831 | 9 |
| 21.48 | 4.13 | 21337 | 100 |
| 22.98 | 3.87 | 1541 | 7 |
| 23.4 | 3.8 | 1285 | 6 |
| 23.99 | 3.71 | 453 | 2 |
| 25.24 | 3.53 | 7146 | 33 |
| 25.41 | 3.5 | 2178 | 10 |
| 25.66 | 3.47 | 2982 | 14 |
| 25.91 | 3.44 | 14221 | 67 |
| 26.14 | 3.41 | 5149 | 24 |
| 26.64 | 3.34 | 1010 | 5 |
| 26.84 | 3.32 | 1890 | 9 |
| 27.48 | 3.24 | 487 | 2 |
| 27.83 | 3.2 | 5416 | 25 |
| 28.11 | 3.17 | 1256 | 6 |
| 28.6 | 3.12 | 1121 | 5 |
| 29.08 | 3.07 | 1429 | 7 |
| 29.4 | 3.04 | 1005 | 5 |
| 30.05 | 2.97 | 1149 | 5 |
| 30.6 | 2.92 | 844 | 4 |
| 30.82 | 2.9 | 523 | 2 |
| 31.09 | 2.87 | 309 | 1 |
| 31.49 | 2.84 | 607 | 3 |
| 31.96 | 2.8 | 752 | 4 |
| 32.81 | 2.73 | 760 | 4 |
| 33.21 | 2.7 | 543 | 3 |
| 33.53 | 2.67 | 721 | 3 |
| 33.9 | 2.64 | 1078 | 5 |
| 36.34 | 2.47 | 494 | 2 |
| 37.59 | 2.39 | 641 | 3 |
| 37.91 | 2.37 | 208 | 1 |
| 38.61 | 2.33 | 529 | 2 |
| 39.59 | 2.27 | 588 | 3 |

In some embodiments, crystalline Compound I Material 7+Form 2 is substantially crystalline. In some embodiments, crystalline Compound I Material 7+Form 2 is characterized by an X-ray powder diffractogram generated by an X-ray powder diffraction analysis with an incident beam of Cu Kα radiation.

In some embodiments, crystalline Compound I Material 7+Form 2 is characterized by an X-ray powder diffractogram having a signal at one or more of 3.8±2 degrees two-theta, 6.01±2 degrees two-theta, 7.63±2 degrees two-theta, 7.76±2 degrees two-theta, 11.44±2 degrees two-theta, 11.62±2 degrees two-theta, ±2 degrees two-theta, 12.87±2 degrees two-theta, 13.09±2 degrees two-theta, 13.32±2 degrees two-theta, 13.53±2 degrees two-theta, 13.78±2 degrees two-theta, 14.19±2 degrees two-theta, 14.36±2 degrees two-theta, 14.78±2 degrees two-theta, 15.3±2 degrees two-theta, 15.55±2 degrees two-theta, 15.81±2 degrees two-theta, 16.22±2 degrees two-theta, 16.47±2 degrees two-theta, 17.02±2 degrees two-theta, 17.77±2 degrees two-theta, 19.11±2 degrees two-theta, 19.74±2 degrees two-theta, 21.48±2 degrees two-theta, 22.98±2 degrees two-theta, 23.4±2 degrees two-theta, 23.99±2 degrees two-theta, 25.24±2 degrees two-theta, 25.41±2 degrees two-theta, 25.66±2 degrees two-theta, 25.91±2 degrees two-theta, 26.14±2 degrees two-theta, 26.64±2 degrees two-theta, 26.84±2 degrees two-theta, 27.48±2 degrees two-theta, 27.83±2 degrees two-theta, 28.11±2 degrees two-theta, 28.6±2 degrees two-theta, 29.08±2 degrees two-theta, 29.4±2 degrees two-theta, 30.05±2 degrees two-theta, 30.6±2 degrees two-theta, 30.82±2 degrees two-theta, 31.09±2 degrees two-theta, 31.49±2 degrees two-theta, 31.96±2 degrees two-theta, 32.81±2 degrees two-theta, 33.21±2 degrees two-theta, 33.53±2 degrees two-theta, 33.9±2 degrees two-theta, 36.34±2 degrees two-theta, 37.59±2 degrees two-theta, 37.91±2 degrees two-theta, 38.61±2 degrees two-theta, and 39.59±2 degrees two-theta.

In some embodiments, crystalline Compound I Material 7+Form 2 is characterized by an X-ray powder diffractogram having a signal at one or more of 13.09±2 degrees two-theta, 13.32±2 degrees two-theta, 13.53±2 degrees two-theta, 14.19±2 degrees two-theta, and 14.78±2 degrees two-theta.

In some embodiments, crystalline Compound I Material 7+Form 2 is characterized by an X-ray powder diffractogram having a signal at one or more of 13.09±2 degrees two-theta, 13.53±2 degrees two-theta, 14.19±2 degrees two-theta, and 14.78±2 degrees two-theta.

In some embodiments, crystalline Compound I Material 7+Form 2 is characterized by an X-ray powder diffractogram having a signal at one or more of 13.09±2 degrees two-theta, 13.53±2 degrees two-theta, and 14.78±2 degrees two-theta.

In some embodiments, crystalline Compound I Material 7+Form 2 is characterized by an X-ray powder diffractogram having a signal at one or more of 13.09±2 degrees two-theta, and 14.78±2 degrees two-theta.

In some embodiments, crystalline Compound I Material 7+Form 2 is characterized by an X-ray powder diffractogram having a signal at 13.09±2 degrees two-theta. In some embodiments, crystalline Compound I Material 7+Form 2 is characterized by an X-ray powder diffractogram having a signal at 13.32±2 degrees two-theta. In some embodiments, crystalline Compound I Material 7+Form 2 is characterized by an X-ray powder diffractogram having a signal at 13.53±2 degrees two-theta. In some embodiments, crystalline Compound I Material 7+Form 2 is characterized by an X-ray powder diffractogram having a signal at 14.19±2 degrees two-theta. In some embodiments, crystalline Compound I Material 7+Form 2 is characterized by an X-ray powder diffractogram having a signal at 14.78±2 degrees two-theta.

In some embodiments, the Compound I Material 7+Form 2 mixture is characterized by an X-ray powder diffractogram substantially similar to FIG. 22 (B).

Another embodiment of the disclosure provides a method of making the Compound I Material 7+Form 2 mixture. In some embodiments, the method of making the Compound I Material 7+Form 2 mixture comprises: dissolving Compound I Form 1 in dimethyl sulfoxide (DMSO), MTBE, and dichloromethane (DCM) at rt for 3 hrs to yield a mixture of Compound I Material 7 and Compound I Form 2.

### H. Compound I Material 8

In some embodiments, the disclosure provides mixtures of Compound I Material 8+Form 2. FIG. 22 (C) provides an X-ray powder diffractogram of the Compound I Material 8+Form 2 mixture with corresponding tabulated data shown in Table 14.

TABLE 14
XRPD Signals for Crystalline Compound I
Material 8 + Form 2

| °2Θ | d-spacing | Intensity | Relative |
|---|---|---|---|
| (±0.2°) | (Å) | (Counts) | Intensity |
| 3.8 | 23.23 | 2092 | 10 |
| 6.01 | 14.69 | 478 | 2 |
| 7.63 | 11.58 | 801 | 4 |
| 7.76 | 11.38 | 3658 | 17 |
| 11.44 | 7.73 | 900 | 4 |
| 11.62 | 7.61 | 1048 | 5 |
| 12.87 | 6.87 | 4468 | 21 |
| 13.09 | 6.76 | 17381 | 81 |
| 13.32 | 6.64 | 1048 | 5 |
| 13.53 | 6.54 | 2078 | 10 |
| 13.78 | 6.42 | 2127 | 10 |
| 14.19 | 6.24 | 1915 | 9 |
| 14.36 | 6.16 | 435 | 2 |
| 14.78 | 5.99 | 4001 | 19 |
| 15.3 | 5.79 | 4548 | 21 |
| 15.55 | 5.69 | 2782 | 13 |
| 15.81 | 5.6 | 651 | 3 |
| 16.22 | 5.46 | 5503 | 26 |
| 16.47 | 5.38 | 1081 | 5 |
| 17.02 | 5.21 | 1664 | 8 |
| 17.77 | 4.99 | 1709 | 8 |
| 19.11 | 4.64 | 843 | 4 |
| 19.74 | 4.49 | 1831 | 9 |
| 21.48 | 4.13 | 21337 | 100 |
| 22.98 | 3.87 | 1541 | 7 |
| 23.4 | 3.8 | 1285 | 6 |
| 23.99 | 3.71 | 453 | 2 |
| 25.24 | 3.53 | 7146 | 33 |
| 25.41 | 3.5 | 2178 | 10 |
| 25.66 | 3.47 | 2982 | 14 |
| 25.91 | 3.44 | 14221 | 67 |
| 26.14 | 3.41 | 5149 | 24 |
| 26.64 | 3.34 | 1010 | 5 |
| 26.84 | 3.32 | 1890 | 9 |
| 27.48 | 3.24 | 487 | 2 |
| 27.83 | 3.2 | 5416 | 25 |
| 28.11 | 3.17 | 1256 | 6 |
| 28.6 | 3.12 | 1121 | 5 |
| 29.08 | 3.07 | 1429 | 7 |
| 29.4 | 3.04 | 1005 | 5 |
| 30.05 | 2.97 | 1149 | 5 |
| 30.6 | 2.92 | 844 | 4 |
| 30.82 | 2.9 | 523 | 2 |
| 31.09 | 2.87 | 309 | 1 |
| 31.49 | 2.84 | 607 | 3 |
| 31.96 | 2.8 | 752 | 4 |
| 32.81 | 2.73 | 760 | 4 |
| 33.21 | 2.7 | 543 | 3 |
| 33.53 | 2.67 | 721 | 3 |
| 33.9 | 2.64 | 1078 | 5 |
| 36.34 | 2.47 | 494 | 2 |
| 37.59 | 2.39 | 641 | 3 |
| 37.91 | 2.37 | 208 | 1 |
| 38.61 | 2.33 | 529 | 2 |
| 39.59 | 2.27 | 588 | 3 |

In some embodiments, crystalline Compound I Material 8+Form 2 is substantially crystalline. In some embodiments, crystalline Compound I Material 8+Form 2 is characterized by an X-ray powder diffractogram generated by an X-ray powder diffraction analysis with an incident beam of Cu Kα radiation.

In some embodiments, crystalline Compound I Material 8+Form 2 is characterized by an X-ray powder diffractogram having a signal at one or more of 3.8±2 degrees two-theta, 6.01±2 degrees two-theta, 7.63±2 degrees two-theta, 7.76±2 degrees two-theta, 11.44±2 degrees two-theta, 11.62±2 degrees two-theta, ±2 degrees two-theta, 12.87±2 degrees two-theta, 13.09±2 degrees two-theta, 13.32±2 degrees two-theta, 13.53±2 degrees two-theta, 13.78±2 degrees two-theta, 14.19±2 degrees two-theta, 14.36±2 degrees two-theta, 14.78±2 degrees two-theta, 15.3±2 degrees two-theta, 15.55±2 degrees two-theta, 15.81±2 degrees two-theta, 16.22±2 degrees two-theta, 16.47±2 degrees two-theta, 17.02±2 degrees two-theta, 17.77±2 degrees two-theta, 19.11±2 degrees two-theta, 19.74±2 degrees two-theta, 21.48±2 degrees two-theta, 22.98±2 degrees two-theta, 23.4±2 degrees two-theta, 23.99±2 degrees two-theta, 25.24±2 degrees two-theta, 25.41±2 degrees two-theta, 25.66±2 degrees two-theta, 25.91±2 degrees two-theta, 26.14±2 degrees two-theta, 26.64±2 degrees two-theta, 26.84±2 degrees two-theta, 27.48±2 degrees two-theta, 27.83±2 degrees two-theta, 28.11±2 degrees two-theta, 28.6±2 degrees two-theta, 29.08±2 degrees two-theta, 29.4±2 degrees two-theta, 30.05±2 degrees two-theta, 30.6±2 degrees two-theta, 30.82±2 degrees two-theta, 31.09±2 degrees two-theta, 31.49±2 degrees two-theta, 31.96±2 degrees two-theta, 32.81±2 degrees two-theta, 33.21±2 degrees two-theta, 33.53±2 degrees two-theta, 33.9±2 degrees two-theta, 36.34±2 degrees two-theta, 37.59±2 degrees two-theta, 37.91±2 degrees two-theta, 38.61±2 degrees two-theta, and 39.59±2 degrees two-theta.

In some embodiments, crystalline Compound I Material 8+Form 2 is characterized by an X-ray powder diffractogram having a signal at one or more of 5.27±2 degrees two-theta, 8.98±2 degrees two-theta, 9.66±2 degrees two-theta, 12.86±2 degrees two-theta, and 13.63±2 degrees two-theta.

In some embodiments, crystalline Compound I Material 8+Form 2 is characterized by an X-ray powder diffractogram having a signal at one or more of 5.27±2 degrees two-theta, 9.66±2 degrees two-theta, 12.86±2 degrees two-theta, and 13.63±2 degrees two-theta.

In some embodiments, crystalline Compound I Material 8+Form 2 is characterized by an X-ray powder diffractogram having a signal at one or more of 5.27±2 degrees two-theta, 12.86±2 degrees two-theta, and 13.63±2 degrees two-theta.

In some embodiments, crystalline Compound I Material 8+Form 2 is characterized by an X-ray powder diffractogram having a signal at one or more of 5.27±2 degrees two-theta, and 13.63±2 degrees two-theta.

In some embodiments, crystalline Compound I Material 8+Form 2 is characterized by an X-ray powder diffractogram having a signal at 5.27±2 degrees two-theta. In some embodiments, crystalline Compound I Material 8+Form 2 is characterized by an X-ray powder diffractogram having a signal at 8.98±2 degrees two-theta. In some embodiments, crystalline Compound I Material 8+Form 2 is characterized by an X-ray powder diffractogram having a signal at 9.66±2 degrees two-theta. In some embodiments, crystalline Compound I Material 8+Form 2 is characterized by an X-ray powder diffractogram having a signal at 12.86±2 degrees two-theta. In some embodiments, crystalline Compound I Material 8+Form 2 is characterized by an X-ray powder diffractogram having a signal at 13.63±2 degrees two-theta.

In some embodiments, the Compound I Material 8+Form 2 mixture is characterized by an X-ray powder diffractogram substantially similar to FIG. 22 (C).

Another embodiment of the disclosure provides a method of making the Compound I Material 8+Form 2 mixture. In some embodiments, the method of making the Compound I Material 8+Form 2 mixture comprises: stirring a solution of Compound I Form 1 in methanol and acetone at rt for 1 day to yield a mixture of Compound I Material 8 and Compound I Form 2.

### I. Compound I Form 9

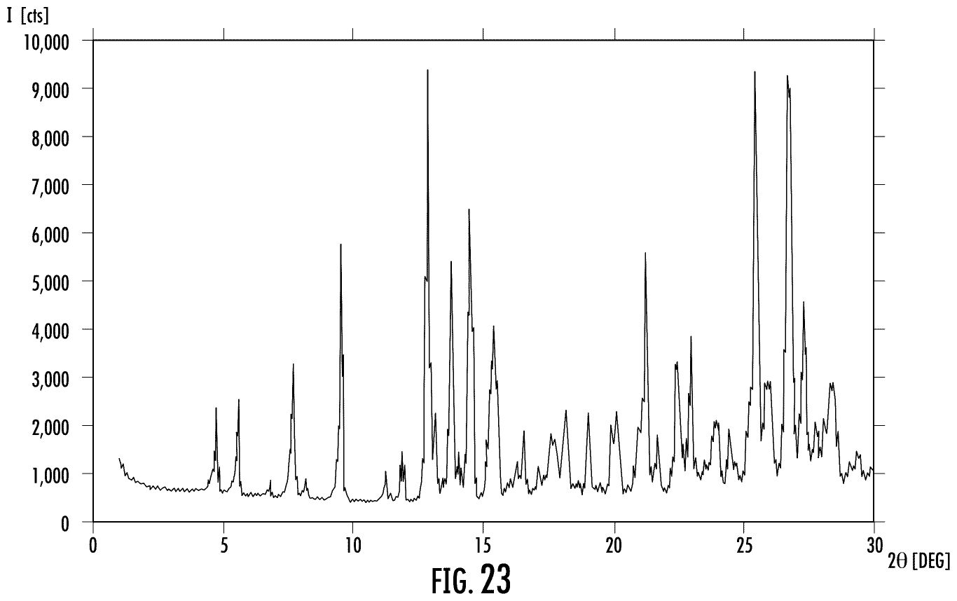

In some embodiments, the disclosure provides crystalline Compound I Form 9. In some embodiments, Compound I Form 9 is a methanol solvate. In some embodiments, Compound I Form 9 is a methanol solvate with up to 1 mole of methanol. FIG. 23 provides an X-ray powder diffractogram of crystalline Compound I Form 9 with corresponding tabulated data for FIG. 23 shown in Table 15.

TABLE 15
XRPD Signals for Crystalline Compound I Form 9

| °2Θ | d-spacing | Intensity | Relative |
|---|---|---|---|
| (±0.2°) | (Å) | (Counts) | Intensity |
| 4.76 | 18.55 | 1472 | 19 |
| 5.62 | 15.71 | 1742 | 22 |
| 6.82 | 12.95 | 259 | 3 |
| 7.69 | 11.49 | 2543 | 32 |
| 8.19 | 10.79 | 387 | 5 |
| 9.54 | 9.26 | 5189 | 65 |
| 11.27 | 7.84 | 579 | 7 |
| 11.9 | 7.43 | 832 | 10 |
| 12.89 | 6.86 | 6706 | 84 |
| 13.17 | 6.72 | 1171 | 15 |
| 13.79 | 6.42 | 4359 | 55 |
| 14.06 | 6.29 | 600 | 8 |
| 14.49 | 6.11 | 5098 | 64 |
| 15.43 | 5.74 | 3244 | 41 |
| 16.58 | 5.34 | 1056 | 13 |
| 16.33 | 5.42 | 391 | 5 |
| 17.13 | 5.17 | 358 | 5 |
| 17.62 | 5.03 | 719 | 9 |
| 18.2 | 4.87 | 1515 | 19 |
| 19.05 | 4.66 | 1542 | 19 |
| 19.94 | 4.45 | 565 | 7 |
| 20.17 | 4.4 | 964 | 12 |
| 21.28 | 4.17 | 4458 | 56 |
| 21.71 | 4.09 | 798 | 10 |
| 22.46 | 3.96 | 2238 | 28 |
| 22.98 | 3.87 | 2775 | 35 |
| 23.93 | 3.72 | 1153 | 15 |
| 24.48 | 3.63 | 890 | 11 |
| 25.47 | 3.49 | 7200 | 91 |
| 26.02 | 3.42 | 1339 | 17 |
| 26.74 | 3.33 | 7944 | 100 |
| 27.34 | 3.26 | 3299 | 42 |
| 27.78 | 3.21 | 749 | 9 |
| 28.43 | 3.14 | 1305 | 16 |
| 29.38 | 3.04 | 450 | 6 |
| 30.73 | 2.91 | 491 | 6 |
| 31.2 | 2.86 | 653 | 8 |
| 32.27 | 2.77 | 723 | 9 |
| 33.51 | 2.67 | 839 | 11 |
| 34.81 | 2.58 | 448 | 6 |
| 35.9 | 2.5 | 350 | 4 |
| 38.77 | 2.32 | 442 | 6 |

In some embodiments, crystalline Compound I Form 9 is substantially pure. In some embodiments, crystalline Compound I Form 9 is substantially crystalline. In some embodiments, crystalline Compound I Form 9 is characterized by an X-ray powder diffractogram generated by an X-ray powder diffraction analysis with an incident beam of Cu Kα radiation.

In some embodiments, crystalline Compound I Form 9 is characterized by an X-ray powder diffractogram having a signal at one or more of 4.76±2 degrees two-theta, 5.62±2 degrees two-theta, 6.82±2 degrees two-theta, 7.69±2 degrees two-theta, 8.19±2 degrees two-theta, 9.54±2 degrees two-theta, ±2 degrees two-theta, 11.27±2 degrees two-theta, 11.9±2 degrees two-theta, 12.89±2 degrees two-theta, 13.17±2 degrees two-theta, 13.79±2 degrees two-theta, 14.06±2 degrees two-theta, 14.49±2 degrees two-theta, 15.43±2 degrees two-theta, 16.58±2 degrees two-theta, 16.33±2 degrees two-theta, 17.13±2 degrees two-theta, 17.62±2 degrees two-theta, 18.2±2 degrees two-theta, 19.05±2 degrees two-theta, 19.94±2 degrees two-theta, 20.17±2 degrees two-theta, 21.28±2 degrees two-theta, 21.71±2 degrees two-theta, 22.46±2 degrees two-theta, 22.98±2 degrees two-theta, 23.93±2 degrees two-theta, 24.48±2 degrees two-theta, 25.47±2 degrees two-theta, 26.02±2 degrees two-theta, 26.74±2 degrees two-theta, 27.34±2 degrees two-theta, 27.78±2 degrees two-theta, 28.43±2 degrees two-theta, 29.38±2 degrees two-theta, 30.73±2 degrees two-theta, 31.2±2 degrees two-theta, 32.27±2 degrees two-theta, 33.51±2 degrees two-theta, 34.81±2 degrees two-theta, and 38.77±2 degrees two-theta.

In some embodiments, crystalline Compound I Form 9 is characterized by an X-ray powder diffractogram having a signal at one or more of 4.76±2 degrees two-theta, 5.62±2 degrees two-theta, 9.54±2 degrees two-theta, 12.89±2 degrees two-theta, and 14.49±2 degrees two-theta.

In some embodiments, crystalline Compound I Form 9 is characterized by an X-ray powder diffractogram having a signal at one or more of 4.76±2 degrees two-theta, 5.62±2 degrees two-theta, 9.54±2 degrees two-theta, and 12.89±2 degrees two-theta.

In some embodiments, crystalline Compound I Form 9 is characterized by an X-ray powder diffractogram having a signal at one or more of 4.76±2 degrees two-theta, 9.54±2 degrees two-theta, and 12.89±2 degrees two-theta.

In some embodiments, crystalline Compound I Form 9 is characterized by an X-ray powder diffractogram having a signal at one or more of 9.54±2 degrees two-theta, and 12.89±2 degrees two-theta.

In some embodiments, crystalline Compound I Form 9 is characterized by an X-ray powder diffractogram having a signal at 4.76±2 degrees two-theta. In some embodiments, crystalline Compound I Form 9 is characterized by an X-ray powder diffractogram having a signal at 5.62±2 degrees two-theta. In some embodiments, crystalline Compound I Form 9 is characterized by an X-ray powder diffractogram having a signal at 9.54±2 degrees two-theta. In some embodiments, crystalline Compound I Form 9 is characterized by an X-ray powder diffractogram having a signal at 12.89±2 degrees two-theta. In some embodiments, crystalline Compound I Form 9 is characterized by an X-ray powder diffractogram having a signal at 14.49±2 degrees two-theta.

In some embodiments, crystalline Compound I Form 9 is characterized by an X-ray powder diffractogram substantially similar to FIG. 23.

In some embodiments, crystalline Compound I Form 9 is characterized by a primitive monoclinic crystal system, P2₁/n space group and the following unit cell dimensions resulting from successful indexing of Form 9 XRPD:

| | | | |
|---|---|---|---|
| a | 7.1 ± 0.1 Å | α | 90 ± 0.1° |
| b | 31.4 ± 0.1 Å | β | 94.8 ± 0.1° |
| c | 23.0 ± 0.1 Å | γ | 90 ± 0.1°. |

In some embodiments, Compound I Form 9 is characterized by a DSC having a small broad endotherm peak temperature of 175° C.

In some embodiments, Compound I Form 9 is characterized by a DSC having an endotherm peak temperature of 184° C.

In some embodiments, Compound I Form 9 is characterized by a DSC having a broad endotherm peak temperature of 224° C.

In some embodiments, Compound I Form 9 is characterized by a DSC having three endotherms at peak temperatures of 175° C., 184° C. and 224° C.

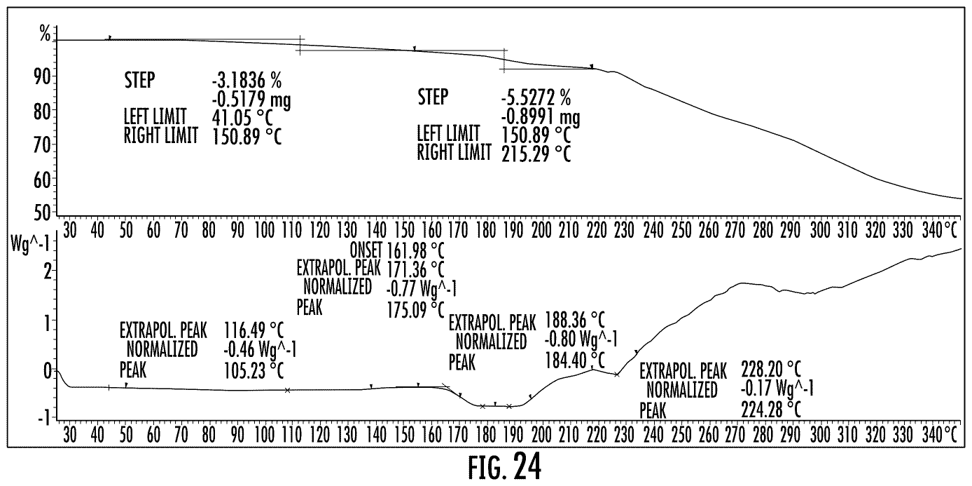

In some embodiments, Compound I Form 9 is characterized by a DSC substantially similar to FIG. 24.

In some embodiments, Compound I Form 9 is also characterized by a weight loss of 3.2% from 41° C. to 151° C., as measured by thermogravimetric analysis. In some embodiments, Compound I Form 9 is also characterized by a weight loss of approximately 5.5% at temperatures 151° C. to 215° C., as measured by thermogravimetric analysis.

In some embodiments, Compound I Form 9 is characterized by a TGA substantially similar to FIG. 24.

Another embodiment of the disclosure provides a method of making crystalline Compound I Form 9. In some embodiments, the method of making crystalline Compound I Form 9 comprises: slowly evaporating Compound I Form 1 in methanol under nitrogen to yield crystalline Compound I Form 9.

### J. Compound I Material 10

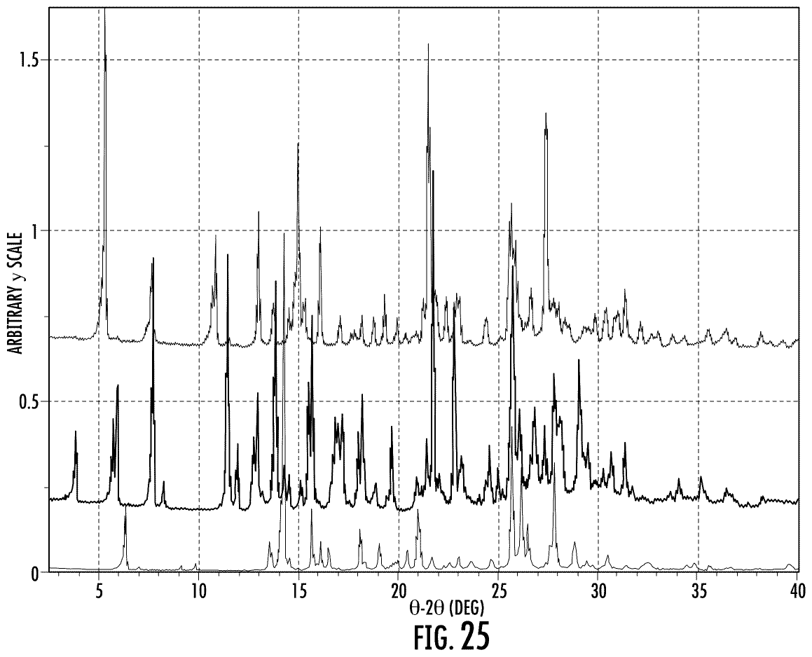

In some embodiments, the disclosure provides mixtures of Forms and Materials of Compound I. In some embodiments, the Compound I Material 10 is an acetone solvate. In some embodiments, Compound I Material 10 is a mixture of Form 2 and acetone solvate. FIG. 25 provides an X-ray powder diffractogram of the Compound I Material 10+Form 2 mixture (top) with corresponding tabulated data for FIG. 25 shown in Table 16.

TABLE 16
XRPD Signals for Crystalline Compound I
Material 10 + Form 2

| °2Θ | d-spacing | Intensity | Relative |
|---|---|---|---|
| (±0.2°) | (Å) | (Counts) | Intensity |
| 5.35 | 16.51 | 13403 | 100 |
| 7.66 | 11.53 | 3243 | 24 |
| 10.71 | 8.25 | 1229 | 9 |
| 10.88 | 8.13 | 3545 | 26 |
| 13.02 | 6.79 | 5391 | 40 |
| 13.76 | 6.43 | 1674 | 12 |
| 14.56 | 6.08 | 1124 | 8 |
| 14.99 | 5.91 | 7250 | 54 |
| 15.36 | 5.76 | 1279 | 10 |
| 16.11 | 5.5 | 3316 | 25 |
| 17.1 | 5.18 | 1206 | 9 |
| 17.65 | 5.02 | 414 | 3 |
| 17.83 | 4.97 | 464 | 3 |
| 18.18 | 4.88 | 997 | 7 |
| 18.79 | 4.72 | 1179 | 9 |
| 19.34 | 4.59 | 1781 | 13 |
| 19.95 | 4.45 | 1046 | 8 |
| 20.39 | 4.35 | 460 | 3 |
| 21.26 | 4.18 | 1206 | 9 |
| 21.51 | 4.13 | 11275 | 84 |
| 22.39 | 3.97 | 1591 | 12 |
| 22.91 | 3.88 | 1859 | 14 |
| 24.4 | 3.65 | 1024 | 8 |
| 25.67 | 3.47 | 3367 | 25 |
| 25.89 | 3.44 | 2233 | 17 |
| 26.62 | 3.35 | 1699 | 13 |
| 27.39 | 3.25 | 8357 | 62 |
| 28.03 | 3.18 | 638 | 5 |
| 29.86 | 2.99 | 861 | 6 |
| 30.42 | 2.94 | 963 | 7 |
| 31.02 | 2.88 | 837 | 6 |
| 31.35 | 2.85 | 2002 | 15 |
| 32.12 | 2.78 | 849 | 6 |
| 33.02 | 2.71 | 591 | 4 |
| 33.73 | 2.66 | 457 | 3 |
| 34.33 | 2.61 | 297 | 2 |
| 35.53 | 2.52 | 585 | 4 |
| 36.45 | 2.46 | 461 | 3 |
| 36.85 | 2.44 | 346 | 3 |
| 38.12 | 2.36 | 585 | 4 |
| 39.26 | 2.29 | 271 | 2 |

In some embodiments, crystalline Compound I Material 10+Form 2 is substantially crystalline. In some embodiments, crystalline Compound I Material 10+Form 2 is characterized by an X-ray powder diffractogram generated by an X-ray powder diffraction analysis with an incident beam of Cu Kα radiation.

In some embodiments, crystalline Compound I Material 10+Form 2 is characterized by an X-ray powder diffractogram having a signal at one or more of 5.35±2 degrees two-theta, 7.66±2 degrees two-theta, 10.71±2 degrees two-theta, 10.88±2 degrees two-theta, 13.02±2 degrees two-theta, 13.76±2 degrees two-theta, ±2 degrees two-theta, 14.56±2 degrees two-theta, 14.99±2 degrees two-theta, 15.36±2 degrees two-theta, 16.11±2 degrees two-theta, 17.1±2 degrees two-theta, 17.65±2 degrees two-theta, 17.83±2 degrees two-theta, 18.18±2 degrees two-theta, 18.79±2 degrees two-theta, 19.34±2 degrees two-theta, 19.95±2 degrees two-theta, 20.39±2 degrees two-theta, 21.26±2 degrees two-theta, 21.51±2 degrees two-theta, 22.39±2 degrees two-theta, 22.91±2 degrees two-theta, 24.4±2 degrees two-theta, 25.67±2 degrees two-theta, 25.89±2 degrees two-theta, 26.62±2 degrees two-theta, 27.39±2 degrees two-theta, 28.03±2 degrees two-theta, 29.86±2 degrees two-theta, 30.42±2 degrees two-theta, 31.02±2 degrees two-theta, 31.35±2 degrees two-theta, 32.12±2 degrees two-theta, 33.02±2 degrees two-theta, 33.73±2 degrees two-theta, 34.33±2 degrees two-theta, 35.53±2 degrees two-theta, 36.45±2 degrees two-theta, 36.85±2 degrees two-theta, 38.12±2 degrees two-theta, and 39.26±2 degrees two-theta.

In some embodiments, crystalline Compound I Material 10+Form 2 is characterized by an X-ray powder diffractogram having a signal at one or more of 5.35±2 degrees two-theta, 7.66±2 degrees two-theta, 10.71±2 degrees two-theta, 10.88±2 degrees two-theta, and 14.56±2 degrees two-theta.

In some embodiments, crystalline Compound I Material 10+Form 2 is characterized by an X-ray powder diffractogram having a signal at one or more of 5.35±2 degrees two-theta, 7.66±2 degrees two-theta, 10.71±2 degrees two-theta, and 10.88±2 degrees two-theta.

In some embodiments, crystalline Compound I Material 10+Form 2 is characterized by an X-ray powder diffractogram having a signal at one or more of 5.35±2 degrees two-theta, 10.71±2 degrees two-theta, and 10.88±2 degrees two-theta.

In some embodiments, crystalline Compound I Material 10+Form 2 is characterized by an X-ray powder diffractogram having a signal at one or more of 5.35±2 degrees two-theta, and 10.88±2 degrees two-theta.

In some embodiments, crystalline Compound I Material 10+Form 2 is characterized by an X-ray powder diffractogram having a signal at 5.35±2 degrees two-theta. In some embodiments, crystalline Compound I Material 10+Form 2 is characterized by an X-ray powder diffractogram having a signal at 7.66±2 degrees two-theta. In some embodiments, crystalline Compound I Material 10+Form 2 is characterized by an X-ray powder diffractogram having a signal at 10.71±2 degrees two-theta. In some embodiments, crystalline Compound I Material 10+Form 2 is characterized by an X-ray powder diffractogram having a signal at 10.88±2 degrees two-theta. In some embodiments, crystalline Compound I Material 10+Form 2 is characterized by an X-ray powder diffractogram having a signal at 14.56±2 degrees two-theta.

In some embodiments, the Compound I Material 10 and Form 2 mixture is characterized by an X-ray powder diffractogram substantially similar to FIG. 25 (top).

In some embodiments, the Compound I Material 10 and Form 2 mixture is characterized by a DSC having a small broad endotherm peak temperature of 104° C.

In some embodiments, Compound I Material 10 and Form 2 mixture is characterized by a DSC having an endotherm peak temperature of 126° C.

In some embodiments, the Compound I Material 10 and Form 2 mixture is characterized by a DSC having an onset of melting temperature of 226° C. In some embodiments, the Compound I Material 10 and Form 2 mixture is characterized by a DSC having a broad endotherm peak temperature of 231° C. In some embodiments, the Compound I Material 10 and Form 2 mixture is characterized by a DSC having an onset of melting temperature of 226° C. and/or a peak temperature of 231° C.

In some embodiments, the Compound I Material 10 and Form 2 mixture is characterized by a DSC having three endotherms at peak temperatures of 104° C., 126° C. and 231° C.

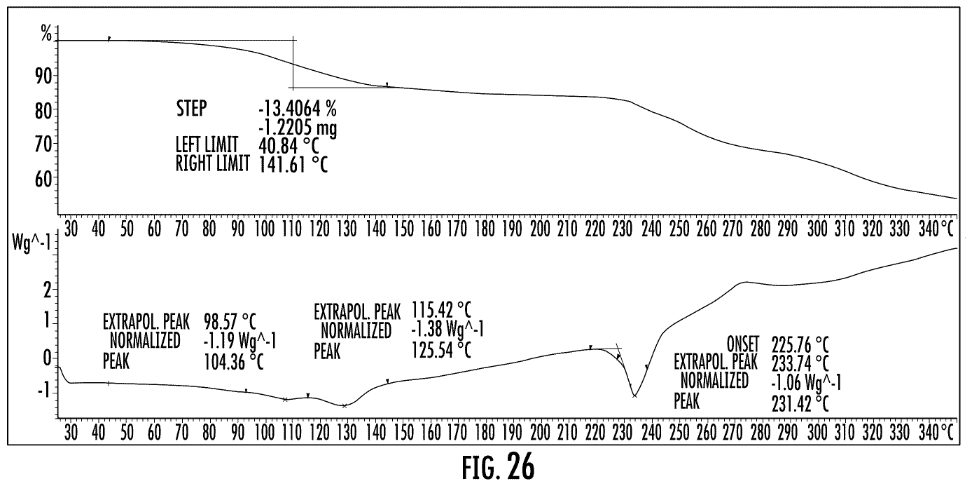

In some embodiments, the Compound I Material 10 and Form 2 mixture is characterized by a DSC substantially similar to FIG. 26.

In some embodiments, the Compound I Material 10 and Form 2 mixture is also characterized by a weight loss of 13.4% from 41° C. to 142° C., as measured by thermogravimetric analysis

In some embodiments, the Compound I Material 10 and Form 2 mixture is characterized by a TGA substantially similar to FIG. 26.

Another embodiment of the disclosure provides a method of making a Compound I Material 10 and Form 2 mixture. In some embodiments, the method of making the Compound I Material 10 and Form 2 mixture comprises: slurrying Compound I Form 1 in acetone at rt for 4 days to yield a mixture of Compound I Material 10 and Compound I Form 2.

### K. Compound I Form 11

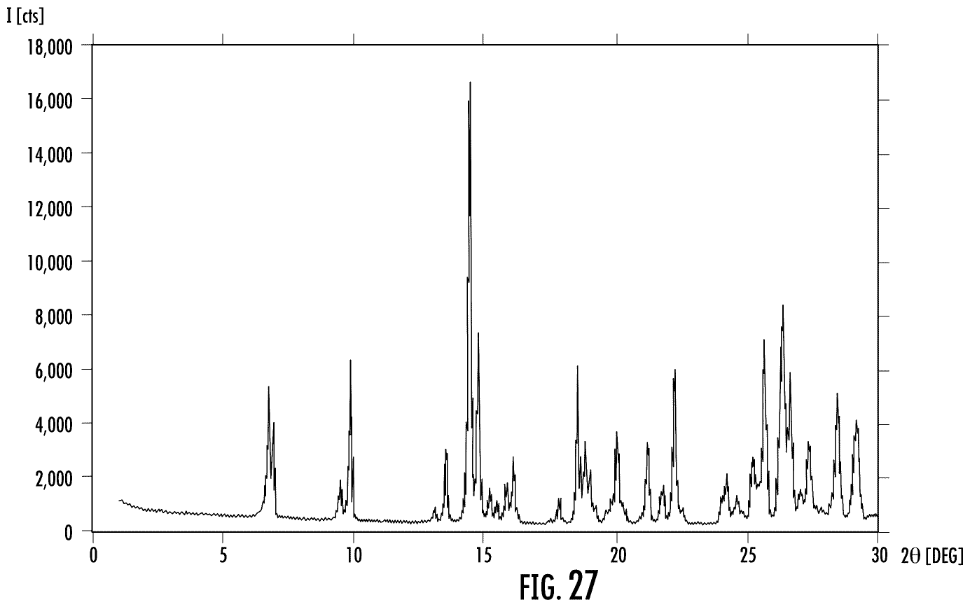

In some embodiments, the disclosure provides crystalline Compound I Form 11. In some embodiments, Compound I Form 11 is an acetonitrile de-solvate. In some embodiments, the Form 11 is observed by desolvation of acetonitrile and isopropanol solvates. In some embodiments, Compound I Form 11 is an anhydrous de-solvate of acetonitrile. In some embodiments, Compound I Form 11 is an acetonitrile de-solvate with up to 1 mole of acetonitrile. FIG. 27 provides an X-ray powder diffractogram of crystalline Compound I Form 11 with corresponding tabulated data for FIG. 27 shown in Table 17.

TABLE 17
XRPD Signals for Crystalline Compound I Form 11

| °2Θ | d-spacing | Intensity | Relative |
|---|---|---|---|
| (±0.2°) | (Å) | (Counts) | Intensity |
| 6.72 | 13.14 | 3359 | 22 |
| 6.91 | 12.78 | 2346 | 16 |
| 9.43 | 9.37 | 1317 | 9 |
| 9.85 | 8.97 | 5266 | 35 |
| 13.02 | 6.79 | 478 | 3 |
| 13.48 | 6.56 | 2438 | 16 |
| 14.38 | 6.15 | 14972 | 100 |
| 14.71 | 6.02 | 6240 | 42 |
| 15.13 | 5.85 | 1006 | 7 |
| 15.41 | 5.75 | 528 | 4 |
| 15.76 | 5.62 | 978 | 7 |
| 16.02 | 5.53 | 1913 | 13 |
| 17.79 | 4.98 | 845 | 6 |
| 18.49 | 4.79 | 4881 | 33 |
| 18.76 | 4.73 | 1785 | 12 |
| 18.96 | 4.68 | 1061 | 7 |
| 19.96 | 4.44 | 1925 | 13 |
| 21.11 | 4.21 | 2872 | 19 |
| 21.71 | 4.09 | 1132 | 8 |
| 22.18 | 4 | 4944 | 33 |
| 24.14 | 3.68 | 1568 | 10 |
| 24.52 | 3.63 | 781 | 5 |
| 25.12 | 3.54 | 1572 | 10 |
| 25.59 | 3.48 | 5231 | 35 |
| 26.31 | 3.38 | 5972 | 40 |
| 26.58 | 3.35 | 3718 | 25 |
| 26.96 | 3.3 | 663 | 4 |
| 27.28 | 3.27 | 2390 | 16 |
| 28.36 | 3.14 | 4238 | 28 |
| 29.12 | 3.06 | 3372 | 23 |
| 30.69 | 2.91 | 1892 | 13 |
| 32.68 | 2.74 | 302 | 2 |
| 33.14 | 2.7 | 401 | 3 |
| 33.66 | 2.66 | 612 | 4 |
| 34.13 | 2.62 | 426 | 3 |
| 35.43 | 2.53 | 332 | 2 |
| 36.15 | 2.48 | 459 | 3 |
| 37.44 | 2.4 | 341 | 2 |
| 38.27 | 2.35 | 420 | 3 |
| 38.59 | 2.33 | 290 | 2 |
| 39.75 | 2.27 | 192 | 1 |

In some embodiments, crystalline Compound I Form 11 is substantially pure. In some embodiments, crystalline Compound I Form 11 is substantially crystalline. In some embodiments, crystalline Compound I Form 11 is characterized by an X-ray powder diffractogram generated by an X-ray powder diffraction analysis with an incident beam of Cu Kα radiation.

In some embodiments, crystalline Compound I Form 11 is characterized by an X-ray powder diffractogram having a signal at one or more of 6.72±2 degrees two-theta, 6.91±2 degrees two-theta, 9.43±2 degrees two-theta, 9.85±2 degrees two-theta, 13.02±2 degrees two-theta, 13.48±2 degrees two-theta, ±2 degrees two-theta, 14.38±2 degrees two-theta, 14.71±2 degrees two-theta, 15.13±2 degrees two-theta, 15.41±2 degrees two-theta, 15.76±2 degrees two-theta, 16.02±2 degrees two-theta, 17.79±2 degrees two-theta, 18.49±2 degrees two-theta, 18.76±2 degrees two-theta, 18.96±2 degrees two-theta, 19.96±2 degrees two-theta, 21.11±2 degrees two-theta, 21.71±2 degrees two-theta, 22.18±2 degrees two-theta, 24.14±2 degrees two-theta, 24.52±2 degrees two-theta, 25.12±2 degrees two-theta, 25.59±2 degrees two-theta, 26.31±2 degrees two-theta, 26.58±2 degrees two-theta, 26.96±2 degrees two-theta, 27.28±2 degrees two-theta, 28.36±2 degrees two-theta, 29.12±2 degrees two-theta, 30.69±2 degrees two-theta, 32.68±2 degrees two-theta, 33.14±2 degrees two-theta, 33.66±2 degrees two-theta, 34.13±2 degrees two-theta, 35.43±2 degrees two-theta, 36.15±2 degrees two-theta, 37.44±2 degrees two-theta, 38.27±2 degrees two-theta, 38.59±2 degrees two-theta, and 39.75±2 degrees two-theta.

In some embodiments, crystalline Compound I Form 11 is characterized by an X-ray powder diffractogram having a signal at one or more of 6.72±2 degrees two-theta, 6.91±2 degrees two-theta, 9.43±2 degrees two-theta, 9.85±2 degrees two-theta, and 13.48±2 degrees two-theta.

In some embodiments, crystalline Compound I Form 11 is characterized by an X-ray powder diffractogram having a signal at one or more of 6.72±2 degrees two-theta, 6.91±2 degrees two-theta, 9.43±2 degrees two-theta, and 9.85±2 degrees two-theta.

In some embodiments, crystalline Compound I Form 11 is characterized by an X-ray powder diffractogram having a signal at one or more of 6.72±2 degrees two-theta, 6.91±2 degrees two-theta, and 9.85±2 degrees two-theta.

In some embodiments, crystalline Compound I Form 11 is characterized by an X-ray powder diffractogram having a signal at one or more of 6.72±2 degrees two-theta, and 6.91±2 degrees two-theta.

In some embodiments, crystalline Compound I Form 11 is characterized by an X-ray powder diffractogram having a signal at 6.72±2 degrees two-theta. In some embodiments, crystalline Compound I Form 11 is characterized by an X-ray powder diffractogram having a signal at 6.91±2 degrees two-theta. In some embodiments, crystalline Compound I Form 11 is characterized by an X-ray powder diffractogram having a signal at 9.43±2 degrees two-theta. In some embodiments, crystalline Compound I Form 11 is characterized by an X-ray powder diffractogram having a signal at 9.85±2 degrees two-theta. In some embodiments, crystalline Compound I Form 11 is characterized by an X-ray powder diffractogram having a signal at 13.48±2 degrees two-theta.

In some embodiments, crystalline Compound I Form 11 is characterized by an X-ray powder diffractogram substantially similar to FIG. 27.

In some embodiments, crystalline Compound I Form 11 is characterized by a primitive monoclinic crystal system, P2₁/c space group and the following unit cell dimensions resulting from successful indexing of Form 11 XRPD:

| | | | |
|---|---|---|---|
| a | 12.8 ± 0.1 Å | α | 90 ± 0.1° |
| b | 7.0 ± 0.1 Å | β | 92.4 ± 0.1° |
| c | 26.3 ± 0.1 Å | γ | 90 ± 0.1°. |

In some embodiments, Compound I Form 11 is characterized by a DSC having a small broad endotherm peak temperature of 167° C.

In some embodiments, Compound I Form 11 is characterized by a DSC having an onset of melting temperature of 221° C. In some embodiments, Compound I Form 11 is characterized by a DSC having a broad endotherm peak temperature of 228° C. In some embodiments, Compound I Form 11 is characterized by a DSC having an onset of melting temperature of 221° C. and/or a peak temperature of 228° C.

In some embodiments, Compound I Form 11 is characterized by a DSC having an onset of melting temperature of 221° C. and/or a peak temperature of 233° C.

In some embodiments, Compound I Form 11 is characterized by a DSC having a double endotherm peak temperature of 228° C. and 233° C.

In some embodiments, Compound I Form 11 is characterized by a DSC having three endotherms at peak temperatures of 167° C., 228° C. and 233° C.

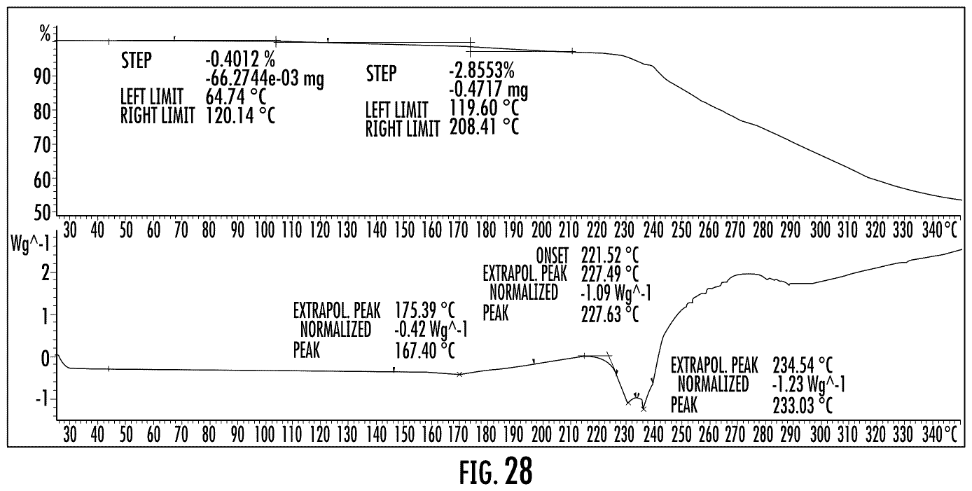

In some embodiments, Compound I Form 11 is characterized by a DSC substantially similar to FIG. 28.

In some embodiments, Compound I Form 11 is also characterized by a weight loss of 0.4% from 65° C. to 120° C., as measured by thermogravimetric analysis. In some embodiments, Compound I Form 11 is also characterized by a weight loss of approximately 2.9% at temperatures 120° C. to 208° C., as measured by thermogravimetric analysis.

In some embodiments, Compound I Form 11 is characterized by a TGA substantially similar to FIG. 28.

Another embodiment of the disclosure provides a method of making crystalline Compound I Form 11. In some embodiments, the method of making crystalline Compound I Form 11 comprises: slowly evaporating Compound I Form 3 in acetonitrile under nitrogen for 4 days to yield crystalline Compound I Form 11.

### L. Compound I Material 12

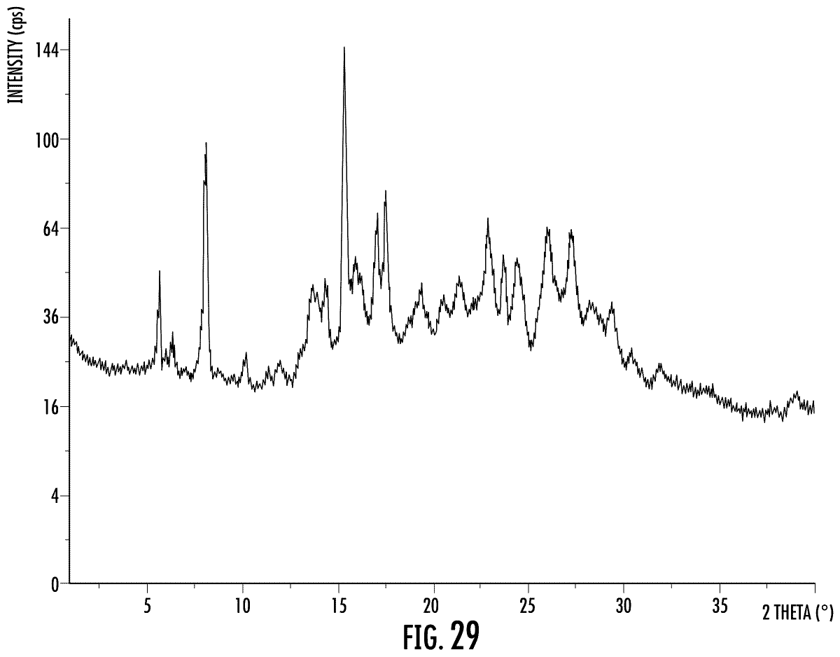

In some embodiments, the disclosure provides crystalline Compound I Material 12. FIG. 29 provides an X-ray powder diffractogram of disordered crystalline Compound I Material 12 with corresponding tabulated data for FIG. 29 shown in Table 17 Table 18.

TABLE 18
XRPD Signals for Crystalline Compound I Form 2

| °2Θ | d-spacing | Intensity | Relative |
|---|---|---|---|
| (±0.2°) | (Å) | (Counts) | Intensity |
| 5.65 | 15.63 | 1834 | 34 |
| 6.32 | 13.97 | 1203 | 22 |
| 8.11 | 10.89 | 3643 | 68 |
| 10.17 | 8.69 | 1005 | 19 |
| 13.63 | 6.49 | 1669 | 31 |
| 14.33 | 6.18 | 1736 | 32 |
| 15.35 | 5.77 | 5370 | 100 |
| 15.91 | 5.57 | 2010 | 37 |
| 17.03 | 5.2 | 2563 | 48 |
| 17.5 | 5.06 | 2897 | 54 |
| 19.37 | 4.58 | 1696 | 32 |
| 20.56 | 4.32 | 1560 | 29 |
| 21.3 | 4.17 | 1772 | 33 |
| 22.85 | 3.89 | 2515 | 47 |
| 23.65 | 3.76 | 2006 | 37 |
| 24.45 | 3.64 | 1978 | 37 |
| 25.92 | 3.43 | 2354 | 44 |
| 27.25 | 3.27 | 2324 | 43 |
| 28.21 | 3.16 | 1504 | 28 |
| 29.35 | 3.04 | 1476 | 27 |
| 30.42 | 2.94 | 1045 | 19 |

In some embodiments, crystalline Compound I Material 12 is characterized by an X-ray powder diffractogram generated by an X-ray powder diffraction analysis with an incident beam of Cu Kα radiation.

In some embodiments, crystalline Compound I Material 12 is characterized by an X-ray powder diffractogram having a signal at one or more of 5.65±2 degrees two-theta, 6.32±2 degrees two-theta, 8.11±2 degrees two-theta, 10.17±2 degrees two-theta, 13.63±2 degrees two-theta, 14.33±2 degrees two-theta, ±2 degrees two-theta, 15.35±2 degrees two-theta, 15.91±2 degrees two-theta, 17.03±2 degrees two-theta, 17.5±2 degrees two-theta, 19.37±2 degrees two-theta, 20.56±2 degrees two-theta, 21.3±2 degrees two-theta, 22.85±2 degrees two-theta, 23.65±2 degrees two-theta, 24.45±2 degrees two-theta, 25.92±2 degrees two-theta, 27.25±2 degrees two-theta, 28.21±2 degrees two-theta, 29.35±2 degrees two-theta, and 30.42±2 degrees two-theta.

In some embodiments, crystalline Compound I Material 12 is characterized by an X-ray powder diffractogram having a signal at one or more of 5.65±2 degrees two-theta, 6.32±2 degrees two-theta, 8.11±2 degrees two-theta, 13.63±2 degrees two-theta, and 14.33±2 degrees two-theta.

In some embodiments, crystalline Compound I Material 12 is characterized by an X-ray powder diffractogram having a signal at one or more of 5.65±2 degrees two-theta, 6.32±2 degrees two-theta, 8.11±2 degrees two-theta, and 14.33±2 degrees two-theta.

In some embodiments, crystalline Compound I Material 12 is characterized by an X-ray powder diffractogram having a signal at one or more of 5.65±2 degrees two-theta, 6.32±2 degrees two-theta, and 8.11±2 degrees two-theta.

In some embodiments, crystalline Compound I Material 12 is characterized by an X-ray powder diffractogram having a signal at one or more of 5.65±2 degrees two-theta, and 8.11±2 degrees two-theta.

In some embodiments, crystalline Compound I Material 12 is characterized by an X-ray powder diffractogram having a signal at 5.65±2 degrees two-theta. In some embodiments, crystalline Compound I Material 12 is characterized by an X-ray powder diffractogram having a signal at 6.32±2 degrees two-theta. In some embodiments, crystalline Compound I Material 12 is characterized by an X-ray powder diffractogram having a signal at 8.11±2 degrees two-theta. In some embodiments, crystalline Compound I Material 12 is characterized by an X-ray powder diffractogram having a signal at 13.63±2 degrees two-theta. In some embodiments, crystalline Compound I Material 12 is characterized by an X-ray powder diffractogram having a signal at 14.33±2 degrees two-theta.

In some embodiments, crystalline Compound I Material 12 is characterized by an X-ray powder diffractogram substantially similar to FIG. 29.

Another embodiment of the disclosure provides a method of making disordered crystalline Compound I Material 12. In some embodiments, the method of making crystalline Compound I Material 12 comprises: vacuum drying mixture of Compound I Form 2 and Material 6 at 75° for 3 days to yield crystalline Compound I Material 12.

### M. Compound I Material 13

In some embodiments, Compound I Material 13 is an acetonitrile solvate. In some embodiments, Compound I Material 13 is a mixture of Form 11 and acetonitrile solvate. In some embodiments, the method of making Compound I Material 13 and Form 11 mixture comprises slurrying Compound I Form 1 in acetonitrile at 70° C.

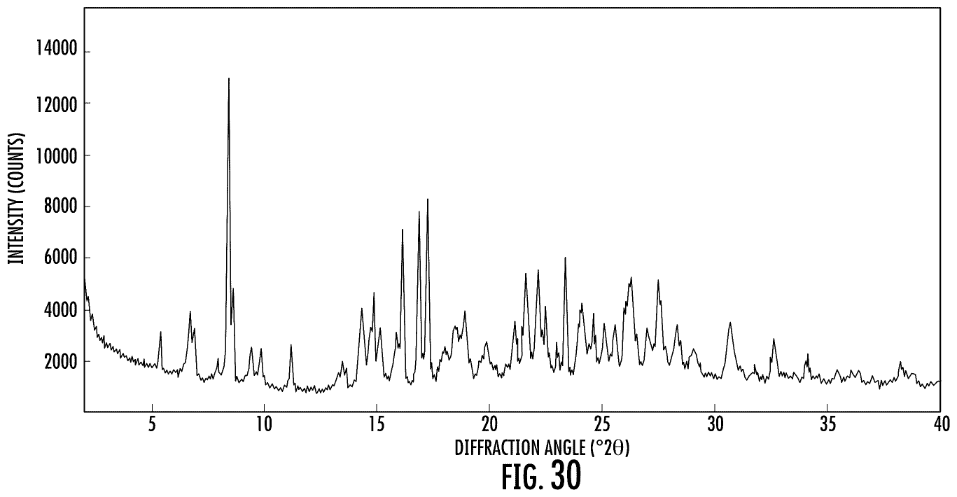

FIG. 30 provides an X-ray powder diffractogram of crystalline Compound I Material 13+Form 11 with corresponding tabulated data for FIG. 30 shown in Table 19.

TABLE 19
XRPD Signals for Crystalline Compound I
Material 13 + Form 11

| °2Θ | d-spacing | Relative |
|---|---|---|
| (±0.2°) | (Å) | Intensity |
| 5.42 | 16.3047 | 44 |
| 6.74 | 13.1142 | 56 |
| 6.94 | 12.7367 | 11 |
| 7.96 | 11.1067 | 8 |
| 8.46 | 10.4514 | 100 |
| 8.66 | 10.2105 | 21 |
| 9.46 | 9.3487 | 15 |
| 9.9 | 8.9342 | 17 |
| 11.24 | 7.8719 | 19 |
| 13.5 | 6.5588 | 8 |
| 13.7 | 6.4635 | 6 |
| 14.4 | 6.1508 | 27 |
| 14.9 | 5.9455 | 36 |
| 15.18 | 5.8365 | 22 |
| 15.9 | 5.5738 | 11 |
| 16.2 | 5.4712 | 55 |
| 16.92 | 5.24 | 77 |
| 17.3 | 5.1257 | 85 |
| 18.02 | 4.9225 | 10 |
| 18.48 | 4.801 | 13 |
| 18.98 | 4.6757 | 21 |
| 19.92 | 4.4571 | 13 |
| 21.18 | 4.1947 | 22 |
| 21.64 | 4.1066 | 44 |
| 22.22 | 4.0007 | 39 |
| 22.54 | 3.9446 | 22 |
| 23.04 | 3.8601 | 11 |
| 23.42 | 3.7983 | 52 |
| 24.14 | 3.6866 | 30 |
| 24.68 | 3.6072 | 20 |
| 25.14 | 3.5422 | 19 |
| 25.62 | 3.4769 | 22 |
| 26.34 | 3.3835 | 50 |
| 27 | 3.3023 | 17 |
| 27.54 | 3.2387 | 31 |
| 28.42 | 3.1404 | 19 |
| 29.12 | 3.0665 | 11 |
| 30.72 | 2.9103 | 26 |
| 32.66 | 2.7418 | 22 |
| 34.16 | 2.6247 | 13 |
| 35.46 | 2.5314 | 7 |
| 35.46 | 2.5314 | 7 |
| 38.30 | 2.35 | 7 |

In some embodiments, crystalline Compound I Material 13+Form 11 is substantially pure. In some embodiments, crystalline Compound I Material 13+Form 11 is substantially crystalline. In some embodiments, crystalline Compound I Material 13+Form 11 is characterized by an X-ray powder diffractogram generated by an X-ray powder diffraction analysis with an incident beam of Cu Kα radiation.

In some embodiments, crystalline Compound I Material 13+Form 11 is characterized by an X-ray powder diffractogram having a signal at one or more of 5.42±0.2 degrees two-theta, 6.74±0.2 degrees two-theta, 6.94±0.2 degrees two-theta, 7.96±0.2 degrees two-theta, 8.46±0.2 degrees two-theta, 8.66±0.2 degrees two-theta, ±0.2 degrees two-theta, 9.46±0.2 degrees two-theta, 9.9±0.2 degrees two-theta, 11.24±0.2 degrees two-theta, 13.5±0.2 degrees two-theta, 13.7±0.2 degrees two-theta, 14.4±0.2 degrees two-theta, 14.9±0.2 degrees two-theta, 15.18±0.2 degrees two-theta, 15.9±0.2 degrees two-theta, 16.2±0.2 degrees two-theta, 16.92±0.2 degrees two-theta, 17.3±0.2 degrees two-theta, 18.02±0.2 degrees two-theta, 18.48±0.2 degrees two-theta, 18.98±0.2 degrees two-theta, 19.92±0.2 degrees two-theta, 21.18±0.2 degrees two-theta, 21.64±0.2 degrees two-theta, 22.22±0.2 degrees two-theta, 22.54±0.2 degrees two-theta, 23.04±0.2 degrees two-theta, 23.42±0.2 degrees two-theta, 24.14±0.2 degrees two-theta, 24.68±0.2 degrees two-theta, 25.14±0.2 degrees two-theta, 25.62±0.2 degrees two-theta, 26.34±0.2 degrees two-theta, 27±0.2 degrees two-theta, 27.54±0.2 degrees two-theta, 28.42±0.2 degrees two-theta, 29.12±0.2 degrees two-theta, 30.72±0.2 degrees two-theta, 32.66±0.2 degrees two-theta, 34.16±0.2 degrees two-theta, 35.46±0.2 degrees two-theta, and 38.3±0.2 degrees two-theta.

In some embodiments, crystalline Compound I Material 13+Form 11 is characterized by an X-ray powder diffractogram having a signal at one or more of 5.42±0.2 degrees two-theta, 6.74±0.2 degrees two-theta, and 8.46±0.2 degrees two-theta.

In some embodiments, crystalline Compound I Material 13+Form 11 is characterized by an X-ray powder diffractogram having a signal at 5.42±0.2 degrees two-theta.

In some embodiments, crystalline Compound I Material 13+Form 11 is characterized by an X-ray powder diffractogram having a signal at 6.74±0.2 degrees two-theta.

In some embodiments, crystalline Compound I Material 13+Form 11 is characterized by an X-ray powder diffractogram having a signal at 8.46±0.2 degrees two-theta.

In some embodiments, crystalline Compound I Material 13+Form 11 is characterized by an X-ray powder diffractogram substantially similar to FIG. 30.

Another embodiment of the disclosure provides a method of making Compound I Material 13+Form 11. In some embodiments, the method of making crystalline Compound I Material 13+Form 11 comprises: stirring Compound I Form 1 in acetonitrile at 70° for 7 days and collecting the solids to yield crystalline Compound I Material 13+Form 11.

### N. Compound I Material 14

In some embodiments, Compound I Material 14 is metastable at ambient conditions. In some embodiments, Compound I Material 14 may be considered a hydrate or solvate.

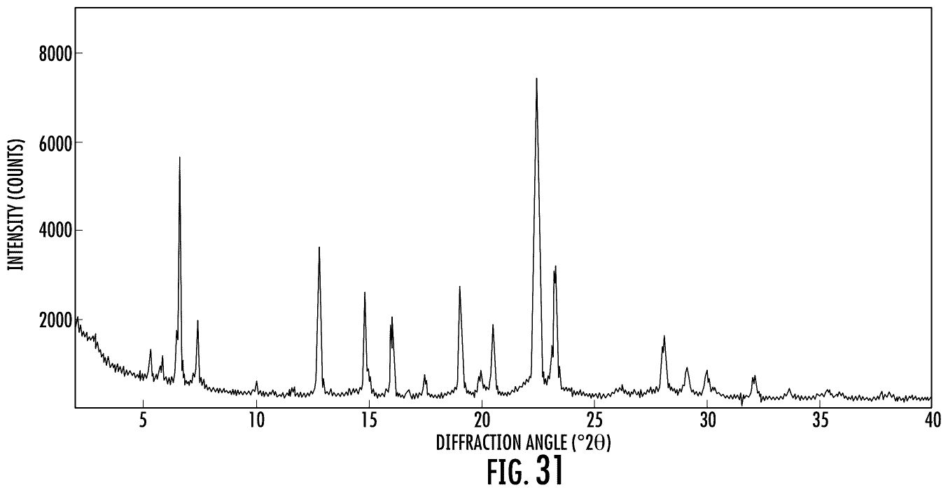

FIG. 31 provides an X-ray powder diffractogram of crystalline Compound I Material 14 with corresponding tabulated data for FIG. 31 shown in Table 20.

TABLE 20
XRPD Signals for Crystalline Compound I Material 14

| °2Θ | d-spacing | Relative |
|---|---|---|
| (±0.2°) | (Å) | Intensity |
| 5.32 | 16.611 | 9 |
| 5.8 | 15.2373 | 8 |
| 6.62 | 13.3516 | 70 |
| 7.4 | 11.946 | 19 |
| 10.02 | 8.8275 | 4 |
| 12.82 | 6.9051 | 48 |
| 14.86 | 5.9614 | 31 |
| 16.04 | 5.5254 | 25 |
| 17.5 | 5.0676 | 6 |
| 19.08 | 4.6514 | 36 |
| 19.96 | 4.4482 | 7 |
| 20.54 | 4.3239 | 22 |
| 22.46 | 3.9584 | 100 |
| 23.28 | 3.8208 | 39 |
| 26.24 | 3.3962 | 3 |
| 28.12 | 3.1732 | 17 |
| 29.16 | 3.0624 | 9 |
| 30 | 2.9785 | 7 |
| 32.14 | 2.7849 | 8 |
| 33.66 | 2.6626 | 3 |
| 35.44 | 2.5328 | 2 |
| 38.12 | 2.3607 | 2 |

In some embodiments, crystalline Compound I Material 14 is substantially pure. In some embodiments, crystalline Compound I Material 14 is substantially crystalline. In some embodiments, crystalline Compound I Material 14 is characterized by an X-ray powder diffractogram generated by an X-ray powder diffraction analysis with an incident beam of Cu Kα radiation.

In some embodiments, crystalline Compound I Material 14 is characterized by an X-ray powder diffractogram having a signal at one or more of 5.32±0.2 degrees two-theta, 5.8±0.2 degrees two-theta, 6.62±0.2 degrees two-theta, 7.4±0.2 degrees two-theta, 10.02±0.2 degrees two-theta, 12.82±0.2 degrees two-theta, ±0.2 degrees two-theta, 14.86±0.2 degrees two-theta, 16.04±0.2 degrees two-theta, 17.5±0.2 degrees two-theta, 19.08±0.2 degrees two-theta, 19.96±0.2 degrees two-theta, 20.54±0.2 degrees two-theta, 22.46±0.2 degrees two-theta, 23.28±0.2 degrees two-theta, 26.24±0.2 degrees two-theta, 28.12±0.2 degrees two-theta, 29.16±0.2 degrees two-theta, 30±0.2 degrees two-theta, 32.14±0.2 degrees two-theta, 33.66±0.2 degrees two-theta, 35.44±0.2 degrees two-theta, 38.12±0.2 degrees two-theta, and 38.12±0.2 degrees two-theta.

In some embodiments, crystalline Compound I Material 14 is characterized by an X-ray powder diffractogram having a signal at one or more of 6.62±0.2 degrees two-theta, 7.4±0.2 degrees two-theta, 12.82±0.2 degrees two-theta, 14.86±0.2 degrees two-theta, and 22.46±0.2 degrees two-theta.

In some embodiments, crystalline Compound I Material 14 is characterized by an X-ray powder diffractogram having a signal at one or more of 14.86±0.2 degrees two-theta and 22.46±0.2 degrees two-theta.

In some embodiments, crystalline Compound I Material 14 is characterized by an X-ray powder diffractogram having a signal at 6.62±0.2 degrees two-theta.

In some embodiments, crystalline Compound I Material 14 is characterized by an X-ray powder diffractogram having a signal at 7.4±0.2 degrees two-theta.

In some embodiments, crystalline Compound I Material 14 is characterized by an X-ray powder diffractogram having a signal at 12.82±0.2 degrees two-theta.

In some embodiments, crystalline Compound I Material 14 is characterized by an X-ray powder diffractogram having a signal at 14.86±0.2 degrees two-theta.

In some embodiments, crystalline Compound I Material 14 is characterized by an X-ray powder diffractogram having a signal at 22.46±0.2 degrees two-theta.

In some embodiments, crystalline Compound I Material 14 is characterized by an X-ray powder diffractogram substantially similar to FIG. 31.

Another embodiment of the disclosure provides a method of making Compound I Material 14. In some embodiments, the method of making crystalline Compound I Material 14 comprises: dissolving Compound I Form 1 in dimethylformamide (DMF), placing in a vial containing dichloromethane (DCM) and leaving at room temperature for vapor diffusion to yield crystalline Compound I Material 14.

### O. Compound I Material 15

In some embodiments, Compound I Material 15 is metastable at ambient conditions. In some embodiments, the method of making Compound I Material 15 comprises precipitating Compound I Form 1 in acetic acid and hexane for one week. In some embodiments, Compound I Material 15 is a mixture of Material 10 and acetic acid solvate.

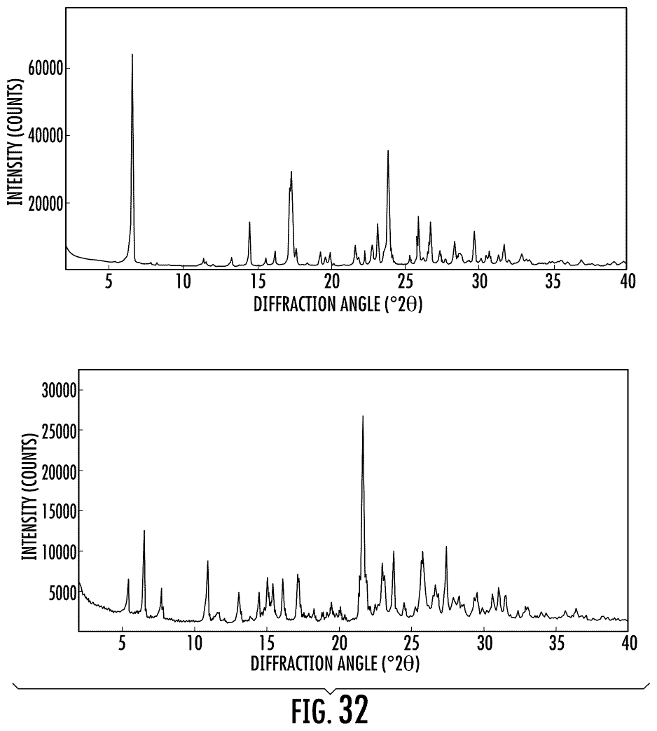

FIG. 32 provides an X-ray powder diffractogram of crystalline Compound I Material 15 with corresponding tabulated data for FIG. 32 shown in Table 21.

TABLE 21
XRPD Signals for Crystalline Compound I Material 15

| °2Θ | d-spacing | Relative |
|---|---|---|
| (±0.2°) | (Å) | Intensity |
| 6.54 | 13.5148 | 100 |
| 7.78 | 11.3633 | 1 |
| 8.20 | 10.7822 | 1 |
| 11.34 | 7.8027 | 4 |
| 13.24 | 6.6870 | 4 |
| 11.50 | 7.6945 | 1 |
| 14.46 | 6.1254 | 21 |
| 15.04 | 5.8905 | 1 |
| 15.52 | 5.7094 | 4 |
| 16.16 | 5.4847 | 6 |
| 17.28 | 5.1316 | 39 |
| 17.58 | 5.0447 | 5 |
| 19.22 | 4.6178 | 5 |
| 19.58 | 4.5337 | 3 |
| 19.86 | 4.4704 | 5 |
| 21.58 | 4.1178 | 7 |
| 21.82 | 4.0731 | 2 |
| 22.22 | 4.0007 | 5 |
| 22.74 | 3.9103 | 8 |
| 23.08 | 3.8535 | 16 |
| 23.82 | 3.7354 | 53 |
| 25.26 | 3.5257 | 4 |
| 25.84 | 3.4478 | 21 |
| 26.18 | 3.4038 | 1 |
| 26.66 | 3.3436 | 19 |
| 27.30 | 3.2667 | 6 |
| 27.68 | 3.2227 | 2 |
| 28.28 | 3.1556 | 10 |
| 28.66 | 3.1147 | 4 |
| 29.60 | 3.0179 | 14 |
| 30.04 | 2.9747 | 2 |
| 30.42 | 2.9384 | 2 |
| 30.62 | 2.9196 | 4 |
| 31.22 | 2.8649 | 3 |
| 31.60 | 2.8313 | 9 |
| 31.94 | 2.8019 | 2 |
| 32.84 | 2.7271 | 4 |
| 33.12 | 2.7047 | 1 |
| 33.38 | 2.6843 | 1 |
| 35.90 | 2.5014 | 1 |
| 36.82 | 2.4410 | 3 |
| 39.04 | 2.3071 | 2 |
| 39.70 | 2.2703 | 1 |

In some embodiments, crystalline Compound I Material 15 is substantially pure. In some embodiments, crystalline Compound I Material 15 is substantially crystalline. In some embodiments, crystalline Compound I Material 15 is characterized by an X-ray powder diffractogram generated by an X-ray powder diffraction analysis with an incident beam of Cu Kα radiation.

In some embodiments, crystalline Compound I Material 15 is characterized by an X-ray powder diffractogram having a signal at one or more of 6.54±0.2 degrees two-theta, 7.78±0.2 degrees two-theta, 8.2±0.2 degrees two-theta, 11.34±0.2 degrees two-theta, 13.24±0.2 degrees two-theta, 11.5±0.2 degrees two-theta, ±0.2 degrees two-theta, 14.46±0.2 degrees two-theta, 15.04±0.2 degrees two-theta, 15.52±0.2 degrees two-theta, 16.16±0.2 degrees two-theta, 17.28±0.2 degrees two-theta, 17.58±0.2 degrees two-theta, 19.22±0.2 degrees two-theta, 19.58±0.2 degrees two-theta, 19.86±0.2 degrees two-theta, 21.58±0.2 degrees two-theta, 21.82±0.2 degrees two-theta, 22.22±0.2 degrees two-theta, 22.74±0.2 degrees two-theta, 23.08±0.2 degrees two-theta, 23.82±0.2 degrees two-theta, 25.26±0.2 degrees two-theta, 25.84±0.2 degrees two-theta, 26.18±0.2 degrees two-theta, 26.66±0.2 degrees two-theta, 27.3±0.2 degrees two-theta, 27.68±0.2 degrees two-theta, 28.28±0.2 degrees two-theta, 28.66±0.2 degrees two-theta, 29.6±0.2 degrees two-theta, 30.04±0.2 degrees two-theta, 30.42±0.2 degrees two-theta, 30.62±0.2 degrees two-theta, 31.22±0.2 degrees two-theta, 31.6±0.2 degrees two-theta, 31.94±0.2 degrees two-theta, 32.84±0.2 degrees two-theta, 33.12±0.2 degrees two-theta, 33.38±0.2 degrees two-theta, 35.9±0.2 degrees two-theta, 36.82±0.2 degrees two-theta, 39.04±0.2 degrees two-theta, and 39.7±0.2 degrees two-theta.

In some embodiments, crystalline Compound I Material 15 is characterized by an X-ray powder diffractogram having a signal at one or more of 6.54±0.2 degrees two-theta, 11.34±0.2 degrees two-theta, 13.24±0.2 degrees two-theta, 14.46±0.2 degrees two-theta, 17.28±0.2 degrees two-theta, and 23.82±0.2 degrees two-theta.

In some embodiments, crystalline Compound I Material 15 is characterized by an X-ray powder diffractogram having a signal at one or more of 11.34±0.2 degrees two-theta, 13.24±0.2 degrees two-theta, and 23.82±0.2 degrees two-theta.

In some embodiments, crystalline Compound I Material 15 is characterized by an X-ray powder diffractogram having a signal at 6.54±0.2 degrees two-theta. In some embodiments, crystalline Compound I Material 15 is characterized by an X-ray powder diffractogram having a signal at 11.34±0.2 degrees two-theta. In some embodiments, crystalline Compound I Material 15 is characterized by an X-ray powder diffractogram having a signal at 13.24±0.2 degrees two-theta. In some embodiments, crystalline Compound I Material 15 is characterized by an X-ray powder diffractogram having a signal at 14.46±0.2 degrees two-theta. In some embodiments, crystalline Compound I Material 15 is characterized by an X-ray powder diffractogram having a signal at 17.28±0.2 degrees two-theta. In some embodiments, crystalline Compound I Material 15 is characterized by an X-ray powder diffractogram having a signal at 23.82±0.2 degrees two-theta.

In some embodiments, crystalline Compound I Material 15 is characterized by an X-ray powder diffractogram substantially similar to FIG. 32.

Another embodiment of the disclosure provides a method of making Compound I Material 15. In some embodiments, the method of making crystalline Compound I Material 15 comprises: dissolving Compound I Form 1 in glacial acetic acid, stirring at room temperature for 1 week, adding hexane, removing the clear solvent, and drying under ambient conditions for 2 day to yield crystalline Compound I Material 15.

In some embodiments, the method of making crystalline Compound I Material 15 comprises: milling Compound I Form 2 with glacial acetic acid diluted in ethyl acetate for 30 minutes to yield crystalline Compound I Material 15+Material 10.

### P. Compound I Material 16

In some embodiments, Compound I Material 16 is an hexafluoroisopropanol (HFIPA) solvate. In some embodiments, Compound I Material 16 is a mixture of Form 2 and HFIPA solvate. In some embodiments, Compound I Material 16 is metastable at ambient conditions. In some embodiments, the method of making Compound I Material 16 and Form 2 mixture comprises slowly evaporating Compound I Form 1 from a HFIPA solution.

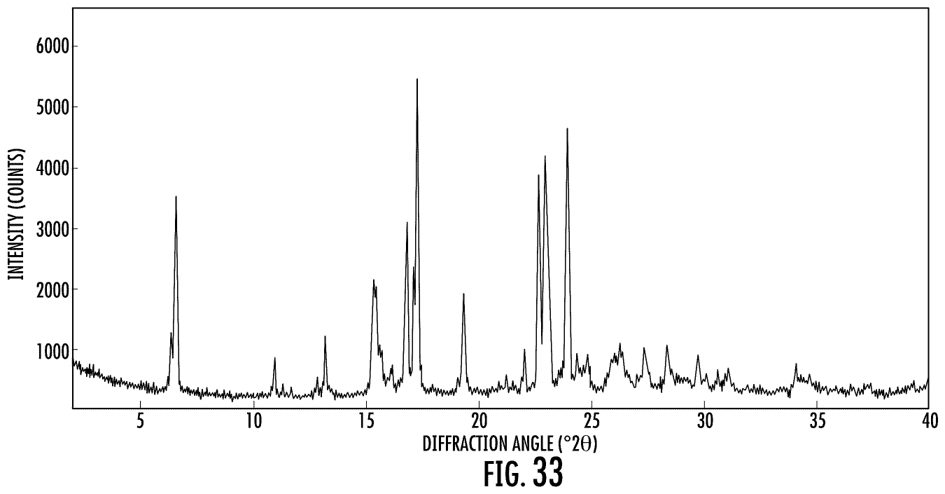

FIG. 33 provides an X-ray powder diffractogram of crystalline Compound I Material 16 with corresponding tabulated data for FIG. 33 shown in Table 22.

TABLE 22
XRPD Signals for Crystalline Compound I Material 16

| °2Θ | d-spacing | Relative |
|---|---|---|
| (±0.2°) | (Å) | Intensity |
| 6.44 | 13.7244 | 12 |
| 6.66 | 13.2715 | 60 |
| 11.04 | 8.0141 | 14 |
| 12.92 | 6.8519 | 6 |
| 13.26 | 6.6769 | 21 |
| 15.42 | 5.7462 | 44 |
| 16.22 | 5.4645 | 5 |
| 16.90 | 5.2461 | 64 |
| 17.18 | 5.1613 | 21 |
| 17.34 | 5.1140 | 86 |
| 18.02 | 4.9225 | 3 |
| 19.40 | 4.5754 | 39 |
| 20.64 | 4.3032 | 2 |
| 20.92 | 4.2462 | 3 |
| 21.28 | 4.1752 | 5 |
| 22.06 | 4.0293 | 17 |
| 22.72 | 3.9137 | 70 |
| 23.00 | 3.8667 | 81 |
| 23.66 | 3.7603 | 2 |
| 24.00 | 3.7078 | 100 |
| 24.40 | 3.6479 | 7 |
| 24.88 | 3.5786 | 8 |
| 26.34 | 3.3835 | 8 |
| 27.38 | 3.2573 | 12 |
| 28.38 | 3.1448 | 16 |
| 29.74 | 3.0040 | 10 |
| 30.12 | 2.9669 | 4 |
| 30.60 | 2.9215 | 7 |
| 31.14 | 2.8720 | 5 |
| 34.08 | 2.6307 | 7 |
| 34.70 | 2.5851 | 5 |
| 36.68 | 2.4500 | 2 |
| 37.40 | 2.4045 | 4 |
| 39.28 | 2.2936 | 3 |

In some embodiments, crystalline Compound I Material 16 is substantially pure. In some embodiments, crystalline Compound I Material 16 is substantially crystalline. In some embodiments, crystalline Compound I Material 16 is characterized by an X-ray powder diffractogram generated by an X-ray powder diffraction analysis with an incident beam of Cu Kα radiation.

In some embodiments, crystalline Compound I Material 16 is characterized by an X-ray powder diffractogram having a signal at one or more of 6.44±0.2 degrees two-theta, 6.66±0.2 degrees two-theta, 11.04±0.2 degrees two-theta, 12.92±0.2 degrees two-theta, 13.26±0.2 degrees two-theta, 15.42±0.2 degrees two-theta, ±0.2 degrees two-theta, 16.22±0.2 degrees two-theta, 16.9±0.2 degrees two-theta, 17.18±0.2 degrees two-theta, 17.34±0.2 degrees two-theta, 18.02±0.2 degrees two-theta, 19.4±0.2 degrees two-theta, 20.64±0.2 degrees two-theta, 20.92±0.2 degrees two-theta, 21.28±0.2 degrees two-theta, 22.06±0.2 degrees two-theta, 22.72±0.2 degrees two-theta, 23±0.2 degrees two-theta, 23.66±0.2 degrees two-theta, 24±0.2 degrees two-theta, 24.4±0.2 degrees two-theta, 24.88±0.2 degrees two-theta, 26.34±0.2 degrees two-theta, 27.38±0.2 degrees two-theta, 28.38±0.2 degrees two-theta, 29.74±0.2 degrees two-theta, 30.12±0.2 degrees two-theta, 30.6±0.2 degrees two-theta, 31.14±0.2 degrees two-theta, 34.08±0.2 degrees two-theta, 34.7±0.2 degrees two-theta, 36.68±0.2 degrees two-theta, 37.4±0.2 degrees two-theta, and 39.28±0.2 degrees two-theta.

In some embodiments, crystalline Compound I Material 16 is characterized by an X-ray powder diffractogram having a signal at one or more of 6.66±0.2 degrees two-theta, 11.04±0.2 degrees two-theta, 13.26±0.2 degrees two-theta, 16.9±0.2 degrees two-theta, 19.4±0.2 degrees two-theta, and 24±0.2 degrees two-theta.

In some embodiments, crystalline Compound I Material 16 is characterized by an X-ray powder diffractogram having a signal at one or more of 6.66±0.2 degrees two-theta, 13.26±0.2 degrees two-theta, and 16.9±0.2 degrees two-theta.

In some embodiments, crystalline Compound I Material 16 is characterized by an X-ray powder diffractogram having a signal at one or more of 11.04±0.2 degrees two-theta, 19.4±0.2 degrees two-theta, and 24±0.2 degrees two-theta.

In some embodiments, crystalline Compound I Material 16 is characterized by an X-ray powder diffractogram having a signal at 6.66±0.2 degrees two-theta. In some embodiments, crystalline Compound I Material 16 is characterized by an X-ray powder diffractogram having a signal at 11.04±0.2 degrees two-theta. In some embodiments, crystalline Compound I Material 16 is characterized by an X-ray powder diffractogram having a signal at 13.26±0.2 degrees two-theta. In some embodiments, crystalline Compound I Material 16 is characterized by an X-ray powder diffractogram having a signal at 16.9±0.2 degrees two-theta. In some embodiments, crystalline Compound I Material 16 is characterized by an X-ray powder diffractogram having a signal at 19.4±0.2 degrees two-theta. In some embodiments, crystalline Compound I Material 16 is characterized by an X-ray powder diffractogram having a signal at 24±0.2 degrees two-theta.

In some embodiments, crystalline Compound I Material 16 is characterized by an X-ray powder diffractogram substantially similar to FIG. 33.

Another embodiment of the disclosure provides a method of making Compound I Material 16. In some embodiments, the method of making crystalline Compound I Material 16 comprises: dissolving Compound I Form 1 in hexafluoroisopropanol (HFIPA) and evaporating at room temperature to yield crystalline Compound I Material 16.

### Q. Compound I Material 17

In some embodiments, Compound I Material 17 is found in slurry of Compound I Form 1 in isopropyl acetate (IPrOAc) at 70° C. In some embodiments, Compound I Material 17 is found in a solution of Compound I Material 10+Form 2 in methyl ethyl ketone (MVEK) at 70° C.

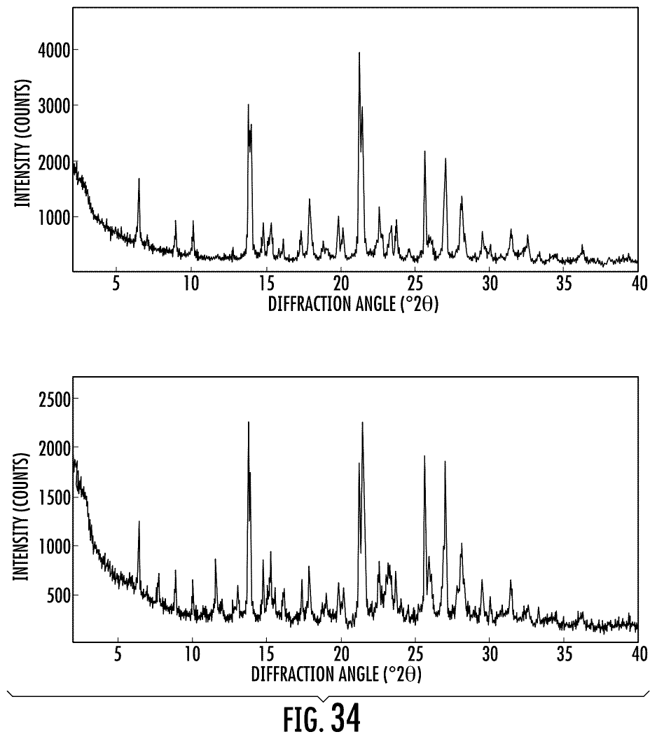

FIG. 34 provides an X-ray powder diffractogram of crystalline Compound I Material 17 with corresponding tabulated data for FIG. 34 shown in Table 23.

TABLE 23
XRPD Signals for Crystalline Compound I Material 17

| °2Θ | d-spacing | Relative |
|---|---|---|
| (±0.2°) | (Å) | Intensity |
| 6.44 | 13.7244 | 51 |
| 6.98 | 12.6638 | 19 |
| 8.9 | 9.9357 | 28 |
| 10.06 | 8.7925 | 26 |
| 12.74 | 6.9483 | 12 |
| 13.82 | 6.4076 | 90 |
| 14.78 | 5.9935 | 27 |
| 15.12 | 5.8595 | 18 |
| 15.32 | 5.7834 | 27 |
| 15.84 | 5.5947 | 12 |
| 16.12 | 5.4982 | 18 |
| 17.36 | 5.1081 | 22 |
| 17.9 | 4.9552 | 38 |
| 18.78 | 4.725 | 16 |
| 19.84 | 4.4749 | 17 |
| 20.16 | 4.4046 | 11 |
| 21.26 | 4.1791 | 100 |
| 22.58 | 3.9377 | 25 |
| 23.4 | 3.8015 | 15 |
| 23.74 | 3.7478 | 15 |
| 24.56 | 3.6245 | 5 |
| 25.68 | 3.4689 | 49 |
| 26.06 | 3.4192 | 6 |
| 27.04 | 3.2975 | 51 |
| 28.14 | 3.171 | 30 |
| 29.52 | 3.0259 | 11 |
| 30.06 | 2.9727 | 6 |
| 30.76 | 2.9066 | 3 |
| 31.44 | 2.8453 | 13 |
| 32.56 | 2.75 | 9 |
| 33.36 | 2.6858 | 4 |
| 34.38 | 2.6084 | 4 |
| 36.22 | 2.48 | 6 |
| 38.02 | 2.3667 | 2 |

In some embodiments, crystalline Compound I Material 17 is substantially pure. In some embodiments, crystalline Compound I Material 17 is substantially crystalline. In some embodiments, crystalline Compound I Material 17 is characterized by an X-ray powder diffractogram generated by an X-ray powder diffraction analysis with an incident beam of Cu Kα radiation.

In some embodiments, crystalline Compound I Material 17 is characterized by an X-ray powder diffractogram having a signal at one or more of 6.44±0.2 degrees two-theta, 6.98±0.2 degrees two-theta, 8.9±0.2 degrees two-theta, 10.06±0.2 degrees two-theta, 12.74±0.2 degrees two-theta, 13.82±0.2 degrees two-theta, ±0.2 degrees two-theta, 14.78±0.2 degrees two-theta, 15.12±0.2 degrees two-theta, 15.32±0.2 degrees two-theta, 15.84±0.2 degrees two-theta, 16.12±0.2 degrees two-theta, 17.36±0.2 degrees two-theta, 17.9±0.2 degrees two-theta, 18.78±0.2 degrees two-theta, 19.84±0.2 degrees two-theta, 20.16±0.2 degrees two-theta, 21.26±0.2 degrees two-theta, 22.58±0.2 degrees two-theta, 23.4±0.2 degrees two-theta, 23.74±0.2 degrees two-theta, 24.56±0.2 degrees two-theta, 25.68±0.2 degrees two-theta, 26.06±0.2 degrees two-theta, 27.04±0.2 degrees two-theta, 28.14±0.2 degrees two-theta, 29.52±0.2 degrees two-theta, 30.06±0.2 degrees two-theta, 30.76±0.2 degrees two-theta, 31.44±0.2 degrees two-theta, 32.56±0.2 degrees two-theta, 33.36±0.2 degrees two-theta, 34.38±0.2 degrees two-theta, 36.22±0.2 degrees two-theta, and 38.02±0.2 degrees two-theta.

In some embodiments, crystalline Compound I Material 17 is characterized by an X-ray powder diffractogram having a signal at one or more of 6.44±0.2 degrees two-theta, 8.9±0.2 degrees two-theta, 10.06±0.2 degrees two-theta, 13.82±0.2 degrees two-theta, 21.26±0.2 degrees two-theta, and 27.04±0.2 degrees two-theta.

In some embodiments, crystalline Compound I Material 17 is characterized by an X-ray powder diffractogram having a signal at one or more of 6.44±0.2 degrees two-theta, 8.9±0.2 degrees two-theta, and 13.82±0.2 degrees two-theta.

In some embodiments, crystalline Compound I Material 17 is characterized by an X-ray powder diffractogram having a signal at one or more of 10.06±0.2 degrees two-theta, 21.26±0.2 degrees two-theta, and 27.04±0.2 degrees two-theta.

In some embodiments, crystalline Compound I Material 17 is characterized by an X-ray powder diffractogram having a signal at 6.44±0.2 degrees two-theta. In some embodiments, crystalline Compound I Material 17 is characterized by an X-ray powder diffractogram having a signal at 8.9±0.2 degrees two-theta. In some embodiments, crystalline Compound I Material 17 is characterized by an X-ray powder diffractogram having a signal at 10.06±0.2 degrees two-theta. In some embodiments, crystalline Compound I Material 17 is characterized by an X-ray powder diffractogram having a signal at 13.82±0.2 degrees two-theta. In some embodiments, crystalline Compound I Material 17 is characterized by an X-ray powder diffractogram having a signal at 21.26±0.2 degrees two-theta. In some embodiments, crystalline Compound I Material 17 is characterized by an X-ray powder diffractogram having a signal at 27.04±0.2 degrees two-theta.

In some embodiments, crystalline Compound I Material 17 is characterized by an X-ray powder diffractogram substantially similar to FIG. 34.

Another embodiment of the disclosure provides a method of making Compound I Material 17. In some embodiments, the method of making crystalline Compound I Material 17 comprises: slurrying Compound I Form 1 in isopropyl acetate at 70° C., stirring at 70° C. for 7 days, and recovering the solids to yield crystalline Compound I Material 17.

In some embodiments, the method of making crystalline Compound I Material 17 comprises: slurrying Compound I Form 1 in methyl ethyl ketone at 70° C., stirring at 70° C. for 7 days, and recovering the solids to yield crystalline Compound I Material 17.

### R. Compound I Amorphous Form

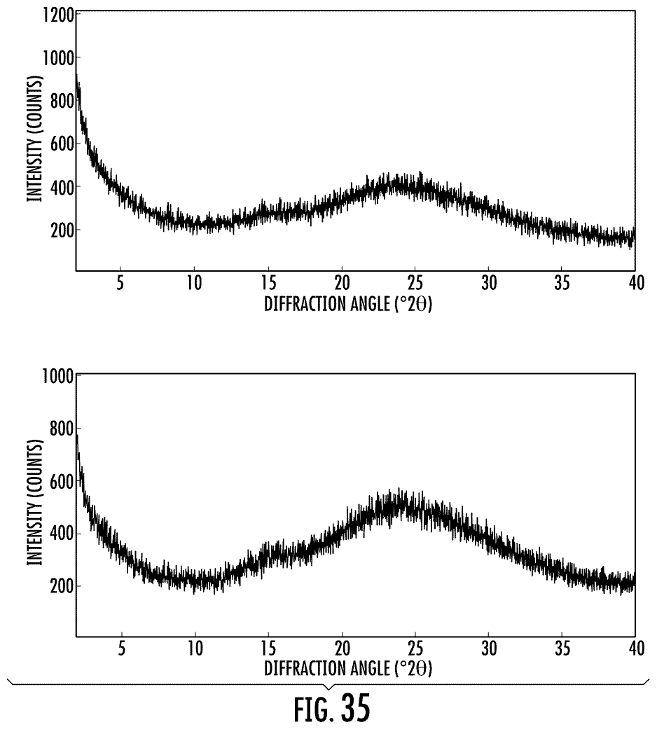

In some embodiments, the disclosure provides an amorphous form of Compound I. In some embodiments, the disclosure provides Compound I Amorphous Form. FIG. 35 provides an X-ray powder diffractogram of Compound I Amorphous Form at room temperature.

In some embodiments, Compound I Amorphous Form is substantially pure. In some embodiments, Compound I Amorphous Form is substantially amorphous. In some embodiments, Compound I Amorphous Form is characterized by an X-ray powder diffractogram generated by an X-ray powder diffraction analysis with an incident beam of Cu Kα radiation.

In some embodiments, Compound I Amorphous Form is characterized by an X-ray powder diffractogram substantially similar to FIG. 35.

In some embodiments, Compound I Amorphous Form is characterized by a DSC having a glass transition temperature of 160° C. In some embodiments, Compound I Amorphous Form is characterized by a DSC having a glass transition temperature of 162° C. In some embodiments, Compound I Amorphous Form is characterized by a DSC having a glass transition temperature of 175° C.

In some embodiments, Compound I Amorphous Form is characterized by a DSC having glass transition temperatures of 160° C., 162° C. and 175° C.

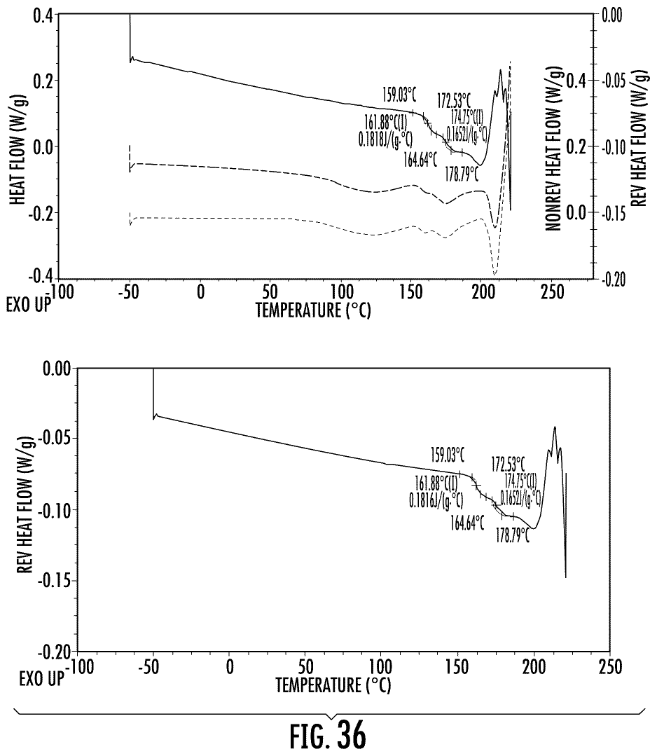

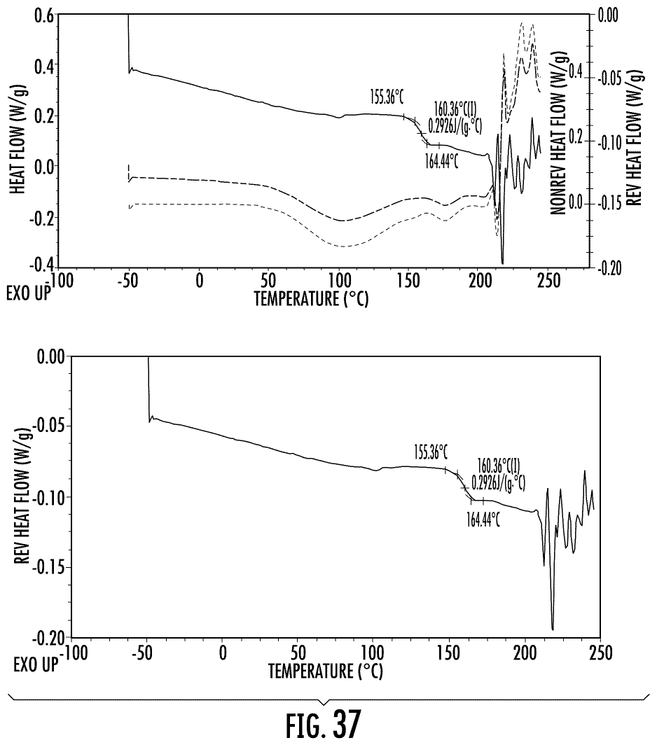

In some embodiments, Compound I Amorphous Form is characterized by a DSC substantially similar to FIG. 36. In some embodiments, Compound I Amorphous Form is characterized by a DSC substantially similar to FIG. 37.

In some embodiments, Compound I Amorphous Form is also characterized by a weight loss of 1.8% up to 150° C., as measured by thermogravimetric analysis. In some embodiments, Compound I Amorphous Form is also characterized by a weight loss of approximately 5.6% at temperatures up to 150° C., as measured by thermogravimetric analysis.

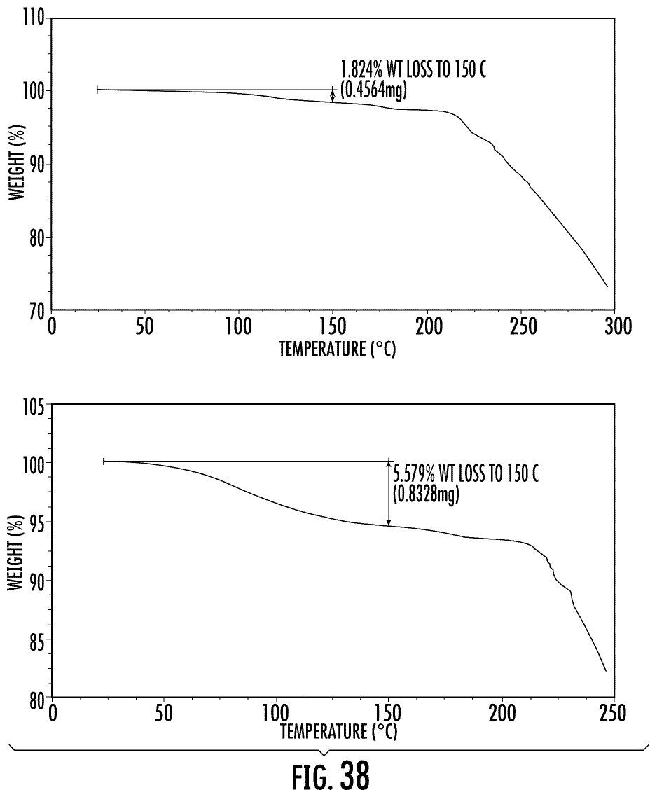

In some embodiments, Compound I Amorphous Form is characterized by a TGA substantially similar to FIG. 38.

Another embodiment of the disclosure provides a method of making Compound I Amorphous Form. In some embodiments, the method of making Compound I Amorphous Form comprises: dissolving Compound I Form 1 in methanol at room temperature with sonicating, filtering, and spray drying to yield Compound I Amorphous Form.

Another embodiment of the disclosure provides a method of making Compound I Amorphous Form. In some embodiments, the method of making Compound I Amorphous Form comprises: dissolving Compound I Form 2 in water, filtering, freezing, and lyophilizing to yield Compound I Amorphous Form.

### S. Compound I Adipic Acid Material 1

In some embodiments, the disclosure provides crystalline Compound I Adipic Acid Material 1. In some embodiments, Compound I Adipic Acid Material 1 is a hydrate. In some embodiments, Compound Adipic Acid Material 1 is a hydrate with about 0.5 moles of adipic acid.

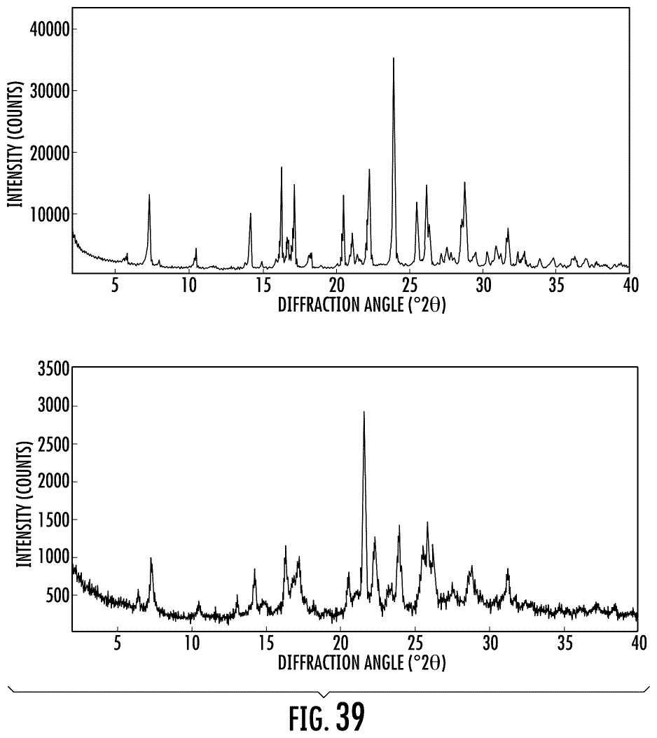

In some embodiments, the disclosure provides Compound I Adipic Acid Material 1. FIG. 39 provides an X-ray powder diffractogram of Compound I Adipic Acid Material 1 with corresponding tabulated data shown in Table 24.

TABLE 24
XRPD Signals for Crystalline Compound I
Adipic Acid Material I

| °2Θ | d-spacing | Relative |
|---|---|---|
| (±0.2°) | (Å) | Intensity |
| 5.54 | 15.9518 | 1 |
| 5.76 | 15.343 | 3 |
| 7.32 | 12.0763 | 30 |
| 7.96 | 11.1067 | 3 |
| 10.46 | 8.4571 | 9 |
| 11.6 | 7.6284 | 1 |
| 11.78 | 7.5123 | 1 |
| 12.86 | 6.8837 | 1 |
| 13.36 | 6.6272 | 1 |
| 13.82 | 6.4076 | 1 |
| 14.16 | 6.2545 | 23 |
| 14.94 | 5.9297 | 3 |
| 15.9 | 5.5738 | 2 |
| 16.28 | 5.4445 | 43 |
| 16.68 | 5.3148 | 11 |
| 17.16 | 5.1672 | 34 |
| 18.14 | 4.8902 | 2 |
| 18.3 | 4.8478 | 3 |
| 19.04 | 4.6611 | 1 |
| 20.06 | 4.4263 | 1 |
| 20.5 | 4.3323 | 30 |
| 21.1 | 4.2104 | 14 |
| 21.46 | 4.1406 | 4 |
| 22.28 | 3.99 | 42 |
| 23.04 | 3.8601 | 1 |
| 23.94 | 3.717 | 100 |
| 25.5 | 3.493 | 27 |
| 26.16 | 3.4064 | 26 |
| 26.36 | 3.381 | 6 |
| 26.9 | 3.3143 | 1 |
| 27.16 | 3.2832 | 4 |
| 27.52 | 3.241 | 6 |
| 27.78 | 3.2113 | 3 |
| 28.06 | 3.1799 | 1 |
| 28.54 | 3.1275 | 7 |
| 28.76 | 3.1041 | 27 |
| 29.48 | 3.0299 | 6 |
| 30.28 | 2.9516 | 7 |
| 30.62 | 2.9196 | 1 |
| 30.88 | 2.8956 | 7 |
| 31.22 | 2.8649 | 4 |
| 31.72 | 2.8208 | 16 |
| 32.38 | 2.7648 | 5 |
| 32.8 | 2.7304 | 5 |
| 33.2 | 2.6984 | 1 |
| 33.86 | 2.6473 | 4 |
| 34.78 | 2.5793 | 3 |
| 35.24 | 2.5467 | 1 |
| 36.26 | 2.4774 | 4 |
| 37 | 2.4295 | 4 |
| 37.36 | 2.4069 | 1 |
| 37.7 | 2.386 | 2 |

In some embodiments, crystalline Compound I Adipic Acid Material 1 is substantially pure. In some embodiments, crystalline Compound I Adipic Acid Material 1 is substantially crystalline. In some embodiments, crystalline Compound I Adipic Acid Material 1 is characterized by an X-ray powder diffractogram generated by an X-ray powder diffraction analysis with an incident beam of Cu Kα radiation.

In some embodiments, crystalline Compound I Adipic Acid Material 1 is characterized by an X-ray powder diffractogram having a signal at one or more of 5.54±0.2 degrees two-theta, 5.76±0.2 degrees two-theta, 7.32±0.2 degrees two-theta, 7.96±0.2 degrees two-theta, 10.46±0.2 degrees two-theta, 11.6±0.2 degrees two-theta, ±0.2 degrees two-theta, 11.78±0.2 degrees two-theta, 12.86±0.2 degrees two-theta, 13.36±0.2 degrees two-theta, 13.82±0.2 degrees two-theta, 14.16±0.2 degrees two-theta, 14.94±0.2 degrees two-theta, 15.9±0.2 degrees two-theta, 16.28±0.2 degrees two-theta, 16.68±0.2 degrees two-theta, 17.16±0.2 degrees two-theta, 18.14±0.2 degrees two-theta, 18.3±0.2 degrees two-theta, 19.04±0.2 degrees two-theta, 20.06±0.2 degrees two-theta, 20.5±0.2 degrees two-theta, 21.1±0.2 degrees two-theta, 21.46±0.2 degrees two-theta, 22.28±0.2 degrees two-theta, 23.04±0.2 degrees two-theta, 23.94±0.2 degrees two-theta, 25.5±0.2 degrees two-theta, 26.16±0.2 degrees two-theta, 26.36±0.2 degrees two-theta, 26.9±0.2 degrees two-theta, 27.16±0.2 degrees two-theta, 27.52±0.2 degrees two-theta, 27.78±0.2 degrees two-theta, 28.06±0.2 degrees two-theta, 28.54±0.2 degrees two-theta, 28.76±0.2 degrees two-theta, 29.48±0.2 degrees two-theta, 30.28±0.2 degrees two-theta, 30.62±0.2 degrees two-theta, 30.88±0.2 degrees two-theta, 31.22±0.2 degrees two-theta, 31.72±0.2 degrees two-theta, 32.38±0.2 degrees two-theta, 32.8±0.2 degrees two-theta, 33.2±0.2 degrees two-theta, 33.86±0.2 degrees two-theta, 34.78±0.2 degrees two-theta, 35.24±0.2 degrees two-theta, 36.26±0.2 degrees two-theta, 37±0.2 degrees two-theta, 37.36±0.2 degrees two-theta, and 37.7±0.2 degrees two-theta.

In some embodiments, crystalline Compound I Adipic Acid Material 1 is characterized by an X-ray powder diffractogram having a signal at one or more of 7.32±0.2 degrees two-theta, 10.46±0.2 degrees two-theta, 14.16±0.2 degrees two-theta, 16.28±0.2 degrees two-theta, and 23.94±0.2 degrees two-theta.

In some embodiments, crystalline Compound I Adipic Acid Material 1 is characterized by an X-ray powder diffractogram having a signal at one or more of 7.32±0.2 degrees two-theta, 14.16±0.2 degrees two-theta, and 16.28±0.2 degrees two-theta.

In some embodiments, crystalline Compound I Adipic Acid Material 1 is characterized by an X-ray powder diffractogram having a signal at one or more of 10.46±0.2 degrees two-theta, and 23.94±0.2 degrees two-theta.

In some embodiments, crystalline Compound I Adipic Acid Material 1 is characterized by an X-ray powder diffractogram having a signal at 7.32±0.2 degrees two-theta. In some embodiments, crystalline Compound I Adipic Acid Material 1 is characterized by an X-ray powder diffractogram having a signal at 10.46±0.2 degrees two-theta. In some embodiments, crystalline Compound I Adipic Acid Material 1 is characterized by an X-ray powder diffractogram having a signal at 14.16±0.2 degrees two-theta. In some embodiments, crystalline Compound I Adipic Acid Material 1 is characterized by an X-ray powder diffractogram having a signal at 16.28±0.2 degrees two-theta. In some embodiments, crystalline Compound I Adipic Acid Material 1 is characterized by an X-ray powder diffractogram having a signal at 23.94±0.2 degrees two-theta.

In some embodiments, crystalline Compound Adipic Acid Material 1 is characterized by an X-ray powder diffractogram substantially similar to FIG. 39.

In some embodiments, Compound I Adipic Acid Material 1 is characterized by a DSC having a broad endotherm peak temperature of 108° C. In some embodiments, Compound I Adipic Acid Material 1 is characterized by a DSC having a large endotherm peak temperature with an onset temperature of 174° C. In some embodiments, Compound I Adipic Acid Material 1 is characterized by a DSC having a large endotherm peak temperature with a peak temperature of 182° C. In some embodiments, Compound I Adipic Acid Material 1 is characterized by a DSC having a broad endotherm peak temperature of 108° C. and/or a large endotherm peak temperature with an onset temperature of 174° C. and a peak temperature of 182° C.

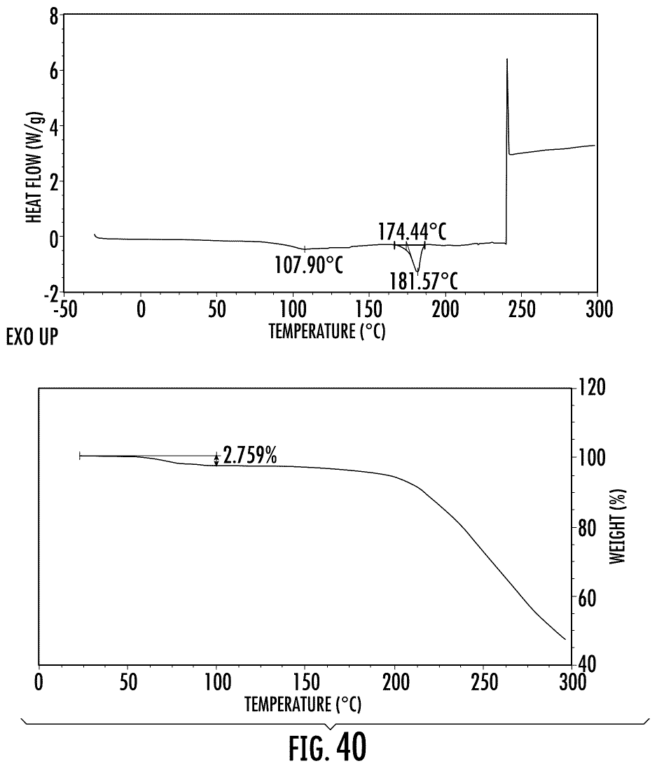

In some embodiments, Compound I Adipic Acid Material 1 is characterized by a DSC substantially similar to FIG. 40.

In some embodiments, Compound I Adipic Acid Material 1 is also characterized by a weight loss of 2.8% up to 100° C., as measured by thermogravimetric analysis.

In some embodiments, Compound I Adipic Acid Material 1 is characterized by a TGA substantially similar to FIG. 40.

In some embodiments, the method of making crystalline Compound I Adipic Acid Material 1 comprises: slurrying Compound I Form 1 and adipic acid in isopropyl acetate at rt for three days, stirring with additional isopropyl acetate for one day, centrifuging, recovering the solids, and drying under a stream of air overnight to yield crystalline Compound I Adipic Acid Material 1.

In some embodiments, the method of making crystalline Compound I Adipic Acid Material 1 comprises: milling Compound I Form 1 and adipic acid with chloroform for 15 minutes to yield crystalline Compound I Adipic Acid Material 1.

### T. Compound I Glutaric Acid Material I+Glutaric Acid

In some embodiments, the disclosure provides crystalline Compound I Glutaric Acid Material 1+glutaric acid. In some embodiments, Compound I Glutaric Acid Material 1+glutaric acid is a mixture.

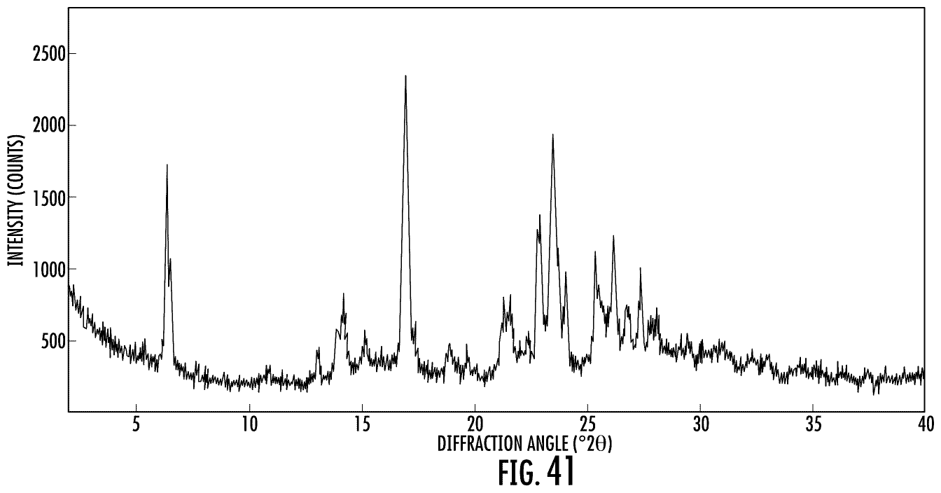

In some embodiments, the disclosure provides Compound I Glutaric Acid Material 1+glutaric acid. FIG. 41 provides an X-ray powder diffractogram of Compound I Glutaric Acid Material 1+glutaric acid with corresponding tabulated data shown in Table 25.

TABLE 25
XRPD Signals for Crystalline Compound I
Glutaric Acid Material 1 + glutaric acid

| °2Θ | d-spacing | Relative |
|---|---|---|
| (±0.2°) | (Å) | Intensity |
| 6.38 | 13.8533 | 76 |
| 5.36 | 16.4871 | 6 |
| 10.88 | 8.1316 | 4 |
| 13.04 | 6.7891 | 11 |
| 13.84 | 6.3984 | 11 |
| 14.22 | 6.2283 | 17 |
| 15.1 | 5.8672 | 12 |
| 16.94 | 5.2338 | 100 |
| 17.34 | 5.114 | 10 |
| 18.88 | 4.7002 | 12 |
| 19.66 | 4.5154 | 12 |
| 21.52 | 4.1292 | 25 |
| 22 | 4.0402 | 6 |
| 22.32 | 3.983 | 9 |
| 22.84 | 3.8934 | 41 |
| 23.42 | 3.7983 | 54 |
| 24.04 | 3.7017 | 29 |
| 25.32 | 3.5174 | 31 |
| 26.12 | 3.4115 | 33 |
| 26.72 | 3.3362 | 15 |
| 27.3 | 3.2667 | 28 |
| 28.02 | 3.1843 | 13 |
| 29.38 | 3.04 | 6 |
| 32.24 | 2.7765 | 8 |
| 37.36 | 2.4069 | 5 |

In some embodiments, crystalline Compound I Glutaric Acid Material 1+glutaric acid is substantially pure. In some embodiments, crystalline Compound I Glutaric Acid Material 1+glutaric acid is substantially crystalline. In some embodiments, crystalline Compound I Glutaric Acid Material 1+glutaric acid is characterized by an X-ray powder diffractogram generated by an X-ray powder diffraction analysis with an incident beam of Cu Kα radiation.

In some embodiments, crystalline Compound I Glutaric Acid Material 1+glutaric acid is characterized by an X-ray powder diffractogram having a signal at one or more of 6.38±0.2 degrees two-theta, 5.36±0.2 degrees two-theta, 10.88±0.2 degrees two-theta, 13.04±0.2 degrees two-theta, 13.84±0.2 degrees two-theta, 14.22±0.2 degrees two-theta, ±0.2 degrees two-theta, 15.1±0.2 degrees two-theta, 16.94±0.2 degrees two-theta, 17.34±0.2 degrees two-theta, 18.88±0.2 degrees two-theta, 19.66±0.2 degrees two-theta, 21.52±0.2 degrees two-theta, 22±0.2 degrees two-theta, 22.32±0.2 degrees two-theta, 22.84±0.2 degrees two-theta, 23.42±0.2 degrees two-theta, 24.04±0.2 degrees two-theta, 25.32±0.2 degrees two-theta, 26.12±0.2 degrees two-theta, 26.72±0.2 degrees two-theta, 27.3±0.2 degrees two-theta, 28.02±0.2 degrees two-theta, 29.38±0.2 degrees two-theta, 32.24±0.2 degrees two-theta, and 37.36±0.2 degrees two-theta.

In some embodiments, crystalline Compound I Glutaric Acid Material 1+glutaric acid is characterized by an X-ray powder diffractogram having a signal at one or more of 6.38±0.2 degrees two-theta, 13.04±0.2 degrees two-theta, 13.84±0.2 degrees two-theta, 14.22±0.2 degrees two-theta, and 16.94±0.2 degrees two-theta.

In some embodiments, crystalline Compound I Glutaric Acid Material 1+glutaric acid is characterized by an X-ray powder diffractogram having a signal at one or more of 6.38±0.2 degrees two-theta, 14.22±0.2 degrees two-theta, and 16.94±0.2 degrees two-theta.

In some embodiments, crystalline Compound I Glutaric Acid Material 1+glutaric acid is characterized by an X-ray powder diffractogram having a signal at one or more of 13.04±0.2 degrees two-theta and 13.84±0.2 degrees two-theta.

In some embodiments, crystalline Compound I Glutaric Acid Material 1+glutaric acid is characterized by an X-ray powder diffractogram having a signal at 6.38±0.2 degrees two-theta. In some embodiments, crystalline Compound I Glutaric Acid Material 1+glutaric acid is characterized by an X-ray powder diffractogram having a signal at 13.04±0.2 degrees two-theta. In some embodiments, crystalline Compound I Glutaric Acid Material 1+glutaric acid is characterized by an X-ray powder diffractogram having a signal at 13.84±0.2 degrees two-theta. In some embodiments, crystalline Compound I Glutaric Acid Material 1+glutaric acid is characterized by an X-ray powder diffractogram having a signal at 14.22±0.2 degrees two-theta. In some embodiments, crystalline Compound I Glutaric Acid Material 1+glutaric acid is characterized by an X-ray powder diffractogram having a signal at 16.94±0.2 degrees two-theta.

In some embodiments, crystalline Compound Glutaric Acid Material 1+glutaric acid is characterized by an X-ray powder diffractogram substantially similar to FIG. 41.

In some embodiments, the method of making crystalline Compound I Glutaric Acid Material 1+glutaric acid comprises: combining Compound I Form 1 and glutaric acid in isopropyl acetate, milling the sample with a stainless-steel ball, and recovering the solids to yield crystalline Compound I Glutaric Acid Material 1+glutaric acid.

### U. Compound I Glutaric Acid Material 2

In some embodiments, the disclosure provides crystalline Compound I Glutaric Acid Material 2. In some embodiments, Compound I Glutaric Acid Material 2 is a hydrate.

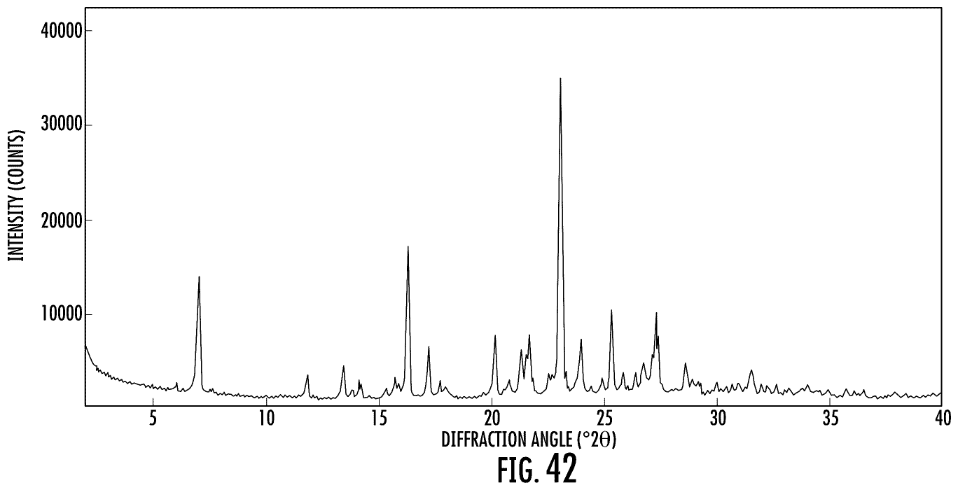

In some embodiments, the disclosure provides Compound I Glutaric Acid Material 2. FIG. 42 provides an X-ray powder diffractogram of Compound I Glutaric Acid Material 2 with corresponding tabulated data shown in Table 26.

TABLE 26
XRPD Signals for Crystalline Compound I
Glutaric Acid Material 2

| °2Θ | d-spacing | Relative |
|---|---|---|
| (±0.2°) | (Å) | Intensity |
| 6.08 | 14.5362 | 8 |
| 7.1 | 12.45 | 39 |
| 7.7 | 11.4812 | 6 |
| 11.88 | 7.4492 | 10 |
| 13.48 | 6.5684 | 13 |
| 13.84 | 6.3984 | 6 |
| 14.18 | 6.2457 | 8 |
| 15.34 | 5.7759 | 6 |
| 15.74 | 5.6301 | 10 |
| 15.94 | 5.5599 | 7 |
| 16.34 | 5.4246 | 48 |
| 17.24 | 5.1434 | 18 |
| 17.72 | 5.0052 | 8 |
| 18 | 4.9279 | 6 |
| 19.68 | 4.5109 | 5 |
| 20.18 | 4.4002 | 22 |
| 20.8 | 4.2705 | 9 |
| 21.34 | 4.1636 | 17 |
| 21.7 | 4.0953 | 22 |
| 22.56 | 3.9411 | 10 |
| 22.88 | 3.8867 | 11 |
| 23.1 | 3.8502 | 100 |
| 23.98 | 3.7109 | 21 |
| 24.4 | 3.6479 | 7 |
| 24.92 | 3.573 | 9 |
| 25.36 | 3.512 | 22 |
| 25.86 | 3.4452 | 4 |
| 26.4 | 3.3759 | 5 |
| 26.76 | 3.3313 | 7 |
| 27.32 | 3.2643 | 18 |
| 28.6 | 3.1211 | 6 |
| 28.92 | 3.0873 | 2 |
| 29.22 | 3.0562 | 2 |
| 29.64 | 3.0139 | 1 |
| 29.96 | 2.9824 | 3 |
| 30.66 | 2.9159 | 2 |
| 30.98 | 2.8865 | 2 |
| 31.58 | 2.833 | 6 |
| 31.98 | 2.7985 | 3 |
| 32.26 | 2.7748 | 1 |
| 32.62 | 2.745 | 2 |
| 33.2 | 2.6984 | 1 |
| 33.98 | 2.6382 | 2 |
| 34.48 | 2.6011 | 1 |
| 34.94 | 2.5679 | 1 |
| 35.7 | 2.515 | 2 |
| 36.48 | 2.463 | 2 |
| 37.88 | 2.3751 | 1 |

In some embodiments, crystalline Compound I Glutaric Acid Material 2 is substantially pure. In some embodiments, crystalline Compound I Glutaric Acid Material 2 is substantially crystalline. In some embodiments, crystalline Compound I Glutaric Acid Material 2 is characterized by an X-ray powder diffractogram generated by an X-ray powder diffraction analysis with an incident beam of Cu Kα radiation.

In some embodiments, crystalline Compound I Glutaric Acid Material 2 is characterized by an X-ray powder diffractogram having a signal at one or more of 6.08±0.2 degrees two-theta, 7.1±0.2 degrees two-theta, 7.7±0.2 degrees two-theta, 11.88±0.2 degrees two-theta, 13.48±0.2 degrees two-theta, 13.84±0.2 degrees two-theta, ±0.2 degrees two-theta, 14.18±0.2 degrees two-theta, 15.34±0.2 degrees two-theta, 15.74±0.2 degrees two-theta, 15.94±0.2 degrees two-theta, 16.34±0.2 degrees two-theta, 17.24±0.2 degrees two-theta, 17.72±0.2 degrees two-theta, 18±0.2 degrees two-theta, 19.68±0.2 degrees two-theta, 20.18±0.2 degrees two-theta, 20.8±0.2 degrees two-theta, 21.34±0.2 degrees two-theta, 21.7±0.2 degrees two-theta, 22.56±0.2 degrees two-theta, 22.88±0.2 degrees two-theta, 23.1±0.2 degrees two-theta, 23.98±0.2 degrees two-theta, 24.4±0.2 degrees two-theta, 24.92±0.2 degrees two-theta, 25.36±0.2 degrees two-theta, 25.86±0.2 degrees two-theta, 26.4±0.2 degrees two-theta, 26.76±0.2 degrees two-theta, 27.32±0.2 degrees two-theta, 28.6±0.2 degrees two-theta, 28.92±0.2 degrees two-theta, 29.22±0.2 degrees two-theta, 29.64±0.2 degrees two-theta, 29.96±0.2 degrees two-theta, 30.66±0.2 degrees two-theta, 30.98±0.2 degrees two-theta, 31.58±0.2 degrees two-theta, 31.98±0.2 degrees two-theta, 32.26±0.2 degrees two-theta, 32.62±0.2 degrees two-theta, 33.2±0.2 degrees two-theta, 33.98±0.2 degrees two-theta, 34.48±0.2 degrees two-theta, 34.94±0.2 degrees two-theta, 35.7±0.2 degrees two-theta, 36.48±0.2 degrees two-theta, and 37.88±0.2 degrees two-theta.

In some embodiments, crystalline Compound I Glutaric Acid Material 2 is characterized by an X-ray powder diffractogram having a signal at one or more of 7.1±0.2 degrees two-theta, 11.88±0.2 degrees two-theta, 13.48±0.2 degrees two-theta, 16.34±0.2 degrees two-theta, 17.24±0.2 degrees two-theta, and 23.1±0.2 degrees two-theta.

In some embodiments, crystalline Compound I Glutaric Acid Material 2 is characterized by an X-ray powder diffractogram having a signal at one or more of 7.1±0.2 degrees two-theta, 16.34±0.2 degrees two-theta, and 17.24±0.2 degrees two-theta.

In some embodiments, crystalline Compound I Glutaric Acid Material 2 is characterized by an X-ray powder diffractogram having a signal at one or more of 11.88±0.2 degrees two-theta, 13.48±0.2 degrees two-theta, and 23.1±0.2 degrees two-theta.

In some embodiments, crystalline Compound I Glutaric Acid Material 2 is characterized by an X-ray powder diffractogram having a signal at 7.1±0.2 degrees two-theta. In some embodiments, crystalline Compound I Glutaric Acid Material 2 is characterized by an X-ray powder diffractogram having a signal at 11.88±0.2 degrees two-theta. In some embodiments, crystalline Compound I Glutaric Acid Material 2 is characterized by an X-ray powder diffractogram having a signal at 13.48±0.2 degrees two-theta. In some embodiments, crystalline Compound I Glutaric Acid Material 2 is characterized by an X-ray powder diffractogram having a signal at 16.34±0.2 degrees two-theta. In some embodiments, crystalline Compound I Glutaric Acid Material 2 is characterized by an X-ray powder diffractogram having a signal at 17.24±0.2 degrees two-theta. In some embodiments, crystalline Compound I Glutaric Acid Material 2 is characterized by an X-ray powder diffractogram having a signal at 23.1±0.2 degrees two-theta.

In some embodiments, crystalline Compound Glutaric Acid Material 2 is characterized by an X-ray powder diffractogram substantially similar to FIG. 42.

In some embodiments, Compound I Glutaric Acid Material 2 is characterized by a DSC having an endotherm peak temperature of 64° C. In some embodiments, Compound I Glutaric Acid Material 2 is characterized by a DSC having a broad endotherm of temperature of 148° C. In some embodiments, Compound I Glutaric Acid Material 2 is characterized by a DSC having an endotherm peak temperature of 64° C. and/or an endotherm peak temperature of 148° C.

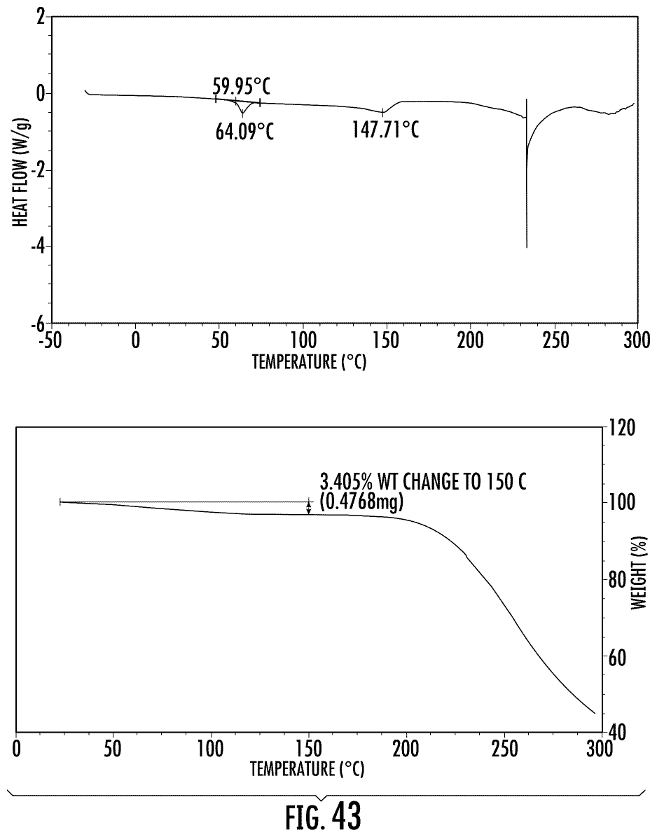

In some embodiments, Compound I Glutaric Acid Material 2 is characterized by a DSC substantially similar to FIG. 43.

In some embodiments, Compound I Glutaric Acid Material 2 is also characterized by a weight loss of 3.4% up to 150° C., as measured by thermogravimetric analysis.

In some embodiments, Compound I Glutaric Acid Material 2 is characterized by a TGA substantially similar to FIG. 43.

In some embodiments, the method of making crystalline Compound I Glutaric Acid Material 2 comprises: slurrying Compound I Form 1 and glutaric acid in isopropyl acetate at rt for four days, centrifuging, and recovering the solids to yield crystalline Compound I Glutaric Acid Material 2.

### V. Compound I Succinic Acid Material 1+Succinic Acid+Possible Compound I Form 1

In some embodiments, the disclosure provides crystalline Compound I Succinic Acid Material 1+succinic acid+possible Compound I Form 1. In some embodiments, Compound I Succinic Acid Material 1+succinic acid+possible Compound I Form 1 is a mixture of Compound I Succinic Acid Material 1 and/or succinic acid and/or possible Compound I Form 1.

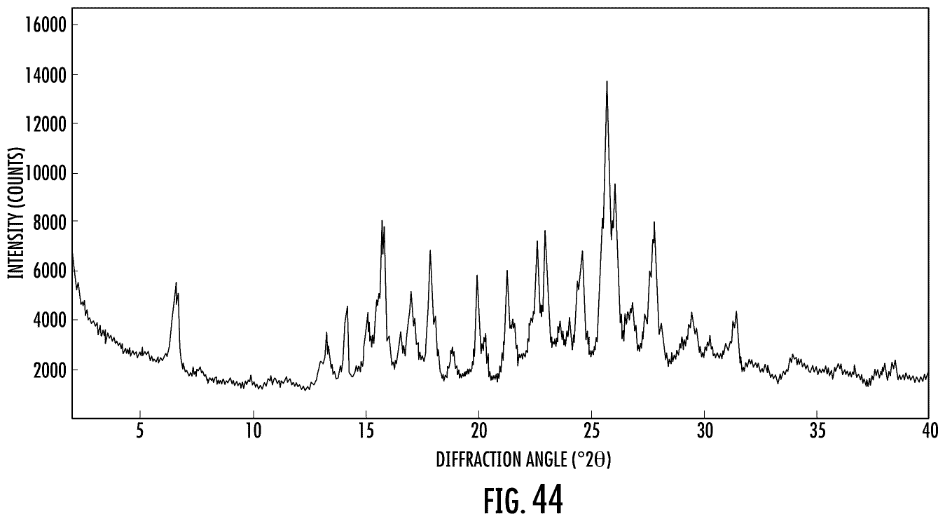

In some embodiments, the disclosure provides Compound I Succinic Acid Material 1+succinic acid+possible Compound I Form 1. FIG. 44 provides an X-ray powder diffractogram of Compound I Succinic Acid Material 1+succinic acid+possible Compound I Form 1 with corresponding tabulated data shown in Table 27.

TABLE 27
XRPD Signals for Crystalline Compound I
Succinic Acid Material 1 + succinic acid +
possible Compound I Form 1

| °2Θ | d-spacing | Relative |
|---|---|---|
| (±0.2°) | (Å) | Intensity |
| 5.4 | 16.3651 | 6 |
| 6.7 | 13.1924 | 55 |
| 7.72 | 11.4515 | 6 |
| 9.1 | 9.7178 | 4 |
| 9.98 | 8.8628 | 6 |
| 10.84 | 8.1615 | 6 |
| 11.68 | 7.5764 | 6 |
| 13.36 | 6.6272 | 29 |
| 14.28 | 6.2022 | 44 |
| 15.18 | 5.8365 | 26 |
| 15.82 | 5.6018 | 83 |
| 16.62 | 5.3339 | 17 |
| 17.1 | 5.1852 | 40 |
| 17.98 | 4.9334 | 82 |
| 18.9 | 4.6953 | 19 |
| 20.02 | 4.435 | 55 |
| 20.36 | 4.3617 | 16 |
| 21.36 | 4.1598 | 45 |
| 21.62 | 4.1103 | 10 |
| 22.7 | 3.9171 | 41 |
| 23.04 | 3.8601 | 66 |
| 23.72 | 3.751 | 15 |
| 24.1 | 3.6927 | 17 |
| 24.66 | 3.6101 | 56 |
| 25.76 | 3.4583 | 100 |
| 26.16 | 3.4064 | 51 |
| 26.9 | 3.3143 | 29 |
| 27.84 | 3.2045 | 69 |
| 29.52 | 3.0259 | 26 |
| 30.32 | 2.9478 | 12 |
| 31.02 | 2.8829 | 6 |
| 31.5 | 2.84 | 34 |
| 33.94 | 2.6412 | 11 |
| 32.1 | 2.7883 | 6 |
| 32.8 | 2.7304 | 6 |
| 36 | 2.4947 | 5 |
| 37.62 | 2.3909 | 4 |
| 38.04 | 2.3655 | 8 |
| 38.5 | 2.3383 | 12 |

In some embodiments, crystalline Compound I Succinic Acid Material 1+succinic acid+possible Compound I Form 1 is substantially pure. In some embodiments, crystalline Compound I Succinic Acid Material 1+succinic acid+possible Compound I Form 1 is substantially crystalline. In some embodiments, crystalline Compound I Succinic Acid Material 1+succinic acid+possible Compound I Form 1 is characterized by an X-ray powder diffractogram generated by an X-ray powder diffraction analysis with an incident beam of Cu Kα radiation.

In some embodiments, crystalline Compound I Succinic Acid Material 1+succinic acid+possible Compound I Form 1 is characterized by an X-ray powder diffractogram having a signal at one or more of 5.4±0.2 degrees two-theta, 6.7±0.2 degrees two-theta, 7.72±0.2 degrees two-theta, 9.1±0.2 degrees two-theta, 9.98±0.2 degrees two-theta, 10.84±0.2 degrees two-theta, ±0.2 degrees two-theta, 11.68±0.2 degrees two-theta, 13.36±0.2 degrees two-theta, 14.28±0.2 degrees two-theta, 15.18±0.2 degrees two-theta, 15.82±0.2 degrees two-theta, 16.62±0.2 degrees two-theta, 17.1±0.2 degrees two-theta, 17.98±0.2 degrees two-theta, 18.9±0.2 degrees two-theta, 20.02±0.2 degrees two-theta, 20.36±0.2 degrees two-theta, 21.36±0.2 degrees two-theta, 21.62±0.2 degrees two-theta, 22.7±0.2 degrees two-theta, 23.04±0.2 degrees two-theta, 23.72±0.2 degrees two-theta, 24.1±0.2 degrees two-theta, 24.66±0.2 degrees two-theta, 25.76±0.2 degrees two-theta, 26.16±0.2 degrees two-theta, 26.9±0.2 degrees two-theta, 27.84±0.2 degrees two-theta, 29.52±0.2 degrees two-theta, 30.32±0.2 degrees two-theta, 31.02±0.2 degrees two-theta, 31.5±0.2 degrees two-theta, 33.94±0.2 degrees two-theta, 32.1±0.2 degrees two-theta, 32.8±0.2 degrees two-theta, 36±0.2 degrees two-theta, 37.62±0.2 degrees two-theta, 38.04 and 38.5±0.2 degrees two-theta.

In some embodiments, crystalline Compound I Succinic Acid Material 1+succinic acid+possible Compound I Form 1 is characterized by an X-ray powder diffractogram having a signal at one or more of 6.7±0.2 degrees two-theta, 13.36±0.2 degrees two-theta, 15.18±0.2 degrees two-theta, 15.82±0.2 degrees two-theta, 17.1±0.2 degrees two-theta, and 25.76±0.2 degrees two-theta.

In some embodiments, crystalline Compound I Succinic Acid Material 1+succinic acid+possible Compound I Form 1 is characterized by an X-ray powder diffractogram having a signal at one or more of 6.7±0.2 degrees two-theta, 15.82±0.2 degrees two-theta, and 17.1±0.2 degrees two-theta.

In some embodiments, crystalline Compound I Succinic Acid Material 1+succinic acid+possible Compound I Form 1 is characterized by an X-ray powder diffractogram having a signal at one or more of 13.36±0.2 degrees two-theta, 15.18±0.2 degrees two-theta, and 25.76±0.2 degrees two-theta.

In some embodiments, crystalline Compound I Succinic Acid Material 1+succinic acid+possible Compound I Form 1 is characterized by an X-ray powder diffractogram having a signal at 6.7±0.2 degrees two-theta. In some embodiments, crystalline Compound I Succinic Acid Material 1+succinic acid+possible Compound I Form 1 is characterized by an X-ray powder diffractogram having a signal at 13.36±0.2 degrees two-theta. In some embodiments, crystalline Compound I Succinic Acid Material 1+succinic acid+possible Compound I Form 1 is characterized by an X-ray powder diffractogram having a signal at 15.18±0.2 degrees two-theta. In some embodiments, crystalline Compound I Succinic Acid Material 1+succinic acid+possible Compound I Form 1 is characterized by an X-ray powder diffractogram having a signal at 15.82±0.2 degrees two-theta. In some embodiments, crystalline Compound I Succinic Acid Material 1+succinic acid+possible Compound I Form 1 is characterized by an X-ray powder diffractogram having a signal at 17.1±0.2 degrees two-theta. In some embodiments, crystalline Compound I Succinic Acid Material 1+succinic acid+possible Compound I Form 1 is characterized by an X-ray powder diffractogram having a signal at 25.76±0.2 degrees two-theta.

In some embodiments, crystalline Compound Succinic Acid Material 1+succinic acid+possible Compound I Form 1 is characterized by an X-ray powder diffractogram substantially similar to FIG. 44.

In some embodiments, the method of making crystalline Compound I Succinic Acid Material 1+succinic acid+possible Compound I Form 1 comprises: milling Compound I Form 1 and succinic acid in ethyl acetate and recovering the solids to yield crystalline Compound I Succinic Acid Material 1+succinic acid+possible Compound I Form 1.

### W. Compound I Vanillin Material 1+Vanillin

In some embodiments, the disclosure provides crystalline Compound I Vanillin Material 1+vanillin. In some embodiments, Compound I Vanillin Material 1+vanillin is a mixture of Compound I Vanillin Material 1 and vanillin.

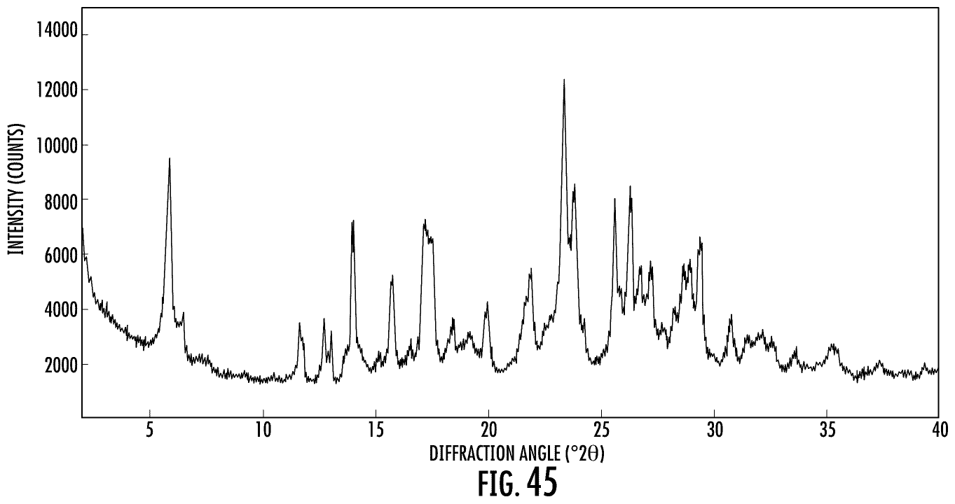

In some embodiments, the disclosure provides Compound I Vanillin Material 1+vanillin. FIG. 45 provides an X-ray powder diffractogram of Compound I Vanillin Material 1+vanillin with corresponding tabulated data shown in Table 28.

TABLE 28
XRPD Signals for Crystalline Compound I
Vanillin Material 1 + vanillin

| °2Θ | d-spacing | Relative |
|---|---|---|
| (±0.2°) | (Å) | Intensity |
| 5.88 | 15.0302 | 85 |
| 6.48 | 13.6398 | 13 |
| 7.3 | 12.1094 | 4 |
| 7.62 | 11.6015 | 2 |
| 9.22 | 9.5915 | 2 |
| 11.66 | 7.5893 | 21 |
| 12.74 | 6.9483 | 25 |
| 13.06 | 6.7787 | 13 |
| 13.66 | 6.4823 | 6 |
| 14.02 | 6.3166 | 68 |
| 15.18 | 5.8365 | 7 |
| 15.76 | 5.623 | 47 |
| 17.18 | 5.1613 | 35 |
| 17.54 | 5.0561 | 18 |
| 18.44 | 4.8113 | 19 |
| 19.18 | 4.6273 | 5 |
| 19.96 | 4.4482 | 29 |
| 21.92 | 4.0547 | 43 |
| 23.38 | 3.8047 | 100 |
| 23.82 | 3.7354 | 49 |
| 25.64 | 3.4743 | 70 |
| 26.28 | 3.3911 | 64 |
| 26.74 | 3.3338 | 19 |
| 27.2 | 3.2784 | 28 |
| 27.84 | 3.2045 | 8 |
| 28.22 | 3.1622 | 9 |
| 28.62 | 3.1189 | 18 |
| 28.92 | 3.0873 | 16 |
| 29.38 | 3.04 | 48 |
| 30.76 | 2.9066 | 20 |
| 31.48 | 2.8418 | 6 |
| 32.1 | 2.7883 | 7 |
| 32.58 | 2.7483 | 9 |
| 33.56 | 2.6703 | 7 |
| 35.32 | 2.5411 | 9 |
| 35.98 | 2.496 | 2 |
| 37.32 | 2.4094 | 4 |
| 39.34 | 2.2902 | 4 |

In some embodiments, crystalline Compound I Vanillin Material 1+vanillin is substantially pure. In some embodiments, crystalline Compound I Vanillin Material 1+vanillin is substantially crystalline. In some embodiments, crystalline Compound I Vanillin Material 1+vanillin is characterized by an X-ray powder diffractogram generated by an X-ray powder diffraction analysis with an incident beam of Cu Kα radiation.

In some embodiments, crystalline Compound I Vanillin Material 1+vanillin is characterized by an X-ray powder diffractogram having a signal at one or more of 5.88±0.2 degrees two-theta, 6.48±0.2 degrees two-theta, 7.3±0.2 degrees two-theta, 7.62±0.2 degrees two-theta, 9.22±0.2 degrees two-theta, 11.66±0.2 degrees two-theta, ±0.2 degrees two-theta, 12.74±0.2 degrees two-theta, 13.06±0.2 degrees two-theta, 13.66±0.2 degrees two-theta, 14.02±0.2 degrees two-theta, 15.18±0.2 degrees two-theta, 15.76±0.2 degrees two-theta, 17.18±0.2 degrees two-theta, 17.54±0.2 degrees two-theta, 18.44±0.2 degrees two-theta, 19.18±0.2 degrees two-theta, 19.96±0.2 degrees two-theta, 21.92±0.2 degrees two-theta, 23.38±0.2 degrees two-theta, 23.82±0.2 degrees two-theta, 25.64±0.2 degrees two-theta, 26.28±0.2 degrees two-theta, 26.74±0.2 degrees two-theta, 27.2±0.2 degrees two-theta, 27.84±0.2 degrees two-theta, 28.22±0.2 degrees two-theta, 28.62±0.2 degrees two-theta, 28.92±0.2 degrees two-theta, 29.38±0.2 degrees two-theta, 30.76±0.2 degrees two-theta, 31.48±0.2 degrees two-theta, 32.1±0.2 degrees two-theta, 32.58±0.2 degrees two-theta, 33.56±0.2 degrees two-theta, 35.32±0.2 degrees two-theta, 35.98±0.2 degrees two-theta, 37.32±0.2 degrees two-theta, and 39.34±0.2 degrees two-theta.

In some embodiments, crystalline Compound I Vanillin Material 1+vanillin is characterized by an X-ray powder diffractogram having a signal at one or more of 5.88±0.2 degrees two-theta, 11.66±0.2 degrees two-theta, 14.02±0.2 degrees two-theta, 15.76±0.2 degrees two-theta, and 23.38±0.2 degrees two-theta.

In some embodiments, crystalline Compound I Vanillin Material 1+vanillin is characterized by an X-ray powder diffractogram having a signal at one or more of 5.88±0.2 degrees two-theta, 14.02±0.2 degrees two-theta, and 15.76±0.2 degrees two-theta.

In some embodiments, crystalline Compound I Vanillin Material 1+vanillin is characterized by an X-ray powder diffractogram having a signal at one or more of 11.66±0.2 degrees two-theta, 23.38±0.2 degrees two-theta, and 27.84±0.2 degrees two-theta.

In some embodiments, crystalline Compound I Vanillin Material 1+vanillin is characterized by an X-ray powder diffractogram having a signal at 5.88±0.2 degrees two-theta. In some embodiments, crystalline Compound I Vanillin Material 1+vanillin is characterized by an X-ray powder diffractogram having a signal at 11.66±0.2 degrees two-theta. In some embodiments, crystalline Compound I Vanillin Material 1+vanillin is characterized by an X-ray powder diffractogram having a signal at 14.02±0.2 degrees two-theta. In some embodiments, crystalline Compound I Vanillin Material 1+vanillin is characterized by an X-ray powder diffractogram having a signal at 15.76±0.2 degrees two-theta. In some embodiments, crystalline Compound I Vanillin Material 1+vanillin is characterized by an X-ray powder diffractogram having a signal at 23.38±0.2 degrees two-theta.

In some embodiments, crystalline Compound Vanillin Material 1+vanillin is characterized by an X-ray powder diffractogram substantially similar to FIG. 45.

In some embodiments, the method of making crystalline Compound I Vanillin Material 1+vanillin comprises: milling Compound I Form 1 and vanillin in chloroform and recovering the solids to yield crystalline Compound I Vanillin Material 1+vanillin.

### X. Compound I Salt Forms

### X-1. Compound I Mono-Maleate Material A

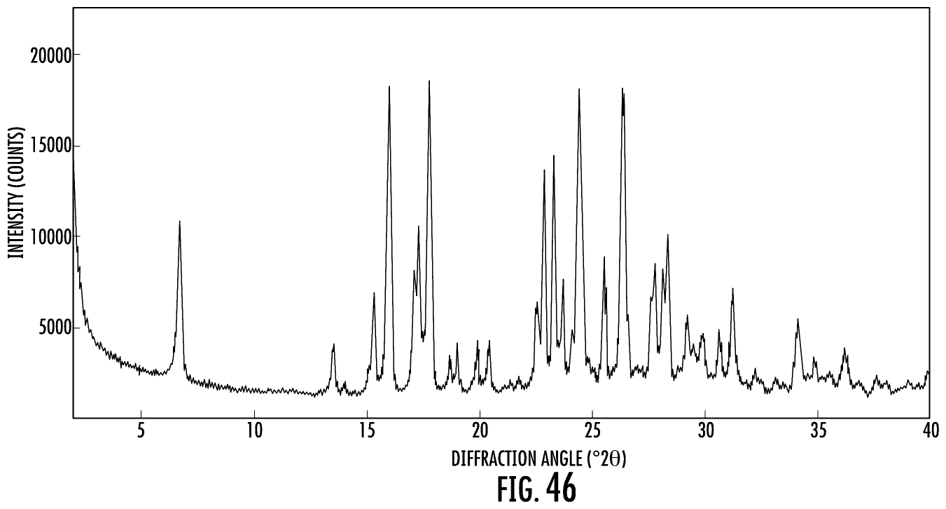

In some embodiments, the disclosure provides crystalline Compound I Mono-Maleate Material A. In some embodiments, the disclosure provides Compound I Mono-Maleate Material A. FIG. 46 provides an X-ray powder diffractogram of Compound I Mono-Maleate Material A with corresponding tabulated data shown in Table 29.

TABLE 29
XRPD Signals for Crystalline Compound I
Mono-Maleate Material A

| °2Θ | d-spacing | Relative |
|---|---|---|
| (±0.2°) | (Å) | Intensity |
| 6.72 | 13.1532 | 59 |
| 13.6 | 6.5108 | 22 |
| 14.04 | 6.3077 | 11 |
| 15.12 | 5.8595 | 16 |
| 15.36 | 5.7685 | 36 |
| 16.02 | 5.5323 | 99 |
| 17.12 | 5.1792 | 43 |
| 17.32 | 5.1199 | 56 |
| 17.82 | 4.9773 | 100 |
| 18.7 | 4.745 | 18 |
| 19.04 | 4.6611 | 23 |
| 19.92 | 4.4571 | 22 |
| 20.44 | 4.3449 | 22 |
| 21.1 | 4.2104 | 10 |
| 21.4 | 4.1521 | 11 |
| 21.78 | 4.0805 | 13 |
| 22.58 | 3.9377 | 35 |
| 22.9 | 3.8834 | 73 |
| 23.34 | 3.8112 | 77 |
| 23.74 | 3.7478 | 42 |
| 24.12 | 3.6896 | 25 |
| 24.48 | 3.6362 | 97 |
| 25.56 | 3.485 | 46 |
| 26.4 | 3.3759 | 97 |
| 27.8 | 3.209 | 45 |
| 28.16 | 3.1688 | 45 |
| 28.4 | 3.1426 | 53 |
| 29.26 | 3.0522 | 31 |
| 29.56 | 3.0219 | 22 |
| 29.94 | 2.9844 | 25 |
| 30.66 | 2.9159 | 27 |
| 31.28 | 2.8595 | 38 |
| 32.26 | 2.7748 | 15 |
| 32.62 | 2.745 | 11 |
| 34.2 | 2.6217 | 30 |
| 34.58 | 2.5938 | 14 |
| 34.9 | 2.5708 | 18 |
| 36.24 | 2.4787 | 21 |
| 36.9 | 2.4359 | 11 |
| 37.64 | 2.3897 | 13 |
| 38.08 | 2.3631 | 11 |
| 39.08 | 2.3049 | 11 |
| 39.48 | 2.2824 | 11 |

In some embodiments, crystalline Compound I Mono-Maleate Material A is substantially pure. In some embodiments, crystalline Compound I Mono-Maleate Material A is substantially crystalline. In some embodiments, crystalline Compound I Mono-Maleate Material A is characterized by an X-ray powder diffractogram generated by an X-ray powder diffraction analysis with an incident beam of Cu Kα radiation.

In some embodiments, crystalline Compound I Mono-Maleate Material A is characterized by an X-ray powder diffractogram having a signal at one or more of 6.72±0.2 degrees two-theta, 13.6±0.2 degrees two-theta, 14.04±0.2 degrees two-theta, 15.12±0.2 degrees two-theta, 15.36±0.2 degrees two-theta, 16.02±0.2 degrees two-theta, ±0.2 degrees two-theta, 17.12±0.2 degrees two-theta, 17.32±0.2 degrees two-theta, 17.82±0.2 degrees two-theta, 18.7±0.2 degrees two-theta, 19.04±0.2 degrees two-theta, 19.92±0.2 degrees two-theta, 20.44±0.2 degrees two-theta, 21.1±0.2 degrees two-theta, 21.4±0.2 degrees two-theta, 21.78±0.2 degrees two-theta, 22.58±0.2 degrees two-theta, 22.9±0.2 degrees two-theta, 23.34±0.2 degrees two-theta, 23.74±0.2 degrees two-theta, 24.12±0.2 degrees two-theta, 24.48±0.2 degrees two-theta, 25.56±0.2 degrees two-theta, 26.4±0.2 degrees two-theta, 27.8±0.2 degrees two-theta, 28.16±0.2 degrees two-theta, 28.4±0.2 degrees two-theta, 29.26±0.2 degrees two-theta, 29.56±0.2 degrees two-theta, 29.94±0.2 degrees two-theta, 30.66±0.2 degrees two-theta, 31.28±0.2 degrees two-theta, 32.26±0.2 degrees two-theta, 32.62±0.2 degrees two-theta, 34.2±0.2 degrees two-theta, 34.58±0.2 degrees two-theta, 34.9±0.2 degrees two-theta, 36.24±0.2 degrees two-theta, 36.9±0.2 degrees two-theta, 37.64±0.2 degrees two-theta, 38.08±0.2 degrees two-theta, 39.08±0.2 degrees two-theta, and 39.48±0.2 degrees two-theta.

In some embodiments, crystalline Compound I Mono-Maleate Material A is characterized by an X-ray powder diffractogram having a signal at one or more of 6.72±0.2 degrees two-theta, 13.6±0.2 degrees two-theta, 15.36±0.2 degrees two-theta, 16.02±0.2 degrees two-theta, and 17.82±0.2 degrees two-theta.

In some embodiments, crystalline Compound I Mono-Maleate Material A is characterized by an X-ray powder diffractogram having a signal at one or more of 6.72±0.2 degrees two-theta, 16.02±0.2 degrees two-theta, and 17.82±0.2 degrees two-theta.

In some embodiments, crystalline Compound I Mono-Maleate Material A is characterized by an X-ray powder diffractogram having a signal at one or more of 13.6±0.2 degrees two-theta and 15.36±0.2 degrees two-theta.

In some embodiments, crystalline Compound I Mono-Maleate Material A is characterized by an X-ray powder diffractogram having a signal at 6.72±0.2 degrees two-theta. In some embodiments, crystalline Compound I Mono-Maleate Material A is characterized by an X-ray powder diffractogram having a signal at 13.6±0.2 degrees two-theta. In some embodiments, crystalline Compound I Mono-Maleate Material A is characterized by an X-ray powder diffractogram having a signal at 15.36±0.2 degrees two-theta. In some embodiments, crystalline Compound I Mono-Maleate Material A is characterized by an X-ray powder diffractogram having a signal at 16.02±0.2 degrees two-theta. In some embodiments, crystalline Compound I Mono-Maleate Material A is characterized by an X-ray powder diffractogram having a signal at 17.82±0.2 degrees two-theta.

In some embodiments, crystalline Compound Mono-Maleate Material A is characterized by an X-ray powder diffractogram substantially similar to FIG. 46.

In some embodiments, Compound I Mono-Maleate Material A is characterized by a DSC having an endotherm peak temperature of 129° C. In some embodiments, Compound I Mono-Maleate Material A is characterized by a DSC having a broad endotherm of temperature of 129° C. and a shoulder at 87° C. In some embodiments, Compound I Mono-Maleate Material A is characterized by a DSC having an endotherm peak temperature of 129° C. and/or an endotherm shoulder temperature of 87° C.

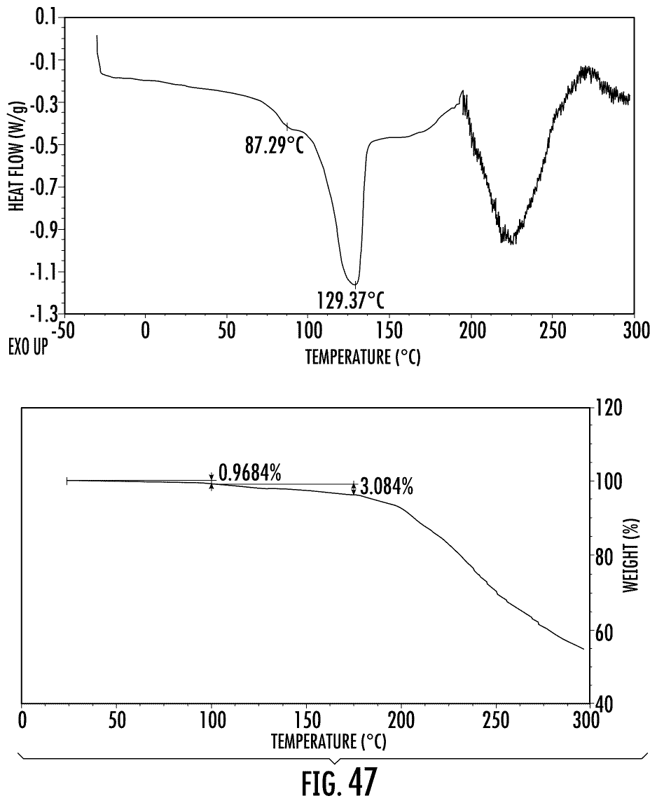

In some embodiments, Compound I Mono-Maleate Material A is characterized by a DSC substantially similar to FIG. 47.

In some embodiments, Compound I Mono-Maleate Material A is also characterized by a weight loss of 1% up to 100° C., as measured by thermogravimetric analysis. In some embodiments, Compound I Mono-Maleate Material A is also characterized by a weight loss of 3% up to 175° C., as measured by thermogravimetric analysis.

In some embodiments, Compound I Mono-Maleate Material A is characterized by a TGA substantially similar to FIG. 47.

In some embodiments, the method of making crystalline Compound I Mono-Maleate Material A comprises: milling Compound I Form 1 and maleic acid in ethyl acetate and recovering the solids to yield crystalline Compound I Mono-Maleate Material A.

### Y. Compound I Free Base Forms

### Y-1. Compound I Form A

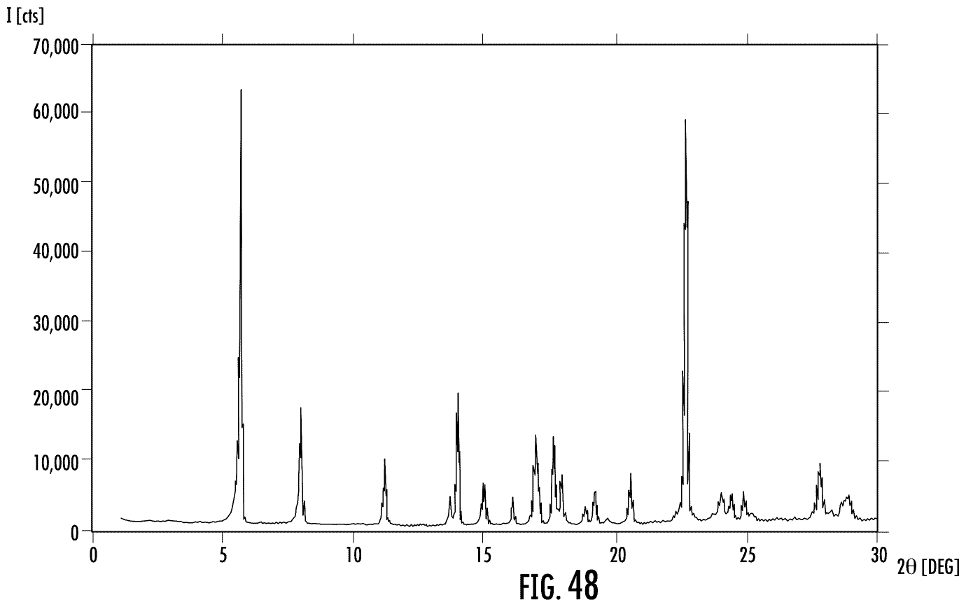

In some embodiments, the disclosure provides crystalline Compound I Form A. In some embodiments, the disclosure provides Compound I Form A. FIG. 48 provides an X-ray powder diffractogram of Compound I Form A.

In some embodiments, crystalline Compound I Form A is substantially pure. In some embodiments, crystalline Compound I Form A is substantially crystalline. In some embodiments, crystalline Compound I Form A is characterized by an X-ray powder diffractogram generated by an X-ray powder diffraction analysis with an incident beam of Cu Kα radiation.

In some embodiments, crystalline Compound Form A is characterized by an X-ray powder diffractogram substantially similar to FIG. 48.

In some embodiments, crystalline Compound I Form A is characterized by a C-centered monoclinic crystal system, Cc space group and the following unit cell dimensions resulting from successful indexing of Form A XRPD:

| | | | |
|---|---|---|---|
| a | 31.2 ± 0.1 Å | α | 90 ± 0.1° |
| b | 11.8 ± 0.1 Å | β | 91.8 ± 0.1° |
| c | 11.2 ± 0.1 Å | γ | 90 ± 0.1°. |

In some embodiments, Compound I Form A is characterized by a DSC having an endotherm peak temperature of 138° C. In some embodiments, Compound I Form A is characterized by a DSC having an endotherm onset temperature of 137° C. In some embodiments, Compound I Form A is characterized by a DSC having an endotherm peak temperature of 138° C. and/or an onset endotherm temperature of 137° C.

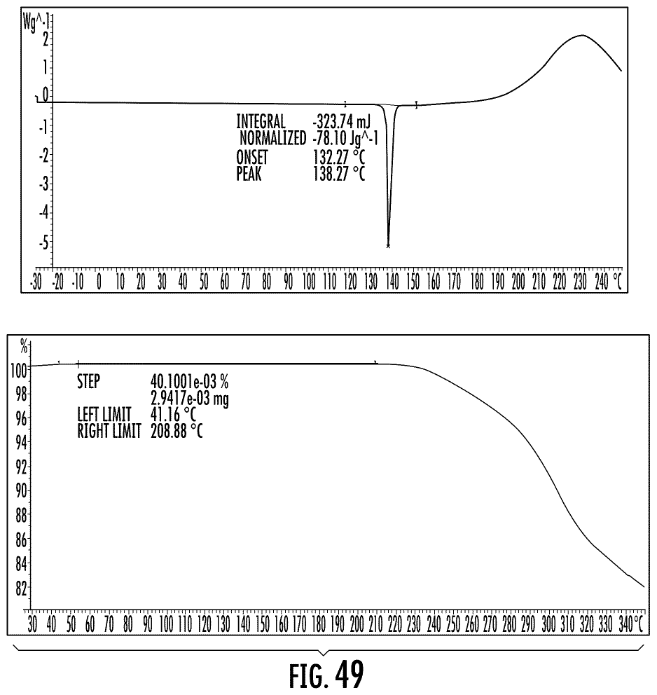

In some embodiments, Compound I Form A is characterized by a DSC substantially similar to FIG. 49.

In some embodiments, Compound I Form A is also characterized by weight loss of 0.1% up to 209° C., as measured by thermogravimetric analysis.

In some embodiments, Compound I Form A is characterized by a TGA substantially similar to FIG. 49.

In some embodiments, Compound I Form A is also characterized as non-hygroscopic, evidenced by water uptake of 0.3% at relative humidity of up to 95%, as measured by DVS. In some embodiments, Compound I Form A is also characterized as non-hygroscopic, evidenced by total water uptake of 0.3% (<0.1 moles), as measured by DVS.

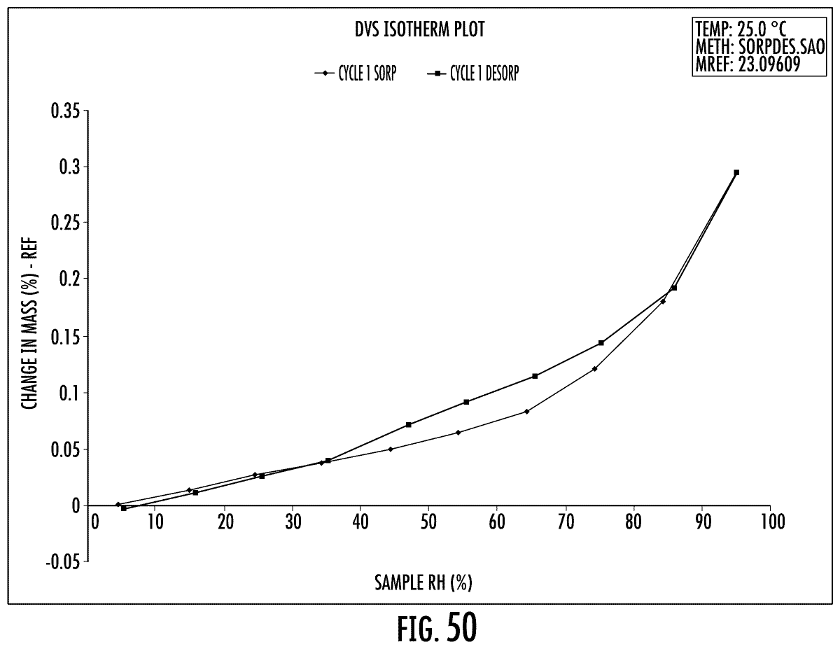

In some embodiments, Compound I Form A is characterized by a DVS substantially similar to FIG. 50 FIG. 11.

In some embodiments, the method of making crystalline Compound I Form A comprises: dissolving Compound I Form 1 in water, adding NaOH, aging at rt for 1 day, and recovering the solids to yield crystalline Compound I Form A.

### Y-2. Compound I Material C

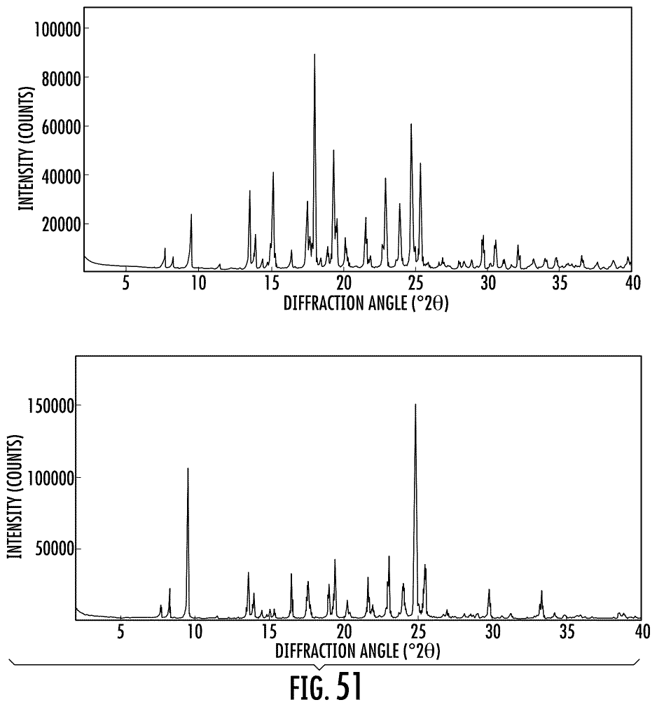

In some embodiments, the disclosure provides crystalline Compound I Material C. In some embodiments, the disclosure provides Compound I Material C. FIG. 51 provides an X-ray powder diffractogram of Compound I Material C with corresponding tabulated data shown in Table 30.

TABLE 30
XRPD Signals for Crystalline Compound I
Material C (sucralose cocrystal attempt)

| °2Θ | d-spacing | Relative |
|---|---|---|
| (±0.2°) | (Å) | Intensity |
| 7.7 | 11.4812 | 9 |
| 8.3 | 10.6525 | 17 |
| 8.62 | 10.2578 | 3 |
| 9.54 | 9.2705 | 89 |
| 11.5 | 7.6945 | 3 |
| 12.3 | 7.1958 | 2 |
| 12.94 | 6.8413 | 2 |
| 13.62 | 6.5012 | 28 |
| 13.96 | 6.3437 | 16 |
| 14.5 | 6.1086 | 6 |
| 14.82 | 5.9774 | 4 |
| 15.06 | 5.8827 | 7 |
| 15.34 | 5.7759 | 7 |
| 16.52 | 5.3659 | 26 |
| 17.62 | 5.0334 | 22 |
| 17.78 | 4.9884 | 9 |
| 18.16 | 4.8849 | 3 |
| 19.04 | 4.6611 | 21 |
| 19.44 | 4.566 | 26 |
| 20.24 | 4.3873 | 8 |
| 20.44 | 4.3449 | 1 |
| 21.66 | 4.1028 | 13 |
| 21.94 | 4.0511 | 5 |
| 22.42 | 3.9654 | 1 |
| 23.04 | 3.8601 | 30 |
| 23.74 | 3.7478 | 1 |
| 24.02 | 3.7048 | 13 |
| 24.86 | 3.5815 | 100 |
| 25.46 | 3.4984 | 22 |
| 26.7 | 3.3387 | 1 |
| 26.98 | 3.3047 | 3 |
| 27.46 | 3.248 | 1 |
| 28.14 | 3.171 | 1 |
| 28.48 | 3.1339 | 1 |
| 28.7 | 3.1104 | 1 |
| 29 | 3.0789 | 3 |
| 29.38 | 3.04 | 1 |
| 29.78 | 3 | 13 |
| 30.64 | 2.9178 | 1 |
| 31.2 | 2.8667 | 3 |
| 33.32 | 2.689 | 15 |
| 34.18 | 2.6232 | 2 |
| 34.82 | 2.5765 | 2 |
| 35.66 | 2.5177 | 1 |
| 35.92 | 2.5001 | 1 |
| 36.22 | 2.48 | 1 |
| 36.7 | 2.4487 | 1 |
| 38.48 | 2.3394 | 3 |
| 38.82 | 2.3197 | 2 |

In some embodiments, crystalline Compound I Material C is substantially pure. In some embodiments, crystalline Compound I Material C is substantially crystalline. In some embodiments, Compound I Material C is a mixture. In some embodiments, Compound I Material C is a mixture of Compound I Material C+L-Proline. In some embodiments, crystalline Compound I Material C is characterized by an X-ray powder diffractogram generated by an X-ray powder diffraction analysis with an incident beam of Cu Kα radiation.

In some embodiments, crystalline Compound I Material C is characterized by an X-ray powder diffractogram having a signal at one or more of 7.7±0.2 degrees two-theta, 8.3±0.2 degrees two-theta, 8.62±0.2 degrees two-theta, 9.54±0.2 degrees two-theta, 11.5±0.2 degrees two-theta, 12.3±0.2 degrees two-theta, ±0.2 degrees two-theta, 12.94±0.2 degrees two-theta, 13.62±0.2 degrees two-theta, 13.96±0.2 degrees two-theta, 14.5±0.2 degrees two-theta, 14.82±0.2 degrees two-theta, 15.06±0.2 degrees two-theta, 15.34±0.2 degrees two-theta, 16.52±0.2 degrees two-theta, 17.62±0.2 degrees two-theta, 17.78±0.2 degrees two-theta, 18.16±0.2 degrees two-theta, 19.04±0.2 degrees two-theta, 19.44±0.2 degrees two-theta, 20.24±0.2 degrees two-theta, 20.44±0.2 degrees two-theta, 21.66±0.2 degrees two-theta, 21.94±0.2 degrees two-theta, 22.42±0.2 degrees two-theta, 23.04±0.2 degrees two-theta, 23.74±0.2 degrees two-theta, 24.02±0.2 degrees two-theta, 24.86±0.2 degrees two-theta, 25.46±0.2 degrees two-theta, 26.7±0.2 degrees two-theta, 26.98±0.2 degrees two-theta, 27.46±0.2 degrees two-theta, 28.14±0.2 degrees two-theta, 28.48±0.2 degrees two-theta, 28.7±0.2 degrees two-theta, 29±0.2 degrees two-theta, 29.38±0.2 degrees two-theta, 29.78±0.2 degrees two-theta, 30.64±0.2 degrees two-theta, 31.2±0.2 degrees two-theta, 33.32±0.2 degrees two-theta, 34.18±0.2 degrees two-theta, 34.82±0.2 degrees two-theta, 35.66±0.2 degrees two-theta, 35.92±0.2 degrees two-theta, 36.22±0.2 degrees two-theta, 36.7±0.2 degrees two-theta, 38.48±0.2 degrees two-theta, and 38.82±0.2 degrees two-theta.

In some embodiments, crystalline Compound I Material C is characterized by an X-ray powder diffractogram having a signal at one or more of 8.3±0.2 degrees two-theta, 9.54±0.2 degrees two-theta, 13.62±0.2 degrees two-theta, 13.96±0.2 degrees two-theta, and 24.86±0.2 degrees two-theta.

In some embodiments, crystalline Compound I Material C is characterized by an X-ray powder diffractogram having a signal at one or more of 8.3±0.2 degrees two-theta, 9.54±0.2 degrees two-theta, and 13.62±0.2 degrees two-theta.

In some embodiments, crystalline Compound I Material C is characterized by an X-ray powder diffractogram having a signal at one or more of 13.96±0.2 degrees two-theta and 24.86±0.2 degrees two-theta.

In some embodiments, crystalline Compound I Material C is characterized by an X-ray powder diffractogram having a signal at 8.3±0.2 degrees two-theta. In some embodiments, crystalline Compound I Material C is characterized by an X-ray powder diffractogram having a signal at 9.54±0.2 degrees two-theta. In some embodiments, crystalline Compound I Material C is characterized by an X-ray powder diffractogram having a signal at 13.62±0.2 degrees two-theta. In some embodiments, crystalline Compound I Material C is characterized by an X-ray powder diffractogram having a signal at 13.96±0.2 degrees two-theta. In some embodiments, crystalline Compound I Material C is characterized by an X-ray powder diffractogram having a signal at 24.86±0.2 degrees two-theta.

In some embodiments, crystalline Compound Material C is characterized by an X-ray powder diffractogram substantially similar to FIG. 51.

In some embodiments, Compound I Material C is characterized by a DSC having an endotherm peak temperature of 139° C. In some embodiments, Compound I Material C is characterized by a DSC having an endotherm of temperature of 151° C. In some embodiments, Compound I Material C is characterized by a DSC having an endotherm peak temperature of 139° C. and/or an endotherm peak temperature of 151° C.

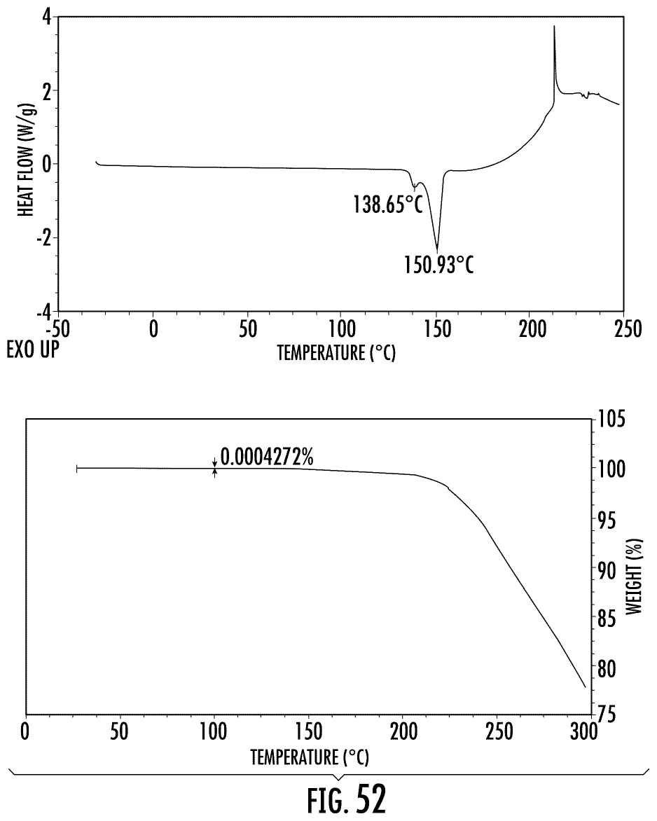

In some embodiments, Compound I Material C is characterized by a DSC substantially similar to FIG. 52.

In some embodiments, Compound I Material C is also characterized by no significant weight loss of up to 200° C., as measured by thermogravimetric analysis.

In some embodiments, Compound I Material C is characterized by a TGA substantially similar to FIG. 52.

In some embodiments, the method of making crystalline Compound I Material C comprises: stirring Compound I Form A (free base), L-Proline, 50:50 isopropyl ether:methyl isobutyl ketone (MBK) at rt for 6 days, adding additional 50:50 isopropyl ether:MBK, stirring for 1 day at rt, centrifuging, and recovering the solids to yield crystalline Compound I Material C.

In some embodiments, the method of making crystalline Compound I Material C comprises: dissolving Compound I Form A (free base) and sucralose in 50:50 acetonitrile:water at reflux, cooling, equilibrating for 3 days, removing solvent, and recovering the solids to yield crystalline Compound I Material C.

### Y-3. Compound I Betaine HCl Material A

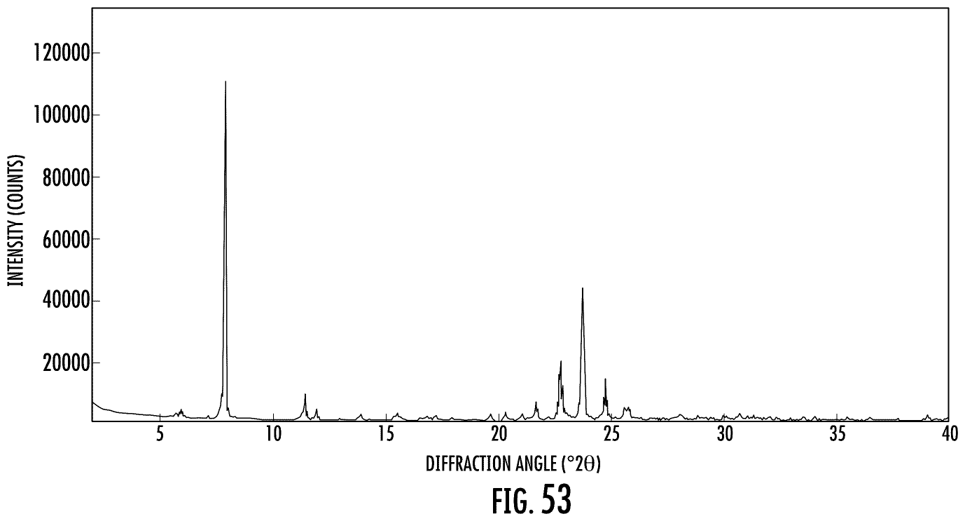

In some embodiments, the disclosure provides crystalline Compound I Betaine HCl Material A. In some embodiments, the disclosure provides Compound I Betaine HCl Material A. FIG. 53 provides an X-ray powder diffractogram of Compound I Betaine HCl Material A with corresponding tabulated data shown in Table 31.

TABLE 31
XRPD Signals for Crystalline Compound I
Betaine HCl Material A

| °2Θ | d-spacing | Relative |
|---|---|---|
| (±0.2°) | (Å) | Intensity |
| 5.72 | 15.4502 | 3 |
| 5.96 | 14.8286 | 4 |
| 7.14 | 12.3804 | 2 |
| 7.92 | 11.1628 | 100 |
| 11.46 | 7.7213 | 7 |
| 11.96 | 7.3996 | 3 |
| 13.9 | 6.3709 | 2 |
| 15.5 | 5.7167 | 2 |
| 16.82 | 5.2709 | 1 |
| 17.22 | 5.1494 | 1 |
| 17.96 | 4.9388 | 1 |
| 19.66 | 4.5154 | 1 |
| 20.34 | 4.366 | 2 |
| 21.06 | 4.2183 | 1 |
| 21.72 | 4.0916 | 4 |
| 22.26 | 3.9936 | 1 |
| 22.8 | 3.9002 | 13 |
| 23.78 | 3.7416 | 36 |
| 24.78 | 3.5929 | 8 |
| 25.64 | 3.4743 | 2 |
| 25.82 | 3.4504 | 2 |
| 28.1 | 3.1755 | 1 |
| 28.88 | 3.0914 | 1 |
| 30.1 | 2.9689 | 1 |
| 30.74 | 2.9085 | 1 |
| 31.36 | 2.8524 | 1 |
| 32.06 | 2.7917 | 1 |
| 32.94 | 2.7191 | 1 |
| 33.58 | 2.6687 | 1 |
| 34.1 | 2.6292 | 1 |
| 35.5 | 2.5287 | 1 |
| 36.5 | 2.4617 | 1 |
| 39.06 | 2.306 | 1 |

In some embodiments, crystalline Compound I Betaine HCl Material A is substantially pure. In some embodiments, crystalline Compound I Betaine HCl Material A is substantially crystalline. In some embodiments, crystalline Compound I Betaine HCl Material A is characterized by an X-ray powder diffractogram generated by an X-ray powder diffraction analysis with an incident beam of Cu Kα radiation.

In some embodiments, crystalline Compound I Betaine HCl Material A is characterized by an X-ray powder diffractogram having a signal at one or more of 5.72±0.2 degrees two-theta, 5.96±0.2 degrees two-theta, 7.14±0.2 degrees two-theta, 7.92±0.2 degrees two-theta, 11.46±0.2 degrees two-theta, 11.96±0.2 degrees two-theta, ±0.2 degrees two-theta, 13.9±0.2 degrees two-theta, 15.5±0.2 degrees two-theta, 16.82±0.2 degrees two-theta, 17.22±0.2 degrees two-theta, 17.96±0.2 degrees two-theta, 19.66±0.2 degrees two-theta, 20.34±0.2 degrees two-theta, 21.06±0.2 degrees two-theta, 21.72±0.2 degrees two-theta, 22.26±0.2 degrees two-theta, 22.8±0.2 degrees two-theta, 23.78±0.2 degrees two-theta, 24.78±0.2 degrees two-theta, 25.64±0.2 degrees two-theta, 25.82±0.2 degrees two-theta, 28.1±0.2 degrees two-theta, 28.88±0.2 degrees two-theta, 30.1±0.2 degrees two-theta, 30.74±0.2 degrees two-theta, 31.36±0.2 degrees two-theta, 32.06±0.2 degrees two-theta, 32.94±0.2 degrees two-theta, 33.58±0.2 degrees two-theta, 34.1±0.2 degrees two-theta, 35.5±0.2 degrees two-theta, 36.5±0.2 degrees two-theta, and 39.06±0.2 degrees two-theta.

In some embodiments, crystalline Compound I Betaine HCl Material A is characterized by an X-ray powder diffractogram having a signal at one or more of 5.72±0.2 degrees two-theta, 5.96±0.2 degrees two-theta, 7.92±0.2 degrees two-theta, 11.46±0.2 degrees two-theta, and 23.78±0.2 degrees two-theta.

In some embodiments, crystalline Compound I Betaine HCl Material A is characterized by an X-ray powder diffractogram having a signal at one or more of 5.96±0.2 degrees two-theta, 7.92±0.2 degrees two-theta, and 11.46±0.2 degrees two-theta.

In some embodiments, crystalline Compound I Betaine HCl Material A is characterized by an X-ray powder diffractogram having a signal at one or more of 5.72±0.2 degrees two-theta and 23.78±0.2 degrees two-theta.

In some embodiments, crystalline Compound I Betaine HCl Material A is characterized by an X-ray powder diffractogram having a signal at 5.72±0.2 degrees two-theta. In some embodiments, crystalline Compound I Betaine HCl Material A is characterized by an X-ray powder diffractogram having a signal at 5.96±0.2 degrees two-theta. In some embodiments, crystalline Compound I Betaine HCl Material A is characterized by an X-ray powder diffractogram having a signal at 7.92±0.2 degrees two-theta. In some embodiments, crystalline Compound I Betaine HCl Material A is characterized by an X-ray powder diffractogram having a signal at 11.46±0.2 degrees two-theta. In some embodiments, crystalline Compound I Betaine HCl Material A is characterized by an X-ray powder diffractogram having a signal at 23.78±0.2 degrees two-theta.

In some embodiments, crystalline Compound Betaine HCl Material A is characterized by an X-ray powder diffractogram substantially similar to FIG. 53.

In some embodiments, Compound I Betaine HCl Material A is characterized by a DSC having an endotherm peak temperature of 110° C.

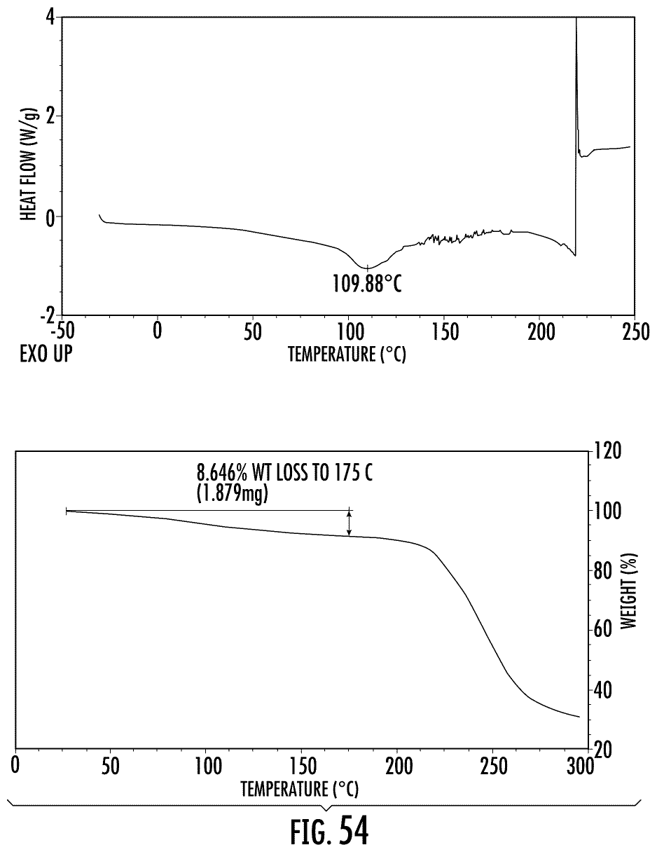

In some embodiments, Compound I Betaine HCl Material A is characterized by a DSC substantially similar to FIG. 54.

In some embodiments, Compound I Betaine HCl Material A is also characterized by weight loss of 8.6% up to 175° C., as measured by thermogravimetric analysis.

In some embodiments, Compound I Betaine HCl Material A is characterized by a TGA substantially similar to FIG. 54.

In some embodiments, the method of making crystalline Compound I Betaine HCl Material A comprises: stirring Compound I Form A (free base), betaine HCl, and water at rt for 5 days, centrifuging, and recovering the solids to yield crystalline Compound I Betaine HCl Material A.

### Y-4. Compound I Methyl Paraben Material A

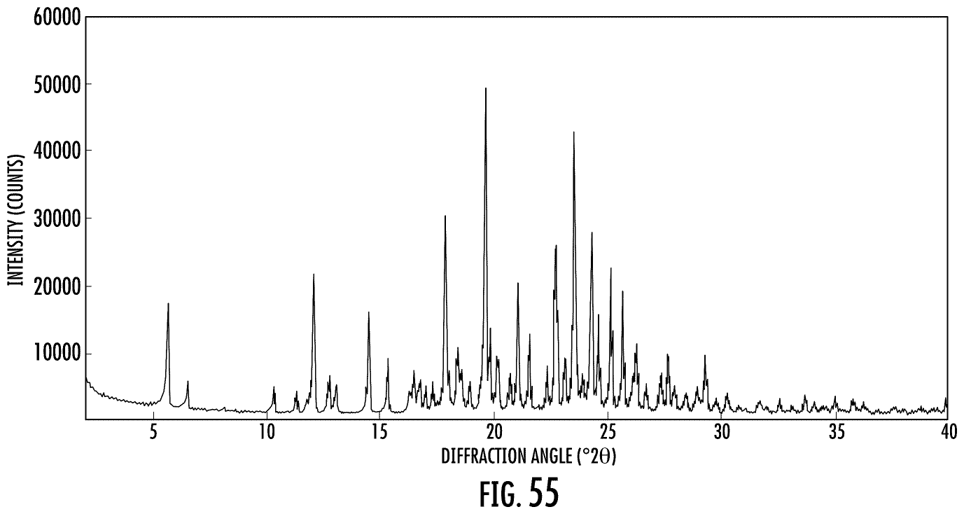

In some embodiments, the disclosure provides crystalline Compound I Methyl Paraben Material A. In some embodiments, the disclosure provides Compound I Methyl Paraben Material A unsolvated. In some embodiments, the disclosure provides Compound I Methyl Paraben Material A anhydrous. FIG. 55 provides an X-ray powder diffractogram of Compound I Methyl Paraben Material A with corresponding tabulated data shown in Table 32.

TABLE 32
XRPD Signals for Crystalline Compound I
Methyl Paraben Material A

| °2Θ | d-spacing | Relative |
|---|---|---|
| (±0.2°) | (Å) | Intensity |
| 5.66 | 15.6139 | 31 |
| 6.52 | 13.5562 | 7 |
| 8.16 | 10.835 | 1 |
| 10.32 | 8.5715 | 8 |
| 11.3 | 7.8303 | 7 |
| 11.76 | 7.525 | 2 |
| 12.08 | 7.3264 | 42 |
| 12.74 | 6.9483 | 10 |
| 13.04 | 6.7891 | 8 |
| 14.5 | 6.1086 | 31 |
| 15.32 | 5.7834 | 17 |
| 16.26 | 5.4512 | 2 |
| 16.46 | 5.3854 | 9 |
| 16.74 | 5.2959 | 7 |
| 16.98 | 5.2216 | 7 |
| 17.3 | 5.1257 | 7 |
| 17.5 | 5.0676 | 2 |
| 17.88 | 4.9607 | 57 |
| 18.4 | 4.8217 | 13 |
| 18.56 | 4.7805 | 6 |
| 18.92 | 4.6903 | 7 |
| 19.62 | 4.5246 | 100 |
| 19.82 | 4.4793 | 16 |
| 20.14 | 4.4089 | 15 |
| 20.7 | 4.2909 | 9 |
| 21.04 | 4.2223 | 35 |
| 21.54 | 4.1254 | 26 |
| 21.94 | 4.0511 | 1 |
| 22.32 | 3.983 | 9 |
| 22.7 | 3.9171 | 52 |
| 23.1 | 3.8502 | 14 |
| 23.5 | 3.7856 | 89 |
| 23.88 | 3.7262 | 8 |
| 24.28 | 3.6657 | 56 |
| 24.58 | 3.6216 | 28 |
| 25.12 | 3.545 | 45 |
| 25.64 | 3.4743 | 36 |
| 26.1 | 3.4141 | 3 |
| 26.26 | 3.3936 | 16 |
| 26.68 | 3.3411 | 8 |
| 27.34 | 3.262 | 10 |
| 27.62 | 3.2295 | 12 |
| 27.92 | 3.1955 | 3 |
| 28.42 | 3.1404 | 5 |
| 28.92 | 3.0873 | 6 |
| 29.28 | 3.0501 | 13 |
| 29.76 | 3.002 | 5 |
| 30.24 | 2.9554 | 5 |
| 30.72 | 2.9103 | 2 |
| 31.02 | 2.8829 | 1 |
| 31.62 | 2.8295 | 4 |
| 31.96 | 2.8002 | 1 |
| 32.24 | 2.7765 | 1 |
| 32.52 | 2.7532 | 4 |
| 33.08 | 2.7079 | 2 |
| 33.62 | 2.6656 | 6 |
| 34.06 | 2.6322 | 3 |
| 34.58 | 2.5938 | 1 |
| 34.94 | 2.5679 | 5 |
| 35.74 | 2.5122 | 2 |
| 36.2 | 2.4814 | 3 |
| 37.62 | 2.3909 | 2 |
| 39.82 | 2.2637 | 5 |

In some embodiments, crystalline Compound I Methyl Paraben Material A is substantially pure. In some embodiments, crystalline Compound I Methyl Paraben Material A is substantially crystalline. In some embodiments, Compound I Methyl Paraben Material A is 1:1 ONC206:Methyl Paraben cocrystal that appears to be anhydrous/unsolvated. In some embodiments, crystalline Compound I Methyl Paraben Material A is characterized by an X-ray powder diffractogram generated by an X-ray powder diffraction analysis with an incident beam of Cu Kα radiation.

In some embodiments, crystalline Compound I Methyl Paraben Material A is characterized by an X-ray powder diffractogram having a signal at one or more of 5.66±0.2 degrees two-theta, 6.52±0.2 degrees two-theta, 8.16±0.2 degrees two-theta, 10.32±0.2 degrees two-theta, 11.3±0.2 degrees two-theta, 11.76±0.2 degrees two-theta, ±0.2 degrees two-theta, 12.08±0.2 degrees two-theta, 12.74±0.2 degrees two-theta, 13.04±0.2 degrees two-theta, 14.5±0.2 degrees two-theta, 15.32±0.2 degrees two-theta, 16.26±0.2 degrees two-theta, 16.46±0.2 degrees two-theta, 16.74±0.2 degrees two-theta, 16.98±0.2 degrees two-theta, 17.3±0.2 degrees two-theta, 17.5±0.2 degrees two-theta, 17.88±0.2 degrees two-theta, 18.4±0.2 degrees two-theta, 18.56±0.2 degrees two-theta, 18.92±0.2 degrees two-theta, 19.62±0.2 degrees two-theta, 19.82±0.2 degrees two-theta, 20.14±0.2 degrees two-theta, 20.7±0.2 degrees two-theta, 21.04±0.2 degrees two-theta, 21.54±0.2 degrees two-theta, 21.94±0.2 degrees two-theta, 22.32±0.2 degrees two-theta, 22.7±0.2 degrees two-theta, 23.1±0.2 degrees two-theta, 23.5±0.2 degrees two-theta, 23.88±0.2 degrees two-theta, 24.28±0.2 degrees two-theta, 24.58±0.2 degrees two-theta, 25.12±0.2 degrees two-theta, 25.64±0.2 degrees two-theta, 26.1±0.2 degrees two-theta, 26.26±0.2 degrees two-theta, 26.68±0.2 degrees two-theta, 27.34±0.2 degrees two-theta, 27.62±0.2 degrees two-theta, 27.92±0.2 degrees two-theta, 28.42±0.2 degrees two-theta, 28.92±0.2 degrees two-theta, 29.28±0.2 degrees two-theta, 29.76±0.2 degrees two-theta, 30.24±0.2 degrees two-theta, 30.72±0.2 degrees two-theta, 31.02±0.2 degrees two-theta, 31.62±0.2 degrees two-theta, 31.96±0.2 degrees two-theta, 32.24±0.2 degrees two-theta, 32.52±0.2 degrees two-theta, 33.08±0.2 degrees two-theta, 33.62±0.2 degrees two-theta, 34.06±0.2 degrees two-theta, 34.58±0.2 degrees two-theta, 34.94±0.2 degrees two-theta, 35.74±0.2 degrees two-theta, 36.2±0.2 degrees two-theta, 37.62±0.2 degrees two-theta, and 39.82±0.2 degrees two-theta.

In some embodiments, crystalline Compound I Methyl Paraben Material A is characterized by an X-ray powder diffractogram having a signal at one or more of 5.66±0.2 degrees two-theta, 6.52±0.2 degrees two-theta, 12.08±0.2 degrees two-theta, 14.5±0.2 degrees two-theta, 15.32±0.2 degrees two-theta, and 19.62±0.2 degrees two-theta.

In some embodiments, crystalline Compound I Methyl Paraben Material A is characterized by an X-ray powder diffractogram having a signal at one or more of 5.66±0.2 degrees two-theta, 12.08±0.2 degrees two-theta, and 14.5±0.2 degrees two-theta.

In some embodiments, crystalline Compound I Methyl Paraben Material A is characterized by an X-ray powder diffractogram having a signal at one or more of 6.52±0.2 degrees two-theta, 15.32±0.2 degrees two-theta, and 19.62±0.2 degrees two-theta.

In some embodiments, crystalline Compound I Methyl Paraben Material A is characterized by an X-ray powder diffractogram having a signal at 5.66±0.2 degrees two-theta. In some embodiments, crystalline Compound I Methyl Paraben Material A is characterized by an X-ray powder diffractogram having a signal at 6.52±0.2 degrees two-theta. In some embodiments, crystalline Compound I Methyl Paraben Material A is characterized by an X-ray powder diffractogram having a signal at 12.08±0.2 degrees two-theta. In some embodiments, crystalline Compound I Methyl Paraben Material A is characterized by an X-ray powder diffractogram having a signal at 14.5±0.2 degrees two-theta. In some embodiments, crystalline Compound I Methyl Paraben Material A is characterized by an X-ray powder diffractogram having a signal at 15.32±0.2 degrees two-theta. In some embodiments, crystalline Compound I Methyl Paraben Material A is characterized by an X-ray powder diffractogram having a signal at 19.62±0.2 degrees two-theta.

In some embodiments, crystalline Compound I Methyl Paraben Material A is characterized by an X-ray powder diffractogram substantially similar to FIG. 55.

In some embodiments, Compound I Methyl Paraben Material A is characterized by a DSC having an endotherm peak temperature of 135° C.

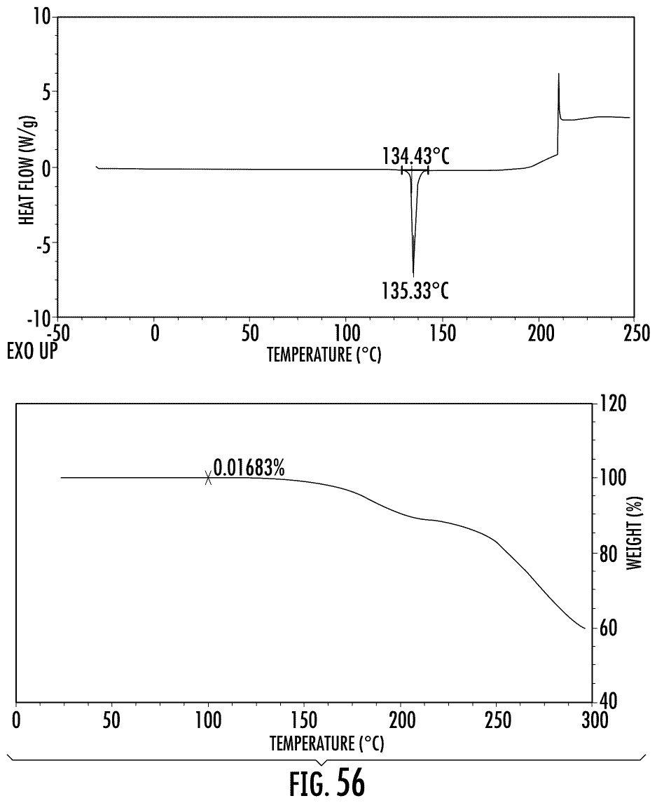

In some embodiments, Compound I Methyl Paraben Material A is characterized by a DSC substantially similar to FIG. 56.

In some embodiments, Compound I Methyl Paraben Material A is also characterized by no substantial weight loss up to 200° C., as measured by thermogravimetric analysis.

In some embodiments, Compound I Methyl Paraben Material A is characterized by a TGA substantially similar to FIG. 56.

In some embodiments, the method of making crystalline Compound I Methyl Paraben Material A comprises: stirring Compound I Form A (free base), methyl paraben, and 50:50 ethanol:hexane at rt for 7 days, centrifuging, and recovering the solids to yield crystalline Compound I Methyl Paraben Material A.

### Y-5. Compound I Propyl Gallate Material A

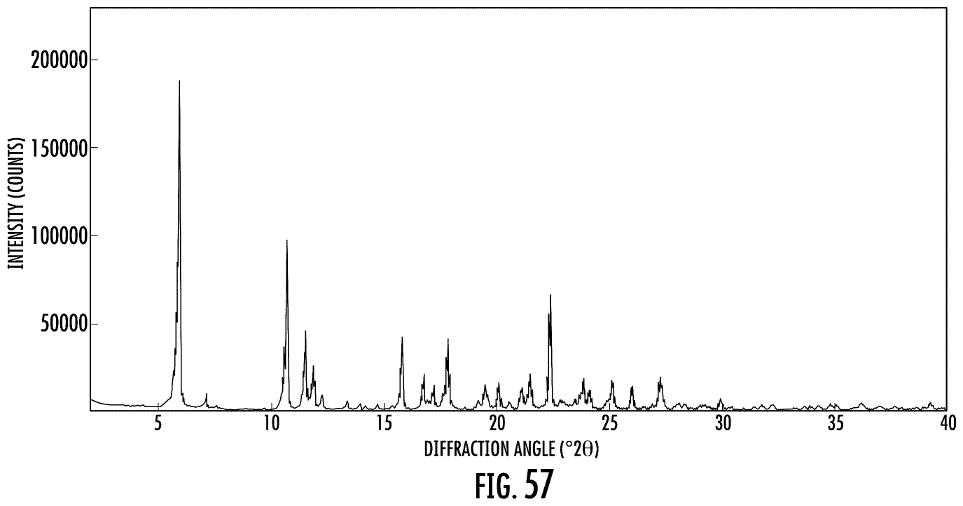

In some embodiments, the disclosure provides crystalline Compound I Propyl Gallate Material A. In some embodiments, the disclosure provides Compound I Propyl Gallate Material A anhydrous. In some embodiments, the disclosure provides Compound I Propyl Gallate Material A unsolvated. FIG. 57 provides an X-ray powder diffractogram of Compound I Propyl Gallate Material A with corresponding tabulated data shown in Table 33.

TABLE 33
XRPD Signals for Crystalline Compound I
Propyl Gallate Material A

| °2Θ | d-spacing | Relative |
|---|---|---|
| (±0.2°) | (Å) | Intensity |
| 5.96 | 14.8286 | 100 |
| 7.12 | 12.4151 | 5 |
| 10.72 | 8.2526 | 51 |
| 11.56 | 7.6547 | 25 |
| 11.88 | 7.4492 | 14 |
| 12.28 | 7.2075 | 4 |
| 13.38 | 6.6173 | 2 |
| 13.9 | 6.3709 | 1 |
| 14.18 | 6.2457 | 1 |
| 14.7 | 6.0259 | 1 |
| 15.34 | 5.7759 | 1 |
| 15.82 | 5.6018 | 18 |
| 16.76 | 5.2896 | 9 |
| 16.96 | 5.2277 | 1 |
| 17.2 | 5.1553 | 6 |
| 17.84 | 4.9718 | 11 |
| 19.18 | 4.6273 | 2 |
| 19.48 | 4.5568 | 6 |
| 20.1 | 4.4176 | 7 |
| 20.56 | 4.3198 | 2 |
| 21.12 | 4.2065 | 5 |
| 21.48 | 4.1368 | 8 |
| 22.38 | 3.9724 | 30 |
| 22.84 | 3.8934 | 2 |
| 23.46 | 3.7919 | 2 |
| 23.84 | 3.7323 | 7 |
| 24.1 | 3.6927 | 4 |
| 25.12 | 3.545 | 8 |
| 25.98 | 3.4296 | 6 |
| 27.22 | 3.2761 | 9 |
| 27.98 | 3.1888 | 2 |
| 28.32 | 3.1513 | 1 |
| 29.16 | 3.0624 | 1 |
| 29.92 | 2.9863 | 3 |
| 30.36 | 2.944 | 1 |
| 31.38 | 2.8506 | 1 |
| 31.74 | 2.8191 | 1 |
| 32.16 | 2.7832 | 1 |
| 33.62 | 2.6656 | 1 |
| 33.88 | 2.6458 | 1 |
| 34.22 | 2.6203 | 1 |
| 34.76 | 2.5808 | 2 |
| 35.02 | 2.5622 | 1 |
| 36.16 | 2.484 | 1 |
| 36.92 | 2.4346 | 1 |
| 37.58 | 2.3934 | 1 |
| 39.18 | 2.2992 | 2 |

In some embodiments, crystalline Compound I Propyl Gallate Material A is substantially pure. In some embodiments, crystalline Compound I Propyl Gallate Material A is substantially crystalline. In some embodiments, ONC206 Propyl Gallate Material A is 1:1 ONC206:propyl gallate cocrystal that may be anhydrous and/or unsolvated. In some embodiments, crystalline Compound I Propyl Gallate Material A is characterized by an X-ray powder diffractogram generated by an X-ray powder diffraction analysis with an incident beam of Cu Kα radiation.

In some embodiments, crystalline Compound I Propyl Gallate Material A is characterized by an X-ray powder diffractogram having a signal at one or more of 5.96±0.2 degrees two-theta, 7.12±0.2 degrees two-theta, 10.72±0.2 degrees two-theta, 11.56±0.2 degrees two-theta, 11.88±0.2 degrees two-theta, 12.28±0.2 degrees two-theta, ±0.2 degrees two-theta, 13.38±0.2 degrees two-theta, 13.9±0.2 degrees two-theta, 14.18±0.2 degrees two-theta, 14.7±0.2 degrees two-theta, 15.34±0.2 degrees two-theta, 15.82±0.2 degrees two-theta, 16.76±0.2 degrees two-theta, 16.96±0.2 degrees two-theta, 17.2±0.2 degrees two-theta, 17.84±0.2 degrees two-theta, 19.18±0.2 degrees two-theta, 19.48±0.2 degrees two-theta, 20.1±0.2 degrees two-theta, 20.56±0.2 degrees two-theta, 21.12±0.2 degrees two-theta, 21.48±0.2 degrees two-theta, 22.38±0.2 degrees two-theta, 22.84±0.2 degrees two-theta, 23.46±0.2 degrees two-theta, 23.84±0.2 degrees two-theta, 24.1±0.2 degrees two-theta, 25.12±0.2 degrees two-theta, 25.98±0.2 degrees two-theta, 27.22±0.2 degrees two-theta, 27.98±0.2 degrees two-theta, 28.32±0.2 degrees two-theta, 29.16±0.2 degrees two-theta, 29.92±0.2 degrees two-theta, 30.36±0.2 degrees two-theta, 31.38±0.2 degrees two-theta, 31.74±0.2 degrees two-theta, 32.16±0.2 degrees two-theta, 33.62±0.2 degrees two-theta, 33.88±0.2 degrees two-theta, 34.22±0.2 degrees two-theta, 34.76±0.2 degrees two-theta, 35.02±0.2 degrees two-theta, 36.16±0.2 degrees two-theta, 36.92±0.2 degrees two-theta, 37.58±0.2 degrees two-theta, and 39.18±0.2 degrees two-theta.

In some embodiments, crystalline Compound I Propyl Gallate Material A is characterized by an X-ray powder diffractogram having a signal at one or more of 5.96±0.2 degrees two-theta, 10.72±0.2 degrees two-theta, 11.56±0.2 degrees two-theta, 11.88±0.2 degrees two-theta, and 15.82±0.2 degrees two-theta.

In some embodiments, crystalline Compound I Propyl Gallate Material A is characterized by an X-ray powder diffractogram having a signal at one or more of 5.96±0.2 degrees two-theta, 10.72±0.2 degrees two-theta, and 11.56±0.2 degrees two-theta.

In some embodiments, crystalline Compound I Propyl Gallate Material A is characterized by an X-ray powder diffractogram having a signal at one or more of 11.88±0.2 degrees two-theta and 15.82±0.2 degrees two-theta.

In some embodiments, crystalline Compound I Propyl Gallate Material A is characterized by an X-ray powder diffractogram having a signal at 5.96±0.2 degrees two-theta. In some embodiments, crystalline Compound I Propyl Gallate Material A is characterized by an X-ray powder diffractogram having a signal at 10.72±0.2 degrees two-theta. In some embodiments, crystalline Compound I Propyl Gallate Material A is characterized by an X-ray powder diffractogram having a signal at 11.56±0.2 degrees two-theta. In some embodiments, crystalline Compound I Propyl Gallate Material A is characterized by an X-ray powder diffractogram having a signal at 11.88±0.2 degrees two-theta. In some embodiments, crystalline Compound I Propyl Gallate Material A is characterized by an X-ray powder diffractogram having a signal at 15.82±0.2 degrees two-theta.

In some embodiments, crystalline Compound I Propyl Gallate Material A is characterized by an X-ray powder diffractogram substantially similar to FIG. 57.

In some embodiments, Compound I Propyl Gallate Material A is characterized by a DSC having an endotherm peak temperature of 109° C. In some embodiments, Compound I Propyl Gallate Material A is characterized by a DSC having an endotherm peak temperature of 127° C. In some embodiments, Compound I Propyl Gallate Material A is characterized by a DSC having an endotherm peak temperature of 136° C. In some embodiments, Compound I Propyl Gallate Material A is characterized by a DSC having an endotherm peak temperature of 109° C., 127° C., and/or. 136° C.

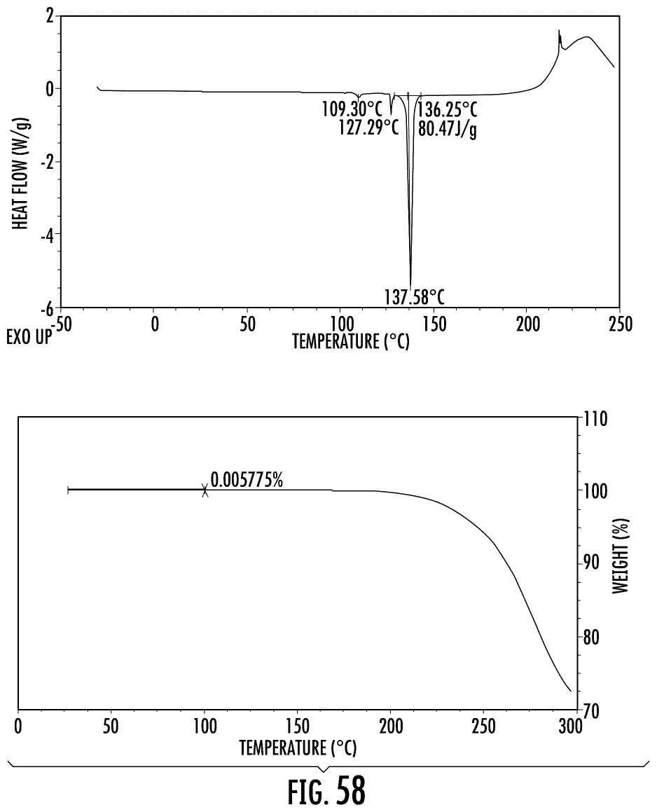

In some embodiments, Compound I Propyl Gallate Material A is characterized by a DSC substantially similar to FIG. 58.

In some embodiments, Compound I Propyl Gallate Material A is also characterized by no substantial weight loss up to 200° C., as measured by thermogravimetric analysis.

In some embodiments, Compound I Propyl Gallate Material A is characterized by a TGA substantially similar to FIG. 58.

In some embodiments, the method of making crystalline Compound I Propyl Gallate Material A comprises: stirring Compound I Form A (free base), propyl gallate, and 50:50 acetonitrile:diethyl ether at rt for 7 days, centrifuging, and recovering the solids to yield crystalline Compound I Propyl Gallate Material A.

### Y-6. Compound I Saccharin Material A+Minor Compound I Form A

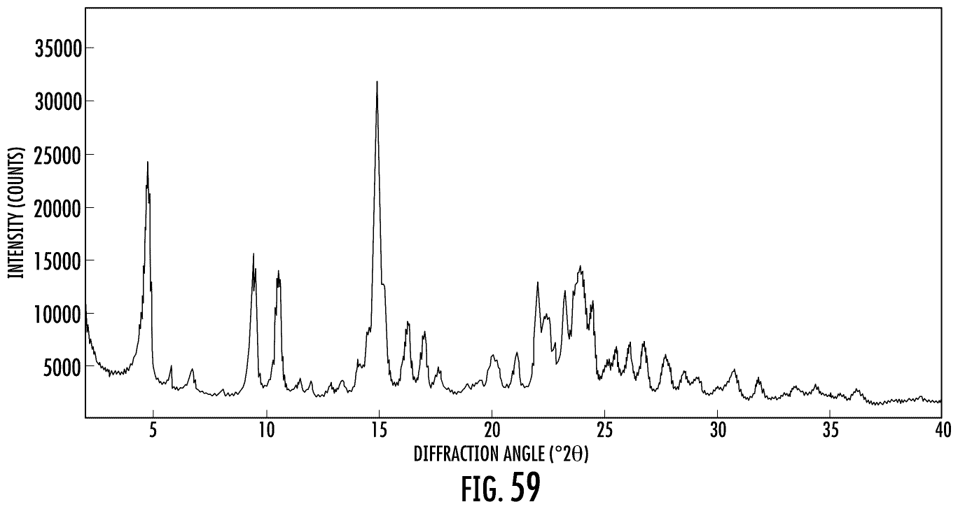

In some embodiments, the disclosure provides crystalline Compound I Saccharin Material A+minor Compound I Form A. In some embodiments, the disclosure provides Compound I Saccharin Material A+minor Compound I Form A. FIG. 59 provides an X-ray powder diffractogram of Compound I Saccharin Material A+minor Compound I Form A with corresponding tabulated data shown in Table 34.

TABLE 34
XRPD Signals for Crystalline Compound I Saccharin
Material A + minor Compound I Form A

| °2Θ | d-spacing | Relative |
|---|---|---|
| (±0.2°) | (Å) | Intensity |
| 4.76 | 18.5639 | 100 |
| 5.78 | 15.29 | 10 |
| 6.7 | 13.1924 | 12 |
| 8.06 | 10.9692 | 3 |
| 9.46 | 9.3487 | 84 |
| 10.56 | 8.3773 | 73 |
| 11.5 | 7.6945 | 8 |
| 12 | 7.375 | 7 |
| 12.86 | 6.8837 | 6 |
| 13.36 | 6.6272 | 7 |
| 14.06 | 6.2988 | 7 |
| 14.48 | 6.117 | 5 |
| 14.94 | 5.9297 | 80 |
| 16.3 | 5.4379 | 30 |
| 17.02 | 5.2094 | 29 |
| 17.66 | 5.022 | 11 |
| 18.9 | 4.6953 | 2 |
| 19.44 | 4.566 | 4 |
| 20.04 | 4.4307 | 17 |
| 21.12 | 4.2065 | 22 |
| 22.04 | 4.0329 | 31 |
| 22.48 | 3.955 | 8 |
| 22.76 | 3.9069 | 5 |
| 23.26 | 3.8241 | 29 |
| 23.92 | 3.72 | 42 |
| 24.46 | 3.6391 | 28 |
| 25.12 | 3.545 | 7 |
| 25.5 | 3.493 | 10 |
| 26.14 | 3.4089 | 21 |
| 26.78 | 3.3289 | 25 |
| 27.68 | 3.2227 | 22 |
| 28.52 | 3.1296 | 10 |
| 29.18 | 3.0603 | 7 |
| 30.02 | 2.9766 | 3 |
| 30.76 | 2.9066 | 12 |
| 31.82 | 2.8122 | 11 |
| 33.42 | 2.6811 | 5 |
| 34.36 | 2.6099 | 5 |
| 35.52 | 2.5273 | 4 |
| 36.22 | 2.48 | 6 |
| 39.04 | 2.3071 | 3 |

In some embodiments, crystalline Compound I Saccharin Material A+minor Compound I Form A is substantially pure. In some embodiments, crystalline Compound I Saccharin Material A+minor Compound I Form A is substantially crystalline. In some embodiments, crystalline Compound I Saccharin Material A+minor Compound I Form A is a mixture of Compound I Saccharin Material A and Compound I Form A (free base). In some embodiments, crystalline Compound I Saccharin Material A+minor Compound I Form A is characterized by an X-ray powder diffractogram generated by an X-ray powder diffraction analysis with an incident beam of Cu Kα radiation.

In some embodiments, crystalline Compound I Saccharin Material A+possible minor Compound I Form A is characterized by an X-ray powder diffractogram having a signal at one or more of 4.76±0.2 degrees two-theta, 5.78±0.2 degrees two-theta, 6.7±0.2 degrees two-theta, 8.06±0.2 degrees two-theta, 9.46±0.2 degrees two-theta, 10.56±0.2 degrees two-theta, ±0.2 degrees two-theta, 11.5±0.2 degrees two-theta, 12±0.2 degrees two-theta, 12.86±0.2 degrees two-theta, 13.36±0.2 degrees two-theta, 14.06±0.2 degrees two-theta, 14.48±0.2 degrees two-theta, 14.94±0.2 degrees two-theta, 16.3±0.2 degrees two-theta, 17.02±0.2 degrees two-theta, 17.66±0.2 degrees two-theta, 18.9±0.2 degrees two-theta, 19.44±0.2 degrees two-theta, 20.04±0.2 degrees two-theta, 21.12±0.2 degrees two-theta, 22.04±0.2 degrees two-theta, 22.48±0.2 degrees two-theta, 22.76±0.2 degrees two-theta, 23.26±0.2 degrees two-theta, 23.92±0.2 degrees two-theta, 24.46±0.2 degrees two-theta, 25.12±0.2 degrees two-theta, 25.5±0.2 degrees two-theta, 26.14±0.2 degrees two-theta, 26.78±0.2 degrees two-theta, 27.68±0.2 degrees two-theta, 28.52±0.2 degrees two-theta, 29.18±0.2 degrees two-theta, 30.02±0.2 degrees two-theta, 30.76±0.2 degrees two-theta, 31.82±0.2 degrees two-theta, 33.42±0.2 degrees two-theta, 34.36±0.2 degrees two-theta, 35.52±0.2 degrees two-theta, 36.22±0.2 degrees two-theta, and 39.04±0.2 degrees two-theta.

In some embodiments, crystalline Compound I Saccharin Material A+possible minor Compound I Form A is characterized by an X-ray powder diffractogram having a signal at one or more of 4.76±0.2 degrees two-theta, 9.46±0.2 degrees two-theta, 10.56±0.2 degrees two-theta, 14.94±0.2 degrees two-theta, and 16.3±0.2 degrees two-theta.

In some embodiments, crystalline Compound I Saccharin Material A+possible minor Compound I Form A is characterized by an X-ray powder diffractogram having a signal at one or more of 4.76±0.2 degrees two-theta, 9.46±0.2 degrees two-theta, and 10.56±0.2 degrees two-theta.

In some embodiments, crystalline Compound I Saccharin Material A+possible minor Compound I Form A is characterized by an X-ray powder diffractogram having a signal at one or more of 14.94±0.2 degrees two-theta and 16.3±0.2 degrees two-theta.

In some embodiments, crystalline Compound I Saccharin Material A+possible minor Compound I Form A is characterized by an X-ray powder diffractogram having a signal at 4.76±0.2 degrees two-theta. In some embodiments, crystalline Compound I Saccharin Material A+possible minor Compound I Form A is characterized by an X-ray powder diffractogram having a signal at 9.46±0.2 degrees two-theta. In some embodiments, crystalline Compound I Saccharin Material A+possible minor Compound I Form A is characterized by an X-ray powder diffractogram having a signal at 10.56±0.2 degrees two-theta. In some embodiments, crystalline Compound I Saccharin Material A+possible minor Compound I Form A is characterized by an X-ray powder diffractogram having a signal at 14.94±0.2 degrees two-theta. In some embodiments, crystalline Compound I Saccharin Material A+possible minor Compound I Form A is characterized by an X-ray powder diffractogram having a signal at 16.3±0.2 degrees two-theta.

In some embodiments, crystalline Compound I Saccharin Material A+minor Compound I Form A is characterized by an X-ray powder diffractogram substantially similar to FIG. 59.

In some embodiments, Compound I Saccharin Material A+minor Compound I Form A is characterized by a DSC having an onset endotherm temperature of 169° C. In some embodiments, Compound I Saccharin Material A+minor Compound I Form A is characterized by a DSC having an endotherm peak temperature of 173° C. In some embodiments, Compound I Saccharin Material A+minor Compound I Form A is characterized by a DSC having an endotherm onset temperature of 169° C. and/or an endotherm peak temperature of 173° C.

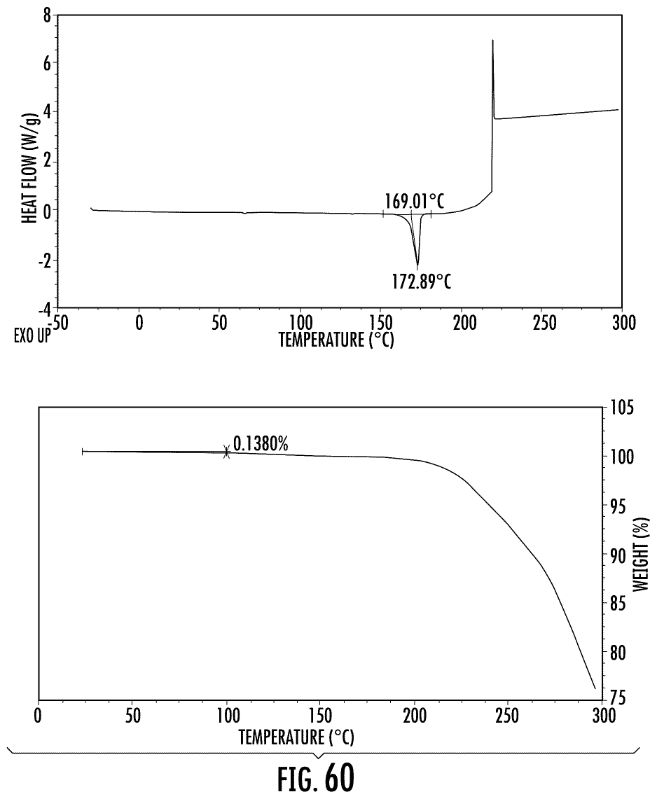

In some embodiments, Compound I Saccharin Material A+minor Compound I Form A is characterized by a DSC substantially similar to FIG. 60.

In some embodiments, Compound I Saccharin Material A+minor Compound I Form A is also characterized by no substantial weight loss up to 200° C., as measured by thermogravimetric analysis.

In some embodiments, Compound I Saccharin Material A+minor Compound I Form A is characterized by a TGA substantially similar to FIG. 60.

In some embodiments, the method of making crystalline Compound I Saccharin Material A+minor Compound I Form A comprises: milling Compound I Form A (free base), saccharin, and MTBE and recovering the solids to yield crystalline Compound I Saccharin Material A+minor Compound I Form A.

### Y-7. Compound I Sorbitol Material A+Sorbitol

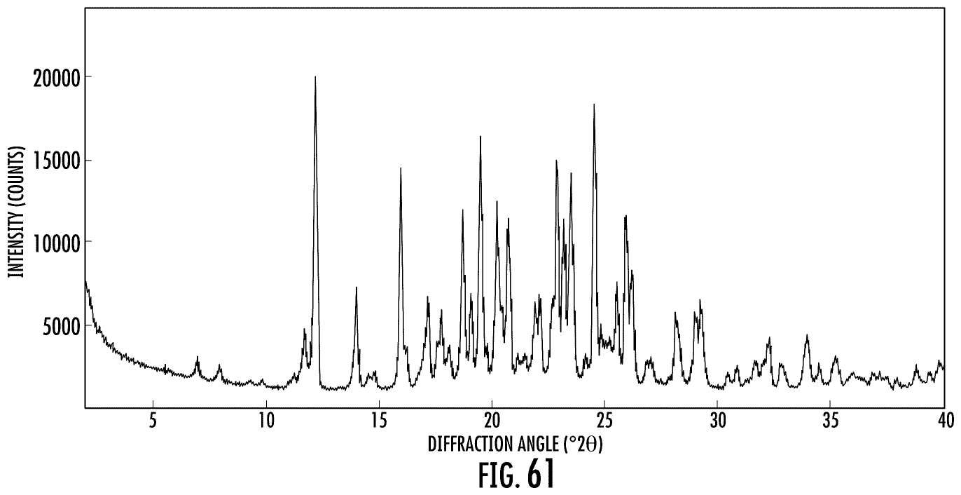

In some embodiments, the disclosure provides crystalline Compound I Sorbitol Material A+sorbitol. In some embodiments, the disclosure provides Compound I Sorbitol Material A+sorbitol. FIG. 61 provides an X-ray powder diffractogram of Compound I Sorbitol Material A+sorbitol with corresponding tabulated data shown in Table 35.

TABLE 35
XRPD Signals for Crystalline Compound I
Sorbitol Material A + sorbitol

| °2Θ | d-spacing | Relative |
|---|---|---|
| (±0.2°) | (Å) | Intensity |
| 7.04 | 12.556 | 7 |
| 8 | 11.0513 | 5 |
| 9.36 | 9.4484 | 2 |
| 9.88 | 8.9522 | 2 |
| 11.28 | 7.8441 | 3 |
| 11.74 | 7.5378 | 14 |
| 12.22 | 7.2427 | 100 |
| 14.04 | 6.3077 | 33 |
| 14.56 | 6.0836 | 3 |
| 14.84 | 5.9694 | 4 |
| 16.02 | 5.5323 | 72 |
| 17.18 | 5.1613 | 28 |
| 17.8 | 4.9829 | 17 |
| 18.14 | 4.8902 | 7 |
| 18.74 | 4.735 | 51 |
| 19.1 | 4.6465 | 21 |
| 19.52 | 4.5475 | 78 |
| 20.24 | 4.3873 | 45 |
| 20.74 | 4.2827 | 45 |
| 21.14 | 4.2025 | 4 |
| 21.46 | 4.1406 | 5 |
| 21.92 | 4.0547 | 13 |
| 22.14 | 4.0149 | 14 |
| 22.68 | 3.9205 | 3 |
| 22.88 | 3.8867 | 47 |
| 23.18 | 3.8371 | 36 |
| 23.52 | 3.7824 | 42 |
| 24.12 | 3.6896 | 6 |
| 24.54 | 3.6274 | 75 |
| 24.82 | 3.5872 | 5 |
| 25 | 3.5617 | 2 |
| 25.22 | 3.5312 | 4 |
| 25.54 | 3.4876 | 24 |
| 25.9 | 3.44 | 44 |
| 26.22 | 3.3987 | 17 |
| 26.78 | 3.3289 | 4 |
| 27.06 | 3.2951 | 3 |
| 28.14 | 3.171 | 21 |
| 28.98 | 3.081 | 12 |
| 29.26 | 3.0522 | 11 |
| 30.38 | 2.9421 | 3 |
| 30.82 | 2.9011 | 6 |
| 31.22 | 2.8649 | 1 |
| 31.62 | 2.8295 | 5 |
| 32.24 | 2.7765 | 15 |
| 32.72 | 2.7369 | 7 |
| 33.9 | 2.6443 | 14 |
| 34.44 | 2.604 | 5 |
| 35.18 | 2.5509 | 8 |
| 35.92 | 2.5001 | 5 |
| 37.1 | 2.4232 | 3 |
| 37.82 | 2.3787 | 3 |
| 38.72 | 2.3255 | 5 |
| 39.32 | 2.2914 | 3 |
| 39.76 | 2.267 | 5 |
| 34.44 | 2.604 | 5 |
| 35.18 | 2.5509 | 8 |
| 35.92 | 2.5001 | 5 |
| 37.1 | 2.4232 | 3 |
| 37.82 | 2.3787 | 3 |
| 38.72 | 2.3255 | 5 |
| 39.32 | 2.2914 | 3 |
| 39.76 | 2.267 | 5 |

In some embodiments, crystalline Compound I Sorbitol Material A+sorbitol is substantially pure. In some embodiments, crystalline Compound I Sorbitol Material A+sorbitol is substantially crystalline. In some embodiments, crystalline Compound I Sorbitol Material A+sorbitol is a mixture of Compound I Sorbitol Material A and Sorbitol. In some embodiments, crystalline Compound I Sorbitol Material A+sorbitol is characterized by an X-ray powder diffractogram generated by an X-ray powder diffraction analysis with an incident beam of Cu Kα radiation.

In some embodiments, crystalline Compound I Sorbitol Material A+sorbitol is characterized by an X-ray powder diffractogram having a signal at one or more of 7.04±0.2 degrees two-theta, 8±0.2 degrees two-theta, 9.36±0.2 degrees two-theta, 9.88±0.2 degrees two-theta, 11.28±0.2 degrees two-theta, 11.74±0.2 degrees two-theta, ±0.2 degrees two-theta, 12.22±0.2 degrees two-theta, 14.04±0.2 degrees two-theta, 14.56±0.2 degrees two-theta, 14.84±0.2 degrees two-theta, 16.02±0.2 degrees two-theta, 17.18±0.2 degrees two-theta, 17.8±0.2 degrees two-theta, 18.14±0.2 degrees two-theta, 18.74±0.2 degrees two-theta, 19.1±0.2 degrees two-theta, 19.52±0.2 degrees two-theta, 20.24±0.2 degrees two-theta, 20.74±0.2 degrees two-theta, 21.14±0.2 degrees two-theta, 21.46±0.2 degrees two-theta, 21.92±0.2 degrees two-theta, 22.14±0.2 degrees two-theta, 22.68±0.2 degrees two-theta, 22.88±0.2 degrees two-theta, 23.18±0.2 degrees two-theta, 23.52±0.2 degrees two-theta, 24.12±0.2 degrees two-theta, 24.54±0.2 degrees two-theta, 24.82±0.2 degrees two-theta, 25±0.2 degrees two-theta, 25.22±0.2 degrees two-theta, 25.54±0.2 degrees two-theta, 25.9±0.2 degrees two-theta, 26.22±0.2 degrees two-theta, 26.78±0.2 degrees two-theta, 27.06±0.2 degrees two-theta, 28.14±0.2 degrees two-theta, 28.98±0.2 degrees two-theta, 29.26±0.2 degrees two-theta, 30.38±0.2 degrees two-theta, 30.82±0.2 degrees two-theta, 31.22±0.2 degrees two-theta, 31.62±0.2 degrees two-theta, 32.24±0.2 degrees two-theta, 32.72±0.2 degrees two-theta, 33.9±0.2 degrees two-theta, 34.44±0.2 degrees two-theta, 35.18±0.2 degrees two-theta, 35.92±0.2 degrees two-theta, 37.1±0.2 degrees two-theta, 37.82±0.2 degrees two-theta, 38.72±0.2 degrees two-theta, 39.32±0.2 degrees two-theta, and 39.76±0.2 degrees two-theta.

In some embodiments, crystalline Compound I Sorbitol Material A+sorbitol is characterized by an X-ray powder diffractogram having a signal at one or more of 7.04±0.2 degrees two-theta, 12.22±0.2 degrees two-theta, 14.04±0.2 degrees two-theta, and 16.02±0.2 degrees two-theta.

In some embodiments, crystalline Compound I Sorbitol Material A+sorbitol is characterized by an X-ray powder diffractogram having a signal at one or more of 12.22±0.2 degrees two-theta, 14.04±0.2 degrees two-theta, and 16.02±0.2 degrees two-theta.

In some embodiments, crystalline Compound I Sorbitol Material A+sorbitol is characterized by an X-ray powder diffractogram having a signal at 7.04±0.2 degrees two-theta. In some embodiments, crystalline Compound I Sorbitol Material A+sorbitol is characterized by an X-ray powder diffractogram having a signal at 12.22±0.2 degrees two-theta. In some embodiments, crystalline Compound I Sorbitol Material A+sorbitol is characterized by an X-ray powder diffractogram having a signal at 14.04±0.2 degrees two-theta. In some embodiments, crystalline Compound I Sorbitol Material A+sorbitol is characterized by an X-ray powder diffractogram having a signal at 16.02±0.2 degrees two-theta.

In some embodiments, crystalline Compound I Sorbitol Material A+sorbitol is characterized by an X-ray powder diffractogram substantially similar to FIG. 61.

In some embodiments, the method of making crystalline Compound I Sorbitol Material A+sorbitol comprises: stirring Compound I Form A (free base), sorbitol, and 50:50 MTBE:ethanol at rt for 7 days, centrifuging, and recovering the solids to yield crystalline Compound I Sorbitol Material A+sorbitol.

### Y-8. Compound I Zinc Chloride Material A+Possible Minor Compound I Form A.

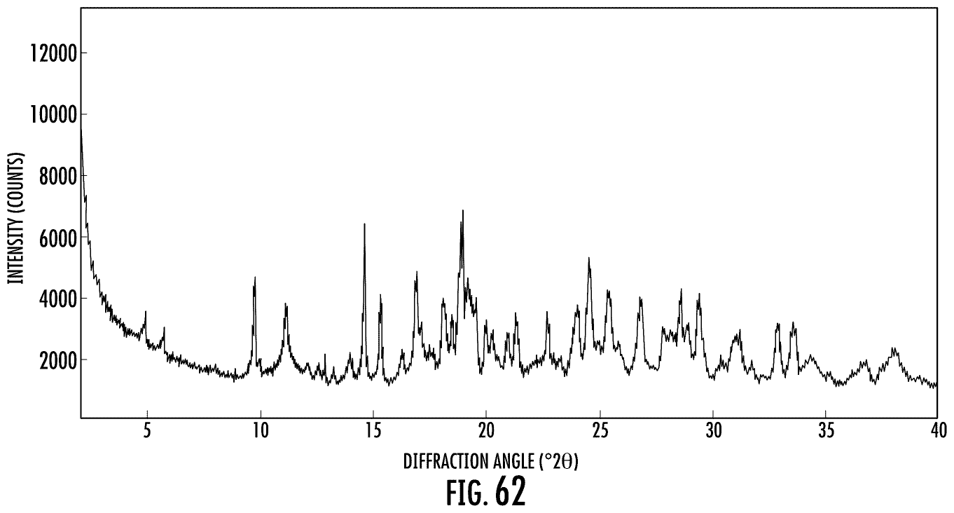

In some embodiments, the disclosure provides crystalline Compound I Zinc Chloride Material A+possible minor Compound I Form A. In some embodiments, the disclosure provides Compound I Zinc Chloride Material A+possible minor Compound I Form A. FIG. 62 provides an X-ray powder diffractogram of Compound I Zinc Chloride Material A+possible minor Compound I Form A with corresponding tabulated data shown in Table 36.

TABLE 36
XRPD Signals for Crystalline Compound I
Zinc Chloride Material A + possible minor
Compound I Form A.

| °2Θ | d-spacing | Relative |
|---|---|---|
| (±0.2°) | (Å) | Intensity |
| 4.92 | 17.9605 | 57 |
| 5.74 | 15.3964 | 49 |
| 8.02 | 11.0238 | 29 |
| 9.78 | 9.0436 | 72 |
| 10.02 | 8.8275 | 32 |
| 11.16 | 7.9282 | 61 |
| 12.14 | 7.2903 | 30 |
| 12.6 | 7.0252 | 31 |
| 12.88 | 6.8731 | 32 |
| 13.28 | 6.6669 | 28 |
| 14.02 | 6.3166 | 36 |
| 14.64 | 6.0505 | 100 |
| 15.34 | 5.7759 | 64 |
| 16.3 | 5.4379 | 37 |
| 16.94 | 5.2338 | 78 |
| 17.68 | 5.0164 | 37 |
| 18.1 | 4.9009 | 64 |
| 18.5 | 4.7959 | 17 |
| 18.98 | 4.6757 | 44 |
| 19.2 | 4.6226 | 16 |
| 19.56 | 4.5383 | 13 |
| 20.02 | 4.435 | 14 |
| 20.28 | 4.3788 | 12 |
| 20.96 | 4.2382 | 14 |
| 21.34 | 4.1636 | 21 |
| 22.72 | 3.9137 | 22 |
| 23.32 | 3.8144 | 6 |
| 24.02 | 3.7048 | 28 |
| 24.58 | 3.6216 | 21 |
| 24.96 | 3.5673 | 5 |
| 25.4 | 3.5065 | 29 |
| 25.84 | 3.4478 | 6 |
| 26.84 | 3.3216 | 32 |
| 27.82 | 3.2068 | 12 |
| 28.62 | 3.1189 | 23 |
| 28.98 | 3.081 | 14 |
| 29.44 | 3.0339 | 36 |
| 30.4 | 2.9402 | 7 |
| 31.2 | 2.8667 | 14 |
| 31.72 | 2.8208 | 8 |
| 32.94 | 2.7191 | 27 |
| 33.58 | 2.6687 | 22 |
| 34.4 | 2.607 | 6 |
| 36.84 | 2.4397 | 10 |
| 38.04 | 2.3655 | 11 |

In some embodiments, crystalline Compound I Zinc Chloride Material A+possible minor Compound I Form A is substantially pure. In some embodiments, crystalline Compound I Zinc Chloride Material A+possible minor Compound I Form A is substantially crystalline. In some embodiments, crystalline Compound I Zinc Chloride Material A+possible minor Compound I Form A is a mixture of Compound I Zinc Chloride Material A and Compound I Form A (free base). In some embodiments, crystalline Compound I Zinc Chloride Material A+possible minor Compound I Form A is characterized by an X-ray powder diffractogram generated by an X-ray powder diffraction analysis with an incident beam of Cu Kα radiation.

In some embodiments, crystalline Compound I Zinc Chloride Material A+possible minor Compound I Form A is characterized by an X-ray powder diffractogram having a signal at one or more of 4.92±0.2 degrees two-theta, 5.74±0.2 degrees two-theta, 8.02±0.2 degrees two-theta, 9.78±0.2 degrees two-theta, 10.02±0.2 degrees two-theta, 11.16±0.2 degrees two-theta, ±0.2 degrees two-theta, 12.14±0.2 degrees two-theta, 12.6±0.2 degrees two-theta, 12.88±0.2 degrees two-theta, 13.28±0.2 degrees two-theta, 14.02±0.2 degrees two-theta, 14.64±0.2 degrees two-theta, 15.34±0.2 degrees two-theta, 16.3±0.2 degrees two-theta, 16.94±0.2 degrees two-theta, 17.68±0.2 degrees two-theta, 18.1±0.2 degrees two-theta, 18.5±0.2 degrees two-theta, 18.98±0.2 degrees two-theta, 19.2±0.2 degrees two-theta, 19.56±0.2 degrees two-theta, 20.02±0.2 degrees two-theta, 20.28±0.2 degrees two-theta, 20.96±0.2 degrees two-theta, 21.34±0.2 degrees two-theta, 22.72±0.2 degrees two-theta, 23.32±0.2 degrees two-theta, 24.02±0.2 degrees two-theta, 24.58±0.2 degrees two-theta, 24.96±0.2 degrees two-theta, 25.4±0.2 degrees two-theta, 25.84±0.2 degrees two-theta, 26.84±0.2 degrees two-theta, 27.82±0.2 degrees two-theta, 28.62±0.2 degrees two-theta, 28.98±0.2 degrees two-theta, 29.44±0.2 degrees two-theta, 30.4±0.2 degrees two-theta, 31.2±0.2 degrees two-theta, 31.72±0.2 degrees two-theta, 32.94±0.2 degrees two-theta, 33.58±0.2 degrees two-theta, 34.4±0.2 degrees two-theta, 36.84±0.2 degrees two-theta, and 38.04±0.2 degrees two-theta.

In some embodiments, crystalline Compound I Zinc Chloride Material A+possible minor Compound I Form A is characterized by an X-ray powder diffractogram having a signal at one or more of 4.92±0.2 degrees two-theta, 5.74±0.2 degrees two-theta, 8.02±0.2 degrees two-theta, 9.78±0.2 degrees two-theta, 11.16±0.2 degrees two-theta, and 14.64±0.2 degrees two-theta.

In some embodiments, crystalline Compound I Zinc Chloride Material A+possible minor Compound I Form A is characterized by an X-ray powder diffractogram having a signal at one or more of 4.92±0.2 degrees two-theta, 5.74±0.2 degrees two-theta, and 9.78±0.2 degrees two-theta.

In some embodiments, crystalline Compound I Zinc Chloride Material A+possible minor Compound I Form A is characterized by an X-ray powder diffractogram having a signal at one or more of 8.02±0.2 degrees two-theta, 11.16±0.2 degrees two-theta, and 14.64±0.2 degrees two-theta.

In some embodiments, crystalline Compound I Zinc Chloride Material A+possible minor Compound I Form A is characterized by an X-ray powder diffractogram having a signal at 4.92±0.2 degrees two-theta. In some embodiments, crystalline Compound I Zinc Chloride Material A+possible minor Compound I Form A is characterized by an X-ray powder diffractogram having a signal at 5.74±0.2 degrees two-theta. In some embodiments, crystalline Compound I Zinc Chloride Material A+possible minor Compound I Form A is characterized by an X-ray powder diffractogram having a signal at 8.02±0.2 degrees two-theta. In some embodiments, crystalline Compound I Zinc Chloride Material A+possible minor Compound I Form A is characterized by an X-ray powder diffractogram having a signal at 9.78±0.2 degrees two-theta. In some embodiments, crystalline Compound I Zinc Chloride Material A+possible minor Compound I Form A is characterized by an X-ray powder diffractogram having a signal at 11.16±0.2 degrees two-theta. In some embodiments, crystalline Compound I Zinc Chloride Material A+possible minor Compound I Form A is characterized by an X-ray powder diffractogram having a signal at 14.64±0.2 degrees two-theta.

In some embodiments, crystalline Compound I Zinc Chloride Material A+possible minor Compound I Form A is characterized by an X-ray powder diffractogram substantially similar to FIG. 62

In some embodiments, Compound I Zinc Chloride Material A+possible minor Compound I Form A is characterized by a DSC having a broad shoulder endotherm temperature of 83° C. In some embodiments, Compound I Zinc Chloride Material A+possible minor Compound I Form A is characterized by a DSC having an endotherm peak temperature of 145° C. In some embodiments, Compound I Zinc Chloride Material A+possible minor Compound I Form A is characterized by a DSC having an endotherm peak temperature of 169° C. In some embodiments, Compound I Zinc Chloride Material A+possible minor Compound I Form A is characterized by a DSC having an endotherm temperature of 83° C., 145° C. and/or an endotherm peak temperature of 169° C.

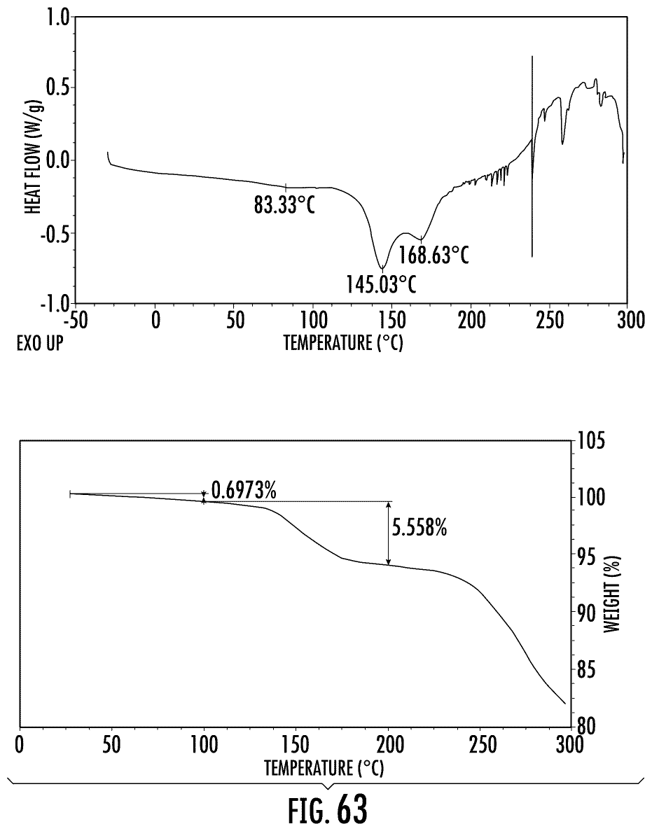

In some embodiments, Compound I Zinc Chloride Material A+possible minor Compound I Form A is characterized by a DSC substantially similar to FIG. 63.

In some embodiments, Compound I Zinc Chloride Material A+possible minor Compound I Form A is also characterized by a weight loss of 0.7% up to 100° C., as measured by thermogravimetric analysis. In some embodiments, Compound I Zinc Chloride Material A+possible minor Compound I Form A is also characterized by a weight loss of 5.6% from 100° C. to 200° C., as measured by thermogravimetric analysis.

In some embodiments, Compound I Zinc Chloride Material A+possible minor Compound I Form A is characterized by a TGA substantially similar to FIG. 63.

In some embodiments, the method of making crystalline Compound I Zinc Chloride Material A+possible minor Compound I Form A comprises: milling Compound I Form A (free base), zinc chloride, and ethanol and recovering the solids to yield crystalline Compound I Zinc Chloride Material A+possible minor Compound I Form A.

### Y-9. Compound I Besylate Form A

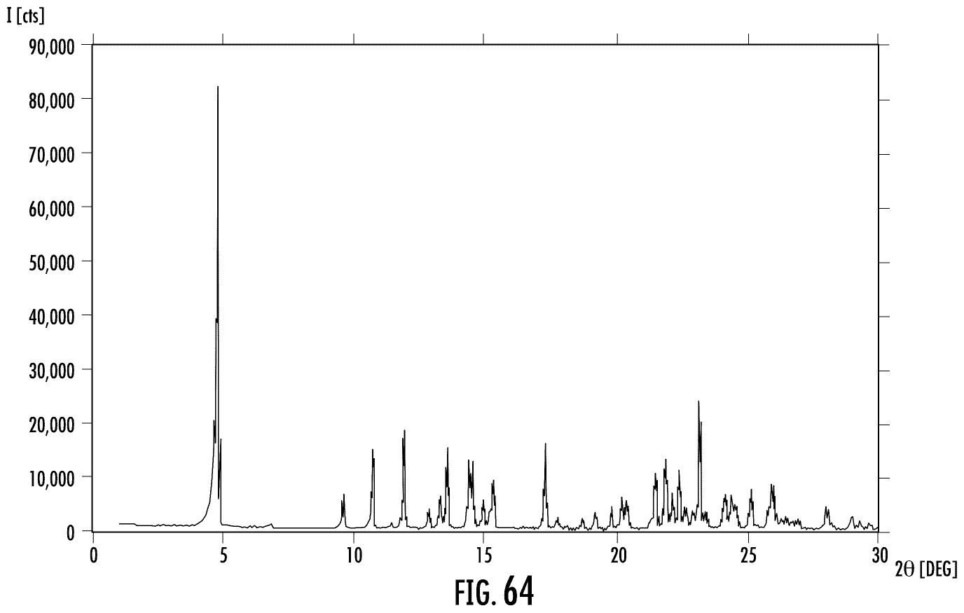

In some embodiments, the disclosure provides crystalline Compound I Besylate Form A. In some embodiments, the disclosure provides Compound I Besylate Form A. FIG. 64 provides an X-ray powder diffractogram of Compound I Besylate Form A.

In some embodiments, crystalline Compound I Besylate Form A is substantially pure. In some embodiments, crystalline Compound I Besylate Form A is substantially crystalline. In some embodiments, crystalline Compound I Besylate Form A is characterized by an X-ray powder diffractogram generated by an X-ray powder diffraction analysis with an incident beam of Cu Kα radiation.

In some embodiments, crystalline Compound Besylate Form A is characterized by an X-ray powder diffractogram substantially similar to FIG. 64.

In some embodiments, crystalline Compound I Besylate Form A is characterized by a Primitive Tetragonal crystal system, P4₂/n space group and the following unit cell dimensions resulting from successful indexing of Besylate Form A XRPD:

| | | | |
|---|---|---|---|
| a | 26.1 ± 0.1 Å | α | 90 ± 0.1° |
| b | 26.1 ± 0.1 Å | β | 90 ± 0.1° |
| c | 8.1 ± 0.1 Å | γ | 90 ± 0.1°. |

In some embodiments, Compound I Besylate Form A is characterized by a DSC having an endotherm peak temperature of 206° C. In some embodiments, Compound I Besylate Form A is characterized by a DSC having an endotherm onset temperature of 205° C. In some embodiments, Compound I Besylate Form A is characterized by a DSC having an endotherm peak temperature of 206° C. and/or an onset endotherm temperature of 205° C.

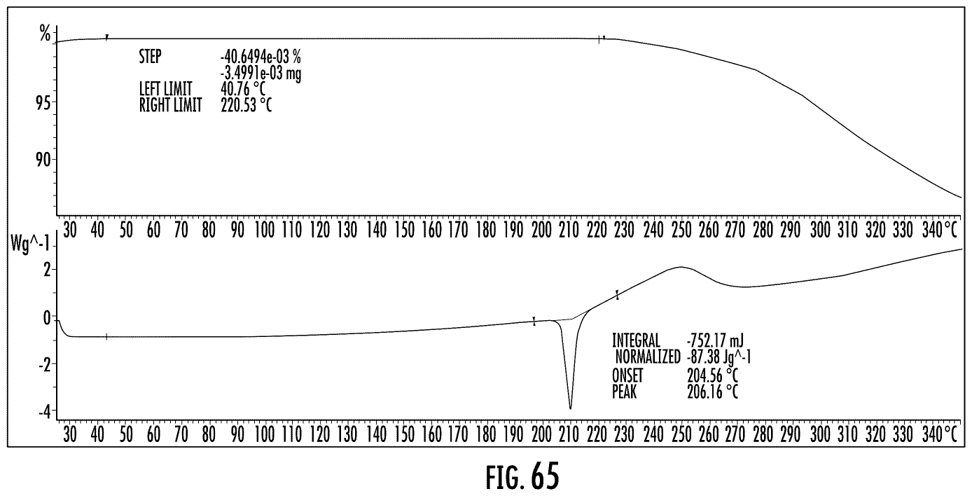

In some embodiments, Compound I Besylate Form A is characterized by a DSC substantially similar to FIG. 65.

In some embodiments, Compound I Besylate Form A is also characterized by no substantial weight loss up to 221° C., as measured by thermogravimetric analysis.

In some embodiments, Compound I Besylate Form A is characterized by a TGA substantially similar to FIG. 65.

In some embodiments, Compound I Besylate Form A is also characterized as non-hygroscopic, evidenced by water uptake of 0.3% at relative humidity of up to 95%, as measured by DVS. In some embodiments, Compound I Besylate Form A is also characterized as non-hygroscopic, evidenced by total water uptake of 0.3% (<0.1 moles), as measured by DVS.

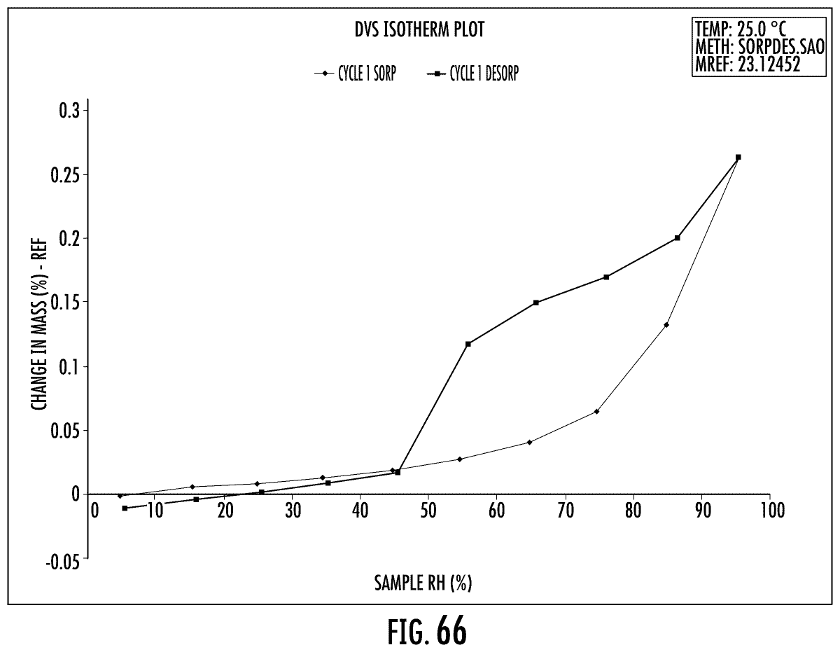

In some embodiments, Compound I Besylate Form A is characterized by a DVS substantially similar to FIG. 66.

In some embodiments, the method of making crystalline Compound I Besylate Form A comprises: adding Compound I Form A and benzenesulfonic acid to ethyl acetate at 50° C., cooling to ambient temperature, and recovering the solids to yield crystalline Compound I Besylate Form A.

### Y-10. Compound I Fumarate Form A

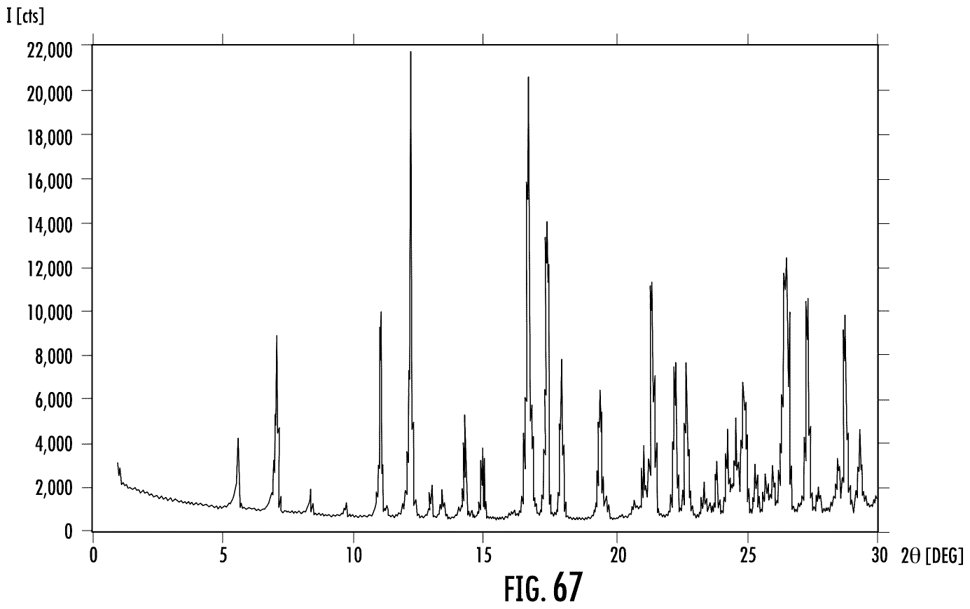

In some embodiments, the disclosure provides crystalline Compound I Fumarate Form A. In some embodiments, the disclosure provides Compound I Fumarate Form A. FIG. 67 provides an X-ray powder diffractogram of Compound I Fumarate Form A.

In some embodiments, crystalline Compound I Fumarate Form A is substantially pure. In some embodiments, crystalline Compound I Fumarate Form A is substantially crystalline. In some embodiments, crystalline Compound I Fumarate Form A is characterized by an X-ray powder diffractogram generated by an X-ray powder diffraction analysis with an incident beam of Cu Kα radiation.

In some embodiments, crystalline Compound I Fumarate Form A is characterized by an X-ray powder diffractogram substantially similar to FIG. 67.

In some embodiments, crystalline Compound I Fumarate Form A is characterized by a Triclinic crystal system, P1 space group and the following unit cell dimensions resulting from successful indexing of Fumarate Form A XRPD:

| | | | |
|---|---|---|---|
| a | 8.1 ± 0.1 Å | α | 97.9 ± 0.1° |
| b | 12.6 ± 0.1 Å | β | 95.1 ± 0.1° |
| c | 15.9 ± 0.1 Å | γ | 97.9 ± 0.1°. |

In some embodiments, Compound I Fumarate Form A is characterized by a DSC having an endotherm peak temperature of 191° C. In some embodiments, Compound I Fumarate Form A is characterized by a DSC having an endotherm onset temperature of 189° C. In some embodiments, Compound I Fumarate Form A is characterized by a DSC having an endotherm peak temperature of 191° C. and/or an onset endotherm temperature of 189° C.

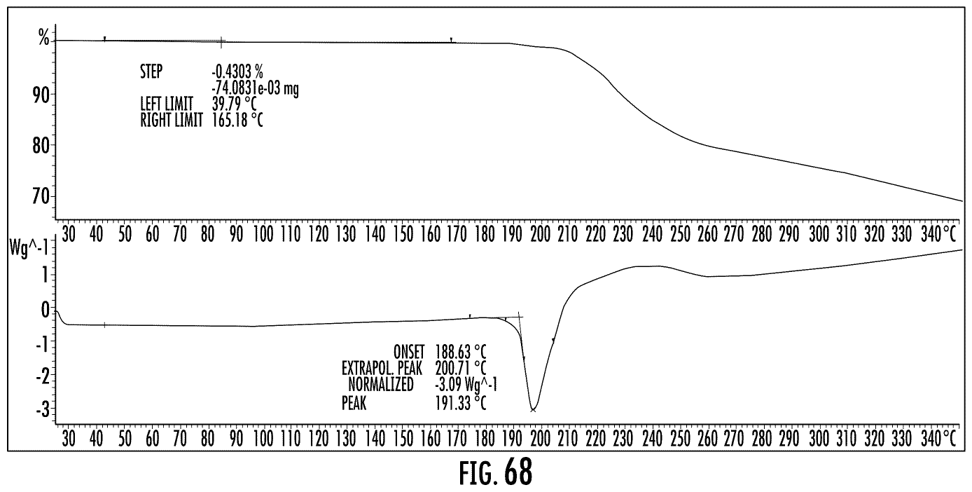

In some embodiments, Compound I Fumarate Form A is characterized by a DSC substantially similar to FIG. 68.

In some embodiments, Compound I Fumarate Form A is also characterized by weight loss of 0.4% up to 165° C., as measured by thermogravimetric analysis.

In some embodiments, Compound I Fumarate Form A is characterized by a TGA substantially similar to FIG. 68.

In some embodiments, Compound I Fumarate Form A is also characterized as slightly hygroscopic, evidenced by water uptake of 0.7% at relative humidity of up to 95%, as measured by DVS. In some embodiments, Compound I Fumarate Form A is also characterized as slightly hygroscopic, evidenced by total water uptake of 0.7% (0.1 moles), as measured by DVS.

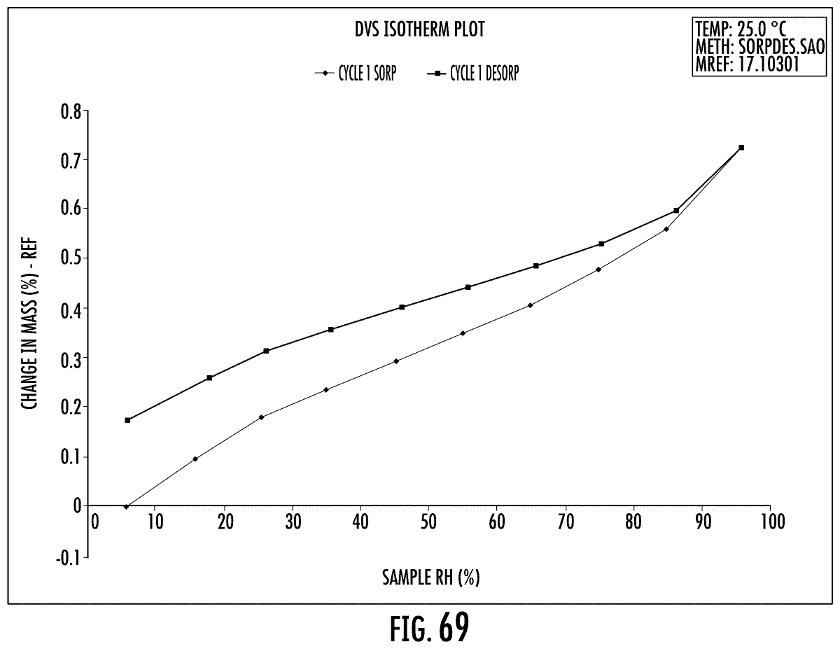

In some embodiments, Compound I Fumarate Form A is characterized by a DVS substantially similar to FIG. 69.

In some embodiments, the method of making crystalline Compound I Fumarate Form A comprises: evaporating Compound I Form A and fumaric acid in THF, slurrying in acetonitrile, and recovering the solids to yield crystalline Compound I Fumarate Form A.

### Y-11. Compound I Sulfate Form A

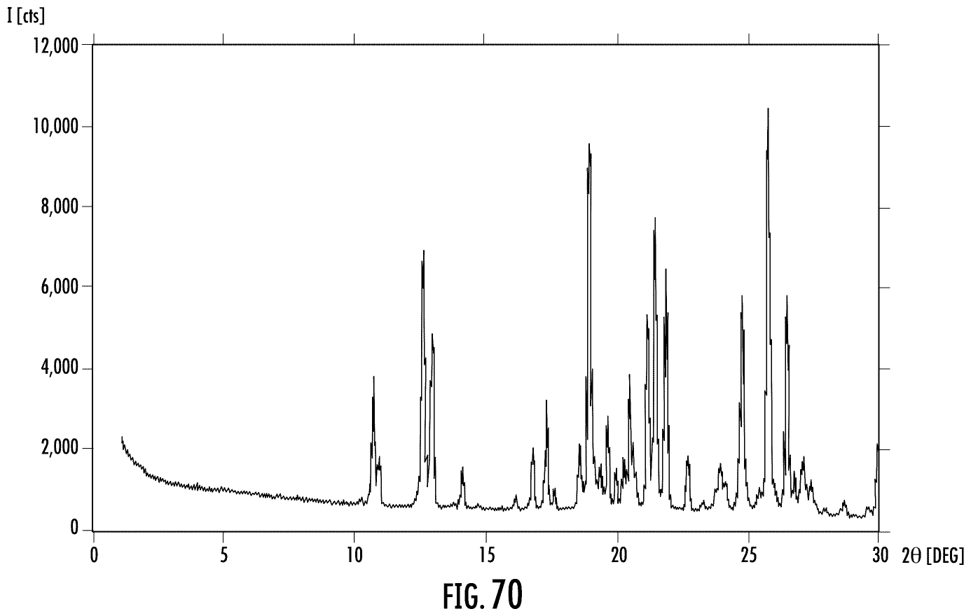

In some embodiments, the disclosure provides crystalline Compound I Sulfate Form A. In some embodiments, the disclosure provides Compound I Sulfate Form A. FIG. 70 provides an X-ray powder diffractogram of Compound I Sulfate Form A.

In some embodiments, crystalline Compound I Form Sulfate A is substantially pure. In some embodiments, crystalline Compound I Form Sulfate A is substantially crystalline. In some embodiments, crystalline Compound I Form Sulfate A is characterized by an X-ray powder diffractogram generated by an X-ray powder diffraction analysis with an incident beam of Cu Kα radiation.

In some embodiments, crystalline Compound Sulfate Form A is characterized by an X-ray powder diffractogram substantially similar to FIG. 70.

In some embodiments, crystalline Compound I Sulfate Form A is characterized by a Primitive monoclinic crystal system, P2₁/c space group and the following unit cell dimensions resulting from successful indexing of Sulfate Form A XRPD:

| | | | |
|---|---|---|---|
| a | 15.3 ± 0.1 Å | α | 90 ± 0.1° |
| b | 11.0 ± 0.1 Å | β | 113.4 ± 0.1° |
| c | 13.7 ± 0.1 Å | γ | 90 ± 0.1°. |

In some embodiments, Compound I Sulfate Form A is characterized by a DSC having an endotherm peak temperature of 224° C. In some embodiments, Compound I Sulfate Form A is characterized by a DSC having an endotherm onset temperature of 220° C. In some embodiments, Compound I Sulfate Form A is characterized by a DSC having an endotherm peak temperature of 224° C. and/or an onset endotherm temperature of 220° C.

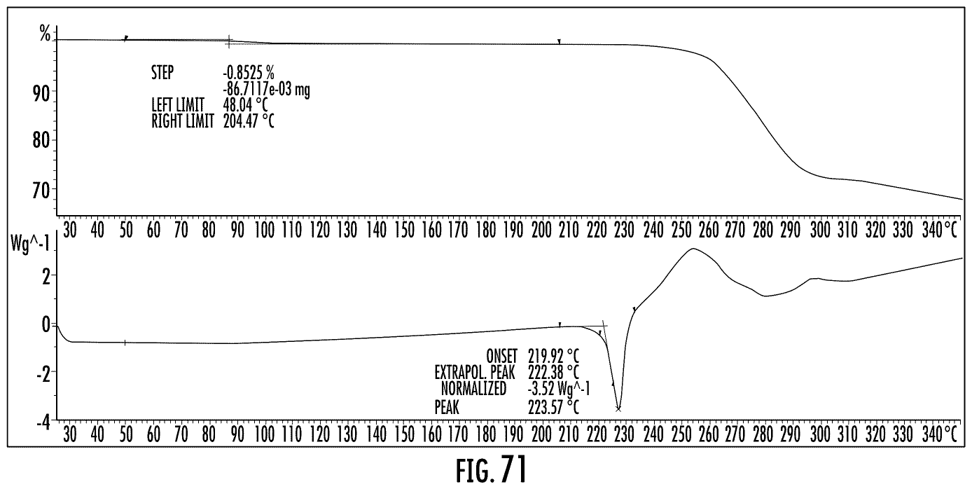

In some embodiments, Compound I Sulfate Form A is characterized by a DSC substantially similar to FIG. 71.

In some embodiments, Compound I Sulfate Form A is also characterized by weight loss of 0.9% up to 204° C., as measured by thermogravimetric analysis.

In some embodiments, Compound I Sulfate Form A is characterized by a TGA substantially similar to FIG. 71.

In some embodiments, Compound I Sulfate Form A is also characterized as slightly hygroscopic, evidenced by water uptake of 2.5% at relative humidity of up to 96%, as measured by DVS. In some embodiments, Compound I Sulfate Form A is also characterized as slightly hygroscopic, evidenced by total water uptake of 2.5% (1.3 moles), as measured by DVS.

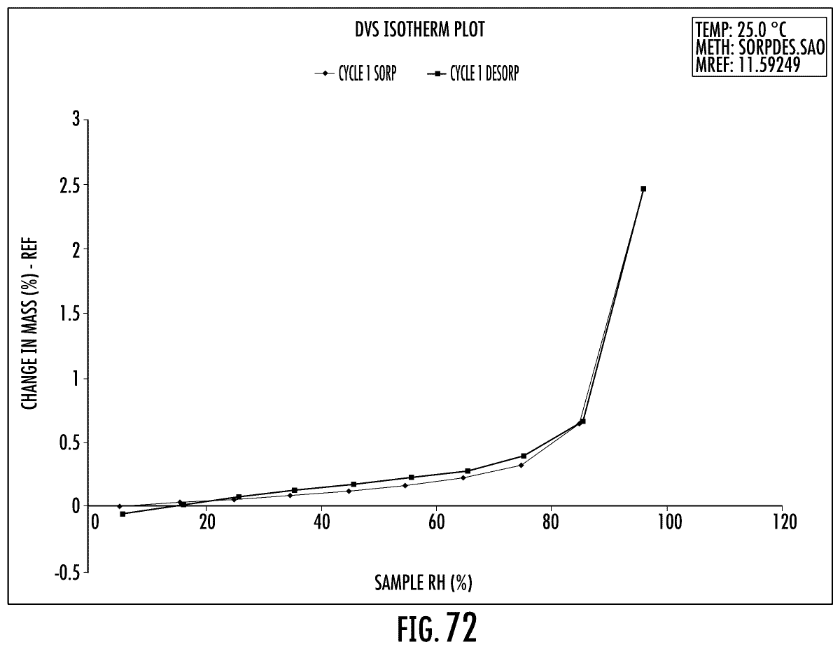

In some embodiments, Compound I Sulfate Form A is characterized by a DVS substantially similar to FIG. 72.

In some embodiments, the method of making crystalline Compound I Sulfate Form A comprises: reacting Compound I Form A with sulfuric acid in ACN, evaporating, slurrying in IPA, slurrying in EtOH, and recovering the solids to yield crystalline Compound I Sulfate Form A.

### Y-12. Compound I Tosylate Form A

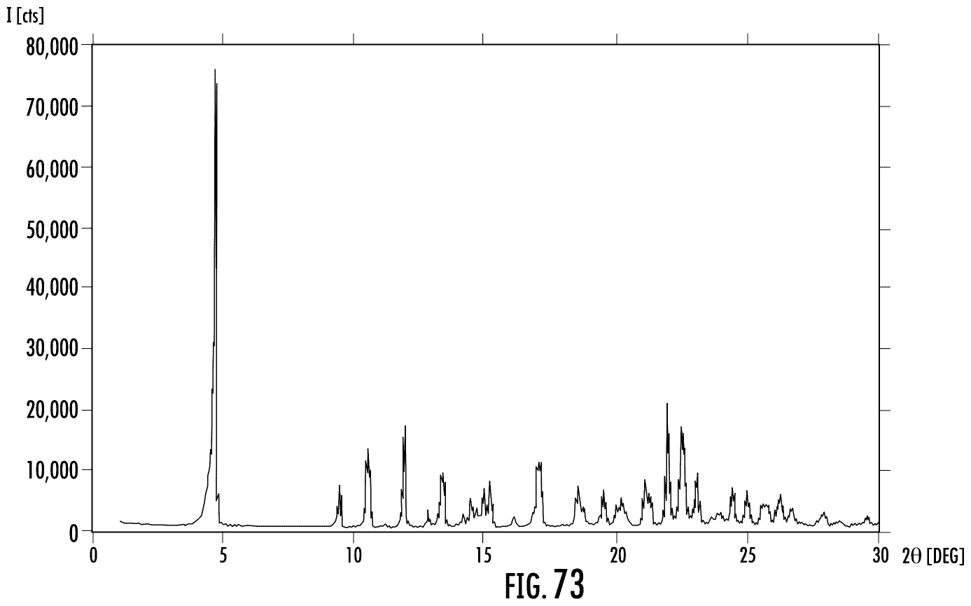

In some embodiments, the disclosure provides crystalline Compound I Tosylate Form A. In some embodiments, the disclosure provides Compound I Tosylate Form A. FIG. 73 provides an X-ray powder diffractogram of Compound I Tosylate Form A.

In some embodiments, crystalline Compound I Tosylate Form A is substantially pure. In some embodiments, crystalline Compound I Tosylate Form A is substantially crystalline. In some embodiments, crystalline Compound I Tosylate Form A is characterized by an X-ray powder diffractogram generated by an X-ray powder diffraction analysis with an incident beam of Cu Kα radiation.

In some embodiments, crystalline Compound Tosylate Form A is characterized by an X-ray powder diffractogram substantially similar to FIG. 73.

In some embodiments, crystalline Compound I Tosylate Form A is characterized by a Primitive monoclinic crystal system, P2₁/c space group and the following unit cell dimensions resulting from successful indexing of Tosylate Form A XRPD:

| | | | |
|---|---|---|---|
| a | 18.8 ± 0.1 Å | α | 90 ± 0.1° |
| b | 8.1 ± 0.1 Å | β | 90.5 ± 0.1° |
| c | 37.4 ± 0.1 Å | γ | 90 ± 0.1°. |

In some embodiments, Compound I Tosylate Form A is characterized by a DSC having an endotherm peak temperature of 197° C. In some embodiments, Compound I Tosylate Form A is characterized by a DSC having an endotherm onset temperature of 193° C. In some embodiments, Compound I Tosylate Form A is characterized by a DSC having an endotherm peak temperature of 197° C. and/or an onset endotherm temperature of 193° C.

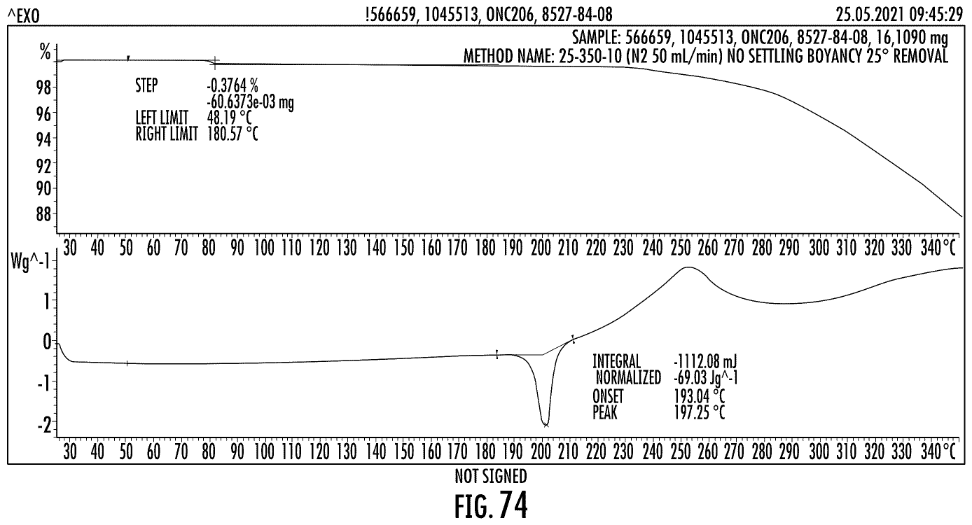

In some embodiments, Compound I Tosylate Form A is characterized by a DSC substantially similar to FIG. 74.

In some embodiments, Compound I Tosylate Form A is also characterized by weight loss of 0.4% up to 181° C., as measured by thermogravimetric analysis.

In some embodiments, Compound I Tosylate Form A is characterized by a TGA substantially similar to FIG. 74.

In some embodiments, Compound I Tosylate Form A is also characterized as slightly hygroscopic, evidenced by water uptake of 1.3% at relative humidity of up to 96%, as measured by DVS. In some embodiments, Compound I Tosylate Form A is also characterized as slightly hygroscopic, evidenced by total water uptake of 1.3% (0.4 moles), as measured by DVS.

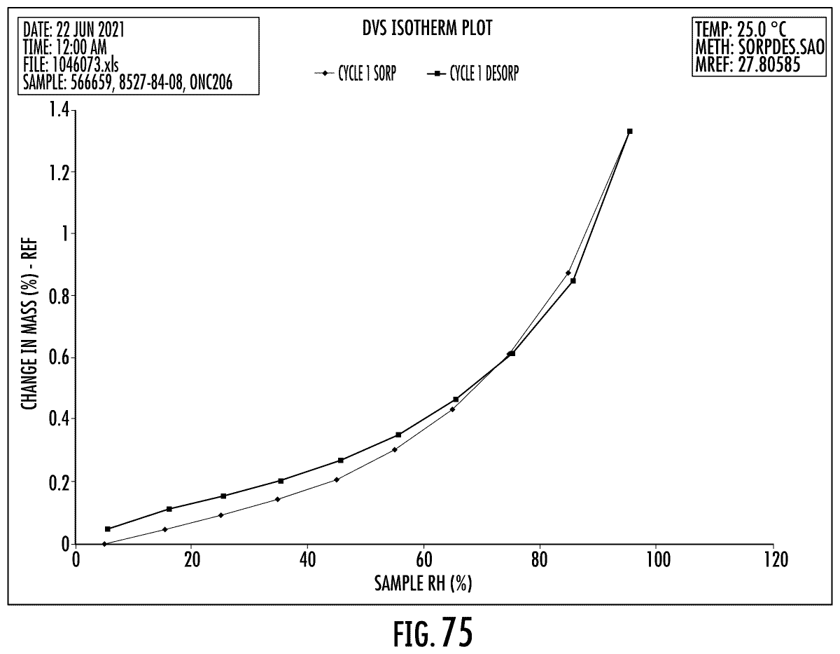

In some embodiments, Compound I Tosylate Form A is characterized by a DVS substantially similar to FIG. 75.

In some embodiments, the method of making crystalline Compound I Tosylate Form A comprises: slurrying Compound I Form A in ethyl acetate, adding p-toluenesulfonic acid, and recovering the solids to yield crystalline Compound I Tosylate Form A.

### Y-13. Compound I Edisylate Form A

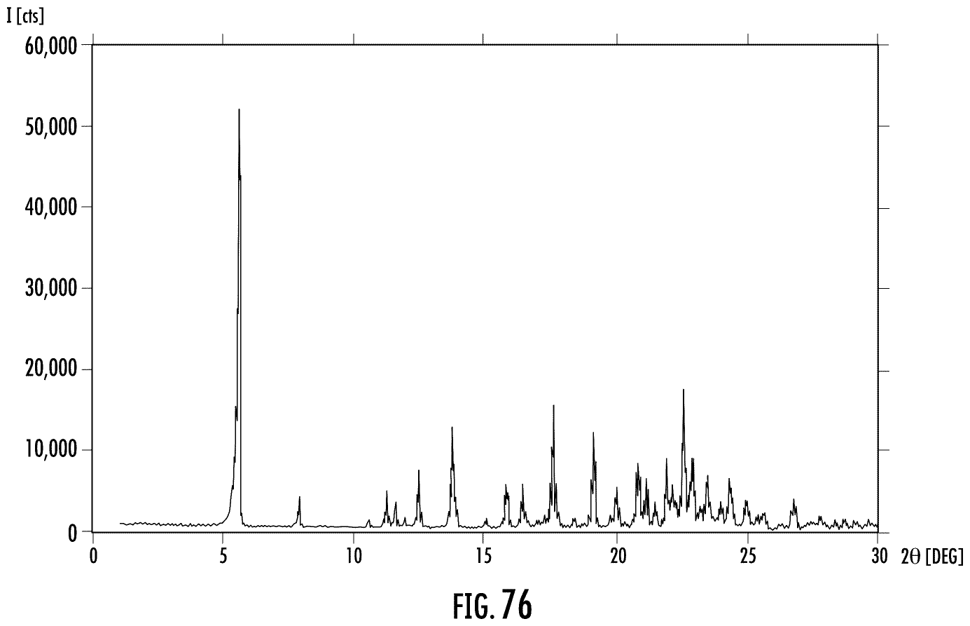

In some embodiments, the disclosure provides crystalline Compound I Edisylate Form A. In some embodiments, the disclosure provides Compound I Edisylate Form A. FIG. 76 provides an X-ray powder diffractogram of Compound I Edisylate Form A.

In some embodiments, crystalline Compound I Edisylate Form A is substantially pure. In some embodiments, crystalline Compound I Edisylate Form A is substantially crystalline. In some embodiments, crystalline Compound I Edisylate Form A is characterized by an X-ray powder diffractogram generated by an X-ray powder diffraction analysis with an incident beam of Cu Kα radiation.

In some embodiments, crystalline Compound Form A is characterized by an X-ray powder diffractogram substantially similar to FIG. 76.

In some embodiments, crystalline Compound I Edisylate Form A is characterized by a Primitive monoclinic crystal system, P2₁/n space group and the following unit cell dimensions resulting from successful indexing of Edisylate Form A XRPD:

| | | | |
|---|---|---|---|
| a | 17.9 ± 0.1 Å | α | 90 ± 0.1° |
| b | 8.3 ± 0.1 Å | β | 109.6 ± 0.1° |
| c | 21.4 ± 0.1 Å | γ | 90 ± 0.1°. |

In some embodiments, Compound I Edisylate Form A is characterized by a DSC having an endotherm peak temperature of 220° C. In some embodiments, Compound I Edisylate Form A is characterized by a DSC having an endotherm peak temperature of 288° C. In some embodiments, Compound I Edisylate Form A is characterized by a DSC having an endotherm peak temperature of 220° C. and/or an endotherm peak temperature of 288° C.

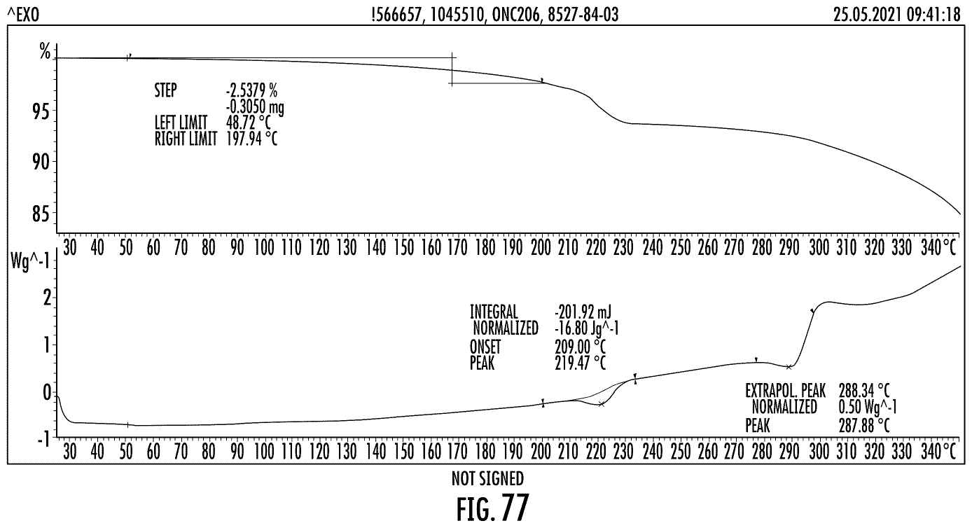

In some embodiments, Compound I Edisylate Form A is characterized by a DSC substantially similar to FIG. 77.

In some embodiments, Compound I Edisylate Form A is also characterized by weight loss of 2.5% up to 198° C., as measured by thermogravimetric analysis.

In some embodiments, Compound I Edisylate Form A is characterized by a TGA substantially similar to FIG. 77.

In some embodiments, the method of making crystalline Compound I Edisylate Form A comprises: slurrying Compound I Form A in ethyl acetate, adding 1,2-ethanedisulfonic acid, and recovering the solids to yield crystalline Compound I Edisylate Form A.

### Y-14. Compound I Gentisate Form A

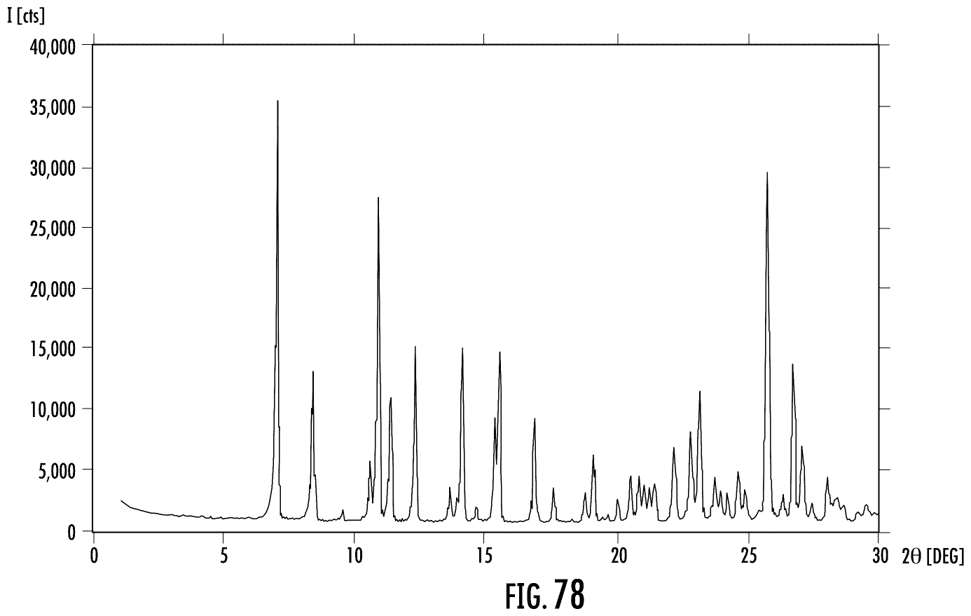

In some embodiments, the disclosure provides crystalline Compound I Gentisate Form A. In some embodiments, the disclosure provides Compound I Gentisate Form A. FIG. 78 provides an X-ray powder diffractogram of Compound I Gentisate Form A.

In some embodiments, crystalline Compound I Gentisate Form A is substantially pure. In some embodiments, crystalline Compound I Gentisate Form A is substantially crystalline. In some embodiments, crystalline Compound I Gentisate Form A is characterized by an X-ray powder diffractogram generated by an X-ray powder diffraction analysis with an incident beam of Cu Kα radiation.

In some embodiments, crystalline Compound I Gentisate Form A is characterized by an X-ray powder diffractogram substantially similar to FIG. 78.

In some embodiments, crystalline Compound I Gentisate Form A is characterized by a Primitive monoclinic crystal system, P2₁/c space group and the following unit cell dimensions resulting from successful indexing of Gentisate Form A XRPD:

| | | | |
|---|---|---|---|
| a | 12.6 ± 0.1 Å | α | 90 ± 0.1° |
| b | 13.8 ± 0.1 Å | β | 96.4 ± 0.1° |
| c | 16.3 ± 0.1 Å | γ | 90 ± 0.1°. |

In some embodiments, Compound I Gentisate Form A is characterized by a DSC having an endotherm peak temperature of 196° C. In some embodiments, Compound I Gentisate Form A is characterized by a DSC having an endotherm onset peak temperature of 194° C. In some embodiments, Compound I Form A is characterized by a DSC having an endotherm peak temperature of 196° C. and/or an onset endotherm temperature of 194° C.

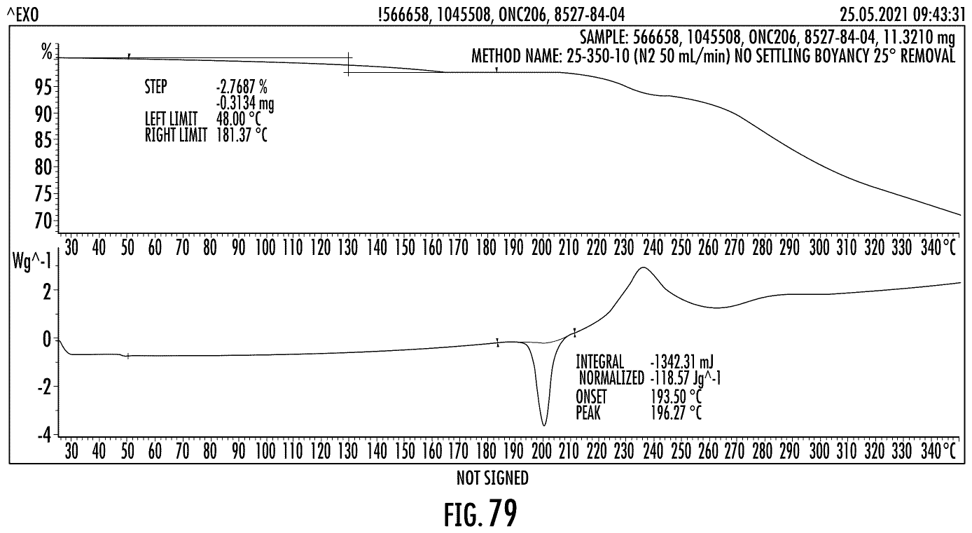

In some embodiments, Compound I Form A is characterized by a DSC substantially similar to FIG. 79.

In some embodiments, Compound I Gentisate Form A is also characterized by weight loss of 2.8% up to 181° C., as measured by thermogravimetric analysis.

In some embodiments, Compound I Gentisate Form A is characterized by a TGA substantially similar to FIG. 79.

In some embodiments, the method of making crystalline Compound I Gentisate Form A comprises: slurrying Compound I Form A in ACN, adding gentisic acid, and recovering the solids to yield crystalline Compound I Gentisate Form A.

### Y-15. Compound I Potential Benzoate Material A

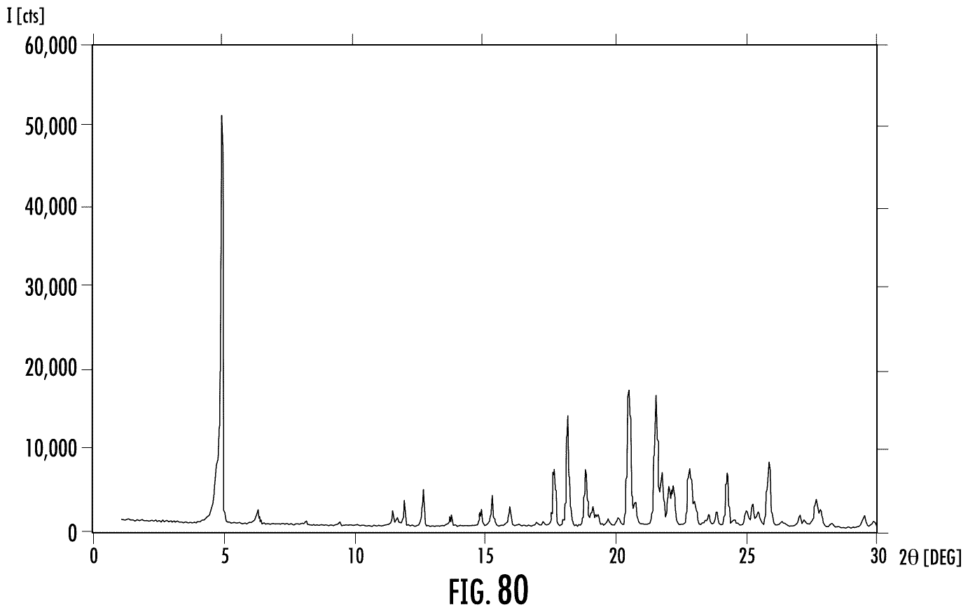

In some embodiments, the disclosure provides crystalline Compound I Potential Benzoate Material A. In some embodiments, the disclosure provides Compound I Potential Benzoate Material A. FIG. 80 provides an X-ray powder diffractogram of Compound I Potential Benzoate Material A.

In some embodiments, crystalline Compound I Potential Benzoate Material A is substantially pure. In some embodiments, crystalline Compound I Potential Benzoate Material A is substantially crystalline. In some embodiments, crystalline Compound I Potential Benzoate Material A is characterized by an X-ray powder diffractogram generated by an X-ray powder diffraction analysis with an incident beam of Cu Kα radiation.

In some embodiments, crystalline Compound I Potential Benzoate Material A is characterized by an X-ray powder diffractogram substantially similar to FIG. 80.

In some embodiments, crystalline Compound I Potential Benzoate Material A is characterized by a Primitive Orthorhombic crystal system, P2₁2₁2 space group and the following unit cell dimensions resulting from successful indexing of Potential Benzoate Material A XRPD:

| | | | |
|---|---|---|---|
| a | 4.9 ± 0.1 Å | α | 90 ± 0.1° |
| b | 15.2 ± 0.1 Å | β | 90 ± 0.1° |
| c | 35.9 ± 0.1 Å | γ | 90 ± 0.1°. |

In some embodiments, the method of making crystalline Compound I Potential Benzoate Material A comprises: dissolving Compound I Form A and benzoic acid in MTBE at 60° C., cooling to rt, and recovering the solids to yield crystalline Compound I Potential Benzoate Material A.

### Y-16. Compound I Potential Mesylate Material A

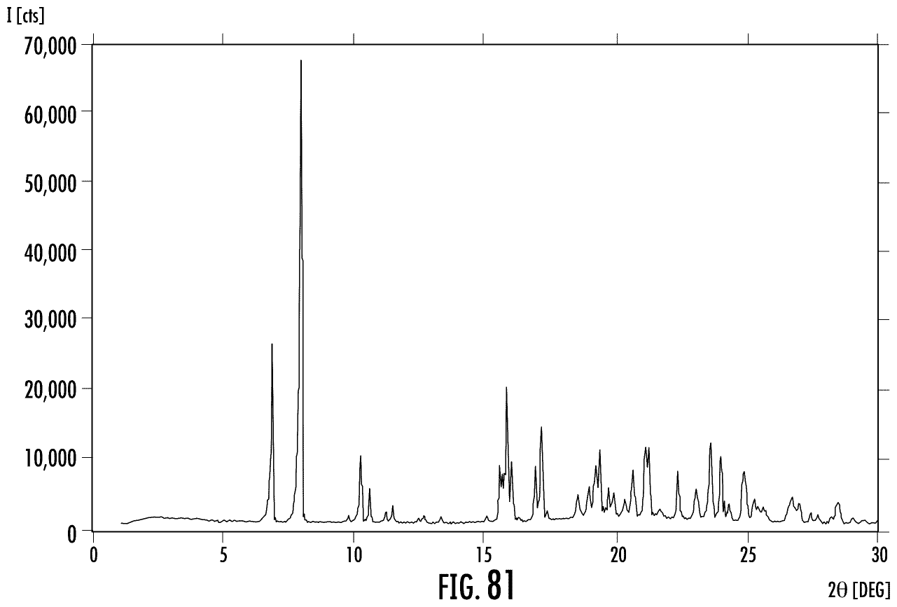

In some embodiments, the disclosure provides crystalline Compound I Potential Mesylate Material A. In some embodiments, the disclosure provides Compound I Potential Mesylate Material A. FIG. 81 provides an X-ray powder diffractogram of Compound I Potential Mesylate Material A.

In some embodiments, crystalline Compound I Potential Mesylate Material A is substantially pure. In some embodiments, crystalline Compound I Potential Mesylate Material A is substantially crystalline. In some embodiments, crystalline Compound I Potential Mesylate Material A is characterized by an X-ray powder diffractogram generated by an X-ray powder diffraction analysis with an incident beam of Cu Kα radiation.

In some embodiments, crystalline Compound I Potential Mesylate Material A is characterized by an X-ray powder diffractogram substantially similar to FIG. 81

In some embodiments, crystalline Compound I Potential Mesylate Material A is characterized by a Triclinic crystal system, P1 space group and the following unit cell dimensions resulting from successful indexing of Potential Mesylate Material A XRPD:

| | | | |
|---|---|---|---|
| a | 11.7 ± 0.1 Å | α | 79 ± 0.1° |
| b | 11.7 ± 0.1 Å | β | 89 ± 0.1° |
| c | 13.1 ± 0.1 Å | γ | 76 ± 0.1°. |

In some embodiments, the method of making crystalline Compound I Potential Mesylate Material A comprises: stirring Compound I Form A in IPA at 50° C., adding methanesulfonic acid, cooling to rt, adding heptane, stirring at rt for 5 days, recovering the solids to yield crystalline Compound I Potential Mesylate Material A.

### Y-17. Compound I Potential Phosphate Material A

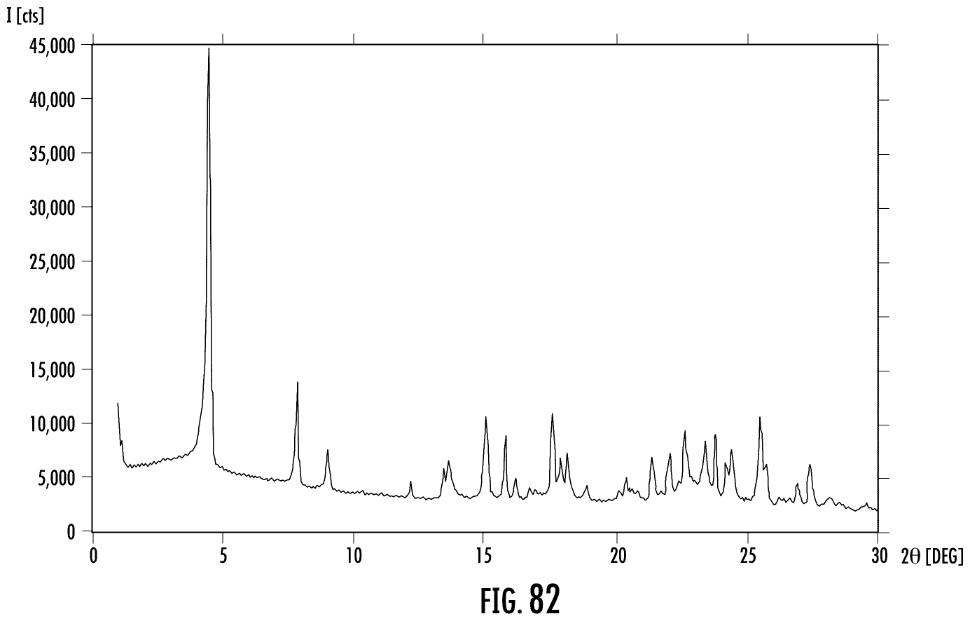

In some embodiments, the disclosure provides crystalline Compound I Potential Phosphate Material A. In some embodiments, the disclosure provides Compound I Potential Phosphate Material A. FIG. 82 provides an X-ray powder diffractogram of Compound I Potential Phosphate Material A.

In some embodiments, crystalline Compound I Potential Phosphate Material A is substantially pure. In some embodiments, crystalline Compound I Potential Phosphate Material A is substantially crystalline. In some embodiments, crystalline Compound I Potential Phosphate Material A is characterized by an X-ray powder diffractogram generated by an X-ray powder diffraction analysis with an incident beam of Cu Kα radiation.

In some embodiments, crystalline Compound Potential Phosphate Material A is characterized by an X-ray powder diffractogram substantially similar to FIG. 82.

In some embodiments, crystalline Compound I Potential Phosphate Material A is characterized by a Primitive Orthorhombic crystal system, Pbcn space group and the following unit cell dimensions resulting from successful indexing of Potential Phosphate Material A XRPD:

| | | | |
|---|---|---|---|
| a | 39.1 ± 0.1 Å | α | 90 ± 0.1° |
| b | 11.7 ± 0.1 Å | β | 90 ± 0.1° |
| c | 13.1 ± 0.1 Å | γ | 90 ± 0.1°. |

In some embodiments, the method of making crystalline Compound I Potential Phosphate Material A comprises: slurrying Compound I Form A in acetonitrile at rt, adding phosphoric acid, heating to 60° C., cooling to rt, stirring at rt for 4 days, recovering the solids to yield crystalline Compound I Potential Phosphate Material A.

### Y-18. Compound I Potential IPA Solvate

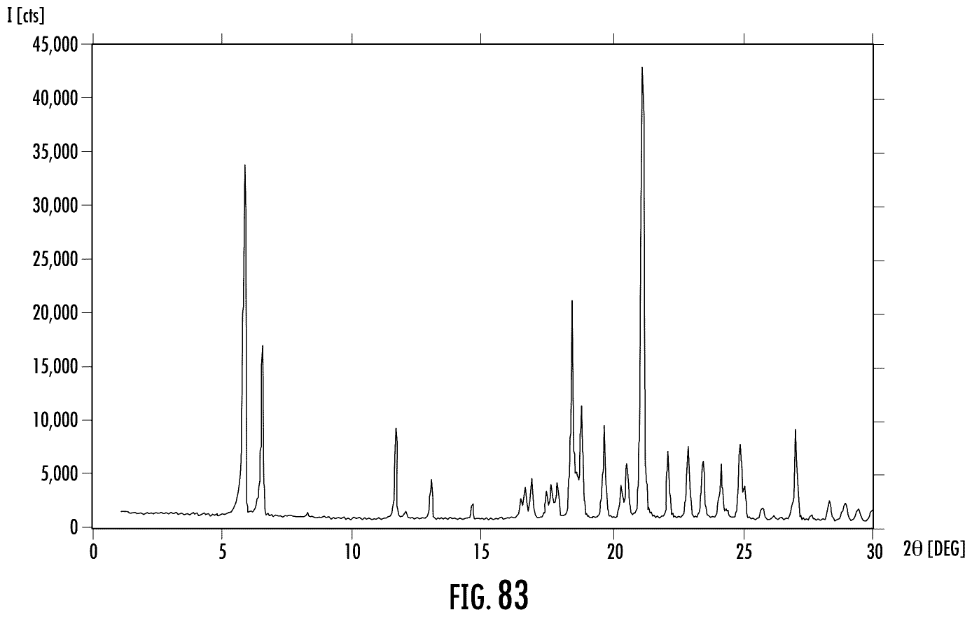

In some embodiments, the disclosure provides crystalline Compound I Potential IPA Solvate. In some embodiments, the disclosure provides Compound I Potential IPA Solvate. FIG. 83 provides an X-ray powder diffractogram of Compound I Potential IPA Solvate.

In some embodiments, crystalline Compound I Potential IPA Solvate is substantially pure. In some embodiments, crystalline Compound I Potential IPA Solvate is substantially crystalline. In some embodiments, crystalline Compound I Potential IPA Solvate is characterized by an X-ray powder diffractogram generated by an X-ray powder diffraction analysis with an incident beam of Cu Kα radiation.

In some embodiments, crystalline Compound Potential IPA Solvate is characterized by an X-ray powder diffractogram substantially similar to FIG. 83.

In some embodiments, crystalline Compound I Potential IPA Solvate is characterized by a Primitive Orthorhombic crystal system, P2₁2₁2 space group and the following unit cell dimensions resulting from successful indexing of Potential IPA Solvate XRPD:

| | | | |
|---|---|---|---|
| a | 15.2 ± 0.1 Å | α | 90 ± 0.1° |
| b | 30.4 ± 0.1 Å | β | 90 ± 0.1° |
| c | 5.3 ± 0.1 Å | γ | 90 ± 0.1°. |

In some embodiments, the method of making crystalline Compound I Potential IPA Solvate comprises: stirring Compound I Form A in EtOAc at 50° C., adding glycolic acid, cooling to rt, adding IPA, stirring at rt for 5 days, recovering the solids to yield crystalline Compound I Potential IPA Solvate.

In some embodiments, the method of making crystalline Compound I Potential IPA Solvate comprises: stirring Compound I Form A in ACN at 50° C., adding hippuric acid, cooling to rt, adding MTBE, stirring at rt for 5 h, adding IPA, stirring at rt for 5 days, recovering the solids to yield crystalline Compound I Potential IPA Solvate.

Y-19. Compound I Form A Unique Material Isolated from Reaction with Oxalic Acid

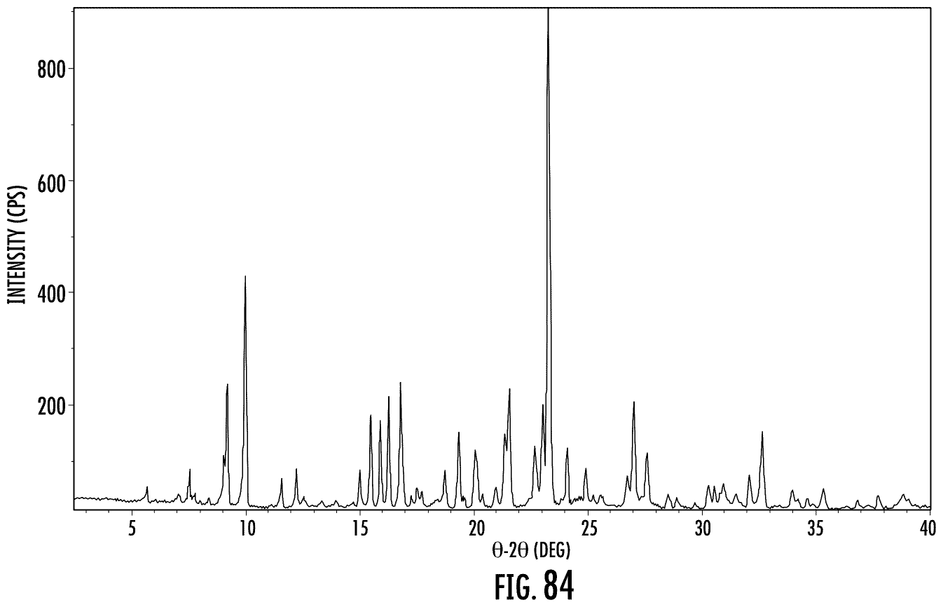

In some embodiments, the disclosure provides crystalline Compound I unique oxalic acid reaction product material. In some embodiments, the disclosure provides Compound I unique oxalic acid reaction product material. FIG. 84 provides an X-ray powder diffractogram of Compound I unique oxalic acid reaction product material.

In some embodiments, crystalline Compound I unique oxalic acid reaction product material is substantially pure. In some embodiments, crystalline Compound I unique oxalic acid reaction product material is substantially crystalline. In some embodiments, crystalline Compound I unique oxalic acid reaction product material is characterized by an X-ray powder diffractogram generated by an X-ray powder diffraction analysis with an incident beam of Cu Kα radiation.

In some embodiments, crystalline Compound unique oxalic acid reaction product material is characterized by an X-ray powder diffractogram substantially similar to FIG. 84.

In some embodiments, the method of making crystalline Compound I unique oxalic acid reaction product material comprises: slurrying Compound I Form A in EtOAc, adding oxalic acid, heating to 60° C., cooling to rt, stirring for 4 days, and recovering the solids to yield crystalline Compound I unique oxalic acid reaction product material.

### EXAMPLES

### Abbreviations and Acronyms

| Category | Abbreviation/Acronym | Full Name/Description |
|---|---|---|
| Solvents/Reagents | AE | Acetone |
| | ACN | Acetonitrile |
| | AcOH | Glacial acetic acid |
| | CLF | Chloroform |
| | DCM | Dichloromethane |
| | DMF | Dimethyl formamide |
| | DMSO | Dimethyl sulfoxide |
| | DOX | 1,4-Dioxane |
| | EtOAc | Ethyl acetate |
| | EtOH | Ethanol |
| | HCl | Hydrochloride |
| | H₂O | Water |
| | HEP | n-Heptane |
| | HEX | Hexanes |
| | HFIPA | Hexafluoro isopropanol |
| | IPA | Isopropanol |
| | IPrOAc | Isopropyl acetate |
| | MEK | Methyl ethyl ketone |
| | MeOH | Methanol |
| | MTBE | Methyl t-butyl ether |
| | NMP | N-methylpyrrolidone |
| | P₂O₅ | Phosphorus pentoxide |
| | PPTS | pyridinium p-toluenesulfonate |
| | OCT | Octane |
| | THF | Tetrahydrofuran |
| Crystallization | S/AS | Antisolvent addition |
| Techniques | FE | Fast Evaporation |
| | GR | Grinding |
| | SE | Slow evaporation |
| | SL | Slurry |
| | VD | Vapor Diffusion |
| Analytical | DSC | Differential Scanning Calorimetry |
| Techniques | DVS | Dynamic Vapor Sorption |
| | FT | Fourier Transform |
| | IR | Infrared |
| | KF | Karl Fischer (titration for water content) |
| | LF | Low frequency |
| | ¹H NMR | Proton Nuclear Magnetic Resonance Spectroscopy |
| | ssNMR | Solid-state nuclear magnetic resonance |
| | TGA | Thermogravimetric Analysis |
| | XRPD | X-ray powder diffraction |
| Miscellaneous | RH | Relative Humidity |
| | Endo | Endo Endotherm |
| | soln | Solution |
| | ST | Sub-ambient temperature |
| | RT | Room/ambient temperature |
| | ET | Elevated temperature |
| | VO | Vacuum oven |
| | D | Days |
| | Hr | Hour |
| | Min | Minute |
| | wt | Weight |
| | NLT | Not less than |
| | NMT | Not more than |

### Analytical Methods

### Differential Scanning Calorimetry (DSC)

Method A: Differential scanning calorimetry was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan lid was pierced then inserted into the DSC cell. A weighed aluminum pan configured as the sample pan was placed on the reference side of the cell. The pan lid was pierced prior to sample analysis. The majority of the data was collected from 25° C. to 350° C. at 10° C./min.

Method B: DSC analyses were carried out using a TA Instruments Q2000 or Q2500 instrument. The instrument temperature calibration was performed using indium. The DSC cell was kept under a nitrogen purge of ~50 mL per minute during each analysis. The sample was placed in a standard, crimped, aluminum pan and was heated at a rate of 10° C. per minute.

Modulated DSC analyses was carried out using a TA Instruments Q2000 Discovery Series instrument. The DSC cell was kept under a nitrogen purge of ~50 mL per minute during the analysis. The sample was placed in a standard, crimped aluminum pan and cooled to −50° C. and held at this temperature for 2 minutes. It was then modulated +0.32° C. every 30 seconds while being heated to 225° C. at a rate of 4° C. per minute.

### Thermogravimetric Analysis (TGA)

The TG analysis was carried out using a TA Instruments Q50 or Q5500 Discovery Series instrument. For the Q50 instrument, the nitrogen purge was typically ~10 mL per minute at the balance and ~90 mL per minute at the furnace. For the Q5500, the nitrogen purge was ~10 mL per minute at the balance and ~25 mL per minute at the furnace. Each sample was placed into a pre-tared platinum pan and heated at a rate of 10° C. per minute.

### LF Raman Spectroscopy

LF Raman spectroscopy was performed using an Ondax CleanLine™ Laser Module and SureBlock™ TR-Probe THz Raman System. The Ondax THz Raman system was paired with a Renishaw inVia™ Qontor™ confocal microscope, equipped with a 1200 lines.mm hol-ographic diffraction grating, a 1-inch charge-coupled device (CCD) detector, and an excita-tion laser operating at 785 nm. Test materials were placed onto a motorized XYZ stage under a 50× microscope objective and analyzed using a 180° backscattering geometry. Each sam-ple was analyzed using a 0.75 second exposure time over the spectral range from 1 cm-1 to 1234 cm-1 (Raman shift) with a spectral resolution of approximately 3 cm-1. 32 accumulations were collected. Data processing was performed using WiRE 5.5 and OMNIC v9.11 software.

### Karl Fisher Volumetric Analysis (KF)

Karl Fischer analysis was carried out using a Mettler-Toledo V20 Volumetric KF titrator equipped with a DM143-SC sensor. The instrument was calibrated using HYDRANAL® Water Standard 10.0. For each water determination measurement, an accurately weighed amount of material was introduced directly into a vessel filled with HYDRANAL®-Methanol Dry methanol solution and titrated at 25° C. with HYDRANAL®-Composite 5. The sample was titrated using a 60 or 180 second mix time. Triplicate analyses were performed for the sample.

### Fourier-Transform (FT) Raman Spectroscopy

Raman spectroscopic analysis was performed using a Nicolet iS50 Raman module that was equipped with a 1064 nm near-infrared laser. The system was configured with an indium gallium arsenide (InGaAs) detector and a calcium fluoride (CaF2) beamsplitter. Each sample was placed onto the automated XYZ stage and analyzed using a laser power that was adjusted to optimize the signal intensity while avoiding damage to the sample. Raman spec-tra were collected with 256 signal-averaged scans at a resolution of 2 cm-1 over the spectral range from 3700 cm-1 to 100 cm-1. Data acquisition and processing were performed using OMNIC v9.11 software.

### Infrared (IR) Spectroscopy

IR spectroscopic analysis was performed using a Thermo Scientific model iS50 Fourier-transform (FT) IR spectrophotometer equipped with a deuterated triglycine sulfate (DTGS) detector, a potassium bromide (KBr) beamsplitter, and a Polaris™ long-life IR source. A diamond attenuated total reflectance (ATR) sampling accessory with a spectral range of 4000 cm⁻¹ to 400 cm⁻¹ was used. Each spectrum was the result of 128 co-added scans acquired at 2 cm⁻¹ resolution. A single beam background scan of air was acquired before the sample scan, allowing presentation of the spectra in log 1/R units. Wavelength calibration was per-formed using polystyrene. OMNIC v9.11 software package (Thermo-Nicolet) was used to acquire, process, and evaluate the spectral data.

¹⁹F ssNMR Spectroscopy

The ¹⁹F direct-polarization (DP) magic-angle-spinning (MAS) NMR spectra were measured on a Bruker NEO 400 MHz (9.4 T) spectrometer using a 4 mm Bruker iProbe at Triclinic Labs. ¹⁹F DP spectra were acquired using a Hahn echo pulse sequence with rotor synchronized echo delays (D4) to avoid a baseline distortion for all spectra. All experiments were performed at room temperature under 15 kHz MAS, and the acquired spectra were processed using TopSpin GxP 4.1.4. The ¹⁹F chemical shift was externally referenced to the polytetrafluoroethylene (Teflon) ¹⁹F signal at −122 ppm on the CClF3 scale. Detailed acquisition parameters are listed in Table below:

### NAMR Acquisition Parameters

| Parameter Name | Parameter Value | |
|---|---|---|
| Sequence | zgecho.jos.bk | |
| Time domain (TD) | 3072 | |
| Spectral width (SW) | 604 | ppm |
| Acquisition time (AQ) | 6.8 | ms |
| Dwell time (DW) | 2.2 | us |
| Relaxation delay (D1) | 3 | s |
| Number of scans (NS) | 64 | |
| Carrier frequency (SFO1) | 376.47 | MHz |
| Carrier frequency offset (O1P) | −114 | ppm |

### Solution Proton Nuclear Magnetic Resonance Spectroscopy (¹H NMR)

The ¹H NMR spectra were acquired on a Bruker Avance NEO 400 spectrometer. Samples were prepared by dissolving material in DMSO-d₆. The solutions were placed into individual 5-mm NMR tubes for subsequent spectral acquisition. The temperature controlled (295K) ¹H NMR spectra acquired on the Avance NEO 400 utilized a 5-mm cryoprobe operating at an observing frequency of 400.18 MHz.

### Dynamic Vapor Sorption (DVS)

Vapor sorption data were collected on a Surface Measurement System DVS Intrinsic instrument. Samples were not dried prior to analysis. Sorption and desorption data were collected over the stated range from at 10% RH increments under a nitrogen purge. The equilibrium criterion used verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Data was collected from 25° C. to 350° C. at 10° C./min.

### X-Ray Powder Diffraction (XRPD)

Method A: XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 5.5.

Method B: The Rigaku Smart-Lab X-ray diffraction system was configured for reflection Bragg-Brentano geometry using a line source X-ray beam. The x-ray source is a Cu Long Fine Focus tube that was operated at 40 kV and 44, 45, or 50 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1°2Θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2° or 3° to 40° (2Θ) using a continuous scan of 6° (2Θ) per minute with an effective step size of 0.02° (2Θ).

It is possible to analyze the materials using a similar XRPD system equipped with a Cu x-ray source, using a Bragg-Brentano geometry and step size of 0.02° 2Θ. It is possible to obtain similar quality XRPD diffractograms by varying incident/receiving slits, step sizes and scan speed.

### X-Ray Powder Diffraction Peak Identification

Under most circumstances, peaks within the range of up to about 40° 2Θ were selected. The location of the peaks along the x-axis (°2Θ) were determined using proprietary software and rounded to one significant figure after the decimal point. Peak position variabilities are given to within ±0.2° 2Θ based upon recommendations outlined in the USP discussion of variability in x-ray powder diffraction. For d-space listings, the wavelength used to calculate d-spacings was 1.540593 Å, the Cu-K_{α1} wavelength.

Per USP guidelines, variable hydrates and solvates may display peak variances greater than 0.2° 2Θ and therefore peak variances of 0.2° 2Θ are not applicable to these materials

### Example 1: Compound I Manufacturing Process Methods

The three batches of Compound I (ONC206·2HCl) manufactured to date are described below. The first two processes consistently produced Form 1 (i.e., desolvated anhydrous form, Form 1). Form 1 is hygroscopic and tends to convert into Form 2 (variable dihydrate) upon exposure to moisture. Since no form change was observed during relative humidity (RH) experiments, Form 2 was used for further development. A third manufacturing process for Form 2 was developed and is demonstrated below.

TABLE 37
Manufacturing of Compound I Polymorphic Forms

| | | Date of | Polymorphic |
|---|---|---|---|
| No | Quantity | Manufacturing | Form |
| 1. | 1.49 Kg | October 2017 | Form 1 |
| 2. | 6.26 Kg | August 2021 | Form 1 |
| 3. | 41.15 Kg | February 2024 | Form 2 |

The details of the manufacturing process followed to produce the above three individual lots of ONC206·2HCl are described in detail below.

### Manufacturing of No. 1 (Form 1)

The chemical synthesis of ONC206 and its conversion to the dihydrochloride (ONC206·2HCl) was accomplished following the process shown in the Scheme below. In this route, the piperidone ester (Compound 2·HCl) was converted to the Compound 2 (free base) and condensed with the benzyl imidazoline (Compound 10) to yield the ONC206 free base. The solution containing ONC206 free base was directly converted to the corresponding dihydrochloride salt (crude ONC206·2HCl), which was crystallized from ethanol during the final step.

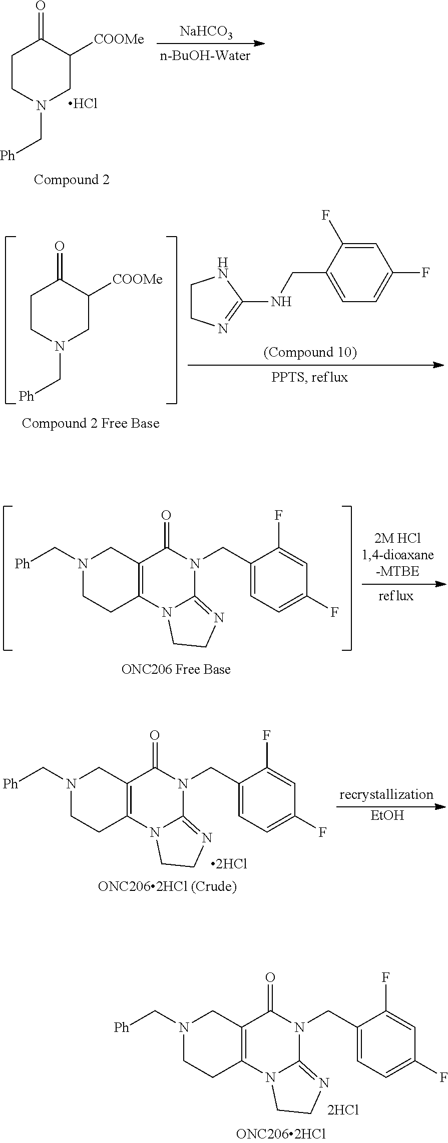

### Brief Description of the Manufacturing Process:

### Step-1 Synthesis of Crude ONC206·2HCl:

To a stirred solution of NaHCO₃ (859 g; 10.2 mol) in 8400 mL of distilled water in a 50 L reactor, was added the benzylpiperidinone ester HCl salt (compound 2 hydrochloride (2743 g, 9.667 mol, 1.70 equiv)) in portions. n-Butanol (8400 mL) was then added to the mixture. The mixture was stirred for 30 min and transferred to a separatory funnel. The organic phase was separated and dried by stirring over 1000 g of MgSO₄ for 2 hr. The MgSO₄ was filtered off and washed with 1000 mL of n-butanol and transferred to a 22 L reactor equipped with mechanical stirring, N₂ inlet, a thermocouple, a condenser, and a Dean-Stark trap. The imidazoline compound 10 (1200 g, 0.5685 mol, 1.00 equiv) and PPTS (71.5 g, 0.237 mol, 4.2 mol %) were added to the reactor and stirred overnight. The mixture was heated to reflux and maintained at reflux until the peak area of the product by HPLC remained constant over time. This condition was met and the mixture allowed to cool to room temperature after 4 h.

The mixture was transferred to a 50 L reactor with a bottom valve and was washed with 8400 mL of water. The organic phase was diluted with MTBE (16800 mL) and washed with water (2×8400 mL) and transferred to a 50 L reactor equipped with mechanical stirring, N₂ inlet, a thermocouple, a condenser, and a Dean-Stark trap. HCl (4 N in dioxane, 3128 mL) was diluted with an equal volume of MBTE (3128 mL) and the solution of 2 N HCl in dioxane-MTBE was added until no more solid precipitated out on the surface at the addition of HCl (5600 mL). The mixture was heated at reflux at 60-65° C. for 2 hr with the water separating in the Dean-Stark trap. After cooling to room temperature, the solid was filtered off using a table top ceramic filter and washed with n-butanol:MTBE (1:2, 7200 mL). The solid was dried in drying trays in a vacuum oven at 55° C. for 4 hr, then room temperature for 60 h, to afford 2305 g (84% yield) of the crude product as a yellow solid.

### Step-2 Purification of ONC206·2HCl:

To a 22 L reactor equipped with mechanical stirring, nitrogen inlet, a thermocouple, and a condenser, the crude solid (2300 g) was added, followed by ethanol (11500 mL). The mixture was heated until it completely dissolved (67° C.) and the hot solution filtered. The solution was cooled slowly to room temperature overnight with stirring, then to 15° C. for 1 hour. The solid was filtered off, washed with ethanol (3×1400 mL), transferred to drying trays, and dried in the vacuum oven at 75° C. until a constant weight was reached. 1492 g of ONC206·2HCl was obtained as an off-white solid in a yield of 65%.

### Manufacturing of No. 2 (Form 1)

The solvent used during the first step of the synthesis was changed from n-butanol to Toluene, and potassium carbonate was chosen as base instead of sodium bicarbonate for the formation of Compound 2 free base. At the end of first step, ONC206 free base was isolated as an intermediate and analyzed for its purity. In step-2, use of toxic solvent (1,4-dioxane) was avoided and instead 2.5 M solution of HCl in EtOH was utilized for the conversion of ONC206 free base to the corresponding dihydrochloride salt (crude), which was purified through reslurrying from refluxing isopropanol (IPA) as depicted in the scheme below.

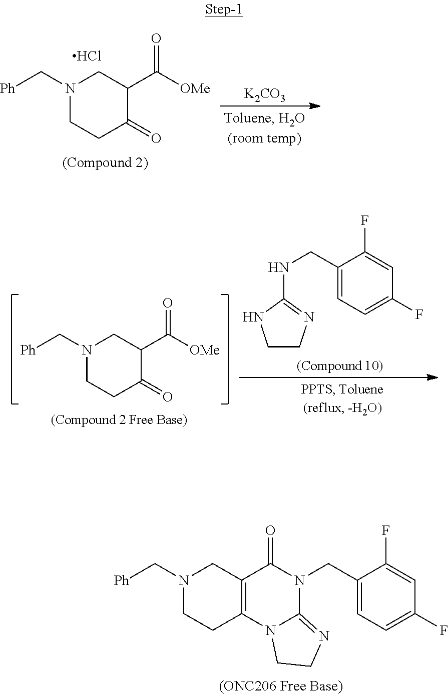

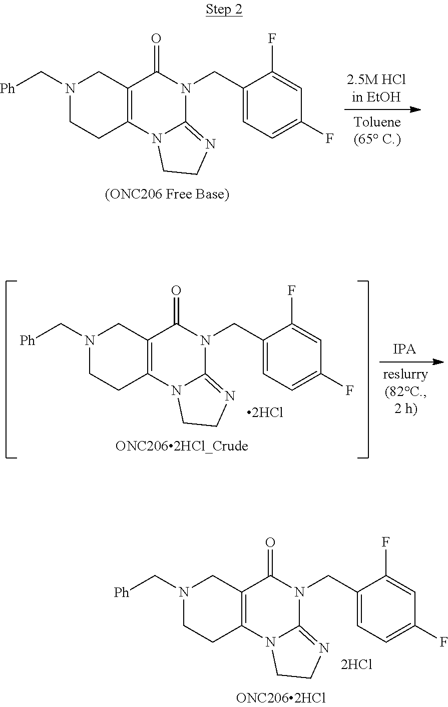

### Brief Description of the Manufacturing Process:

### Step-1 Synthesis of ONC206 Free Base:

To a stirred solution K₂CO₃ (2.84 kg) in distilled water (17 L) at room temperature, was sequentially added toluene (33 kg) and Compound 2 hydrochloride (5.64 kg) and the contents were stirred for 30 minutes. The layers were separated, and the organic layer was washed with water (2×10 kg). To the organic layer, containing Compound 2 free base, was sequentially charged Compound 10 (3.34 kg) and PPTS (200 g) and the reaction mixture was refluxed at 110° C. for at least 3 h until reaction completion.

The contents were cooled to room temperature, filtered through an in-line filter, and diluted with water (20 L). The reaction mixture is stirred for 15 minutes and allowed to settle for 15 minutes. The layers were separated, and the organic layer was distilled to remove toluene partially. The organic solution containing ONC206 free base was heated to 75° C. to obtain a clear solution. To the hot solution was added MTBE (19 kg) while maintaining the temperature around 67° C., and the contents were slowly cooled to room temperature. The solids were filtered, and the wet cake was washed with MTBE (2×5 kg), dried under vacuum at 40° C. until constant weight is achieved affording ONC206 free base as yellow solid (4.97 kg, 80% yield).

Note: Another batch of ONC206 free base (1.85 kg, 80% yield) was prepared following the above process. These two lots of ONC206 free base were combined and converted into ONC206·2HCl during the next step.

### Step-2, Synthesis of ONC206·2HCl:

First, ~2.5 M HCl solution in EtOH was prepared by slow addition of HCl gas (1.6 kg) to anhydrous EtOH (16 L). The solution was titrated and confirmed that the concentration of HCl in EtOH solution is 2.7 M.

ONC206 FB (6.8 kg) in toluene (35.5 kg) was heated at 65° C. until complete dissolution and the hot solution was filtered through an in-line filter. To the hot solution was slowly added HCl in EtOH (14.4 L) while maintaining the reaction temperature of around 60° C. The reaction mixture was gradually cooled to room temperature overnight. The solids obtained were filtered, washed with IPA (2×6 Kg) and dried under vacuum at 75° C. until constant weight is achieved.

The solid (~7.3 Kg) obtained above was reslurried in refluxing IPA (21.3 L) at ~82° C. for 2 h. The reaction mixture was gradually cooled to 20° C., filtered, washed with IPA (6 L) and dried under vacuum at 75° C. for several days until constant weight is achieved, resulting the final product as a solid (6.26 Kg, 78% yield).

### Manufacturing of No. 3 (Form 2)

The same experimental conditions that were used for the synthesis of ONC206 free base No. 2 was followed here. However, a simple and robust process was developed for direct conversion of ONC206 free base to ONC206·2HCl Form 2 as shown in the scheme below.

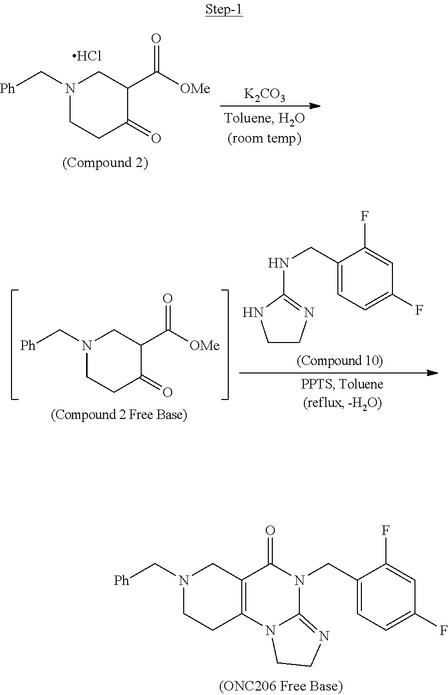

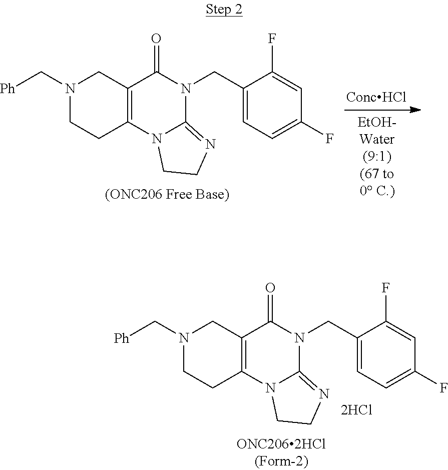 [SMILES: O=C1C2=C(CCN(Cc3ccccc3)C2)N2CCN=C2N1Cc1ccc(F)cc1F ; O=C1C2=C(CCN(Cc3ccccc3)C2)N2CCN=C2N1Cc1ccc(F)cc1F]

### Brief Description of the Manufacturing Process:

### Step-1 Synthesis of ONC206 Free Base:

To a stirred solution K₂CO₃ (23 Kg) in distilled water (135 L) at room temperature, was sequentially added toluene (234.1 Kg) and Compound 2 hydrochloride (45.4 Kg) and the contents were stirred for 30 minutes. The layers were separated, and the organic layer was washed with water (2×89.1 Kg). To the organic layer (containing Compound 2 free base), was sequentially charged Compound 10 (27 Kg) and PPTS (1.6 Kg) and the reaction mixture was refluxed at 110° C. for at least 8 h until reaction completion.

The contents were cooled to room temperature and diluted with water (157 Kg). The reaction mixture is stirred for 15 minutes and allowed to settle for 15 minutes. The layers were separated, and the organic layer was washed with water (157 Kg). The organic layer was distilled to remove toluene partially. The organic solution containing ONC206 free base was heated to 80° C. to obtain a clear solution. The reaction mixture was cooled to 65° C. and to the hot solution was added MTBE (180 Kg) while maintaining the temperature around 65° C. The mixture was slowly cooled to room temperature and the solids were filtered. The wet cake was washed with MTBE (2×30 Kg), dried under vacuum at 40° C. until constant weight is achieved affording ONC206 free base as a solid (36.5 Kg, 70% yield).

### Step-2, Synthesis of ONC206·2HCl (Form-2):

A mixture of ONC206 FB (36.5 Kg) in EtOH (231 Kg) and water (19.7 Kg) was heated at 70° C. until complete dissolution, and the hot solution was filtered through an in-line filter. To the hot solution, was slowly added Conc·HCl (23.9 Kg) while maintaining the reaction temperature of around 67° C. The reaction mixture was cooled to 50° C. and held for 1 h. The contents were further cooled to 0° C. over 12 h period and filtered. The wet solid was washed with EtOH-Water (9:1 mixture, 59.5 Kg), and dried under vacuum affording the final product as white solid (41.1 Kg, 86.8% yield).

TABLE 38
Comparison of the Manufacturing Processes

| | No. 1 | No. 2 | No. 3 |
|---|---|---|---|
| | (Form 1) | (Form 1) | (Form 2) |
| ONC206 Free | n-Butanol was used as | Toluene was used as | Toluene was used as |
| Base formation: | the reaction solvent, and | reaction solvent, and | reaction solvent, and |
| | NaHCO₃ was used as | K₂CO₃ was used as | K₂CO₃ was used as |
| | base. | base. | base. |
| | ONC206 Free base was | ONC206 Free base was | ONC206 Free base |
| | not isolated. | isolated as process | was isolated as process |
| | | intermediate. | intermediate. |
| ONC206•2HCl | 4M HCl in 1,4-dioxane | 2.5M HCl in EtOH was | Conc. HCl was used as |
| salt formation: | was used as reagent. | used as reagent. | reagent. |
| | Salt formation was | Salt formation was | Salt formation was |
| | performed in n-Butanol- | performed in Toluene | performed in EtOH- |
| | MTBE solvent system. | solvent system. | water solvent system. |
| Final | ONC206•2HCl was | ONC206•2HCl was | Additional purification |
| purification of | recrystallized from | reslurried in hot | is not required. Robust |
| the product. | ethanol. | isopropanol. | salt formation step |
| | | | allows to control both |
| | | | the purity and physical |
| | | | attributes (particle |
| | | | size) of the final |
| | | | product. |
| | Since the solid tends to | Since the solid tends to | Since the product is the |
| | form solvates, the final | form solvates, the final | hydrate, the solids are |
| | solids are dried under | solids are dried under | dried at <40° C. under |
| | vacuum and at high | vacuum and at high | vacuum. |
| | temperature (75° C., | temperature (75° C., | |
| | several days) to remove | several days) to remove | |
| | residual solvents. | residual solvents. | |

### Example 2: Summary of ONC206 Polymorphic Form Preparations

Form used when the chemical structure was confirmed and the material was shown to be a single phase by indexing.

### Preparation of ONC206·2HCl Form 1

The sample was recrystallized from ethanol at manufacturing scale and dried under vacuum at 75° C. for 6 days.

### Isopropyl Acetate Sample

ONC206·2HCl, (52.0 mg) was contacted with 0.5 mL of isopropyl acetate producing a white slurry. The sample was stirred at room temperature for seven days. Some solids were observed above the solvent level after equilibration. The stir bar was removed and the sample was centrifuged and the supernatant removed prior to analysis.

### Preparation of ONC206·2HCl Form 2

### Ethanol-Water Sample

103.1 mg of ONC206·2HCl (Form 1) was contacted with 0.5 mL of a 90.66% ethanol in water solution producing a white slurry. The sample was allowed to slurry 7 days at room temperature. The sample was centrifuged and the supernatant removed prior to analysis.

### 2 g Scale

2.0008 g of ONC206·2HCl (Form 1) was added to a 50 mL Erlenmeyer flask along with 18 mL of ethanol and 2 mL of HPLC grade water. A stir bar was added, the flask was sealed, and the sample allowed to stir at room temperature producing a white slurry. The sample was stirred three days and filtered using a paper filter producing a colorless filtrate and a white residue. The solids were placed in a vacuum oven at room temperature to dry overnight producing a white, free-flowing powder.

### 500 g Scale

ONC206 Form A (free base) was dissolved in an ethanol-water (9-1) solvent mixture at ~70° C., and treated with aqueous HCl (31%). The contents were gradually cooled to 0° C., and the solids obtained were filtered. The wet solid was dried under vacuum at around 40° C.

### 40 kg Scale

ONC206 Form A (free base) was dissolved in an ethanol-Water (9-1) solvent mixture at ~70° C., and treated with aqueous HCl (31%). The contents were gradually cooled to 0° C., and the solids obtained were filtered. The wet solid was dried under vacuum at around 40° C.

### Preparation of ONC206·2HCl Form 3

Form 3 was isolated from slurrying Form 1 in acetonitrile at ambient temperature for 3 days. Slurry was prepared by adding sufficient solid to the solvent at ambient temperature such that undissolved solids were present. The mixture was then stirred in a closed vial.

### Preparation of ONC206·2HCl Form 4

Form 4 was isolated from slurrying Form 1 in isopropanol at ambient temperature for 3 days. Slurry was prepared by adding sufficient solid to the solvent at ambient temperature such that undissolved solids were present. The mixture was then stirred in a closed vial.

### Preparation of ONC206·2HCl Form 5

Form 5 was isolated from slurrying Form 1 in N-methylpyrrolidone (NMP) at ambient temperature for 3 days. Slurry was prepared by adding sufficient solid to the solvent at ambient temperature such that undissolved solids were present. The mixture was then stirred in a closed vial.

### Preparation of ONC206·2HCl Form 2+Material 6

Form 2+Material 6 was isolated from slurrying Form 2 in ethanol (EtOH) at ambient temperature for 10 days. Slurry was prepared by adding sufficient solid to the solvent at ambient temperature such that undissolved solids were present. The mixture was then stirred in a closed vial.

Form 2+Material 6 was also isolated from solutions of ONC206·2HCl prepared in EtOH and filtered through a 0.2 or 0.45 μm nylon or PTFE syringe filter. The ONC206·2HCl solution was added to the aliquots of heptane antisolvent (reverse addition) and stirred at RT for 1 day until precipitates formed.

### Preparation of ONC206·2HCl Material 7+Form 2

Material 7+Form 2 was isolated from solutions of ONC206·2HCl prepared in DMSO and MTBE followed by DCM antisolvent addition which was stirred at RT for 3 hours.

### Preparation of ONC206·2HCl Material 8+Form 2

Material 8+Form 2 was isolated from solutions of ONC206·2HCl prepared in MeOH to which acetone antisolvent was added and stirred at RT for 1 day.

### Preparation of ONC206·2HCl Form 9

Form 9 was isolated from solutions of ONC206·2HCl prepared in MeOH at ambient temperature. The solution was then allowed to evaporate in a loosely closed vial at ambient temperature inside a box with a nitrogen stream.

### Preparation of ONC206·2HCl Material 10+Form 2

Material 10+Form 2 was isolated from slurrying Form 2 in acetone at ambient temperature for 4 days. Slurry was prepared by adding sufficient solid to the solvent at ambient temperature such that undissolved solids were present. The mixture was then stirred in a closed vial.

### Preparation of ONC206·2HCl Form 11

Form 11 was isolated from drying Form 3 in ACN for 6 days under nitrogen stream.

### Preparation of ONC206·2HCl Material 13+ONC206·2HCl Form 11

ONC206·2HCl (100.3 mg, Form 1) was weighed into a 1-dram vial charged with a stir bar, into which 1.5 mL acetonitrile was added. The resulting white slurry was stirred at 70° C. for 7 days, following which the vial was centrifuged, supernatant pipetted out, and the wet white residue was analyzed.

### Preparation of ONC206·2HCl Material 14

ONC206·2HCl (50.1 mg, Form 1) was weighed into a 1-dram vial. The sample was dissolved in 0.5 mL dimethylformamide and filtered through a 0.22 μM PTFE syringe filter into a new 1-dram vial. The vial was left uncapped and placed into a larger vial containing dichloromethane. The larger vial was capped to allow vapor diffusion to occur at room temperature, resulting in a white crystalline residue.

### Preparation of ONC206·2HCl Material 15

### Anti-Solvent Addition (ONC206·2HCl Material 15)

ONC206·2HCl (50.1 mg, Form 1) was dissolved in 0.5 mL glacial acetic acid and stirred at room temperature for 1 week. To this clear solution, 1.0 mL of hexane was added which first resulted in a clear binary mixture followed by precipitation of a white waxy solid after 5 minutes. The clear solvent was pipetted out and the residue dried under ambient conditions for two days to produce dry solids.

### Grinding (ONC206·2HCl Material 15+ONC206·2HCl Material 10)

ONC206·2HCl (26.2 mg, Form 2) was milled with acetic acid using a model MM200 Retsch mill with grinding cups and a stainless-steel ball. 34 μL of acetic acid was diluted to 100 μL using ethyl acetate. 10 μL of the resulting solution was included in the grind (~1.1 equivalents). The sample was milled at maximum power for 30 minutes resulting in a free-flowing white powder.

### Preparation of ONC206·2HCl Material 16

ONC206·2HCl (98.9 mg, Form 1) was dissolved in 1.0 mL hexafluoroisopropanol and filtered through a 0.22 μM PTFE syringe filter into a 1-dram vial. The vial was loosely capped and the clear colorless solution was allowed to slowly evaporate at RT, resulting in a dry white residue.

### Preparation of ONC206·2HCl Material 17

### From Isopropyl Acetate (ONC206·2HCl Material 17)

ONC206·2HCl (101.3 mg, Form 1) was combined with 1.5 mL of isopropyl acetate at 70° C. producing a white slurry. The sample was allowed to stir at elevated temperature for 7 days. The clear solvent was pipetted out and the solids recovered.

### From Methyl Ethyl Ketone (ONC206·2HCl Material 17+Peaks [Possible ONC206·2HCl Material 10])

ONC206·2HCl (98.3 mg, Form 1) was combined with 1.5 mL of methyl ethyl ketone at 70° C. producing a white slurry. The sample was allowed to stir at elevated temperature for 7 days. The clear solvent was pipetted out and the solids recovered.

### Preparation of ONC206·2HCl Adipic Acid Material 1

### Slurry

44.3 mg of ONC206·2HCl (Form 1) and 8.1 mg of adipic acid (~0.6 molar equivalents) were slurried in 0.5 mL of isopropyl acetate at room temperature. After three days of stirring, a thick, poorly mobile, suspension was produced and an additional 0.5 mL of isopropyl acetate was added. The sample was allowed to stir one additional day at room temperature. The resulting sample was centrifuged and the supernatant removed. The resulting solids were dried under a stream of air overnight prior to analysis.

### Grind

45.1 mg of ONC206·2HCl (Form 1) and 14.3 mg of adipic acid (~1 molar equivalent) were milled with 20 μL of chloroform using a model MM200 Retsch mill with grinding cups and a stainless-steel ball. The sample was milled twice for 15 minutes and scraped off the walls of the cup in between.

### Preparation of ONC206·2HCl Glutaric Acid Material 1+Glutaric Acid

ONC206·2HCl (46.5 mg, Form 1) was combined with ~1 molar equivalent (12.5 mg) of glutaric acid and 20 μl of isopropyl acetate. The sample was milled on a model MM200 Retsch mill with a grinding cup and stainless-steel ball. The sample was milled twice for 15 minutes and scraped off the walls of the cup in between.

### Preparation of ONC206·2HCl Glutaric Acid Material 2

46.2 mg of ONC206·2HCl (Form 1) and 13.9 mg of glutaric acid (~1 molar equivalent) were slurried in 0.6 mL of isopropyl acetate at room temperature. After four days of equilibration, a white slurry was produced. The sample was centrifuged and the supernatant removed.

### Preparation of ONC206·2HCl Succinic Acid Material 1+Succinic Acid+Possible ONC206·2HCl Form 1

ONC206·2HCl (45.1 mg, Form 1) and 11.2 mg of succinic acid (~1 molar equivalent) were milled with 20 μL of ethyl acetate using a model MM200 Retsch mill with grinding cups and a stainless-steel ball. Solids were scraped from the walls and milled an additional 15 minutes.

### Preparation of ONC206·2HCl Vanillin Material 1+Vanillin

ONC206·2HCl (44.9 mg, Form 1) and 13.9 mg of vanillin (~1 molar equivalent) were milled with 20 μL of chloroform using a model MM200 Retsch mill with grinding cups and a stainless-steel ball at 25 Hz for 15 minutes. Solids were scraped from the walls and milled an additional 15 minutes.

### Preparation of Amorphous ONC206·2HCl

### Spray Dried

313.6 mg of ONC206·2HCl Form 1 was dissolved in 6 mL of methanol at room temperature with sonication. Sample was filtered using a 0.22 μm Nylon syringe filter. Solution was spray dried using ProCept spray dryer with 0.4 m3/min inlet gas flow rate, 75° C. inlet gas temperature, 100 rpm solution pump speed, 0.3 bar nozzle gas pressure and 2 bar cyclone gas pressure. The spray drying yielded 224 mg of solids that were stored in a vacuum desiccator prior to analysis.

### Lyophilized

302.7 mg of ONC206·2HCl Form 2 was dissolved in 10 mL HPLC water. The clear colorless solution was then filtered through a 0.22 μm hydrophilic PTFE filter into a 50 mL round bottom (RB) flask and subsequently frozen in an acetone-dry ice bath. The flask with its frozen contents was then placed on a Labconco FreeZone 1 lyophilizer pre-equilibrated to approximately −52° C. and left for one day. The lyophilization generated 270 mg of fluffy white solids.

### Preparation of ONC206 Mono-Maleate Material A

45.7 mg of ONC206·2HCl (Form 1) was milled with 11.5 mg (~1 molar equivalent) of maleic acid and 20 μL of ethyl acetate. The starting materials were added to a grinding cup along with a stainless-steel ball and milled on a model MM200 Retsch mill at 25 Hz for 15 minutes. Solids were scraped from the walls and milled an additional 15 minutes.

### Preparation of ONC206 Form A

ONC206 free base was prepared from the as-received di-HCl salt (Form 1). A small scale experiment was conducted initially by dissolving the di-HCl salt in water and then adding 2 molar equivalents of NaOH. Precipitation was observed after the addition of NaOH and the slurry was aged at ambient temperature for 1 day. Solids were isolated by filtration and washed with water.

### Preparation of ONC206 Material C

### ONC206 Material C+L-Proline (L-Proline Cocrystal Attempt)

50.2 mg of ONC206 Form A (free base), 15.6 mg of L-proline (~1.1 molar equivalents), and 0.5 mL of 50:50 isopropyl ether:methyl isobutyl ketone (MIBK) producing a light yellow to off-white slurry. The sample was stirred at room temperature for six days resulting in a thick suspension and partial evaporation of solvent. An additional 1 mL of 50:50 isopropyl ether:MIBK was added and the sample was stirred at room temperature one additional day. The sample was centrifuged and the supernatant removed.

### ONC206 Material C (Sucralose Cocrystal Attempt)

51.3 mg of ONC206 Form A (free base) and 54.9 mg of sucralose (~1.1 molar equivalent) were dissolved in in 1.5 mL of 50:50 acetonitrile:water at reflux. The sample was cooled by transferring the sample to the refrigerator. After 3 days of equilibration in the refrigerator large crystals were observed. The remaining solvent was removed and the crystals were crushed and air dried prior to analysis.

### Preparation of ONC206 Betaine HCl Material A

50.4 mg of ONC206 Form A (free base), 94.8 mg of betaine HCl (~5 molar equivalents), and 0.5 mL of water were combined. The sample was initially clear before turning into a slurry. The sample was stirred at room temperature for five days producing a white suspension. The sample was centrifuged and the supernatant removed.

### Preparation of ONC206 Methyl Paraben Material A

49.8 mg of ONC206 Form A (free base), 20.4 mg of methyl paraben (~1.1 molar equivalents), and 1 mL of 50:50 ethanol:hexane were combined producing a light yellow to off-white slurry. The sample was stirred at room temperature for seven days producing an off-white suspension. The sample was centrifuged and the supernatant removed.

### Preparation of ONC206 Propyl Gallate Material A

50.3 mg of ONC206 Form A (free base), 28.7 mg of propyl gallate (~1.1 molar equivalents), and 1 mL of 50:50 acetonitrile:diethyl ether were combined producing a light yellow to off-white slurry. The sample was stirred at room temperature for seven days producing an off-white suspension. The sample was centrifuged and the supernatant removed.

### Preparation of ONC206 Saccharin Material A+Possible Minor ONC206 Form A

28.4 mg of ONC206 Form A (free base) and 14.0 mg of saccharin (~1.1 molar equivalent) were milled with 20 μL of MTBE using a model MM200 Retsch mill with grinding cups and a stainless-steel ball. The sample was milled for 15 minutes.

### Preparation of ONC206 Sorbitol Material A+Sorbitol

50.3 mg of ONC206 Form A (free base), 24.7 mg of sorbitol (~1.1 molar equivalents), and 0.5 mL of 50:50 MTBE:ethanol were combined producing a light yellow to off-white slurry. The sample was stirred at room temperature for seven days producing an off-white suspension. The sample was centrifuged and the supernatant removed.

### Preparation of ONC206 Zinc Chloride Material A+Possible Minor ONC206 Form A

30.0 mg of ONC206 Form A (free base) and 11.0 mg of zinc chloride (~1.1 molar equivalent) were milled with 20 μL of ethanol using a model MM200 Retsch mill with grinding cups and a stainless-steel ball. The sample was milled for 15 minutes producing sticky yellow solids.

### Preparation of ONC206 Besylate Form A

The besylate salt of ONC206 was prepared by adding 1 molar equivalent of benzenesulfonic acid to an ethyl acetate solution of ONC206 free base at 50° C. Precipitation was observed upon addition of the acid and the slurry was cooled to ambient temperature at 6° C./h. The slurry was aged for 2 days at ambient temperature and then the solids were isolated. XRPD analysis indicated the solids were composed of a unique crystalline material and minor FB Form A. The unique crystalline material was designated Besylate Form A. To remove the residual FB Form A, the material was slurried in EtOAc at ambient temperature for 3 days.

### Preparation of ONC206 Fumarate Form A

The fumarate salt of ONC206 was prepared by Dissolving ONC206 (86 mg) and 3 molar equivalents of fumaric acid in 1.5 ml THF at 60° C. The solution was cooled to RT, 6° C./h, yielding a clear solution. SE. Solids. Stirred in ACN (2 ml), RT, 4 d.

### Preparation of ONC206 Sulfate Form A

The sulfate salt of ONC206 was prepared by dissolving ONC206 (99 mg) in ACN (1 ml) at 50° C. Added 1 molar equivalent of sulfuric acid. Precipitation. Cooled to RT, 6° C./h. Gel. FE. Few solids. Yellow gel/film. Added IPA (1 ml). Stirred, RT, 5 d.

### Preparation of ONC206 Tosylate Form A

The tosylate salt of ONC206 was prepared by slurrying ONC206 (94 mg) in EtOAc (1 ml) at RT. Added 1 molar equivalent of p-toluenesulfonic acid. Heated 60° C. Thick slurry. Added EtOAc (1 ml). Mobile slurry. Cooled to RT, 6° C./h. Stirred, RT, 4 d.

### Preparation of ONC206 Edisylate Form A

The edisylate salt of ONC206 was prepared by slurrying ONC206 (102 mg) in EtOAc (1 ml) at RT. Added 1 molar equivalent of acid. Heated 60° C. Slurry, some solid stuck on bottom of vial. Cooled to RT, 6° C./h. Stirred, RT, 4 d.

### Preparation of ONC206 Gentisate Form A

The Gentisate salt of ONC206 was prepared by slurrying Slurry ONC206 (91 mg) in ACN (1 ml) at RT. Added 1 molar equivalent of gentisic acid. Heated 60° C. Good slurry. Cooled to RT, 6° C./h. Stirred, RT, 4 d.

### Preparation of ONC206 Potential Benzoate Material A

The potential benzoate salt of ONC206 was prepared by dissolving ONC206 (100 mg) in acetone (1 ml) at RT. Added 1 molar equivalent of benzoic acid. Clear solution. Stirred, RT, 1 d. Clear solution. Added heptane (1 ml). Clear solution. Stirred, RT, 5 h. Clear solution. FE. Few solids. Yellow gel/film. Added IPA. Stirred, RT, 5 d.

### Preparation of ONC206 Potential Mesylate Material A

The potential mesylate salt of ONC206 was prepared by stirring ONC206 (94 mg) in IPA (1 ml) at 50° C. Added 2 molar equivalents of methanesulfonic acid. Clear solution. Cooled to RT, 6° C./h. Clear solution. Added heptane (1 ml). Small amount of gel. Stirred, RT, 5 d.

### Preparation of ONC206 Potential Phosphate Material A

The potential phosphate salt of ONC206 was prepared by slurrying ONC206 (90 mg) in ACN (1 ml) at RT. Added 1 molar equivalent of phosphoric acid. Heated 60° C. Thin slurry, some solids stuck on bottom of vial. Cooled to RT, 6° C./h. Stirred, RT, 4 d. Sticky solids. SE (partial). Filtered. Washed with 0.5 ml ACN.

### Preparation of ONC206 Potential IPA Solvate

The potential IPA solvate of ONC206 was prepared by two methods:

Dissolved ONC206 (95 mg) in EtOAc (1 ml) at 50° C. Added 1 molar equivalent of glycolic acid. Clear solution. Cooled to RT, 6° C./h. Slightly cloudy. FE. Few solids. Clear film Added IPA. Stirred, RT, 5 d.

Dissolved ONC206 (102 mg) in ACN (1 ml) at 50° C. Added 1 molar equivalent of hippuric acid. Clear solution. Cooled to RT, 6° C./h. Clear solution. Added MTBE (1 ml). Clear solution. Stirred, RT, 5 h. Clear solution. FE. Few solids. Yellow gel/film. Added IPA. Stirred, RT, 5 d.

Preparation of ONC206 Unique Reaction Product with Oxalic Acid

The unique reaction product material with oxalic acid and ONC206 was prepared by slurrying Slurry ONC206 (94 mg) in EtOAc (1 ml) at RT. Added 1 molar equivalent of oxalic acid. Heated 60° C. Gel. Cooled to RT, 6° C./h. Stirred, RT, 4 d. Sticky solids. SE (partial). Filtered. Washed with 0.5 ml MTBE.

### Example 3: Stability Testing of ONC206

The purpose of the ICH-compliant stability study was to evaluate the performance to specifications of ONC206-HCl Drug Substance under specified container closure configuration, storage conditions, and intervals.

The study was performed according to ICH Q1A(R2) and QlE Guidance documents.

The stability conditions were:
−20° C.,
2-8° C.,
30 2° C./60%±5% RH: long term storage conditions,
30±2° C./65%±5% RH: intermediate conditions, and
40±2° C./75%±5% RH: accelerated conditions.

The Container Closure Systems were:
Primary container: LDPE bag, 4×8 inches (Source: Nexus—64378); bag closure PE 180 nm (Source: Entourage),
Secondary container: LDPE bag, 4×8 inches (Source: Nexus—64378); bag closure PE 180 nm (Source: Entourage),
Tertiary container: Heat-sealed foil bag, 15×30 (Source: SERCALIA S.L.—10234), and
Outer container: Plastic drum, 6 L (Source: Curtec, HDPE ref 54021) or Plastic drum, 3.6 L (Source: Curtec).

Over the course of the study, there was no significant change for appearance at all conditions, no significant change for Polymorphic form (XRPD) at all conditions, no significant evolution for Assay HPLC, no significant evolution for Purity and total impurities at all conditions, no significant evolution for Water Content at all conditions, and no significant evolution for Particle Size (DlO, D50 and D90) at all conditions. All microbial results are in specifications

The results obtained confirm the stability of ONC206-HCl over a period of 6 months at 2-8° C. (Table 39) and 40° C. (Table 40) conditions and over a period of 12 months at −20° C. (Table 41), 25° C. (Table 42) and 30° C. (Table 43) conditions. The

study continues up to 60 months.

TABLE 39
Stability of ONC206-HCl at 2-8° C.

| Duration in Months | 0 | 1 | 2 | 3 | 6 |
|---|---|---|---|---|---|
| Appearance | C | C | C | C | C |
| (White to off-white solid) | | | | | |
| Polymorphic form (XRPD) | C | C | C | C | C |
| (Consistent with form 2 reference | | | | | |
| diffractogram) | | | | | |
| Assay by HPLC-UV | 101.6 | 101.3 | 100.3 | 100.3 | 99.6 |
| (Anhydrous & Solvent Free basis) | | | | | |
| 97.0-103.0% w/w | | | | | |
| Purity by HPLC | 100.0 | 100.0 | 100.0 | 100.0 | 100.0 |
| (NLT 98.0% area) | | | | | |
| Each unidentified impurity | C | C | C | C | C |
| (NMT 0.5% area) | | | | | |
| (Report RRT and % area for peaks ≥0.10% area) | / | / | / | / | / |
| Total impurities | 0.00 | 0.00 | 0.00 | 0.00 | 0.00 |
| (NMT 2.0% area) | | | | | |
| Water Content by Kf | 9.3 | 9.2 | 9.4 | 9.5 | 9.3 |
| (Report result % w/w) | | | | | |
| Particle Size (Malvern) D10 | 28.2 | 27.8 | 29.4 | 28.3 | 28.3 |
| (Report result μm) | | | | | |
| Particle Size (Malvern) D50 | 91.4 | 89.8 | 91.6 | 90.9 | 91.1 |
| (Report result μm) | | | | | |
| Particle Size (Malvern) D90 | 201.3 | 196.0 | 189.0 | 197.0 | 199.0 |
| (Report result μm) | | | | | |
| Toral aerobic microbial count | | | | | <200 |
| (NMT 1000 CFU/g) | | | | | |
| Total Combined yeast & molds Count | | | | | <5 |
| (NMT 1000 CFU/g) | | | | | |
| Escherichia Coli | | | | | A |
| (Absence in 1 g) | | | | | |

*C = Conforms and A = Absence

TABLE 40
Stability of ONC206-HCl at Stability conditions: 40 ± 2° C./75% ± 5% RH: accelerated conditions

| Duration in Months | 0 | 3 | 6 |
|---|---|---|---|
| Appearance | C | C | C |
| (White to off-white solid) | | | |
| Polymorphic form (XRPD) | C | C | C |
| (Consistent with form 2 reference diffractogram) | | | |
| Assay by HPLC-UV | 101.6 | 100.1 | 100.2 |
| (Anhydrous & Solvent Free basis) | | | |
| 97.0-103.0% w/w | | | |
| Purity by HPLC | 100.0 | 100.0 | 100.0 |
| (NLT 98.0% area) | | | |
| Each unidentified impurity | C | C | C |
| (NMT 0.5% area) | | | |
| (Report RRT and % area for peaks ≥0.10% area) | / | RRT 1.30 | / |
| | | 0.10% aire | |
| Total impurities | 0.00 | 0.00 | 0.00 |
| (NMT 2.0% area) | | | |
| Water Content by Kf | 9.3 | 9.5 | 9.5 |
| (Report result % w/w) | | | |
| Particle Size (Malvern) D10 | 28.2 | 28.1 | 29.2 |
| (Report result μm) | | | |
| Particle Size (Malvern) D50 | 91.4 | 91.5 | 92.6 |
| (Report result μm) | | | |
| Particle Size (Malvern) D90 | 201.3 | 199.0 | 201.0 |
| (Report result μm) | | | |
| Toral aerobic microbial count | | | <200 |
| (NMT 1000 CFU/g) | | | |
| Total Combined yeast & molds Count | | | <5 |
| (NMT 1000 CFU/g) | | | |
| Escherichia Coli | | | A |
| (Absence in 1 g) | | | |

*C = Conforms and A = Absence

TABLE 41
Stability of ONC206-HCl at −20° C.

| Duration in Months | 0 | 1 | 2 | 3 | 6 | 9 | 12 |
|---|---|---|---|---|---|---|---|
| Appearance | C | C | C | C | C | C | C |
| (White to off-white solid) | | | | | | | |
| Polymorphic form (XRPD) | C | C | C | C | C | C | C |
| (Consistent with form 2 reference | | | | | | | |
| diffractogram) | | | | | | | |
| Assay by HPLC-UV | 101.6 | 101.5 | 100.2 | 100.0 | 100.1 | 100.4 | 99.9 |
| (Anhydrous & Solvent Free basis) | | | | | | | |
| 97.0-103.0% w/w | | | | | | | |
| Purity by HPLC | 100.0 | 100.0 | 100.0 | 100.0 | 100.0 | 100.0 | 100.0 |
| (NLT 98.0% area) | | | | | | | |
| Each unidentified impurity | C | C | C | C | C | C | C |
| (NMT 0.5% area) | | | | | | | |
| (Report RRT and % area for | / | / | / | / | / | / | / |
| peaks ≥0.10% area) | | | | | | | |
| Total impurities | 0.00 | 0.00 | 0.00 | 0.00 | 0.00 | 0.00 | 0.00 |
| (NMT 2.0% area) | | | | | | | |
| Water Content by Kf | 9.3 | 9.3 | 9.4 | 9.5 | 9.3 | 9.3 | 9.2 |
| (Report result % w/w) | | | | | | | |
| Particle Size (Malvern) Dl0 | 28.2 | 27.5 | 28.8 | 27.2 | 27.5 | 29.5 | 28.7 |
| (Report result μm) | | | | | | | |
| Particle Size (Malvern) D50 | 91.4 | 89.5 | 90.6 | 89.4 | 89.0 | 91.8 | 92.2 |
| (Report result μm) | | | | | | | |
| Particle Size (Malvern) D90 | 201.3 | 193.0 | 189.0 | 195.0 | 192.0 | 196.0 | 201.0 |
| (Report result μm) | | | | | | | |
| Toral aerobic microbial count | | | | | <200 | | <200 |
| (NMT 1000 CFU/g) | | | | | | | |
| Total Combined yeast & molds Count | | | | | <5 | | <5 |
| (NMT 1000 CFU/g) | | | | | | | |
| Escherichia Coli | | | | | A | | A |
| (Absence in lg) | | | | | | | |

*C = Conforms and A = Absence

TABLE 42
Stability of ONC206-HCl at 25 ± 2° C./60% ± 5% RH: long term storage conditions

| Duration in Months | 0 | 3 | 6 | 9 | 12 |
|---|---|---|---|---|---|
| Appearance | C | C | C | C | C |
| (White to off-white solid) | | | | | |
| Polymorphic form (XRPD) | C | C | C | C | C |
| (Consistent with form 2 reference | | | | | |
| diffractogram) | | | | | |
| Assay by HPLC-UV | 101.6 | 99.1 | 100.2 | 100.4 | 99.6 |
| (Anhydrous & Solvent Free basis) | | | | | |
| 97.0-103.0% w/w | | | | | |
| Purity by HPLC | 100.0 | 100.0 | 100.0 | 100.0 | 100.0 |
| (NLT 98.0% area) | | | | | |
| Each unidentified impurity | C | C | C | C | C |
| (NMT 0.5% area) | | | | | |
| (Report RRT and % area for peaks ≥0.10% area) | / | / | / | / | / |
| Total impurities | 0.00 | 0.00 | 0.00 | 0.00 | 0.00 |
| (NMT 2.0% area) | | | | | |
| Water Content by Kf | 9.3 | 9.5 | 9.3 | 9.3 | 9.3 |
| (Report result % w/w) | | | | | |
| Particle Size (Malvern) D10 | 28.2 | 28.3 | 26.8 | 30.2 | 28.1 |
| (Report result μm) | | | | | |
| Particle Size (Malvern) D50 | 91.4 | 91.4 | 88.5 | 92.2 | 91.3 |
| (Report result μm) | | | | | |
| Particle Size (Malvern) D90 | 201.3 | 199.0 | 196.0 | 192.0 | 203.0 |
| (Report result μm) | | | | | |
| Toral aerobic microbial count | | | <200 | | <200 |
| (NMT 1000 CFU/g) | | | | | |
| Total Combined yeast & molds Count | | | <5 | | <5 |
| (NMT 1000 CFU/g) | | | | | |
| Escherichia Coli | | | A | | A |
| (Absence in 1 g) | | | | | |

*C = Conforms and A = Absence

TABLE 43
Stability of ONC206-HCl at 30 ± 2° C./65% ± 5% RH: intermediate conditions

| Duration in Months | 0 | 6 | 9 | 12 |
|---|---|---|---|---|
| Appearance | C | C | C | C |
| (White to off-white solid) | | | | |
| Polymorphic form (XRPD) | C | C | C | C |
| (Consistent with form 2 reference diffractogram) | | | | |
| Assay by HPLC-UV | 101.6 | 99.2 | 100.2 | 100.0 |
| (Anhydrous & Solvent Free basis) | | | | |
| 97.0-103.0% w/w | | | | |
| Purity by HPLC | 100.0 | 100.0 | 100.0 | 100.0 |
| (NLT 98.0% area) | | | | |
| Each unidentified impurity | C | C | C | C |
| (NMT 0.5% area) | | | | |
| (Report RRT and % area for peaks ≥0.10% area) | / | / | / | / |
| Total impurities | 0.00 | 0.00 | 0.00 | 0.00 |
| (NMT 2.0% area) | | | | |
| Water Content by Kf | 9.3 | 9.3 | 9.3 | 9.3 |
| (Report result % w/w) | | | | |
| Particle Size (Malvern) D10 | 28.2 | 29.3 | 30.5 | 29.7 |
| (Report result μm) | | | | |
| Particle Size (Malvern) D50 | 91.4 | 92.1 | 93.2 | 93.7 |
| (Report result μm) | | | | |
| Particle Size (Malvern) D90 | 201.3 | 199.0 | 197.0 | 203.0 |
| (Report result μm) | | | | |
| Toral aerobic microbial count | | <200 | | <200 |
| (NMT 1000 CFU/g) | | | | |
| Total Combined yeast & molds Count | | <5 | | <5 |
| (NMT 1000 CFU/g) | | | | |
| Escherichia Coli | | A | | A |
| (Absence in 1 g) | | | | |

*C = Conforms and A = Absence

The following presents numbered embodiments of the present disclosure:
1. A crystal form of 7-benzyl-4-(2,4-difluorobenzyl)-2,4,6,7,8,9-hexahydroimidazo[1,2-a]pyrido[3,4-e]pyrimidin-5(1H)-one or a salt form thereof.
2. The crystalline form of embodiment 1, wherein the crystalline form is a crystalline polymorph form of 7-benzyl-4-(2,4-difluorobenzyl)-2,4,6,7,8,9-hexahydroimidazo[1,2-a]pyrido[3,4-e]pyrimidin-5(1H)-one di-hydrochoride salt.
3. The crystalline polymorph of embodiment 1 or 2, wherein the crystalline form is a hydrate or solvate.
4. A crystal form of 7-benzyl-4-(2,4-difluorobenzyl)-2,4,6,7,8,9-hexahydroimidazo[1,2-a]pyrido[3,4-e]pyrimidin-5(1H)-one or a salt form thereof, as a variable hydrate.
5. The crystal form of embodiment 4, which contains up to 3 moles of water.
6. The crystal form of embodiment 4 or 5, which contains from between about 2 and 3 moles of water.
7. A crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a powder X-ray diffraction pattern comprising a peak at diffraction angle (2Θ) of 21.8±0.2.
8. A crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a powder X-ray diffraction pattern comprising a peak at diffraction angle (2Θ) of 15.8±0.2.
9. The crystalline polymorph of embodiment 7 or 8, wherein the crystalline polymorph form has a powder X-ray diffraction pattern comprising a peak at diffraction angle (2Θ) of 21.8±0.2 and 15.8±0.2.
10. The crystalline polymorph of any of embodiments 7 to 9, wherein the crystalline polymorph form has a powder X-ray diffraction pattern comprising a peak at diffraction angles (2Θ) of 7.8±0.2, 11.4±0.2, and 15.6±0.2.
11. The crystalline polymorph of any of embodiments 7 to 10, wherein the crystalline polymorph form has a powder X-ray diffraction pattern comprising a peak at diffraction angles (2Θ) 7.8±0.2, 11.4±0.2, 15.6±0.2, 15.8±0.2. and 21.8±0.2.
12. The crystalline polymorph of any of embodiments 7 to 11, wherein the crystalline polymorph form has a powder X-ray diffraction pattern comprising a peak at diffraction angles (2Θ) of at one or more of 3.96±0.2 degrees two-theta, 5.8±0.2 degrees two-theta, 6.06±0.2 degrees two-theta, 7.82±0.2 degrees two-theta, 8.32±0.2 degrees two-theta, 11.54±0.2 degrees two-theta, ±0.2 degrees two-theta, 12.04±0.2 degrees two-theta, 12.84±0.2 degrees two-theta, 13.06±0.2 degrees two-theta, 13.96±0.2 degrees two-theta, 14.38±0.2 degrees two-theta, 14.6±0.2 degrees two-theta, 15.18±0.2 degrees two-theta, 15.58±0.2 degrees two-theta, 15.78±0.2 degrees two-theta, 16.66±0.2 degrees two-theta, 16.92±0.2 degrees two-theta, 17.08±0.2 degrees two-theta, 17.28±0.2 degrees two-theta, 18.04±0.2 degrees two-theta, 18.28±0.2 degrees two-theta, 18.92±0.2 degrees two-theta, 19.48±0.2 degrees two-theta, 19.76±0.2 degrees two-theta, 20.98±0.2 degrees two-theta, 21.5±0.2 degrees two-theta, 21.82±0.2 degrees two-theta, 22.88±0.2 degrees two-theta, 23.26±0.2 degrees two-theta, 24.12±0.2 degrees two-theta, 24.64±0.2 degrees two-theta, 25.08±0.2 degrees two-theta, 25.3±0.2 degrees two-theta, 25.8±0.2 degrees two-theta, 26.14±0.2 degrees two-theta, 26.4±0.2 degrees two-theta, 26.86±0.2 degrees two-theta, 27.4±0.2 degrees two-theta, 27.84±0.2 degrees two-theta, 28.16±0.2 degrees two-theta, 29.1±0.2 degrees two-theta, 29.56±0.2 degrees two-theta, 29.9±0.2 degrees two-theta, 30.32±0.2 degrees two-theta, 30.72±0.2 degrees two-theta, 31.4±0.2 degrees two-theta, 31.8±0.2 degrees two-theta, 33.7±0.2 degrees two-theta, 34.1±0.2 degrees two-theta, 35.18±0.2 degrees two-theta, and 36.48±0.2 degrees two-theta.
13. The crystalline polymorph form of any one of embodiments 7 to 12, wherein the crystalline polymorph form has a powder X-ray diffraction pattern comprising one or more peaks essentially the same as shown in FIG. 8.
14. The crystalline polymorph form of any one of embodiments 7 to 13, having a DSC thermogram substantially the same as FIG. 10.
15. A crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a FT-IR spectrum comprising a signal at wavenumber 2584 cm⁻¹.
16. The crystalline polymorph of embodiment 15, wherein the crystalline polymorph form has an FT-IR spectrum having a signal at one or more of wavenumber 861 cm⁻¹, 878 cm⁻¹, 1265 cm⁻¹, 1704 cm⁻¹, 1305 cm⁻¹, and 2584 cm⁻¹.
17. The crystalline polymorph of embodiment 15 or 16, wherein the crystalline polymorph form has an FT-IR spectrum substantially the same as FIG. 14.
18. A crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a FT-Raman spectrum comprising a signal at wavenumber 3055 cm⁻¹.
19. The crystalline polymorph of embodiment 18, wherein the crystalline polymorph form has an FT-Raman spectrum having a signal at one or more of wavenumber 239 cm⁻¹, 790 cm⁻¹, 1704 cm⁻¹, and 3055 cm⁻¹.
20. The crystalline polymorph of embodiment 18 or 19, wherein the crystalline polymorph form has an FT-Raman spectrum substantially the same as FIG. 15.
21. A crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a LF-Raman spectrum comprising a signal at wavenumber 149 cm⁻¹.
22. A crystalline polymorph Form 2 of 7-benzyl-4-(2,4-difluorobenzyl)-2,4,6,7,8,9-hexahydroimidazo[1,2-a]pyrido[3,4-e]pyrimidin-5(1H)-one having a LF-Raman spectrum comprising a signal at wavenumber 240 cm⁻¹.
23. The crystalline polymorph of embodiment 21 or 22, wherein the crystalline polymorph form has an FT-Raman spectrum having a signal at one or more of wavenumber 92 cm⁻¹, 149 cm⁻¹, 240 cm⁻¹, and 790 cm⁻¹.
24. The crystalline polymorph of any of embodiments 21 to 23, wherein the crystalline polymorph form has an LF-Raman spectrum substantially the same as FIG. 16.
25. A hydrated or solvated crystalline form of 7-benzyl-4-(2,4-difluorobenzyl)-2,4,6,7,8,9-hexahydroimidazo[1,2-a]pyrido[3,4-e]pyrimidin-5(1H)-one or a salt thereof.
26. The crystalline form of embodiment 25, comprising a hydrate.
27. The crystalline form of embodiment 25 or 26, comprising a di-hydrate.
28. The crystalline form of embodiment 25 or 26 comprising a tri-hydrate.
29. The crystalline form of embodiment 25 or 26 comprising a variable hydrate.
30. The crystalline form of any one of embodiments 25 to 29, desiccated to an alternative dehydrate.
31. The crystalline form of embodiment 30, wherein the dehydrate form is partially crystalline.
32. The form of embodiment 30 or 31, hydrated to an original crystalline form.
33. A form of any one of embodiments 1 to 32, further comprising a form selected from the group consisting of an initial form and one or more different forms converted from the initial form during manufacture, storage, or use, or a combination thereof.
34. The form of any one of embodiments 1 to 33, characterized as soluble according to ICH guidelines.
35. The form of embodiment 34, wherein the form is characterized as having intermediate aqueous solubility.
36. The form of embodiment 34, wherein the form is characterized as having low aqueous solubility.
37. The form of any one of embodiments 1 to 36, wherein a single therapeutic dose is completely soluble in 250 mL or less of aqueous media in the pH range of 1.2 to 6.8 at 37° C.
38. A pharmaceutical composition comprising the crystalline form of any one of embodiments 1 to 37, and one or more pharmaceutically acceptable excipients.
39. The pharmaceutical composition of embodiment 38, wherein one or more of the pharmaceutically acceptable excipients has desiccating properties.
40. The pharmaceutical composition of embodiment 38 or 39, wherein the composition is packaged with a desiccant.
41. The pharmaceutical composition of any one of embodiments 38 to 40, substantially free of decomposition impurities.
42. The pharmaceutical composition of any one of embodiments 38 to 41, characterized as stable according to ICH guidelines.
43. The pharmaceutical composition of any one of embodiments 38 to 42, characterized as stable according to requirements in 21 CFR Part 211.166, or equivalent thereof.
44. The pharmaceutical composition of embodiment 38 to 43, wherein the composition is stable over one, three, or six months, based on accelerated testing performed at 25° C./60% RH.
45. The pharmaceutical composition of embodiment 38 to 43, wherein the composition is stable over one, three, or six months, based on accelerated testing performed at 40° C./75% RH.
46. The form or composition of any one of claims 1 to 45, wherein the form may be partially crystalline and the crystalline order is disrupted based on a hydration level.

All publications, patents, and patent applications cited in this specification are incorporated herein by reference for the teaching to which such citation is used.

Test compounds for the experiments described herein were employed in free or salt form.

The specific responses observed may vary according to and depending on the particular active compound selected or whether there are present carriers, as well as the type of formulation and mode of administration employed, and such expected variations or differences in the results are contemplated in accordance with practice of the present disclosure.

Although specific embodiments of the present disclosure are herein illustrated and described in detail, the invention is not limited thereto. The above detailed descriptions are provided as exemplary of the present disclosure and should not be construed as constituting any limitation of the invention. Modifications will be obvious to those skilled in the art, and all modifications that do not depart from the spirit of the invention are intended to be included with the scope of the appended claims.

1. A crystalline form of 7-benzyl-4-(2,4-difluorobenzyl)-2,4,6,7,8,9-hexahydroimidazo[1,2-a]pyrido[3,4-e]pyrimidin-5(1H)-one or a salt form thereof.

2. The crystalline form of claim 1, wherein the crystalline form is a crystalline polymorph form of 7-benzyl-4-(2,4-difluorobenzyl)-2,4,6,7,8,9-hexahydroimidazo[1,2-a]pyrido[3,4-e]pyrimidin-5(1H)-one di-hydrochoride salt.

3. The crystalline form of claim 1, wherein the crystalline form is a hydrate or solvate.

4. The crystalline form of claim 2, as a variable hydrate.

5. The crystalline form of claim 4, which contains from between about 2 and about 3 moles of water.

6. The crystalline form of claim 4, wherein the crystalline form of 7-benzyl-4-(2,4-difluorobenzyl)-2,4,6,7,8,9-hexahydroimidazo[1,2-a]pyrido[3,4-e]pyrimidin-5(1H)-one is Form 2, having a powder X-ray diffraction pattern comprising a peak at diffraction angle (2Θ) of 21.8±0.2 or 15.8±0.2.

7. The crystalline polymorph of claim 6, wherein the crystalline polymorph form has a powder X-ray diffraction pattern comprising a peak at diffraction angles (2Θ) at one or more of 3.96±0.2 degrees two-theta, 5.8±0.2 degrees two-theta, 6.06±0.2 degrees two-theta, 7.82±0.2 degrees two-theta, 8.32±0.2 degrees two-theta, 11.54±0.2 degrees two-theta, ±0.2 degrees two-theta, 12.04±0.2 degrees two-theta, 12.84±0.2 degrees two-theta, 13.06±0.2 degrees two-theta, 13.96±0.2 degrees two-theta, 14.38±0.2 degrees two-theta, 14.6±0.2 degrees two-theta, 15.18±0.2 degrees two-theta, 15.58±0.2 degrees two-theta, 15.78±0.2 degrees two-theta, 16.66±0.2 degrees two-theta, 16.92±0.2 degrees two-theta, 17.08±0.2 degrees two-theta, 17.28±0.2 degrees two-theta, 18.04±0.2 degrees two-theta, 18.28±0.2 degrees two-theta, 18.92±0.2 degrees two-theta, 19.48±0.2 degrees two-theta, 19.76±0.2 degrees two-theta, 20.98±0.2 degrees two-theta, 21.5±0.2 degrees two-theta, 21.82±0.2 degrees two-theta, 22.88±0.2 degrees two-theta, 23.26±0.2 degrees two-theta, 24.12±0.2 degrees two-theta, 24.64±0.2 degrees two-theta, 25.08±0.2 degrees two-theta, 25.3±0.2 degrees two-theta, 25.8±0.2 degrees two-theta, 26.14±0.2 degrees two-theta, 26.4±0.2 degrees two-theta, 26.86±0.2 degrees two-theta, 27.4±0.2 degrees two-theta, 27.84±0.2 degrees two-theta, 28.16±0.2 degrees two-theta, 29.1±0.2 degrees two-theta, 29.56±0.2 degrees two-theta, 29.9±0.2 degrees two-theta, 30.32±0.2 degrees two-theta, 30.72±0.2 degrees two-theta, 31.4±0.2 degrees two-theta, 31.8±0.2 degrees two-theta, 33.7±0.2 degrees two-theta, 34.1±0.2 degrees two-theta, 35.18±0.2 degrees two-theta, and 36.48±0.2 degrees two-theta.

8. A crystalline form of 7-benzyl-4-(2,4-difluorobenzyl)-2,4,6,7,8,9-hexahydroimidazo[1,2-a]pyrido[3,4-e]pyrimidin-5(1H)-one or a salt thereof which is hydrated or solvated.

9. The crystalline form of claim 8, comprising a variable hydrate.

10. The crystalline form of claim 9, desiccated to an alternative dehydrate.

11. The crystalline form of claim 10, wherein the dehydrated form is partially crystalline.

12. The crystalline form of claim 10, hydrated to an original crystalline form.

13. The crystalline form of claim 8, further comprising a form selected from the group consisting of an initial form and one or more different forms converted from the initial form during manufacture, storage, or use, or a combination thereof.

14. The crystalline form of claim 8, characterized as soluble according to ICH guidelines.

15. The crystalline form of claim 8, wherein a single therapeutic dose is completely soluble in 250 mL or less of aqueous media in the pH range of 1.2 to 6.8 at 37° C.

16. A pharmaceutical composition comprising the crystalline form of claim 8 and one or more pharmaceutically acceptable excipients.

17. The pharmaceutical composition of claim 16, wherein one or more of the pharmaceutically acceptable excipients has desiccating properties.

18. The pharmaceutical composition of claim 16, characterized as stable according to ICH guidelines.

19. The pharmaceutical composition of claim 16, characterized as stable according to requirements in 21 CFR Part 211.166, or equivalent thereof.

20. The pharmaceutical composition of claim 16, wherein the composition is stable over one, three, six, nine, or twelve months, based on accelerated testing performed at 25° C. to 40° C./60% RH to 75% RH.
